(19) United States
(12) Patent Application Publication
(10) Pub. No.: US 2024/0082342 A1
(43) Pub. Date: Mar. 14, 2024

(54) PEPTIDE THERAPEUTICS FOR INCREASING LUNG CELL VIABILITY

(71) Applicant: BOARD OF REGENTS, THE UNIVERSITY OF TEXAS SYSTEM, Austin, TX (US)

(72) Inventor: Sreerama SHETTY, Tyler, TX (US)

(73) Assignee: BOARD OF REGENTS, THE UNIVERSITY OF TEXAS SYSTEM, Austin, TX (US)

(21) Appl. No.: 18/494,638

(22) Filed: Oct. 25, 2023

Related U.S. Application Data
(63) Continuation-in-part of application No. 18/172,756, filed on Feb. 22, 2023.
Application No. 18/172,756 is a continuation of application No. 17/615,524, filed on Nov. 30, 2021.
(60) Provisional application No. 62/770,508, filed on Nov. 21, 2018.

(51) Int. Cl. A61K 38/08; A61P 11/00

(52) U.S. Cl. CPC A61K 38/08; A61P 11/00

(57) ABSTRACT

Chronic tobacco smoke exposure (TSE)-induced lung injury includes increased alveolar and airway inflammation, type II alveolar epithelial cells (A₂Cs) senescence and apoptosis, and mucus hypersecretion by airway epithelial cells (AECs) that can be treated by the caveolin-1 peptide CSP7 (SEQ ID NO:1). Caveolin-1 and p53 mediated induction of plasminogen activator inhibitor-1 (PAI-1) expression by interleukin 17A, TSE, pollution and other causes leads to lung injury, which can be abrogated by CSP7 treatment, which abolishes A₂Cs senescence and apoptosis and AEC mucus hypersecretion. CSP7 treatment of lung tissue of patients with TSE-induced lung injury decreases A₂C apoptosis and AEC mucus hypersecretion. Lung injury with A₂Cs senescence and apoptosis and AEC mucus hypersecretion caused by TSE-induced PAI-1 expression in lung tissue of patients is abolished by CSP7.

### CROSS-REFERENCE TO RELATED APPLICATIONS

This application is a continuation-in-part of U.S. application Ser. No. 18/172,756, filed Feb. 22, 2023, which is a continuation of U.S. application Ser. No. 17/615,524, filed Nov. 30, 2021, which is a 35 U.S.C. 371 National Phase Entry Application from PCT/US2019/062543, filed Nov. 21, 2019, which claims the benefit of U.S. Provisional Application No. 62/770,508 filed on Nov. 21, 2018, the disclosures of which are incorporated herein in their entirety by reference.

The instant application contains a Sequence Listing which has been submitted via EFS-Web and is hereby incorporated by reference in its entirety. Said Sequence Listing, created on Sep. 7, 2023, is named “4842-110US3_ST26.XML” and is 22,937 bytes in size.

### BACKGROUND OF THE INVENTION

### Field of the Invention

The present invention in the field of biochemistry and medicine is directed to methods and composition for increasing lung cell viability through inhibition of senescence and apoptosis, reducing mucin, interleukin 17A (IL-17A), p53 and plasminogen activator inhibitor-1 (PAI-1) and increasing urokinase plasminogen activator (uPA), uPA receptor (uPAR) and expression of the gene of the forkhead family, FOXA1 (which encodes Hepatocyte nuclear factor 3-α) in airway and alveolar epithelial cells and reducing smooth muscle activation, and for treating chronic obstructive pulmonary disease (COPD)/emphysema, severe asthma, al anti-trypsin deficiency, cystic fibrosis, bronchiectasis, sarcoidosis, bronchiolitis obliterans, lung allograft fibrogenesis and lung transplant rejection,

### Description of the Background Art

COPD affects up to 24 million people and is the third leading cause of death in the U.S. (Hurd S, Chest, 2000; 117: 1S-4S; Ford E S et al., Chest, 2013; 144: 284-305). Chronic tobacco smoke exposure (TSE) is a major risk factor for COPD. There are currently no interventions available to reverse the progression of COPD-related lung injury. Acute exacerbations of COPD are the second leading cause of hospital stays and incur costs of >$18 billion annually in the US (Ford E S, et al., Chest, 2015; 147: 31-45.

Airway epithelial cells (AECs) and alveolar type II epithelial cells (A₂Cs) are common targets for damage from TSE and from mediators/cytokines released from inflammatory cells. COPD pathogenesis has been directly linked to a loss of alveolar structure due to A₂C senescence and apoptosis (Shetty S K et al., Am J Respir Cell Mol Biol. 2012; 47:474-83; Park J-W, et al., COPD. 2007; 4:347-53; Tsuji T et al., Am J Respir Cell Mol Biol. 2004; 31:643-49). Further, TSE causes airway inflammation and mucus hypersecretion leading to airway plugging. The reports of the present inventor and colleagues (Shetty S K et al., supra; Bhandary Y P et al., PLoS One. 2015; 10: e0123187; Tiwari N et al., Am J Physiol Lung Cell Mol Physiol. 2016; 310:L496-506; Marudamuthu A S et al., Am J Pathol. 2015; 185: 55-68; Shetty S et al., J Biol Chem. 2008; 283: 19570-80; Bhandary Y P et al., Toxicol Appl Pharmacol. 2015; 283: 92-98; Shetty S K et al., Am J Pathol. 2017; 187:1016-34) and their preliminary data indicate that TSE lung injury primarily involves increased alveolar and airway inflammation, A₂Cs senescence and apoptosis, and mucus hypersecretion by AECs. These changes are intricately linked to induction of p53 and PAI-1, telomere dysfunction in A₂Cs, and mucous cell metaplasia (MCM) and overexpression of the Mucin 5AC (Muc5AC or M5Ac) gene/protein by AECs and all are clinically relevant and occur in COPD patients. Supporting this point, the present inventors' findings and publications using A₂Cs and AECs, or lung sections of COPD patients and mouse model of TSE lung injury link these findings. These studies showed that p53-mediated induction of PAI-1 expression in A₂Cs and AECs augmented lung inflammation and A₂Cs senescence and apoptosis, mucus hypersecretion in AECs and predisposed to respiratory infection, which often occurs in COPD. Further, a deficiency in p53 or PAI-1 leaves mice resistant to TSE lung injury (Shetty S K et al., supra; Bhandary et al, supra)

p53-induced PAI-1 expression, alveolar fibrinolysis and A₂Cs apoptosis: Lung lavage fluids exhibit high levels of uPA activity and contribute to alveolar proteolysis (Idell S et al., J Clin Invest. 1989; 84: 695-705; Barazzone C et al., J Clin Invest. 1996; 98:2666-73; Olman M A et al., J Clin Invest. 1995; 96:1621-30). However, impaired fibrinolysis is mainly attributable to local over-expression of PAI-1 (major inhibitor of uPA) injury (Barazzone et al., supra; Olman et al., supra; Chapman H A et al., Am Rev Respir Dis. 1986; 133:437-43; Chapman H A. J Clin Invest. 2004; 113:148-57; Hasday J D et al., Exp Lung Res. 1988; 14: 261278; Bertozzi P et al., N Engl J Med. 1990; 322: 890-97; Bachofen M et al., Clin Chest Med. 1982; 3:35-56; Idell S et al., supra; Eitzman D T et al., J Clin Invest. 1996; 97:232-37; Lardot C G et al., Am J Respir Crit Care Med. 1998; 157:617-28; Xu X et al., Exp Lung Res. 2009; 35:795-805; Hu X et al., Chin Med J (Engl). 2009; 122: 2380-85; Zidovetzki R et al., Stroke J Cereb Circ. 1999; 30:651-55).

p53, by binding through its C-terminal amino acid residues 296-393 with a 70-nucleotide (nt) destabilization determinant of PAI-1 3′UTR mRNA has been shown to induce PAI-1 (Shetty, S, 2008, supra; Shetty P et al., Am J Respir Cell Mol Biol. 2008; 39:364-72; Shetty S et al., Mol Cell Biol. 2007; 27:5607-18). p⁵³ also binds PAI-1 promoter and increases PAI-1 mRNA transcription (Kunz C et al., Nuc Acids Res. 1995; 23:3710-17; Bhandary Y P et al., Am J Pathol. 2013; 183:131-43). The present inventors and colleagues further found that TSE of A₂Cs and AECs increased p53 and PAI-1 expression, and reduced cell viability, which was reversed by inhibition of p53 binding to endogenous PAI-1 mRNA, and tissues from COPD patients also showed elevated p53 and PAI-1 in A₂Cs (Shetty S K, 2012, supra; Bhandary Y P et al., PLoS One. 2015, supra; Tiwari et al., supra; Marudamuthu A S et al., supra).

### Role of IL-17A in TSE Lung Injury:

Studies in COPD patients revealed that the accumulation of pulmonary lymphoid follicles and IL-17A⁺ mast cells were associated with severe COPD (Roos A B et al., Am J Respir Crit Care Med. 2015; 191:1232-41. These cells secreted IL-17A, which then set up an inflammatory positive feedback loop as well as MMP12, a potent enzyme that predisposes to emphysema. The present inventors' preliminary findings (Tiwari N et al., supra) and recent reports (Zou Y et al., Int J Chron Obstruct Pulmon Dis. 2017; 12:1247-1254; Chang Y et al., Respir Res. 2014; 15:145) revealed that IL-17A was markedly elevated in the lung and sputum of COPD patients. IL-17A levels were significantly increased in TSE mice, while those mice lacking IL-17A resisted TSE injury. According to the present invention, IL-17A augments p53 and PAI-1 in A₂Cs. Further, literature suggested that IL-17A promoted MCM and M5Ac overexpression (Xia W et al., PLoS ONE. 2014; 9)

According to the present invention, IL-17A, p53 and PAI-1 affect TSE-induced telomere dysfunction in A₂Cs and emphysema, and M5Ac overexpression by AECs and airway/lung remodeling.

In summary, AECs and A₂Cs are the common targets of damage from chronic TSE and inflammatory cells in humans and in pre-clinical COPD models. COPD and TSE lung injury is also characterized by lung inflammation, telomere dysfunction, and senescence and apoptosis in A₂Cs and M5Ac overexpression by AECs.

The present inventors and colleagues have linked these findings, showing that A₂Cs and AECs express p53 and PAI-1, and that p53 induces PAI-1 to increase lung injury (references cited above and Eren M et al., Proc Natl Acad Sci USA. 2014; 111: 7090-95 and Bhandary Y P et al., Am J Physiol Lung Cell Mol Physiol. 2012; 302: L463-73).

A deficiency of p53 or PAI-1 makes mice highly resistant to TSE lung injury (supra), implying that changes in p53 and PAI-1 in A₂Cs and AECs, and consequent telomere dysfunction, alveolar injury and mucus hypersecretion are important contributors to COPD. These data further suggest that TSE or IL-17A augments p53 and PAI-1 expression, and the process involves increased caveolin-1 (Cav1). According to the present invention, systemic, or airway delivery of the heptapeptide CSP7 (described below) in liquid or DP formulation mitigates deleterious effects of TSE.

### Telomere Failure

A subset of patients with age-associated pathology such as idiopathic pulmonary fibrosis (IPF) manifests mutations in the gene of telomerase reverse transcriptase (TERT), or in its RNA component (TERC) (Armanios M Y et al., N Engl J Med 2007; 356:1317-26; Tsakiri K D et al., Proc Natl Acad Sci USA 2007; 104:7552-57) the mutation in TERT can be familial as well as non-familial (Tsakiri et al., supra), suggesting that factors contributing to mutations can in turn affect telomere failure and associated pathologies.

The mechanisms by which telomere defects provoke lung disease are not understood, but a number of observations have pointed to lung-intrinsic factors and epithelial dysfunction as candidate events (Alder J K et al., Proc Natl Acad Sci USA 2015; 112:5099-5104). For example, in telomerase-null mice, DNA damage preferentially accumulates in the air-exposed epithelium after environmentally induced injury, such as with cigarette smoke. The additive effect of environmental injury and telomere dysfunction has been suggested to contribute to the susceptibility to emphysema seen in these mice (Alder et al., supra). Pulmonary fibrosis and emphysema patients have also been noted to have abnormally short telomeres in A₂Cs (Liu T et al., Am J Respir Cell Mol Biol 2013; 49:260-68).

As noted, in COPD/emphysema, chronic inflammation leads to muc5A overexpression and mucus hypersecretion AECs, narrowing of small airways due to inflammation and airway remodeling and smooth muscle proliferation, and alveolar wall destruction due to death of A₂Cs. This is also true in wild-type (WT) mice exposed to 20 weeks of tobacco smoke. Further, levels of the cytokine IL-17A are significantly elevated in the peripheral lung tissues of patients with severe COPD and in the lungs of TSE mice (genetically WT).

Shortening of the telomere due to increased expression of SIAH-1, a p53-inducible E3 ubiquitin ligase that is known to downregulate the telomere repeat binding factor 2 (TRF2) was observed in A₂Cs of COPD patients. Downregulation of telomerase reverse transcriptase (TERT) was observed, and was correlated with the reduced TRF2 and upregulation of TRF1 expression in the COPD lung tissues.

The additive effect of environmental injury and telomere dysfunction has been suggested to contribute to the susceptibility to emphysema (Alder et al. Am J Crit Care Med 184:904-912,2011). In emphysema patients, telomeres in A₂Cs are abnormally short. This is also true in A₂Cs from WT mice subjected to tobacco smoke. However, the mice exposed to smoke and received Cav1 scaffolding domain peptide; CSP7, resisted telomere shortening. Increase in the protein expression of p53, cleaved caspase-3 and β-galactosidase, pointing to A₂C death. However, the A₂Cs from the CSP7 treated mice showed significant decreases in p53, cleaved caspase-3 and β-galactosidase expression. CSP7 treatment also restored TRF2 expression and the enzyme activity of TERT.

There is presently no cure or effective treatment intervention for mucus hypersecretion, telomere shortening associated with COPD/emphysema and bronchiolitis obliterans associated with transplant rejection. In view of the poor prognosis and lack of therapeutic approaches for these conditions, there is an urgent need for new interventions to reverse or at least slow the progression of disease. This critical therapeutic gap is addressed by the present invention.

Airway mucus hypersecretion is one of the cardinal features of several chronic lung diseases including COPD, which results in airway obstruction and contributes significantly to morbidity and mortality (Hogg J C et al., N Engl J Med 350: 2645-53, 2004; Hogg J C et al., Annu Rev Pathol 4: 435-59, 2009). Clinically, muco-active drugs have been shown to effectively reduce exacerbation of COPD and improve to upsurge the quality of life of patients (Curran D R et al., Am J Respir Cell Mol Biol 42:268-75, 2010; Decramer M et al., Eur Respir Rev 19:134-40, 2010), demonstrating the usefulness of targeting mucus hypersecretion in COPD therapy. Chronic TSE is the most common identifiable risk factor for COPD, with smokers known to have a greater COPD mortality rate than non-smokers (Kohansal R et al., Am J Respir Crit Care Med.;180:3-10, 2009). The pathogenesis of COPD remains poorly understood but involves aberrant cellular and inflammatory responses of the lung to TSE, resulting in the disruption of AEC function. Such disruption has been attributed to a reduction in epithelial cell cilia length and AEC death, followed by re-epithelialization by goblet cells, subsequent excess mucus production finally leading to impaired mucociliary clearance.

In total, 21 genes are reported to encode mucins in the human genome. Mucin 5Ac (MUC5AC) is expressed at high levels in the airway system (Thornton D J et al., Annu Rev Physiol 70: 459-86, 2008; Rose M C et al., Physiol Rev 86: 245-78, 2006). Mucus may alter the normal structure and status of goblet cells after failing to incorporate with MUC5AC. Without the normal reaction between MUC5AC and mucus, the airway viscoelasticity becomes vulnerable to plugging (Bonser L R et al., J Clin Med 6: E112, 2017; Woodruff P G et al., Am J Respir Crit Care Med 180:388-95, 2009). Goblet cell differentiation is dictated by a large network of genes, in which transcription factors sterile α motif- (SAM)-pointed domain containing ETS-like transcription factor (SPDEF) and forkhead box protein A2 (FOXA2) are two key regulators. SPDEF (encoded in humans by the SPDEF gene; Genbank Gene ID 25803) is required for goblet cell differentiation and mucus production, including the major secreted airway mucin MUC5AC (Park K S et al., J Clin Invest. 117:978-88, 2007; Chen G et al., J Clin Invest 119:2914-24, 2009; Rajavelu P et al., J Clin Invest. 125:2021-31, 2015), whereas FOXA2 is a potent inhibitor of goblet cell differentiation in the lung (Wan H et al., Development. 131:953-64, 2004; Chen G et al., J Immunol. 184:6133-41 2010; Tang X et al., Am J Respir Cell Mol Biol. 49:960-70, 2013). Forkhead box protein A3 (FOXA3) was highly expressed in airway goblet cells from COPD patients. Because FOXA3 bound to and induced SPDEF, a gene required for goblet cell differentiation in the airway epithelium, the observed effects of FOXA3 on mucus-related gene expression are likely mediated, at least in part, by its ability to induce SPDEF (et al., Am J Respir Crit Care Med. 2014 Feb. 1; 189:301-13).

Breakdown of the ciliated cells also further contributes to mucociliary dysfunction. AECs exposed to TSE have an over 70% decrease in the number of ciliated cells and show a shortening of the cilia. One mechanism under investigation involves autophagy that is dependent on histone deacetylase 6 (HDAC6). HDAC6 is upregulated in the airways of COPD patients where it may act to target damaged and misfolded proteins for proteasomal degradation. In the case of ciliary shortening, HDAC6 was found to co-localize with alpha tubulin then associated with LC3B, a protein active in autophagy. Recently demonstrated an increased expression of autophagy markers in the development of COPD (Kim H P et al., Autophagy, 4:887-95, 2008; Ryter S W et al., Autophagy 5:235-7, 2009).

Caveolae are vesicular invaginations of the plasma membrane. Cav1 is the structural protein component of caveolae. Cav1 participates in signal transduction processes by acting as a scaffolding protein that concentrates, organizes and functional regulates signaling molecules within caveolar membranes. These studies, combined with the close association between MUC5AC secretion and airway inflammation, led us to hypothesize that Cav1 may be an important regulator involved in TSE-induced MUC5AC production in lung epithelial cells. Currently, few advances have been made to alleviate mucociliary clearance (MCC) disruption and bronchitis associated with the pathogenesis of COPD due to elevation of Cav1. In the present study, the inventor investigated Cay1 binding to the catalytic unit (PP2AC) of protein phosphatase-2A (PP2A), which in turn downregulated PP2AC activity and led to increased expression of cancerous inhibitor of protein phosphatase 2A (CIP2A). Increased CIP2A leads to phosphorylation of the serine/threonine-selective protein kinase (ERK), and secretion of matrix metalloproteinase-12 (MMP12). Indeed, Cav1 elevated p53 and PAI-1 expression in AECs and increased susceptibility to and exacerbation of respiratory infections which all are associated with COPD.

According to the present invention, Cav1 is a key player of a novel signaling pathway that links TSE to mucus hypersecretion and ciliary disassembly. A 7-mer deletion fragment of Cav1 scaffolding domain peptides CSP referred to as CSP7 (having the sequence FTTFTVT (SEQ ID NO:1) mitigates cilia shortening and impaired MCC by inhibiting Cav1. These findings provide both new insights on how CSP7 regulates complex interrelationships between p53, PAI-1, autophagy and primary cilia, but also provides a basis for treatment of ciliopathy-associated mucus hypersecretion. The present results provide new therapeutic targets for improving airway function during chronic lung diseases such as COPD through the maintenance of epithelial cell proteostasis and modulation of the autophagic pathway.

### Cav1-Derived Peptides

The present inventors first discovered that a 20 residue peptide DGIWKASFTTFTVTKYWFYR, (SEQ ID NO:2) which is the scaffolding domain of Cav1 (Cav1; SEQ ID NO:3, shown below) protected lung or airway epithelial cells (LECs/AECs) from bleomycin (“BLM”)-induced apoptosis in vitro and in vivo and prevented subsequent pulmonary fibrosis by attenuating lung epithelial damage (Shetty et al., U.S. patent application Ser. No. 12/398,757 published as U.S. 2009-0227515A1 (Sep. 10, 2009) and issued as U.S. Pat. No. 8,697,840 (4/15/14) and Shetty et al., PCT Pub. WO2014/145389 (9/18/2014), corresponding to U.S. application Ser. No. 14/775,895 published as U.S. Pat. Publ. 2016/0272678 (9/22/2016) and issued as U.S. Pat. No. 9,630,990 (4/25/2017), all of which are hereby incorporated by reference in their entirety.

The present inventors also discovered that a 17 residue peptide NYHYLESSMTALYTLGH (SEQ ID NO:4), termed PP-2, also protected LECs from BLM-induced apoptosis in vitro and in vivo and prevented subsequent pulmonary fibrosis by attenuating lung epithelial damage.

Shetty et al., 2009 and 2014 (supra) also describes biologically active substitution, addition and deletion variants of these peptides as well as peptide multimers and deliverable polypeptides comprising the above peptides, and pharmaceutical compositions comprising the foregoing peptides, variants and multimers. Those compositions inhibit apoptosis of injured or damaged lung epithelial cells and treating acute lung injury and consequent pulmonary fibrosis/IPF.

Shetty et al. 2014 (U.S. Pat. No. 9,630,990) identified a particular 7 residue fragment of CSP now termed CSP7, which has the sequence FTTFTVT (SEQ ID NO:1) and which has the biological activity of CSP. More recently the present inventors' group has described formulations of CSP7 as an inhaled peptide therapeutic for, inter alia, idiopathic pulmonary fibrosis (Surasaranga et al., Drug Devel. Indust. Pharmacy, 2018; 44:184-98) which peptide is also used in the present methods. The present invention constitutes, in part, an extension of the inventors' earlier findings as disclosed in the above patents and patent publications (S. Shetty et al., 2007, 2008 & 2009, 2014, supra).

### SUMMARY OF THE INVENTION

The present invention is directed to methods using the heptapeptide CSP7 (FTTFTVT, SEQ ID NO:1) which is the smallest functional fragment of the 20 residue peptide DGIWKASFTTFTVTKYWFYR (SEQ ID NO:2) which is the scaffolding domain (CSP or CSP1) of Cav1.

CSP7 blocks, inhibits, attenuates or reduces
induction of p53 and PAI-1,
telomere shortening and dysfunction,
senescence and apoptosis in alveolar type II epithelial cells (A₂Cs),
expression of forkhead box protein A3 (FOXA3)
expression of sterile a motif- (SAM)-pointed domain containing ETS-like factor (SPDEF)
expression cancerous inhibitor of protein phosphatase 2A (CIP2A);
expression of histone deacetylase 6 (HDAC6),
mucous cell metaplasia (MCM),
mucus hypersecretion and M5Ac overexpression such as that induced by tobacco smoke in AECs,
airway remodeling,
IL-17A and IL-17A-mediated mucus hypersecretion and resultant lung inflammation,
epithelial cell injury,
autophagic activity,
airway ciliary disassembly, shortening or ciliopathy,
transplant rejection and
lung allograft fibrogenesis.
   CSP7 inhibits tobacco-smoke-induced MucSAc expression by AECs and telomere shortening by suppressing p53-miR-34a feed-forward induction and protecting sheltrin complex proteins in A₂Cs.

CSP7 increases expression of, or upregulates:
forkhead A2 (FOXA2)
catalytic subunit (of protein phosphatase-2A (PP2AC).

Therefore, CSP7, preferably in a formulation for administration by inhalation/lung instillation as described herein (see, also, Surasaranga et al., supra) is an effective agent for treating inflammatory lung diseases such as COPD/emphysema, severe asthma, al anti-trypsin deficiency, cystic fibrosis, sepsis, bronchiectasis, sarcoidosis and other airway diseases. Since increased IL-17A contributes to bronchiolitis obliterans, inhibition of IL-17A by treatment with CSP7 reduces or prevents transplant rejection including that stimulated by or resulting from allograft fibrogenesis.

The present invention is directed to a method for
(A) blocking, reducing or attenuating:
   (i) induction of p53 and PAI-1;
   (ii) telomere dysfunction;
   (iii) senescence and apoptosis in A₂Cs;
   (iv) expression of FOXA3
   (v) expression of SPDEF
   (vi) mucous cell metaplasia (MCM);
   (vii) mucus hypersecretion mediated by overexpression of M5Ac or by TSE or IL-17A by AECs;
   (viii) expression CIP2A;
   (ix) expression of HDAC6;
   (x) autophagic activity; or
   (xi) ciliary disassembly, shortening or ciliopathy;
   or
(B) increasing expression of or upregulation of:
   (xii) expression of forkhead box protein A2 (FOXA2);
   (xiii) expression of catalytic subunit of protein phosphatase-2A (PP2AC);
comprising providing to A₂Cs or AECs in a subject, preferably a human subject, an effective amount of a compound or composition that is:
   (a) a peptide designated CSP7 the sequence of which is FTTFTVT (SEQ ID NO:1);
   (b) an addition variant of (a) that includes 1-5 amino acids of additional sequence at the N- and/or C-terminus
   (c) a covalently-modified chemical derivative of the peptide of (a) or (b),
   (d) a peptide multimer of (a), (b) or (c);
   (e) a deliverable peptide or polypeptide composition comprising the peptide, variant derivative or multimer of any of (a)-(d) bound to or associated with a delivery or translocation-molecule or moiety;
   wherein said variant, chemical derivative or multiimer has at least 20% of the biological
   or biochemical activity of said CSP7 in an in vitro or in vivo assay.
      The above method preferably results in a reduction of lung inflammation and treatment, attenuation or reduction of an inflammatory lung disease in said subject.

The peptide variant, chemical derivative or multimer described above or below preferably has the following activity relative to the activity CSP7: at least about 20%, 30%, 40%, 50%, 60%, 65%, 70%, 75%, 80%, 85%, 90%, about 95%, 97%, 99%, and any range derivable therein, such as, for example, from about 70% to about 80%, and more preferably from about 81% to about 90%; or even more preferably, from about 91% to about 99%. The peptide variant chemical derivative or multimer may have 100% or greater than 100% of the activity of CSP7. This relative activity may be based on any method disclosed herein or known in the art for evaluating such activity.

A preferred compound is the heptapeptide CSP7, FTTFTVT (SEQ ID NO:1).

A preferred peptide multimer comprises at least two monomers, each monomer being the CSP7 peptide, the variant of (b) above or the chemical derivative of (c) above, which multimer:
(a) has the formula P¹ₙ wherein
   (i) P¹ is the peptide, variant or chemical derivative as above, and
   (ii) n=2-5, or
(b) has the formula (P¹-Xₘ)ₙ—P², wherein
   (i) each of P¹ and P² is, independently, the peptide, variant of chemical derivative as above,
   (ii) each of P¹ and P² is the same or different peptide, variant or derivative
   (iii) X is C₁-C₅ alkyl, C₁-C₅ alkenyl, C₁-C₅ alkynyl, C₁-C₅ polyether containing up to 4 oxygen atoms;
   (iv) m=0 or 1; and
   (v) n=1-7,
(c) has the formula (P¹-Gly_{z})ₙ-P², wherein:
   (i) each of P¹ and P² is, independently, the peptide, variant or derivative,
   (ii) each of P¹ and P² is the same or different peptide or variant or derivative;
   (iii) z=0-6; and
   (iv) n=1-25,
      The peptide multimer preferably has at least 20% of the biological, biochemical or pharmacological activity of the CSP7 peptide in an in vitro or in vivo assay.

In the above method, the peptide, addition variant, chemical derivative, multimer, or deliverable peptide or polypeptide is provided in vivo.

Also provided is a method for treating a mammalian subject, preferably a human, having an inflammatory lung disease or condition, preferably selected from the group consisting of COPD/emphysema, severe asthma, al anti-trypsin deficiency, cystic fibrosis, bronchiectasis, sarcoidosis, bronchiolitis obliterans, lung allograft fibrogenesis and lung transplant rejection. The method comprises administering to the subject in need thereof and effective amount of
(a) a pharmaceutical composition comprising a compound or composition selected from the group consisting of:
   (i) a peptide designated CSP7 the sequence of which is FTTFTVT (SEQ ID NO:1);
   (ii) an addition variant of (i) that preferably does not exceed 20 residues and preferably includes 1-5 amino acids of additional sequence at the N-terminus, the C-terminus, or both;
   (iii) a covalently-modified chemical derivative of the peptide of (i) or (ii),
   (iv) a peptide multimer of (i), (ii) or (iii); and
   (v) a deliverable peptide or polypeptide composition comprising the peptide, variant derivative or multimer of any of (i)-(iv) bound to or associated with or admixed with a delivery or translocation-molecule or moiety.
   wherein said addition variant, chemical derivative or multiimer has at least 20% of the biological, biochemical and pharmacological activity of said CSP7 in an in vitro or in vivo assay, and
(b) a pharmaceutically acceptable carrier or excipient.

In preferred embodiment of the above method, the compound is the CSP7 peptide of SEQ ID NO:1. In another embodiment of the method, the compound is the peptide multimer, preferably one that comprises monomers of the CSP7 peptide (SEQ ID NO:1).

Preferably, when the above method uses a peptide multimer:
(a) the peptide multimer has the formula P¹n wherein
   (i) P¹ is the peptide, variant or chemical derivative, and
   (ii) n=2-5, or
(b) the peptide multimer has the formula (P¹—Xₘ)ₙ—P², wherein
   (i) each of P¹ and P² is, independently, the peptide, variant or chemical derivative;
   (ii) each of P¹ and P² is the same or different peptide, variant or derivative;
   (iii) X is C₁-C₅ alkyl, C₁-C₅ alkenyl, C₁-C₅ alkynyl, C₁-C₅ polyether containing up to 4 oxygen atoms;
   (iv) m=0 or 1; and
   (v) n=1-7, or*
(c) the peptide multimer has the formula (P¹-Gly_{z})ₙ—P², wherein:
   (i) each of P¹ and P² is, independently, the peptide, variant or derivative,
   (ii) each of P¹ and P² is the same or different peptide or variant or derivative;
   (iii) z=0-6; and
   (iv) n=1-25,
      Preferably the multimer has at least 20% of the biological, biochemical or pharmacological activity of CSP7 peptide in an in vitro or in vivo assay.

The invention also provides a use of compound or composition for treating COPD/emphysema, severe asthma, al anti-trypsin deficiency, cystic fibrosis, bronchiectasis, sarcoidosis, bronchiolitis obliterans, lung allograft fibrogenesis and lung transplant rejection, which compound or composition comprises;
(a) a peptide designated CSP7 the sequence of which is FTTFTVT (SEQ ID NO:1);
(b) an addition variant of (a) that includes 1-5 amino acids of additional sequence at the N-and/or C-terminus;
(c) a covalently-modified chemical derivative of the peptide of (a) or (b),
(d) a peptide multimer of (a), (b) or (c); and
(e) a deliverable peptide or polypeptide composition comprising the peptide, variant derivative or multimer of any of (a)-(d) bound to or associated with a delivery or translocation-molecule or moiety.
wherein said addition variant, chemical derivative or multiimer has at least 20% of the biological, biochemical or pharmacological activity of said CSP7 in an in vitro or in vivo assay.

Also provided is the use of a compound or composition for the manufacture of a medicament for treatment of COPD/emphysema, severe asthma, al anti-trypsin deficiency, cystic fibrosis, bronchiectasis, sarcoidosis, bronchiolitis obliterans, lung allograft fibrogenesis and lung transplant rejection, which compound or composition comprises:
(a) a peptide designated CSP7 the sequence of which is FTTFTVT (SEQ ID NO:1);
(b) an addition variant of (a) that includes 1-5 amino acids of additional sequence at the N- and/or C-terminus;
(c) a covalently-modified chemical derivative of the peptide of (a) or (b),
(d) a peptide multimer of (a), (b) or (c); and
(e) a deliverable peptide or polypeptide composition comprising the peptide, variant derivative or multimer of any of (a)-(d) bound to or associated with a delivery or translocation-molecule or moiety.
wherein said addition variant, chemical derivative or multiimer has at least 20% of the biological or biochemical activity of said CSP7 in an in vitro or in vivo assay.

In some specific embodiments, the invention relates to:
1. A method for blocking, reducing or attenuating:
(i) telomere dysfunction;
(ii) senescence and apoptosis in alveolar type II epithelial cells (A₂Cs);
(iii) mucus hypersecretion mediated by overexpression of M5Ac or by IL-17A in airway epithelial cells (AECs); or
(iv) disassembly, shortening or ciliopathy of airway cilia;
comprising administering by DP inhaler to A₂Cs or AECs in a subject a composition comprising:
(a) 0.2 mg to 10 mg/day of a peptide having the amino acid sequence FTTFTVT (SEQ ID NO:1); or
(b) 0.2 mg to 10 mg/day of an addition variant of (a) that includes 1-5 amino acids of additional sequence at the N- and/or C-terminus.
2. The method of item 1, further comprising reducing lung inflammation in said subject.
3. The method of item 1, wherein the peptide is FTTFTVT (SEQ ID NO:1).
4. The method of item 1, wherein the subject is a human.
5. The method of item 1, wherein telomere dysfunction is measured by measuring telomere shortening by TeloTAGGG assay or qPCR.
6. The method of item 5, wherein there is at least a 3% increase in telomere length in A₂Cs after 6 months of administration.
7. The method of item 1, wherein reducing senescence in A₂Cs is determined by measuring beta-galactosidase in A₂Cs after 6 months of administration.
8. The method of item 7, wherein there is at least a 5% reduction in beta-galactosidase in A₂Cs.
9. The method of item 1, wherein reducing senescence in A₂Cs is determined by measuring beta-galactosidase activity by colorimetric assay, X-gal staining, flow cytometry or beta-galactosidase staining using an antibody in A₂Cs.
10. The method of item 9, wherein there is at least a 3% reduction in activated caspase-3 expression after 6 months of administration.
11. The method of item 1, wherein apoptosis in A₂Cs is determined by measuring activated caspase-3 expression.
12. The method of item 11, wherein there is a 3% reduction in activated caspase-3 expression after 6 months of administration.
13. The method of item 1, wherein mucus hypersecretion mediated by overexpression of Muc5Ac in AECs is measured by periodic acid Schiff (PAS) staining for mucin, immunohistochemistry using an antibody for Muc5Ac or mucin, Western blotting, or real-time PCR.
14. The method of item 13, wherein there is at least a 20% reduction in Muc5Ac expression after 6 months of administration.
15. The method of item 1, wherein mucus hypersecretion is mediated by overexpression of IL-17A in AECs and is measured by periodic acid Schiff (PAS) staining for mucin, immunohistochemistry using an antibody for Muc5Ac or mucin, Western blotting, or real-time PCR.
16. The method of item 15, wherein there is at least a 10% reduction in IL-17A expression after 6 months of administration.
17. The method of item 1, wherein lung and airway inflammation is analyzed by immunohistochemistry using an antibody against myeloperoxidase (MPO) or measuring MPO activity by colorimetric assay, using an antibody against airway and alveolar polymorphonucleocytes (PMN), using an antibody against airway and alveolar inflammatory macrophages, or an antibody against airway and alveolar CD4+ and CD8+ cells.
18. The method of item 1, wherein apoptosis and senescence are mediated by overexpression of IL-17A in A2Cs.
19. The method of item 18, wherein there is at least a 40% reduction in IL-17A expression after 6 months of administration.
20. The method of item 19, wherein the IL-17A expression is measured by ELISA, Western blotting, or real-time PCR.
21. The method of item 1, wherein miR-34a expression is inhibited.
22. The method of item 21, wherein there is at least a 15% reduction in miR-34a expression after 6 months of administration.
23. The method of item 22, wherein the miR-34a expression is measured by real-time PCR.
24. The method of item 1, wherein disassembly of airway cilia is measured by measuring number of ciliated cells.
25. The method of item 24, wherein there is at least a 20% increase in the number of ciliated cells after 6 months of administration.
26. The method of item 1, wherein shortening of airway cilia is measured by microscope.
27. The method of item 26, wherein there is at least a 20% increase in cilia length after 6 months of administration.
28. The method of item 1, wherein ciliopathy of airway cilia is measured by measuring number of Ac-tubulin positive cells.
29. The method of item 28, wherein there is at least a 20% increase in Ac-tubulin positive cells after 6 months of administration.
30. The method of item 1, comprising administering 2.5 mg/day, 5 mg/day, or 10 mg/day of said peptide or addition variant thereof.

### (See, e.g., FIGS. 44A-44C.)

In embodiments of the foregoing method, the peptide, variant or chemical derivative is capped at its N-terminus, C-terminus or both with a capping group as described herein or otherwise known in the art.

In addition to the “standard” L-amino acids, D-amino acids or non-standard, modified or unusual amino acids which are well-defined in the art are also contemplated for use in the present invention for the purpose of protecting the peptide from proteolytic degradation in vivo.

### BRIEF DESCRIPTION OF THE DRAWINGS

FIGS. 1A-1F show shortening of telomere length of A₂Cs obtained from human fibrotic lung. (A) TeloTAGGG assay was conducted for estimating the telomere length of the isolated genomic DNA. The southern blot data shows the telomere shortening of the A₂Cs from fibrotic lung. (B) Bar graph shows the relative quantification of the shortening occurred in the A₂Cs. (C) Western blot analysis was conducted to analyze the protein expression of telomerase enzyme (TERT), and the apoptosis pathway related proteins. (D) Bar graph showing relative telomere length of the A₂Cs analyzed by qPCR after extracting the genomic DNA. E. Gel shows the telomerase enzyme activity as analyzed by the TRAPeze enzyme assay. (F) Bar graph shows the quantitation of the relative TRAPeze enzyme activity.

FIGS. 2A-2F show shortening of telomere length of A₂Cs isolated from human COPD lungs. (A). TeloTAGGG assay was conducted for estimating the telomere length of the isolated genomic DNA. The southern blot data shows the telomere shortening of the AECs from COPD lung. (B) Bar graph shows the relative quantification of the shortening occurred in the A₂Cs. (C) Bar graph shows relative telomere length of the A₂Cs analyzed by qPCR after extracting the genomic DNA. (D) Western blot analysis was conducted to analyze the protein expression of telomerase enzyme (TERT), and the apoptosis pathway related proteins. (E) Gel shows the telomerase enzyme activity as analyzed by the TRAPeze enzyme assay. (F) Bar graph shows the quantitation of the relative TRAPeze enzyme activity.

FIGS. 3A-3D show that passive cigarette smoke exposure led to decrease in telomerase expression and shortening of telomere in A₂Cs of WT mice. WT mice were exposed to tobacco smoke for 20 weeks, and then treated with peptide CSP7 or a control peptide (CP) and the A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity.

FIGS. 4A-4D show that repeated bleomycin exposure led to decrease in telomerase expression and shortening of telomere in A₂Cs of WT mice. WT mice were exposed to intranasal bleomycin once in two weeks for 16 weeks. CSP7 or control peptide (CP) treatment started at 14^{th} week and was continued daily till the end of the experiment, at which time A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the trapeze enzyme activity is shown.

FIGS. 5A-5E show that A₂Cs of mice deficient in miR-34a expression were protected from telomere shortening induced by passive cigarette smoke. SP-CCRE-miR-34a^{cKO} and SP-CCRE-miR-34a^{fl/fl} mice were exposed to smoke for 20 weeks and later treated with CSP7 or control peptide (CP) after which A₂Cs were isolated. (A) miR-34a expression by qPCR. (B) Relative telomere length of the A₂Cs was analyzed by qPCR after extracting the genomic DNA. (C) Western blot analysis was conducted to analyze the protein expression of telomerase enzyme (TERT), and the apoptosis pathway related proteins. (D) Gel shows the telomerase enzyme activity as analyzed by the TRAPeze enzyme assay. (E) Bar graph shows the quantitation of the TRAPeze enzyme activity.

FIGS. 6A-6D show that passive cigarette smoke exposure led to decrease in telomerase expression and shortening of telomere in A₂Cs of uPA^{-/-} mice. uPA^{-/-} mice were exposed to tobacco smoke for 20 weeks and treated with the CSP7 or control peptide (CP) after which the A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity is shown.

FIGS. 7A-7D show that repeated bleomycin exposure led to decrease in telomerase expression and shortening of telomere in A₂Cs of uPA^{-/-} mice. uPA^{-/-} mice were exposed to intranasal bleomycin once every two weeks for 16 weeks. CSP7 or control peptide (CP) treatment was started at 14^{th} week and continued daily till the end of the experiment at which time the A₂Cs were isolated. (A) Relative telomere length of the AECs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity is shown.

FIGS. 8A-8D show that A₂Cs of PAI-1^{-/-} mice were resistant to telomere shortening induced by passive cigarette smoke. PAI-1^{-/-} mice were exposed to tobacco smoke for 20 weeks, and then treated with the CSP7 or control peptide (CP) and the A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity.

FIGS. 9A-9D show that A₂Cs of PAI-1^{-/-} mice were resistant to telomere shortening induced by treatment with repeated dose of bleomycin. PAI-1^{-/-} mice were exposed to intranasal bleomycin once every two weeks for 16 weeks. CSP7 or control peptide (CP) treatment started at 14^{th} week and continued daily until the end of the experiment, at which time the A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity is shown.

FIG. 10 is a schematic illustration of how tobacco smoke exposure-induces airway mucus hypersecretion and ciliary disassembly and COPD and its attenuation by CSP7.

FIGS. 11A-11C show that differential expression of MUC5AC, FOXA2, FOXA3, HDAC6, and SPDEF in AECs isolated from COPD lungs. (A) Bar graph showing increased mean linear intercept (MLI) observed in lung tissue sections. Results of IHC (not shown) indicated increased MUC5Ac and HDAC6 in lung sections. (B) Western blot showing differential expression of MUC5AC, FOXA2, FOXA3, HDAC6, Cav1, PAI-1, p53, AC-TUB and SPDEF in AECs isolated from NL and COPD lungs. (C) Bar graphs showing increased expression of MUC5Ac (measure of mucin), HDAC6, FOXA3, and HDAC6, and decreased FOXA2 mRNA expression in AECs isolated from COPD lungs. These results data reveal increased MUC5Ac and HDAC6, and reduced FOXA2 protein and mRNA expression in AECs of human COPD lungs compared to their basal expressions in NL AECs.

FIGS. 12A-12C show that histone deacetylase 6 (HDAC6) affected selective autophagy and regulates COPD-associated cilia dysfunction. (A) Accumulation of LC3-II and expression of Beclin-1, ATG5 and p62 were determined by western blotting for NL and COPD AECs. (B) Bar graphs show increased expression of LC3, Beclin1 and Atg5 in COPD lungs compare to normal (NL). (C) Immunohistochemical (IHC) staining for MAP-LC3 indicated increased expression in COPD lung tissue.

FIGS. 13A-13D show that CSP7 mitigates the induction of mucus hypersecretion and cilia shortening in COPD AECs. AECs were isolated from NL and COPD lungs. AECs from COPD lungs were treated with or without CSP7 or CP in vitro for 48 h. (A) Western Blot images show increased expression of MUC5AC, HDAC6, PAI-1, p53, Cav1, FOXA3, SPDEF and decreased acetylated tubulin (AC-Tubulin; for cilia length) and FOXA2 expression in AEC lysates of COPD lungs that are reversed with CSP7 treatment. (B) Bar graphs show increased expression of MUC5AC, HDAC6, FOXA3 and Cav1 mRNA, and decreased expression of FOXA2 mRNA in COPD AECs analyzed by qPCR; this is reversed by CSP7 treatment. Immunofluorescence staining (not shown) revealed increased co-localization of MUC5AC and HDAC6 in AEC lysates of COPD lungs that are reversed with CSP7 treatment. (C) Immunoblotting performed for LC3, Beclin1, ATG5, p62 in AEC lysates of COPD lungs was reversed with CSP7 treatment (results not shown). AECs from COPD lungs were treated with or without CSP7 or CP in vitro for 6 h. Fluorescence microscopy (results not shown) was performed with acridine orange staining (acidic vesicle). Immunofluorescence staining with acridine orange (not shown) revealed increased co-localization of Ac-Tub/LC3 in AECs exposed to COPD vs. diffused staining in PBS treated controls. CSP7 reversed the co-localization of the AC-Tubulin/LC3. (D) Bar graphs showing the number of ciliated cell and cilia length of in AEC lysates of COPD lungs and indicate reversal with CSP7 treatment.

FIGS. 14A-14D show that TSE-induced mucus hypersecretion and cilia dysfunction were reduced by CSP7 (A) Western Blot images showing increased expression of MUC5AC, HDAC6, FOXA3, SPDEF, Beclin-1, LC3 and decreased expression of FOXA2 and AC-Tubulin in AECs lysates from normal human lungs (NL) and cells treated with TS extract (TSE) in vitro for 48 h; this effect was reversed with CSP7 treatment. (B) Bar graph of qPCR data showing increased MUC5AC, HDAC6, FOXA3, and reduced FOXA2 mRNA expression in AECs isolated from NL treated with TSE and reversal of this expression by CSP7 treatment. (C) Western Blot images show increased expression of LC3, Beclin-1, ATG5 and decreased expression of P62 in AECs lysates from human NL treated with TS extract (TSE) in vitro for 48 h, which is reversed by CSP7 treatment. (D) Bar graphs depicting significant decrease in cilia length and number of ciliated cells in TSE AECs, suggesting MCM, that are significantly improved after treatment with CSP7. Immunofluorescence staining (not shown) indicated increased co-localization of MU5AC and HDAC6 in AECs exposed to TS extract (TSE) vs diffused staining in PBS treated controls. Treatment of TSE-exposed AECs with CSP7 reversed such co-localization. Further, immunofluorescence staining (not shown) revealed increased co-localization of ACTub/LC3 in AECs exposed to TSE vs diffused staining in PBS treated controls. Treatment of TSE-exposed AECs with CSP7 reversed this co-localization.

FIGS. 15A-15C show that CSP7 delivered by intraperitoneal (IP) injection or nebulization (Neb) mitigated TSE lung injury in mice. WT mice (n=10/group) were kept in ambient AIR or TSE for 4 hrs/day 5 days/week. After 16 weeks, TSE WT mice were left untreated (None) or exposed to formulated CSP7 (5.8 mg) in 30 ml of PBS containing lactose monohydrate (154 mg) or placebo (Pbo) alone 2 hours daily, 5 days/week for 4 weeks using a NEB tower, or injected IP with 1.5 mg/kg of CSP7 or CP daily 5 days/week for 4 weeks. (A) All mice were subjected to micro-CT and lung volume measurements 20 weeks after TSE. Results showed that systemic (IP) or local (Neb) administration of CSP7 reduced lung volume, compliance, Elastance, Resistance. Representative H&E staining (not shown) of tissue sections of 20 weeks TSE WT mice, which was reversed in CSP7 (Neb and IP) treated WT mice. (B) A bar graph shows increased mean linear intercept (MLI) observed in lung tissue sections. (C) Lung parameters of 20-week TSE WT mice, which were reversed in CSP7 (Neb and IP) treated WT mice: Lung volume, elastance, compliance and resistance are shown.

FIGS. 16A-16B show that CSP7 delivered by Neb or IP injection mitigated TSE lung injury in mice. (A) WT mice (n=10/group) were kept in ambient AIR or TSE for 4 h/d 5 day a week as described. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 days/week for 4 weeks using a Neb tower, or were injected IP with 1.5 mg/kg of CSP7 or CP daily 5 d a week for 4 weeks. (A&B) show Total lung homogenate analyzed for RNA and protein level for Mucus hypersecretion and a metaplasia marker. IHC (results not shown) revealed increased expression of MUC5Ac and HDAC6 in lung sections of 20 weeks TSE WT mice, which was reversed by CSP7 (Neb and IP) treatment. Immunofluorescence staining (not shown) indicated increased colocalization of MUC5AC and HDAC6 in lung sections of 20 weeks TSE WT mice, which was reversed by CSP7 treatment (Neb and IP).

FIG. 17 shows that CSP7 delivered by Neb or IP injection increased acetylated α-tubulin expression. WT mice (n=10/group) were kept in ambient AIR or TSE for 4 h/d 5 days a week as described. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 d a week for 4 weeks using a Neb tower, or were injected IP with 1.5 mg/kg of CSP7 or CP daily 5 d/week for 4 weeks. IHC (results not shown) revealed increased in expression of Ac-Tub (cilia) and LC3 in lung sections of 20 week TSE WT mice, which was reversed by CSP7 (Neb and IP) treatment. Tissue staining for Ac-Tub (results not shown) in lung trachea sections of 20 weeks TSE WT mice was reversed by CSP7 treatment. Immunofluorescence (images not shown) using acetylated/α-tubulin (cilia) demonstrated after isolation of MTEC a decrease in number of ciliated cell (Ac-Tub) isolated from 20 weeks TSE WT mice, which was reversed in CSP7 treatment (shown in bar graph).

FIGS. 18A-18B shows that gene and protein overexpression in COPD is mitigated by CSP7. Human (n=4) tissues from control donors (NL) and from COPD patient lungs (n=4) were treated with PBS or 10 μM CSP or CSP7 ex vivo in dishes for 72 h. (A) Bar graphs showing increased expression of MUC5AC, HDAC6, Caveolin1 and FOXA3 mRNA, and decreased expression of FOXA2 mRNA analyzed by qPCR. (B) Western Blot images show increased MUC5Ac, HDAC6, SPDEF, and decreased acetylated tubulin and FOXA2 level in the COPD lung homogenates, which were reversed by treatment with CSP or CSP7.

FIGS. 19A-19B show the role of Cav1 in Mucin hypersecretion and ciliary disassembly. WT mice (n=10/group) were kept in ambient AIR or TSE for 4 h/d 5 days a week as described. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 d/week for 4 weeks using a Neb tower, or IP injected with 1.5 mg/kg of CSP7 or CP daily 5 d a week for 4 weeks. (A) IHC images show increased expression of CAV1 in lung sections of 20 weeks TSE WT mice, which was reversed in CSP7 (Neb and IP) treatment. (B) AECs isolated from NL transduced with Ad-Ev or Ad-CAV were examined in a Western Blot that showed increased MUC5AC, HDAC6, SPDEF, FOXA3, Cav1, LC3, Beclin1, ATG5 and decreased FOXA2, AC-Tubulin and p62 expression in TSE-treated AECs. This elevation was greater than that observed in TSE treated AECs transduced with Ad-CAV1.

FIGS. 20A-20C. Role of p53 and PAI-1 in TSE induced mucin hypersecretion and cilia dysfunction in a mouse model. (A) A bar graph shows increased MUC5AC mRNA expression in TSE-treated AECs, which was absent in TSE treated AECs transduced with Lvp53 shRNA. (B) A Western blot shows increased expression of MUC5AC, HDAC6, SPDEF, and FOXA3, and decreased expression of FOXA, AC-Tub (cilia) expression in TSE-treated AECs, which is absent in MUC5AC, HDAC6, SPDEF and elevation in FOXA2, AC-Tubulin in TSE treated AECs transduced with Lvp53 shRNA. (C) A Western blot shows increased expression of MUC5AC, HDAC6, SPDEF, and FOXA3, and decreased expression of FOXA2 and AC-Tub(cilia) in AECs isolated from the TSE (20 weeks) WT mice, which was reversed in WT mice kept in ambient AIR, as well as in TSE p53^{-/-} and PAI-1^{-/-} mice. IHC (images not shown) revealed increased expression of MUC5AC in the lung sections of TSE (20 weeks) WT mice, which was absent in WT mice kept in ambient AIR, and in TSE p53^{-/-} and PAI-1^{-/-} mice.

FIGS. 21A-21E show the mechanism of CSP7 attenuation of mucus hypersecretion and ciliary disassembly. (A) AECs were isolated from NL and COPD lungs. AECs from COPD lungs were treated with or without CSP7 or CP in vitro for 48 h. Bar graph shows decreased PP2AC and its reversal by CSP7. (B) Bar graph shows elevation of CIP2A and its reversal by CSP7. (C) Western blot shows that levels of protein PP2AC CIP2A, ERK1/2 and MMP12 were reversed by CSP7. (D) COPD lung tissues with CSP7 (ex vivo) have reduced protein phosphatase 2A (PP2A) signaling and which was reversed by CSP7. Serine-threonine phosphatase activity for PP2A was determined for each individual and is represented on the Y axis as pm of phosphate liberated per minute. (E) WT mice (n=10/group) were kept in ambient AIR or TSE for 4 h/day, 5 day a week as described. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 d a week for 4 weeks using a Neb tower, or were injected IP with 1.5 mg/kg of CSP7 or CP daily 5 d/week for 4 weeks, TSE exposure reduced protein phosphatase 2A (PP2A) signaling and this was reversed by CSP7. Serine-threonine phosphatase activity for PP2A was determined for each individual and is represented on the y-axis as pm phosphate liberated per minute.

FIGS. 22A-22F show that the telomere of A₂Cs from human fibrotic lung is shortened. A₂Cs were isolated from the fibrotic lungs of human patients. (A) TeloTAGGG assay was conducted for estimating the telomere length of the isolated genomic DNA. The southern blot data shows the telomere shortening of the A₂Cs from fibrotic lung. (B) Bar graph shows the relative quantification of the shortening occurred in the AECs. (C) Western blot analysis was conducted to analyze the protein expression of telomerase enzyme (TERT), and the apoptosis pathway related proteins the genomic DNA. (D) Bar graph showing relative telomere length of the A₂Cs was analyzed by qPCR after extracting. (E) Gel shows the telomerase enzyme activity as analyzed by the TRAPEZE enzyme assay. (F) Bar graph shows the quantitation of the relative trapeze enzyme activity. (G) Immunohistochemical (IHC) analysis of the TRF2, p53 and PAI-1 protein expression in the lung sections are shown.

FIGS. 23A-23G show that telomeres of A₂Cs from human COPD lung are shortened. A₂Cs were isolated from the COPD lungs of human patients. (A) TeloTAGGG assay was conducted for estimating the telomere length of the isolated genomic DNA. The southern blot data shows the telomere shortening of the A₂Cs from COPD lung. (B) Bar graph shows the relative quantification of the shortening occurred in the A₂Cs. (C) Bar graph showing relative telomere length of the A₂Cs was analyzed by qPCR after extracting the genomic DNA. (D) Western blot analysis was conducted to analyze the protein expression of telomerase enzyme (TERT), and the apoptosis pathway related proteins. (E) Gel shows the telomerase enzyme activity as analyzed by the TRAPEZE enzyme assay. (F) Bar graph shows the quantitation of the relative trapeze enzyme activity. (G) Immunohistochemical (IHC) analysis of the TRF2, p53 and PAI-1 protein expression in the lung sections are shown.

FIGS. 24A-24E show that passive cigarette smoke exposure leads to decrease in telomerase expression and shortening of telomere in A₂Cs of wild type mice. Wild type mice were exposed for 20 weeks of smoke and later treated with the peptide (CSP7) or control peptide (CP) and the A₂Cs were isolated. (A) Relative telomere length of the A₂Cs was analyzed by qPCR after extracting the genomic DNA. (B) Gel shows the telomerase enzyme activity as analyzed by the TRAPEZE enzyme assay. (C) Bar graph shows the quantitation of the trapeze enzyme activity is shown. (D) Western blot analysis was conducted to analyze the protein expression of telomerase enzyme (TERT), and the apoptosis pathway related proteins. (E) Immunohistochemical (IHC) analysis of the TRF2, p53 and PAI-1 protein expression of the lung sections are shown.

FIGS. 25A-25E show that repeated bleomycin exposure leads to decrease in telomerase expression and shortening of telomere in A2Cs of wild type mice. Wild type mice were exposed for bleomycin once in two weeks for 16 weeks as intranasal administration. Peptide (CSP7) or control peptide (CP) treatment started at 14^{th} week and continued daily till the end of the experiment, and the AECs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPEZE enzyme assay. (D) Bar graph shows the quantitation of the trapeze enzyme activity is shown. (E) Immunohistochemical (IHC) analysis of the TRF2, p53 and PAI-1 protein expression of the lung sections are shown.

FIGS. 26A-26C show the effect of 20 weeks of TS on mice lacking IL-17A expression. (A) qPCR analysis of A₂Cs isolated from IL-17A^{-/-} mice exposed to 20 weeks of TS for telomere length suggests that IL-17A^{-/-} mice resist telomere dysfunction and CSP7 treatment had limited effects on baseline telomerase activity in these mice. (B) Western blotting of A₂C lysates indicated that expression of p53, active caspase-3, β-galactosidase, TRF2, TRF1 and PNUTS remained unchanged, suggesting that IL-17A-deficient mice resist TSE-induced A₂C injury. (C) Immunohistochemistry (IHC) analysis of the TRF2 and TRF1 protein expression of the lung sections of IL-17A^{-/-} mice kept in ambient AIR, exposed to 20 weeks of TS with or without CSP7 or CP treatment are shown.

FIGS. 27A-27E shows that A₂Cs of mice deficient in miR-34a expression are protected from telomere shortening induced by passive cigarette smoke. Induction of p53 by TS exposure caused a significant increase in miR-34a expression A₂Cs of WT or miR-34a floxed (SP-C^{Cre}-miR-34a^{fl/fl}) mice which is resisted by miR-34a^{cKO} (Sp-C^{Cre}-miR-34a^{cKO} mice). SP-C^{Cre}-miR-34a^{cKO} and SP-C^{Cre}-miR-34a^{fl/fl} mice were exposed to smoke for 20 weeks, and later treated with CSP7 or control peptide (CP) and the A2Cs were isolated. (A) Gene expressions of miR-34a^{fl/fl} mice and their conditional knockout counterparts exposed to smoke and treated with CSP7 or CP. (B) Relative telomere length of the AECs was analyzed by qPCR after extracting the genomic DNA. (C) Western blot analysis was conducted to analyze the protein expression of telomerase enzyme (TERT), and the apoptosis pathway related proteins. (D). Gel shows the telomerase enzyme activity as analyzed by the TRAPEZE enzyme assay. (E) Bar graph shows the quantitation of the trapeze enzyme activity.

FIGS. 28A-28E show that passive cigarette smoke exposure leads to decrease in telomerase expression and shortening of telomere in A₂Cs of uPA^{-/-} mice. uPA^{-/-} mice were exposed for 20 weeks tobacco smoke, and later treated with the peptide (CSP7) or control peptide (CP) and the A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPEZE enzyme assay. (D) Bar graph shows the quantitation of the trapeze enzyme activity is shown. (E) Immunohistochemistry (IHC) analysis for TRF2 and TRF1 protein of the lung sections from uPA^{-/-} mice kept in ambient AIR, exposed to 20 weeks of TS with CSP7 treatment is shown.

FIGS. 29A-29D show that repeated bleomycin exposure leads to decrease in telomerase expression and shortening of telomere in A₂Cs of uPA-1^{-/-} mice. uPA^{-/-} mice were exposed for bleomycin once in two weeks for 16 weeks as intranasal administration. Peptide (CSP7) or control peptide (CP) treatment started at 14^{th} week and continued daily till the end of the experiment, and the A2Cs were isolated. (A) Relative telomere length of the A2Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPEZE enzyme assay. (D) Bar graph shows the quantitation of the trapeze enzyme activity is shown.

FIGS. 30A-30E show that AECs of PAI-1^{-/-} mice resist telomere shortening induced by passive cigarette smoke. PAI-1^{-/-} mice were exposed for 20-week smoke, and later treated with the peptide (CSP7) or control peptide (CP) and the AECs were isolated. (A) Relative telomere length of the AECs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPEZE enzyme assay. (D) Bar graph shows the quantitation of the trapeze enzyme activity is shown. (E) Immunohistochemistry (IHC) analysis for TRF2 and TRF1 protein of the lung sections from PAI-1^{-/-} mice kept in ambient AIR, exposed to 20 weeks of TS with CSP7 or CP treatment is shown.

FIGS. 31A-31D show that A₂Cs of PAI-1^{-/-} mice resist telomere shortening induced by treatment with repeated dose of bleomycin. PAI-1^{-/-} mice were exposed for bleomycin once in two weeks for 16 weeks as intranasal administration. Peptide (CSP7) or control peptide (CP) treatment started at 14^{th} week and continued daily until the end of the experiment, and the AECs were isolated. (A) Relative telomere length of the A₂Cs was analyzed by qPCR after extracting the genomic DNA. (B) Western blot analysis A₂Cs was conducted to analyze the protein expression of telomerase enzyme (TERT), and the apoptosis pathway related proteins. (C) Gel shows the telomerase enzyme activity as analyzed by the TRAPEZE enzyme assay. (D) Bar graph shows the quantitation of the trapeze enzyme activity is shown.

FIG. 32 shows Regulation of telomere shortening through p53-induced uPA-fibrinolytic system cross talk in ATII cells (A₂Cs). IPF, COPD, BLM and CS induce p53 expression in ATII cells. p53 can activate SIAH and miR-34a. Expression of the SIAH leads to the ubiquitination and degradation of the TRF2 which in turn downregulates the expression of TERT. TRF1 upregulation also results in TERT downregulation.

FIGS. 33A-33D show that passive cigarette smoke exposure leads to decrease in telomerase expression and shortening of telomere in AECs of wild type mice, which is reversed by CSP7 delivered via dry powder inhalation (CSP7^{DPI}). WT mice were exposed for 20 weeks tobacco smoke, and later treated with inhalation of CSP7 dry powder (CSP7^{DPI}) and the A₂Cs were isolated. (A) Relative telomere length of the A₂Cs was analyzed by qPCR after extracting the genomic DNA. (B) Gel shows the telomerase enzyme activity as analyzed by the TRAPEZE enzyme assay. (C) TeloTAGGG assay was conducted for estimating the telomere length of the isolated genomic DNA. (D) Western blot analysis of A₂C lysate was conducted to analyze the protein expression of telomerase enzyme, and the apoptosis pathway related proteins.

FIGS. 34A-34C show that CSP7 mitigates pathways of mucus hypersecretion (MH) and cilia shortening in airway epithelial cells (AECs) from COPD lungs. (A) AECs isolated from nL and COPD lungs were treated with or without CSP7 or CP in vitro. AEC lysates were immunoblotted for MUC5AC, HDAC6, FOXA2, FOXA3, SPDEF, acetylated tubulin (Ac-Tub), CAV1, p53, PAI-1, LC3 and □-actin (to assess equal loading). Representative image from two independent experiments is shown. (B) Total RNA from AECs (n=4) treated as in FIG. 34A were analyzed for MUC5AC, HDAC6, FOXA2, FOXA3 and CAV1 mRNA by qPCR. (C) Immunofluorescence staining (magnification 40× (scale bar 50 μM)) and co-localization of MUC5AC and HDAC6 in AECs from nL, and COPD lungs treated with or without CSP7. Representative image from two experiments were shown. Each experiment was repeated at least 2-3 times and data are presented as mean+SD and **P<0.01 and ***P<0.001 were obtained by one-way Anova with Tukey's multiple comparison test and log-rank tests, respectively.

FIGS. 35A-35E show that CSP7 alleviates MH and cilia shortening in AECs from COPD lungs. (A) IHC images showing increased MUC5Ac staining in the lung sections of patients with COPD (magnification 4×, 20× and 100× (scale bar 500 μM, 100 μM and 20 μ)). (B) Acridine orange staining and fluorescence microscopy of AECs from nL and COPD. AECs from COPD lungs were left untreated or treated with CSP7 in vitro for 6 h, stained with acridine orange (acidic vesicles) and subjected to fluorescence microscopy (magnification 4× (scale bar 500 μM)). (C) Immunofluorescence staining and co-localization of Ac-Tub/LC3 in AECs of nL, and COPD lungs treated as in FIG. 34C (magnification 40× (scale bar 50 μM)). The image represents the findings of two independent experiments. Bar graph showing (D) number of ciliated cells and (E) cilia length in AECs from nL, and COPD lungs treated with or without CSP7 or CP. Each experiment was repeated at least 2-3 times and data are presented as mean+SD and ***P<0.001 were obtained by one-way Anova with Tukey's multiple comparison test and log-rank tests, respectively.

FIGS. 36A-36F show that CSP7 mitigates cigarette smoke extract (CSE)-induced MH and cilia dysfunction. (A) Western blot images showing expression of MUC5AC, HDAC6, FOXA2, FOXA3, SPDEF, acetylated tubulin (Ac-Tub), CAV1, p53, PAI-1, and LC3 in lysates of naive AECs from human nL or nL AECs exposed to CSE left untreated (None) or treated with CSP7 or CP for 48 h in vitro. Same samples were analyzed for R-actin to assess equal loading. Representative findings from two independent experiments are presented. (B) Total RNA from nL AECs (n=4) treated as in FIG. 36A tested for MUC5AC, HDAC6, FOXA2, FOXA3 and CAV1 mRNA expression by qPCR. Immunofluorescence staining of AECs from nL treated as FIG. 35A for co-localization of (C) MU5AC and HDAC6 (magnification 20× (scale bar 100 μM)) or (D) Ac-Tub and LC3 (magnification 40× (scale bar 50 μM)). Representative image from two independent experiments were shown. Bar graph depicting (E) number of ciliated cell and (F) cilia length in AECs from nL treated as in FIG. 35. Each experiment was repeated at least 2-3 times and data are presented as mean+SD and **P<0.01 and ***P<0.001 were obtained by one-way Anova with Tukey's multiple comparison test and log-rank tests, respectively.

FIGS. 37A-37I show that CSP7 mitigates chronic cigarette smoke induced lung injury (CS-LI) in WT mice. WT mice (n=10/group) were kept in ambient AIR or exposed to CS (CSE) for 4 h/day 5 days a week as described (Schroeder et al., Am J Respir Cell Mol Biol 47:178-185, 2012. doi: 10.1165/rcmb.2011-04210C, Wu et al., Autophagy 16: 435-450, 2020. doi: 10.1080/15548627.2019.1628536.) After 16 weeks, WT mice exposed to CS were left untreated (None) or treated with CSP7 (5.8 mg) in 30 ml of PBS containing lactose monohydrate (154 mg) or placebo (Pbo) alone 2 h/day 5 days a week for 4 weeks using a nebulization tower, or by IP injection of 1.5 mg/kg of CSP7 or CP/day 5 days a week for 4 weeks. All mice were subjected to (A) lung volume measurements 20 weeks after CS exposure. Total long homogenates were analyzed for MCM, autophagy marker and R-actin (B) proteins and their (C) mRNAs. (D) Lung sections of above mice were subjected to IHC staining for (magnification 20× (scale bar 100 μM)) MUC5AC. (E) Lung sections of above mice were subjected to IHC staining for (magnification 20× (scale bar 100 μM)) HDAC6. (F) Periodic acid-Schiff (PAS) staining of the airway to visualize goblet cells in the lung sections of WT mice (magnification 100× (scale bar 20 μM)). (G) Immuno-fluorescence staining (magnification 40× (scale bar 50 μM)) for MUC5AC and HDAC6 co-localization. Total RNA from tracheal epithelial cells isolated from mice (n=4) treated and was analyzed for (H) MUC5AC and (I) HDAC6 mRNA by qPCR. Each experiment was repeated at least 2 times and data are presented as mean+SD and **P<0.01 and ***P<0.001 were obtained by one-way Anova with Tukey's multiple comparison test and log-rank tests, respectively.

FIGS. 38A-38F show that CSP7 treatment attenuates airway epithelial injury in WT mice with CS-LI. IHC images (magnification 20× (scale bar 100 μM)) showing the expression of (A) acetylated α-tubulin (cilia) and (B) LC3 in lung sections of WT mice kept in ambient AIR or exposed CS (CSE) for 20 weeks with or without CSP7 (Neb and IP) treatment. (C) IHC staining (magnification 20× (scale bar 100 μM)) for CAV1 in lung sections of CSE WT mice left untreated or treated with CSP7 (Neb) or placebo (Pbo) via airways by nebulization or treated with CSP7 or CP by IP injection. Ambient Air kept mice were used as controls. (D) IHC analyses of lung trachea sections showed decrease in acetylated α-tubulin staining in CSE WT mice, which is reversed by CSP7 treatment (magnification 10× (scale bar 200 μM)). ( ) Immunofluorescence imaging for acetylated α-tubulin (Ac-TUB) for cilia in TECs isolated from CSE WT mice treated as in FIG. 38A (magnification 4× (scale bar 500 μM)). (F) Graph showing number of Ac-Tub-positive cells. Each experiment was repeated at least 2 times and data are presented as mean+SD and *P<0.01, **P<0.001 and ***P<0.0001 were obtained by one-way Anova with Tukey's multiple comparison test and log-rank tests, respectively.

FIGS. 39A-39D show that CSP7 mitigates cilia shortening in human AECs exposed to CSE in vitro and macrophages accumulation in CS-LI mice. (A) Immunofluorescence staining (magnification 4× (scale bar 500 μM)) revealed decrease in acetylated α-tubulin (cilia) expression in AECs exposed to CSE that is reversed with CSP7 treatment. (B) Bar graph represents the changes in the number of ciliated cells under various treatment conditions. (C) Bronchoalveolar lavage (BAL) fluids were collected from WT mice (n=10/group) kept in ambient AIR or exposed to CS and treated with formulated CSP7 or placebo (Pbo) alone 2 h daily 5 days a week for 4 weeks using a Neb tower, or IP injected with 1.5 mg/kg of CSP7 or CP daily 5 days a week for 4 weeks. The BAL cells were stained with Diff-quick staining 10× (scale bar 200 μM)). (D) Bar graph represents the changes in the number of macrophages under various treatment conditions. Each experiment was repeated at least 2-3 times and data are presented as mean+SD and *P<0.05, **P<0.01 and ***P<0.0001 were obtained by one-way Anova with Tukey's multiple comparison test and log-rank tests, respectively.

FIGS. 40A-40D show the effects CSP7 on MUC5AC and MCC markers in human COPD tissues and AECs ex vivo. Human nL (n=4) tissues from control donors, and COPD lung (n=4) tissues were left untreated or treated with 10 μM CSP7 or CP in dishes for 72 h. (A) Western blotting of COPD lung tissues homogenates MUC5AC, HDAC6, FOXA2, FOXA3, SPDEF, acetylated tubulin (Ac-Tub), CAV1, p53, PAI-1, LC3 and R-actin proteins. (B) Total RNA from nL and COPD tissues treated as in FIG. 40A were analysed for MUC5AC, HDAC6, FOXA2, FOXA3 and CAV1 mRNAs by qPCR. (C) AECs isolated from nL tissues were treated with control siRNA (siControl) or CAV1 siRNA (siCAV1) and cells were left untreated or treated with cigarette smoke extract (CSE). The lysates were immunoblotted for MUC5AC, HDAC6, FOXA3, Ac-Tub, CAV1, p53, PAI-1 LC3 and β-actin. (D) AECs isolated from nL were transduced with Ad-Ev or Ad-Cav1. These cells were later treated with or without CSP7 and the lysates were tested for MUC5AC and other listed proteins by Western blotting. Each experiment was repeated at least 2 times and data are presented as mean+SD and *P<0.05, **P<0.01 and ***P<0.001 were obtained by one-way Anova with Tukey's multiple comparison test and log-rank tests, respectively.

FIGS. 41A-41F show the role of p53 and PAI-1 in CS induced MH and cilia dysfunction in mice. (A) AECs isolated from nL were transduced with Lv-p53 shRNA or control non-specific shRNA. These cells were treated with or without cigarette smoke extract in PBS (CSE). AEC lysates were immunoblotted for MUC5AC, HDAC6, FOXA3, acetylated tubulin (Ac-Tub), CAV1, p53, PAI-1 and LC3. Same samples were analyzed for R-actin to assess equal loading. (B) AECs from nL transduced with Ad-Ev or Ad-p53 were left untreated or treated with CSP7. Lysates of Ad-Ev, Ad-p53 and Ad-p53+CSP7 treated AECs were immunoblotted for listed proteins. (C) AECs from nL transduced with Lv-PAI-1 shRNA or control non-specific shRNA were later treated with or without CSE. The lysates were tested for the above proteins by Western blotting. (D) AECs transduced with Ad-Ev or Ad-PAI-1, and Ad-PAI-1 exposed AECs were later treated with or without CSP7. Lysates were immunoblotted for listed proteins. (E) AECs isolated from ambient AIR kept or CS exposed (CS) WT, and p53- and PAI-1-deficient mice were immunoblotted for MUC5AC, HDAC6, Ac-Tub, CAV1, LC3 and α-actin. (F) Lung sections of WT, and p53- and PAI-1-deficient mice treated as in FIG. 41E were subjected to IHC staining for MUC5AC (magnification 20× (scale bar 100 μM)).

FIGS. 42A-42J show that CSP7 restores PP2A activity by altering PP2A and CIP2A expression in COPD tissues and in mice exposed to CS. (A) AECs from nL, and COPD lungs were treated with or without CSP7 or CP for 48 h and tested for PP2A activity. (B) AEC lysates treated as in FIG. 42A were immunoblotted for PP2A, CIP2A, ERK1/2 and MMP12. Same samples were analyzed for R-actin to assess equal loading. Total RNA from AECs treated as in FIG. 42A were analyzed for (C) PP2A and (D) CIP2A mRNA. (E) Human nL (n=5) and COPD lung (n=5) tissues treated with PBS or 10 μM CSP7 or CP ex vivo in dishes for 72 h were tested for PP2A activity. RNA from nL and COPD tissues treated as in FIG. 42E were analyzed for (F) PP2A and (G) CIP2A mRNA. (H) The lung homogenates from WT mice (n=10/group) kept in ambient AIR or exposed to CS (CS) left untreated (None) or treated with CSP7 or placebo by nebulization, or by IP injection as in FIG. 37 were tested for PP2A activity. RNA for ambient AIR and CS mice treated with or without CSP7 or CP were analyzed for (I) PP2A or (J) CIP2A mRNA by qPCR. Each experiment was repeated at least 2-3 times and data are presented as mean+SD and **P<0.01 and ***P<0.001 were obtained by one-way Anova with Tukey's multiple comparison test and log-rank tests, respectively.

FIGS. 43A-43L show that CSP7 inhibits p53 and improves viability in AT2 cells (A2Cs) from mice with CSE-LI and in human COPD lung tissues. WT mice (n=10/group) were exposed to CS for 4 h/day 5 days a week. After 16 weeks, CS exposed (CSE) WT mice were left untreated (None) or treated via airways to formulated CSP7 (Neb) or placebo (Pbo) alone 2 h daily 5 days a week for 4 weeks using a nebulization tower. WT mice with CS-induced lung injury and IP injected CSP7 or CP daily 5 days a week for 4 weeks were used as controls for comparison. (A) Compliance of respiratory system (Crs), (B) Elastance of respiratory system (Ers) and (C) Resistance of respiratory system (Rrs). ( ) Microscopic images of H&E-stained sections (magnification 10× (scale bar 200 μM)) and (E) bar graph showing increased MLI in COPD lung than nL. (F) Microscopic images of H&E-stained lung sections magnification 1Ox (scale bar 200 μM) and (G) bar graph showing MLI in mice with CS-LI vs those kept in ambient AIR. (H) AT2 cells isolated from nL and COPD lung tissues were analyzed for p53, PAI-1, senescence (β-Gal), apoptosis (Cl. Cas-3) and R-actin by Western blotting. (I) AT2 cells isolated from human control (nL) and COPD lung tissues left untreated (None) or treated with CSP7 or CP for 72 h ex vivo were immunoblotted for p53, SP-C, Cl. Cas-3, Cas-3 and β-actin. (J) Lysates of AT2 cells isolated from nL and COPD lung tissues treated in vitro were immunoblotted for p53, SP-C, apoptosis, and β-actin. AT2 cells isolated from ambient AIR kept, or CS (K) WT or (L) PAI-1-deficient mice left untreated or treated with CSP7 or CP by IP injection were analyzed by above proteins by Western blotting. Each experiment was repeated at least 2 times and data are presented as mean+SD and **P<0.01 and ***P<0.001 were obtained by one-way Anova with Tukey's multiple comparison test and log-rank tests, respectively.

FIG. 44A shows fold change in Cleaved Caspase-3 after CSP7 treatment. FIG. 44B shows fold change in beta-galactosidase after CSP7 treatment. FIG. 44C shows fold change in IL-17A in mice with TSE-induced injury after CSP7 treatment.

FIG. 45 shows inhibition of p53-induced PAI-1 by CSP inhibits PTS-induced A₂EC apoptosis in WT mice. Four weeks after initiation of PTS exposure, the WT mice were administered an intraperitoneal injection of vehicle or CSP (18.75 mg/kg body wt) or control peptide (CP) once a week for the next 4 weeks. Exposure to PTS was continued for another 12 weeks. BAL fluid and lysates (CL) from A₂ECs isolated from PTS exposed WT mice treated with vehicle, CSP or CP were immunoblotted for p53 and PAI-1 and activation of caspase-3. -β-actin was tested for similar loading. This demonstrates that treatment of WT mice exposed to PTS with CSP mitigates A₂EC apoptosis. The process involves inhibition of p53-induced PAI-1 expression.

FIGS. 46A-46B show PTS injury induces cav1 expression and its interaction with the PP2Ac in A₂ECs in vivo. (A) CSP inhibits PTS-induced acetylation of p53 and P¹⁵S-p53, and induces Sirt1. A₂EC lysates from WT mice in AIR or exposed to PTS, PTS+CSP, or PTS+CP were immunoblotted for Cav1, acetylated p53 and ^{P15S}-p53, Sirt1 and β-actin. (B) Lysates of A₂ECs were immunoprecipitated (IP) for PP2Ac and immunoblotted (IB) for Cav1 to assess their interaction. These data show that PTS induces Cav1 in WT mice (A), and CSP inhibits the A₂EC Cav1 interaction with PP2Ac (B) in WT mice. These results indicate that CSP-mediated changes are associated with inhibition of the Cav1 interaction with PP2Ac, an ATM kinase inhibitor that prevents ^{P15S}p53 and facilitates degradation of p53 by mdm2. Further, CSP-mediated inhibition of A₂EC apoptosis involves restoration of Sirt1 which is otherwise reduced after PTS injury. This strongly suggests a new link between miR-34a-mediated control of Sirt1 expression and acetylation of p53 (A) regulates p53 levels and downstream induction of PAI-1 by A₂ECs leading to apoptosis after PTS injury.

FIGS. 47A-47C show IL-17A is increased in COPD lungs. (A) Tissue sections from the lungs of patients with COPD and control donors were subjected to immunohistochemical (IHC) analysis using anti-IL-17A antibody. (B) Homogenates prepared from the control and COPD lung tissues were analyzed for IL-17A levels by Western blotting. (C) RNA isolated from control and COPD lung tissues were subjected to real-time quantitative PCR to assess IL-17A mRNA levels.

FIGS. 48A-48D show IL-17A induced during PTS exposure augments p53, PAI-1 expression, apoptosis in A₂ECs. IL-17A accumulates in lungs during PTS injury. (A) Lung sections of WT mice in AIR or exposed to PTS for 20 weeks were subjected to IHC for IL-17A. (B) Lung homogenates or RNA were tested for IL-17A protein and mRNA (Bar graph). (C) IL-17A induces p53, PAI-1 and apoptosis in A₂ECs in vitro. A₂ECs isolated from uninjured WT and p53^{-/-} mice were treated (0-100 ng/ml) with IL-17A for 24 h and the A₂EC lysates were immunoblotted for p53, PAI-1, Cl./caspase-3 and β-actin. (D) IL-17A induces acetylated and total p53, Cav1 expression, apoptosis, and inhibits Sirt1 in A2ECs in vivo. WT mice received saline or IL-17A intranasally (3 μg in 50 μl saline). 24 h later A2ECs were isolated, and lysates were immunoblotted for acetylated and total p53, Sirt1, cav1, and Cl. caspase-3 to assess apoptosis, and β-actin for loading. The levels of IL-17A (red stain) are markedly increased after PTS lung injury (A, B). IL-17A mimics the effect of PTS exposure in WT A₂ECs in vitro (C), and in vivo (D), while IL-17A failed to induce PAI-1 or apoptosis in p53′A₂ECs in vitro, indicating PTS-induced IL-17A is likely to play a pivotal role in p53-mediated A₂EC apoptosis and lung injury.

FIGS. 49A-49B show the role of IL-17A in PTS-induced p53, PAI-1 expression, and apoptosis in A₂ECs. (A) IL-17A^{-/-} mice resist PTS-induced p53 expression and apoptosis in A₂ECs. Mice were kept in ambient AIR or exposed to 20 weeks of PTS. A₂ECs isolated from WT and IL-17A^{-/-} mice in AIR or exposed to PTS were analyzed for acetylated/total p53, Sirt1 and activated caspase-3 to assess apoptosis by Western blotting. (B) CSP inhibits IL-17A-induced p53 and PAI-1 expression and apoptosis in A₂ECs in vitro. A₂ECs isolated from WT mice were treated with PBS or IL-17A (100 ng/ml) in the presence or absence of CSP or control peptide, CP for 24 h in culture dishes. A₂EC lysates were immunoblotted for p53, PAI-1, Cl./total caspase-3 and β-actin. WT mice exposed to IL-17A (FIG. 48D) and PTS (FIG. 46A) induce p53 acetylation and Cav1 expression, while inhibiting Sirt1 in A₂ECs. However, IL-17A′ mice exposed to PTS failed to induce p53 or apoptosis or inhibit Sirt1 in A₂ECs (A). This indicates that IL-17A plays a pivotal role in p53 mediated A₂EC apoptosis and lung injury and that the process involves PTS-induced p53 acetylation by inhibiting Sirt1 in A₂ECs. Further consistent with A₂EC injury by PTS, this data (B) suggests that CSP inhibits IL-17A-induced p53 and PAI-1 expression, and apoptosis in A₂ECs in vitro.

FIGS. 50A-50C shows p53-mediated induction of PAI-1 contributes to lung inflammation in mice exposed to PTS. WT, p53^{-/-} and PAI-1^{-/-} mice (n=5/gp) in AIR or exposed to PTS for 20 weeks. Lung sections were subjected to IHC analysis for neutrophils (polymorphonucleocyte; PMN) using anti-myeloperoxidase (MPO) antibody and for macrophages using anti-F4/80 antibody. Neutrophils (A) and macrophages (B) were counted in 10 high-power fields (HPF) are shown as bar graph. (C) Lung homogenates were immunoblotted for changes in the levels of MPO using anti-MPO antibody. These data reveal that p53^{-/-} or PAI-1^{-/-} mice resist PTS-induced accumulation of leukocytes in the lungs.

FIGS. 51A-51C show CSP inhibits PTS-induced induced MPO and neutrophil elastase in mice. WT mice in AIR or exposed to PTS or PTS+CSP or PTS+CP. After 20 weeks of PTS exposure, mice were euthanized. (A) Lung sections of the mice were subjected to IHC analysis using anti-MPO antibodies. Neutrophils were quantified by counting positive cells in 10 high-power fields (HPF) are shown as bar graph. Lung homogenates were immunoblotted for MPO and β-actin antigens (B), MPO levels by colorimetric assay (C). This figure further suggests that inhibition of PTS-mediated induction of p53 and downstream PAI-1 by CSP significantly suppresses lung inflammation due to reduced pulmonary PMN accumulation.

FIGS. 52A-52B show p53 and PAI-1 are prominently linked to PTS-induced CXCL1, CXCL2 and CXCR2 expression. (A) Lung homogenates from WT, p53^{-/-} and PAI-1^{-/-} mice in AIR or exposed to 20 weeks of PTS were immunoblotted for CXC chemokines (CXCL1 & CXCL2) and their receptor, CXCR2. The same membrane was later stripped and analyzed for (3-actin to assess comparable loading. (B) Lysates from WT A₂ECs treated with PBS, or TSE with or without CSP or control peptide, CP were analyzed for CXCL1, CXCL2, CXCR2 and R-actin by Western blotting. These data reveal an intricate link between p53-mediated induction of PAI-1 and lung inflammation due to exacerbated chemokine expression in A₂ECs. Since induction of p53 and miR-34a depend on IL-17A, targeting this pathway using CSP7 is a means to treat lung inflammation associated with chronic TS exposure. Protection appears to involve changes in p53-mediated downstream in PAI-1 expression in A2ECs by interfering with cav1/PP2Ac interactions.

FIG. 53 shows IL-17A induced serine phosphorylation of p53 augments ATII apoptosis and PAI-1 expression. IL-17A is induced in passive tobacco smoke (PTS) exposed lung injury. To demonstrate that IL-17A induced ^{p15S}p53 contributes to epithelial cell apoptosis and PAI-1 expression, ATII cells isolated from IL-17A (3 μg/20 g) treated mice were screened. IL-17A treatment induced the serine phosphorylation of p53 which also augmented the total p53 expression. Further, IL-17A induced ^{p15S}p53 was inhibited following treatment of mice CSP7 when compared with those treated with saline and control peptide. To further test whether IL-17A induced ^{p15S}p53 alters PAI-1 expression and ATII apoptosis, ATII cells of IL-17A treated mice for PAI-1 and apoptotic markers were analyzed. IL-17A induced the expression of PAI-1 and cl.Cas-3, and inhibited Sirt-1 expression in ATII cells. Further, CSP7 treatment suppressed IL-17A-induced cl.Cas-3, PAI-1 and induced Sirt-1 expression. Consistent with the above results, PTS exposure failed to induce expression of total p53 and PAI-1 in IL-17A-deficient mice which resisted lung injury. These results indicate that IL-17A induced by PTS stabilizes p53 by serine phosphorylation thus induces epithelial cell injury and apoptosis. In addition, CSP7 treatment found to inhibit serine phosphorylation of p53 by neutralizing elevated IL-17A due to PTS exposure.

FIG. 54 shows Sirt-1 expression in p53 acetylation. To test the importance of miR-34a mediated deacetylation of p53 IL-17A or PTS induced lung injury, miR34a conditional knockout mice were treated with tamoxifen. miR34a conditional knockout mice treated with IL-17A (3 μg/mice) was administered with CSP7 and CP a day later. ATII cells isolated from these mice were screened for PAI-1, Sirt-1 and apoptotic markers. Results showed that IL-17A failed to inhibit Sirt1, or induce acetylated p53 (Acp53) and PAI-1 expression or apoptosis in ATII cells of miR-34a^{cKo} mice lacking miR-34a expression in ATII cells, suggesting that mice lacking miR34a expression in ATII cells resists IL-17A induced lung injury and apoptosis. Further, CSP7 treatment had little effect in miR-34a^{cKo} mice since these mice resist IL-17A or PTS injury.

FIG. 55 shows wild type mice either treated with IL-17A (3 μg/mice) or passive tobacco smoke (10 weeks) were treated with CSP7 at 24 hours after IL-17A treatment, and 4^{th} and 8^{th} weeks of PTS were euthanized for bronchoalveolar lavage fluids (BALF) which were analyzed for their myeloperoxidase (MPO) activity to assess inflammation. BALF MPO activity was significantly increased in mice exposed to PTS or IL-17A when compared to the MPO activity in the BALF collected from ambient AIR kept or saline treated control mice. However, treatment of mice with IL-17A or PTS injury with CSP7 significantly reduced the MPO activity.

FIGS. 56A-56C show p53 and PAI-1 are prominently linked to PTS-induced IL-17A and IL-17R expression and alveolar type II epithelial cells (A₂EC) apoptosis in mouse lungs. WT, p53- and PAI-1-deficient mice in ambient AIR or exposed to PTS (3 cigarettes/daily cycle over 2 h: particulate load was 90 mg/m³/cigarette) for 20 weeks. (A) Lung sections were subjected to IHC for CD4 and CD8 or IL-17A antigens. (B) Lung homogenates and (C) total RNA from WT, and p53- and PAI-1-deficient mice were analyzed for IL-17A by IHC, Western blotting and qRT-PCR.

FIGS. 57A-57C show inhibition of PTS-induced IL-17A by CSP in WT mice. Four weeks after initiation of PTS exposure as in FIG. 56, the WT mice were administered an intraperitoneal (IP) injection of vehicle or CSP (18.75 mg/kg body wt) or control peptide (CP) once a week for the next 4 weeks. Exposure to PTS was continued for another 12 weeks. Lung sections (A), lung homogenates (B) and total RNA (C) were analyzed for IL-17A and IL-17R mRNA expression by qRT-PCR.

FIGS. 58A-58B show a 7-amino acid fragment of CSP, CSP7 Inhibits IL-17A-induced A₂EC apoptosis. (A) Fragments of CSP were synthesized as shown. (B) A₂ECs were treated with CSP or its fragments, or CP, 2 h after exposure of cells to IL-17A. A₂EC lysates were immunoblotted for acetylated/total p53 and active/total caspase-3 24 h after IL-17A exposure. β-actin levels were evaluated to assess equal loading.

FIG. 59 shows inhibition of cigarette smoke extract in PBS (CSE)-induced apoptosis by CSP and CSP7 in A2ECs. A2ECs were treated with PBS, CSP or its fragment, CSP7 or control peptide (CP), 2 h after exposure of cells to CSE. A₂EC lysates were immunoblotted for acetylated/total p53 and active/total caspase-3 24 h after CSE exposure. β-actin levels were evaluated to assess equal loading.

FIGS. 60A-60B show inhibition of PTS-induced p53^{AC}, p53^{S15} and p53, PAI-1, apoptosis and senescence by CSP7 in WT mice. Four weeks after initiation of PTS exposure as in FIG. 56, the WT mice were IP injected with vehicle or CSP7 or CP once a week for the next 4 weeks. Exposure to PTS was continued for another 12 weeks. (A) Lung sections were subjected IHC analysis for p53 and PAI-1. (B) A₂ECs isolated from the lungs of these mice were immunoblotted for above mentioned proteins.

FIGS. 61A-61B shows A₂EC apoptosis is linked to induction of miR-34a and p53-positive feedback during PTS lung injury. (A) PTS injury induces miR-34a in A₂ECs in vivo. A₂ECs isolated from WT mice exposed to 20 weeks of PTS with or without CSP or CP were analyzed for miR-34a by Northern blotting (inset) and qRTPCR (Bar graph). (B) Mouse A₂ECs were treated with PBS, cigarette smoke extract in PBS (CSE) or CSE+control miRNA (Ctr-miR) or CSE+miR-34a-AS or CSE+CSP, or pre-miR-34a alone for 24 h in vitro. The lysates were immunoblotted for p53, active caspase-3 (for apoptosis) and β-actin.

FIGS. 62A-62C shows miR-34a^{cKO} mice resist PTS-induced p53 and PAI-1 expression, apoptosis and senescence in A₂ECs. (A) miR-34a^{fl/fl} mice were crossed with SP-C^{Cre/mTmG} mice and the offspring were genotyped for SP-C° ^{re} (1 kb), miR-34a (318 bp) and mTmG (250 bp) by PCR using specific primers. Mice in lanes 2, 4 & 5 are positive for all three. (B) Mice homozygous for miR-34a and SP-C^{Cre/mTmG} positive were treated with tamoxifen to induce Cre recombinase. miR-34a^{fl/fl} and miR-34a^{cKO} mice were exposed to 20 weeks of PTS with or without CSP7 or CP. Control mice were kept in ambient AIR. Total RNA isolated from AECs of these mice were analyzed for miR-34a expression by qRT-PCR. (C) miR-34a^{fl/fl} and miR-34a^{cKO} mice were exposed to PTS with or without CSP7 or CP treatment for 20 weeks. Control mice were kept in ambient AIR. A₂ECs were isolated from these mice. A₂EC lysates were immunoblotted for changes in p53, PAI-1, apoptosis and senescence by Western blotting.

FIGS. 63A-63B show increased IL-17A expression in lung tissues of COPD patients and mice with TSE lung injury. Western blot (WB) and bar graph revealing upregulated IL-17A protein and mRNA expression in (A) lung tissues of COPD patients vs healthy donors (normal lung, nL) and (B) TSE WT mice vs ambient air exposed (AIR) WT mice.

FIGS. 64A-64G show IL-17A-mediated induction of p53 and PAI-1 regulates A2Cs apoptosis. (A) WB showing IL-17A inducing p53, PAI-1, Cav1 and Cl.Cas-3/Cas-3 (for apoptosis) in A2Cs of IL-17A treated WT mice. (B) WB showing increased p53, PAI-1 and apoptosis in WT mice A2Cs treated with IL-17A in vitro. (C) WB showing lack of induction of PAI-1 and Cl.Cas-3 (apoptosis) in IL-17A treated A₂Cs from p53^{-/-} mice. (D) p53 protein fold change vs. IL-17A (μg/mice). (E) p53 protein fold change vs. IL-17A (ng/ml treated A₂Cs) (F) Cl.Cas3 protein fold change vs. IL-17A (μg/mice). (G) Cl.Cas3 protein fold change vs. IL-17A (ng/ml treated A₂Cs).

FIGS. 65A-65H show CSP7 inhibits IL-17A-induced p53, PAI-1 and apoptosis in injured A₂Cs. (A) WB showing resistance of IL-17A^{-/-} mice to TSE-induced p53, ^{Ac}p53, Sirt-1 and Cl.Cas-3/Cas-3 in A2Cs in vivo. (B) WB showing CSP inhibiting IL-17A-induced p53, PAI-1 and Cl.Cas-3 in in vitro IL-17A treated WT mice A2Cs. CP: Control peptide. (C) Diagram showing the sequence of CSP fragments generated. (D) WB showing CSP7 inhibiting IL-17A-induced p53, ^{Ac}p53 and apoptosis in WT mice A₂Cs treated in vitro. (E) p53 protein fold change vs. IL-17A (μg/mice). (F) p53 protein fold change vs. IL-17A (ng/ml treated A₂Cs) (65, G) Cl.Cas3 protein fold change vs. IL-17A (μg/mice). (H) Cl.Cas3 protein fold change vs. IL-17A (ng/ml treated A₂Cs).

FIGS. 66A-66F shows differential IL-17A and IL-17A receptor (IL-17R) expression in lungs of TSE WT, p53 and PAI-1-deficient mice, and CSP inhibits TSE-induced IL-17A expression in WT mice. (A) Lung sections of WT, and p53- and PAI-1-deficient mice kept in ambient AIR or exposed to TS for 20 weeks (TSE) were subjected to IHC staining for IL17A. (B) Lung sections of WT mice kept in ambient AIR, or lung sections of WT mice with TSE-induced lung injury left untreated or treated with CSP or control peptide (CP) were subjected to IHC staining for IL-17A. (C, D) Western blot (WB) revealing upregulated IL-17A and IL-17R protein expression. Lung homogenates from WT, and p53- and PAI-1-deficient mice were analyzed for IL-17A and IL-17R by Western blotting. (E, F) Bar graph showing upregulated IL-17A mRNA expression. Total RNA isolated from lung tissues of WT, and p53- and PAI-1-deficient mice kept in ambient AIR or TSE with or without CSP or CP treatment were analyzed for IL-17A mRNA by qRT-PCR.

FIGS. 67A-67B shows inhibition of cigarette smoke (or tobacco smoke) extract-induced apoptosis by CSP and CSP7 in A₂Cs and resistance of IL-17A deficient mice TSE-induced lung injury. (A) A₂Cs were treated with CSP or its fragment, CSP7 or control peptide (CP), 2 h after exposure of cells to cigarette smoke extract in PBS (CSE). A₂C lysates were immunoblotted for acetylated/total p53 and active/total caspase-3 and Sirt1 24 h after CSE exposure. β-actin levels were evaluated to assess equal loading. (B) WT and IL-17A′ mice were kept in ambient AIR or exposed to 20 weeks of passive tobacco smoke (TSE). A₂Cs isolated from WT and IL-17A^{-/-} mice in AIR or exposed to passive tobacco smoke were analyzed for acetylated/total p53, Sirt1 and activated caspase-3 to assess apoptosis by Western blotting.

### EMBODIMENTS

The present inventors conceived that induction of p53 and downstream PAI-1 augments senescence and apoptosis in A₂Cs, and alveolar injury. Their data reveal a newly recognized contribution of increased IL-17A and PAI-1 to the outcomes of A₂C telomere dysfunction and alveolar damage, and M5Ac/mucus hypersecretion by AECs, and airway inflammation during chronic TSE intervention by administration of CSP7 and is variants, derivatives, multimers, etc., as described herein acts to block telomere dysfunction in A₂Cs and AECs mucus hypersecretion.

Such activity can be examined using primary A2Cs and AECs isolated from control subjects and patients with COPD, and take advantage of local delivery of CSP7 liquid or DP formulation. Also useful in better understanding the mechanisms involved in the disease process being addressed are lentiviral vectors (Lv) harboring AEC or A₂C specific promoter expressing p53 binding 3′UTR sequences, WT and IL-17A^{-/-}, p53^{-/-} and PAI-1^{-/-}, and p53^{cKO}, PAI-1^{cKO} and Trf2^{cKO} mice lacking their expression in A₂Cs or AECs.

The specificity of CSP7 effects at the molecular level in A₂Cs or AECs can be further confirmed using p53 binding 3′UTR sequences as a decoy that targets p53 binding with endogenous PAI-1 mRNAs without inhibiting p53 expression in mice.

CSP7 (a competitor for Cav1-mediated signaling) delivered via airways in liquid or DP formulation, is shown to mitigate A₂Cs telomere dysfunction, senescence/apoptosis, air sac enlargement, and AEC metaplasia/mucus hypersecretion in TSE lung injury.

### Peptides Based on the Cav1 Sequence

The Caveolin-1 (Cav1) scaffolding domain or peptide (also referred to as CSD or CSP) interferes with Cav1 interaction with Src kinases mimics the combined effect of uPA and anti-β1-integrin antibody as discussed in more detail below. Native human Cav1 has a length of 178 amino acids and a molecular weight of 22 kDa. The amino acid sequence of Cav1 is shown below SE ID NO:3).

| | |
|---|---|
| 1 | MSGGKYVDSE GHLYTVPIRE QGNIYKPNNK AMADELSEKQ VYDAHTKEID LVNRDPKHLN |
| 61 | DDVVKIDFED VIAEPEGTHS FDGIWKASFT TFTVTKYWFY RLLSALFGIP MALIWGIYFA |
| 121 | ILSFLHIWAV VPCIKSFLIE IQCISRVYSI YVHTVCDPLF EAVGKIFSNV RINLQKEI |

As noted above, CSP is the 20 residue peptide underlined above, and has the sequence DGIWKASFTTFTVTKYWFYR (SEQ ID NO:2). The preferred peptide of the present invention, designated CSP7 is the heptapeptide fragment FTTFTVT (SEQ ID NO:1) of CSP and is shown double-underlined within the Cav1 sequence above. CSP7 has the activities shown in the Examples and Figures, below.

In studies disclosed herein, a control peptide for CSP7, which is termed “CP” is a scrambled peptide with the same amino acid composition as the larger CSP (SEQ ID NO:2), but has a different sequence: WGIDKAFFTTSTVTYKWFRY (SEQ ID NO:5).

Modifications and changes may be made in the structure of CSP7, and to create molecules with similar or otherwise desirable characteristics. Such functional derivatives or biologically active derivatives (which terms are used interchangeably) are encompassed within the present invention.

Preferred functional derivatives are addition variants and peptide oligomers/multimers, and the like.

These may be generated synthetically but also by recombinant production, and tested for biological activity of CSP7. A preferred way to measure the activity of the variant is in a competitive binding assay wherein the ability of the peptide variant to compete with binding of soluble Cav1, such as one that is detectably labeled, with soluble uPAR (“suPAR”).

It is understood that distinct derivatives of CSP7 and longer polypeptides comprising CSP7 may easily be made in accordance with the invention, either by chemical (synthetic) methods or by recombinant means (preferred for longer polypeptides).

Included in within the definition of functional variants of CSP7 are addition which preferably comprise an additional 1-5 amino acids at either terminus or at both termini. In other embodiments (which are intended to be distinct from the peptide multimers discussed below), further additional residues may be added, up to about 20 residues. In the addition variant of CSP7, the additional residues N-terminal to, and/or C-terminal to SEQ ID NO:1 (the core CSP7 peptide) may include some of those in the order in which they occur in the native sequence in Cav1 (SEQ ID NO:4). However, an addition variant cannot be SEQ ID NO:3. Alternatively, other amino acids can be added at either terminus of SEQ ID NO:1, with the understanding that the addition variant must maintains the biological activity and binding activity of CSP7 (at least 20% of the activity, or preferably greater, as is set forth below).

Preferred substitutions variants of CSP7 are conservative substitutions in which 1 or 2 residues have been substituted by different residue. For a detailed description of protein chemistry and structure, see Schultz G. E. et al., Principles of Protein Structure, Springer-Verlag, New York, 1979, and Creighton, T. E., Proteins: Structure and Molecular Properties, 2^{nd} ed., W.H. Freeman & Co., San Francisco, 1993, which are hereby incorporated by reference.

Conservative substitutions and are defined herein as exchanges within one of the following groups:

Phe may be substituted by a large aromatic residue: Tyr, Trp.

Thr may be substituted by a small aliphatic, nonpolar or slightly polar residues: e.g., Ala, Ser, or Gly.

Val may be substituted by a large aliphatic, nonpolar residue: Met, Leu, Ile, Cys.

Even when it is difficult to predict the exact effect of a substitution in advance of doing so, one skilled in the art will appreciate that the effect can be evaluated by routine screening assays, preferably the biological and biochemical assays described herein. The activity of a cell lysate or purified polypeptide or peptide variant is screened in a suitable screening assay for the desired characteristic.

In addition to the 20 “standard” L-amino acids, D-amino acids or non-standard, modified or unusual amino acids which are well-defined in the art are also contemplated for use in the present invention. These include, for example, in the substitution variant or addition variant, β-alanine (β-Ala) and other ω-amino acids such as 3-aminopropionic acid, 2,3-diaminopropionic acid (Dpr), 4-aminobutyric acid and so forth; α-aminoisobutyric acid (Aib); ε-aminohexanoic acid (Aha); 6-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); norleucine (Nle); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); homo-arginine (hArg); N-acetyl lysine (AcLys); 2,4-diaminobutyric acid (Dbu); 2,4-diaminobutyric acid (Dab); p-aminophenylalanine (Phe(pNH2)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

Other compounds may be designed by rational drug design to function in manner similar to CSP7. The goal of rational drug design is to produce structural analogs of biologically active compounds. By creating such analogs, it is possible to produce drugs that are more active or more stable than the natural molecules (i.e., peptides), lower susceptibility to alterations which affect functions. One approach is to generate a three-dimensional structure of CSP7 for example, by NMR or X-ray crystallography, computer modeling or by a combination. An alternative approach is to replace randomly functional groups in the CSP7 sequence, and determine the effect on function.

Moreover, a biologically active derivative has the activity of CSP7 in an in vitro or in vivo assay of binding or of biological activity, such as assays described herein. Preferably the polypeptide inhibits or prevents apoptosis of LECs induced by BLM in vitro or in vivo with activity at least about 20% of the activity of CSP7, or at least about 30%, 40%, 50%, 60%, 65%, 70%, 75%, 80%, 85%, 90%, about 95%, 97%, 99%, and any range derivable therein, such as, for example, from about 70% to about 80%, and more preferably from about 81% to about 90%; or even more preferably, from about 91% to about 99%. The derivative may have 100% or even greater activity than CSP7.

The peptide may be capped at its N and C termini with an acyl (abbreviated “Ac”)-and an amido (abbreviated “Am”) group, respectively, for example acetyl (CH₃CO—) at the N terminus and amido (—NH₂) at the C terminus. A broad range of N-terminal capping functions, preferably in a linkage to the terminal amino group are contemplated

The C-terminal capping function can either be in an amide or ester bond with the terminal carboxyl. Any of a number of capping functions that provide for an amide bond are contemplated.

Capping functions that provide for an ester bond are also contemplated.

Either the N-terminal or the C-terminal capping function, or both, may be of such structure that the capped molecule functions as a prodrug (a pharmacologically inactive derivative of CSP7) that undergoes spontaneous or enzymatic transformation within the body in order to release the active drug and that has improved delivery properties over CSP7 (Bundgaard H, Ed: Design of Prodrugs, Elsevier, Amsterdam, 1985).

Judicious choice of capping groups allows the addition of other activities on the peptide. For example, the presence of a sulfhydryl group linked to the N- or C-terminal cap will permit conjugation of the derivatized peptide to other molecules. The presence of a capping group contributes to the stability or in vivo half-life of the peptide.

### Chemical Derivatives of CSP7

In addition to capping groups as described above which are considered “chemical derivatives” of CSP7, the preferred chemical derivatives of CSP7 may contain additional chemical moieties not normally a part of a protein or peptide which can be introduced to CSP7 (or to an addition variant of CSP7) by known means to constitute the chemical derivative as defined herein. Covalent modifications of the peptide are included within the scope of this invention. Such derivatized moieties may improve the solubility, absorption, biological half-life, and the like. Moieties capable of mediating such effects are disclosed, for example, Gennaro, AR, Remington: The Science and Practice of Pharmacy, Lippincott Williams & Wilkins Publishers; 21st Ed, 2005 (or latest edition)

Such modifications may be introduced into the molecule by reacting targeted amino acid residues of the peptide with an organic derivatizing agent that is capable of reacting with selected side chains or terminal residues. Another modification is cyclization of the peptide—which is generally accomplished by adding terminal Cys residues which can be bonded via a disulfide bond to generate the cyclic peptide. Alternative, a cross-linkable Lys (K) is added at one terminus and a Glu (E) at the other terminus.

Cysteinyl residues (added, e.g., for cyclizing purposes) most commonly are reacted with α-haloacetates (and corresponding amines) to give carboxymethyl or carboxyamidomethyl derivatives. Cysteinyl residues also are derivatized by reaction with bromotrifluoroacetone, α-bromo-β-(5-imidozoyl) propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitro-phenol, or chloro-7-nitrobenzo-2-oxa-1,3-diazole.

Added lysinyl residues (e.g., for cyclizing) and the amino terminal residue can be derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Derivatization with bifunctional agents is useful for cross-linking the peptide or oligomer or multimer to a water-insoluble support matrix or other macromolecular carrier. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

### Multimeric or Oligomeric CSP7 Peptides

The present invention also includes longer peptides built from repeating units of CSP7 (or a functional derivative thereof) that has the anti-apoptotic and protective activity of CSP7. The preferred peptide unit of such a multimer is FTTFTVT (SEQ ID NO:1). Addition variants of this peptide that may be the “unit” of the multimer preferably include from 1-4 additional amino acids.

A peptide multimer may comprise different combinations of peptide monomers (which may include either or both of SEQ ID NO:1 or addition variants thereof or a chemically derivatized form of the peptide. Such oligomeric or multimeric peptides can be made by chemical synthesis or by recombinant DNA techniques as discussed herein. When produced by chemical synthesis, the oligomers preferably have from 2-5 repeats of a core peptide sequence, and the total number of amino acids in the multimer should not exceed about 160 residues, preferably not more than 100 residues (or their equivalents, when including linkers or spacers).

A preferred synthetic chemical peptide multimer has the formula

P¹ₙ

wherein the core peptide P¹ is SEQ ID NO:1, and wherein n=2-5, and wherein the core peptide alone or in oligo- or multimeric form has the biological activity of CSP7 as disclosed herein in an in vitro or in vivo bioassay of such activity.

In another embodiment, a preferred synthetic chemical peptide multimer has the formula

(P¹-Xₘ)ₙ-P²
P¹ and P² are the core peptides described above, including additional variants, wherein
(a) P¹ and P² may be the same or different; moreover, each occurrence of P^{I} in the multimer may be a different peptide (or variant);
(b) X is a spacer which comprises or consists of:
   (i) a short organic chain, preferably C₁-C₅ alkyl, C₁-C₅ alkenyl, C₁-C₅ alkynyl, C₁_C₅ polyether containing up to 4 oxygen atoms, wherein m=0 or 1 and n=1-7; or
   (ii) Gly_{z} wherein, z=1-6, and wherein the core peptide alone or in multimeric form has the biological activity of CSP7 as disclosed herein in an in vitro or in vivo assay of such activity.

When produced recombinantly, a preferred spacer is Gly_{z} as described above, where z=1-6, and the multimers may have as many repeats of the core peptide sequence as the expression system permits, for example from two to about 25 repeats. A preferred recombinantly produced peptide multimer has the formula:

(P¹-Gly_{z})ₙ-P²
wherein:
(a) P¹ and P² are, independently, SEQ ID NO:1 or 3 or an addition variant or derivatized form thereof, wherein P¹ and P² may be the same or different; moreover, each occurrence of P¹ in the multimer may be a different peptide (or variant);
wherein
   n=1-25 and z=0-6; (preferred ranges of n include n=1-5, 1-10, 1-15, or 1-20)
and wherein the core peptide alone or in multimeric form has the biological activity of CSP7 as disclosed herein in an in vitro or in vivo bioassay of such activity.

In the present peptide multimers, either P¹ or P² is preferably SEQ ID NO:1. The multimer is optionally capped. It is understood that such multimers may be built from any of the peptides or variants described herein. It is also understood that the peptide multimer should be different from SEQ ID NO:3 (i.e., not native human Cav1 and should not be a native mammalian Cav1 homologue).

### Peptidomimetics

Also included within the scope of this invention is a peptidomimetic compound which mimics the biological effects of CSP7. A peptidomimetic agent may be an unnatural peptide or a non-peptide agent that recreates the stereospatial properties of the binding elements of CSP7 such that it has the binding activity and biological activity of CSP7. Similar to a biologically active CSP7 peptide, peptide multimer, a peptidomimetic will have a binding face (which interacts with any ligand to which CSP7 binds) and a non-binding face. Again, similar to CSP7, the non-binding face of a peptidomimetic will contain functional groups which can be modified by coupling various therapeutic moieties without modifying the binding face of the peptidomimetic. A preferred embodiment of a peptidomimetic would contain an aniline on the non-binding face of the molecule. The NH₂-group of an aniline has a pKa˜4.5 and could therefore be modified by any NH₂— selective reagent without modifying any NH₂ functional groups on the binding face of the peptidomimetic. Other peptidomimetics may not have any NH₂ functional groups on their binding face and therefore, any NH₂, without regard for pKₐ could be displayed on the non-binding face as a site for conjugation. In addition, other modifiable functional groups, such as —SH and —COOH could be incorporated into the non-binding face of a peptidomimetic as a site of conjugation. A therapeutic moiety could also be directly incorporated during the synthesis of a peptidomimetic and preferentially be displayed on the non-binding face of the molecule.

This invention also includes compounds that retain partial peptide characteristics. For example, any proteolytically unstable bond within a peptide of the invention could be selectively replaced by a non-peptidic element such as an isostere (N-methylation; D-amino acid) or a reduced peptide bond while the rest of the molecule retains its peptidic nature.

Peptidomimetic compounds, either agonists, substrates or inhibitors, have been described for a number of bioactive peptides/polypeptides such as opioid peptides, VIP, thrombin, HIV protease, etc. Methods for designing and preparing peptidomimetic compounds are known in the art (Hruby, V J, Biopolymers 33:1073-1082 (1993); Wiley, R A et al., Med. Res. Rev. 13:327-384 (1993); Moore et al., Adv. in Pharmacol 33:91-141 (1995); Giannis et al., Adv. in Drug Res. 29:1-78 (1997). Certain mimetics that mimic secondary structure are described in Johnson et al., In: Biotechnology and Pharmacy, Pezzuto et al., Chapman and Hall (Eds.), NY, 1993. These methods are used to make peptidomimetics that possess at least the binding capacity and specificity of the CSP7 peptide and preferably also possess the biological activity. Knowledge of peptide chemistry and general organic chemistry available to those skilled in the art are sufficient, in view of the present disclosure, for designing and synthesizing such compounds.

For example, such peptidomimetics may be identified by inspection of the three-dimensional structure of a peptide of the invention either free or bound in complex with a ligand (e.g., soluble uPAR or a fragment thereof). Alternatively, the structure of a peptide of the invention bound to its ligand can be gained by the techniques of nuclear magnetic resonance spectroscopy. Greater knowledge of the stereochemistry of the interaction of the peptide with its ligand or receptor will permit the rational design of such peptidomimetic agents. The structure of a peptide or polypeptide of the invention in the absence of ligand could also provide a scaffold for the design of mimetic molecules.

### Deliverable Peptides and Peptide Multimers

One embodiment of the invention comprises a method of introducing the peptide of the invention into animal cells, such as human cells. Compositions useful for this method, referred to as “deliverable” or “cell-deliverable” or “cell-targeted” peptides or polypeptides comprise a biologically active peptide according to the invention, preferably CSP7, or a functional derivative thereof, or a peptide multimer thereof, that has attached thereto or is associated with, a further component which serves as an “internalization sequence” or cellular delivery system. The term “associated with” may include chemically bonded or coupled to, whether by covalent or other bonds or forces, or combined with, as in a mixture. As used herein, “delivery’ refers to internalizing a peptide/polypeptide in a cell. Delivery molecules contemplated herein include peptides/polypeptides used by others to effect cellular entry. See for example, Morris et al., Nature Biotechnology, 19:1173-6, 2001). A preferred strategy is as follows: an apoptosis-inhibiting (“biologically active”) peptide of the invention is bonded to or mixed with a specially designed peptide which facilitates its entry into cells, preferably human cells. This delivery system does not require the delivery peptide to be fused or chemically coupled to biologically active peptide or polypeptide (although that is preferred), nor does biologically active peptide or polypeptide have to be denatured prior to the delivery or internalization process. A disadvantage of earlier delivery systems is the requirement for denaturation of the “payload” protein prior to delivery and subsequent intracellular renaturation. These embodiments are based on known approaches for promoting protein translocation into cells.

One type of “delivery” peptide/polypeptide which promotes translocation/internalization includes the HIV-TAT protein (Frankel, A D et al., Cell 55:1189-93 (1998), and the third a helix from the Antennapedia homeodomain (Derossi et al., J. Biol. Chem. 269:10444-50 (1994); Lindgren, M et al., Trends Pharm. Sci. 21:99-103 (2000); Lindgren et al., Bioconjug Chem. Sep-11:619-26 (2000); Maniti O et al., PLoS ONE 5e15819 (2010). The latter peptide, also known as “penetratin” is a 16-amino acid peptide with the wild-type sequence RQIKIWFQNRRMKWKK (SEQ ID NO:6) or two analogues/variants designated W48F (RQIKIFFQNRRMKWKK, SEQ ID NO:7) and W56F (RQIKIWFQNRRMKFKK, SEQ ID NO:8) (Christiaens B et al., Eur J Biochem 2002, 269:2918-2926). Another variant with both of the above mutations is RQIKIFFQNRRMKFKK (SEQ ID NO:9). Transportan, a cell-penetrating peptide is a 27 amino acid-long peptide containing 12 functional amino acids from the N-terminus of the neuropeptide galanin linked by an added Lys residue to the sequence of mastoparan (Pooga, M et al., FASEB J. 12:67-77 (1998)). The sequence of transportan is GWTLNSAGYLLGKINLKALAALAKKIL (SEQ ID NO:10). Analogues of penetratin and transportan are described by Lindgren et al., (Bioconjug Chem. 2000, supra).

Another protein (family) includes VP22, a herpes simplex virus protein that has the remarkable property of intercellular transport and distributes a protein to many surrounding cells (Elliott, G et al., 1997, Cell 88:223-33; O'Hare et al., U.S. Pat. No. 6,017,735). For example, VP22 linked to p53 (Phelan, A. et al., 1998, Nat Biotechnol 16:440-3) or thymidine kinase (Dilber, M S et al., 1999, Gene Ther 6:12-21) facilitating the spread of linked proteins to surrounding cells in vitro. Also useful are VP22 homologues in other herpes viruses, such as the avian Marek's Disease Virus (MDV) protein UL49, that shares homology with HSV-1 VP22 (Koptidesova et al., 1995, Arch Virol. 140:355-62) and has been shown to be capable of intercellular transport after exogenous application (Dorange et al., 2000, J Gen Virol. 81:2219). All these proteins share the property of intercellular spread that provide an approach for enhancing cellular uptake of the peptides, variants, and multimers of this invention.

Also included are “functional derivatives” of the above intercellular spreading or “delivery” “delivery” or “internalization” proteins and peptides such as HIV-TAT or VP22 which include homologous amino acid substitution variants, fragments or chemical derivatives, which terms are herein for the biologically active peptides. A functional derivative retains measurable translocation or intercellular spreading (VP22-like) activity that promotes entry of the desired polypeptide, which promotes the utility of the present biologically active peptide e.g., for therapy. “Functional derivatives” encompass variants (preferably conservative substitution variants) and fragments regardless of whether the terms are used in the conjunctive or the alternative.

Because the above transport proteins are said to work best when conjugated or otherwise bound to the peptide they are transporting, such as CSP7 or a variant or multimer thereof, there are a number of disadvantages to using them. A more effective delivery polypeptide that can be admixed with the biologically active peptide and does not need to be chemically bonded for its action is described in Morris et al., supra, as “Pep-1” which has the amphipathic amino acid sequence KETWWETWWTEWSQPKKKRKV (SEQ ID NO:11). Pep-1 consists of three domains:
(1) a hydrophobic Trp-rich motif containing five Trp residues KETWWETWWTEW (SEQ ID NO:12) (residues 1-12 of SEQ ID NO:11, above). This motif is desirable, or required, for efficient targeting to cell membrane and for entering into hydrophobic interactions with proteins;
(2) a hydrophilic Lys-rich domain KKKRKV (SEQ ID NO:13) (the 6 C-terminal residues of SEQ ID NO:11) which is derived from the nuclear localization sequence of SV40 virus large T antigen, and improves intracellular delivery and peptide solubility; and (3) a spacer “domain” SQP (3 internal residues of SEQ ID NO:11) which and separate the two active domains above and include a Pro that improves flexibility and integrity of both the hydrophobic and hydrophilic domains.
   Accordingly, another embodiment of the invention is a deliverable peptide or polypeptide comprising CSP7 or a functional derivative thereof as described above, and a delivery or translocation-molecule or moiety bound thereto or associated therewith. The delivery molecule may be a peptide or polypeptide, e.g.,
(a) HIV-TAT protein or a translocationally active derivative thereof,
(b) penetratin having the sequence RQIKIWFQNRRMKWKK (SEQ ID NO:6),
(c) a penetratin variant W48F having the sequence RQIKIFFQNRRMKWKK (SEQ ID NO:7)
(d) a penetratin variant W56F having the sequence RQIKIWFQNRRMKFKK, SEQ ID NO:8)
(e) a penetratin variant having the sequence RQIKIFFQNRRMKFKK, SEQ ID NO:9)
(f) transportan having the sequence GWTLNSAGYLLGKINLKALAALAKKIL (SEQ ID NO:10)
(g) herpes simplex virus protein VP22 or a translocationally-active homologue thereof from a different herpes virus such as MDV protein UL49; or
(h) Pep-1, having the sequence KETWWETWWTEWSQPKKKRKV (SEQ ID NO:11).

When a delivery moiety, such as the peptides and proteins discussed above, is conjugated or fused to the biologically active peptide of the invention, it is preferred that the delivery moiety is N-terminal to the biologically active peptide.

### In Vitro Testing of Compositions

The compounds of this invention are tested for their biological activity, e.g., anti-apoptotic activity, their ability to affect expression of uPA, uPAR and PAI-1 mRNAs, inhibit apoptosis and senescence of AECs and A₂Cs, etc. using any one of the assays described and/or exemplified herein or others well-known in the art.

### In Vivo Testing of Compositions

The ability of a CSP7 variant or multimers to inhibit emphysema, mucus hypersecretion, lung fibrosis in TSE or BLM-treated mice is a preferred test for assessing the functional activity of the compound. Other tests known in the art that measure the same type of activity may also be used.

Pharmaceutical and Therapeutic Compositions and their Administration

The compounds that may be employed in the pharmaceutical compositions of the invention the peptide compounds described above, as well as the pharmaceutically acceptable salts of these compounds. “Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. Chemical modification of a pharmaceutical compound (i.e., drug) into a salt is a technique well known to pharmaceutical chemists to obtain improved physical and chemical stability, hygroscopicity, flowability and solubility of compounds. See, e.g., H. Ansel et al., Pharmaceutical Dosage Forms and Drug Delivery Systems (6^{th} Ed. 1995) at pp. 196 and 1456-1457.

The compounds of the invention, as well as the pharmaceutically acceptable salts thereof, may be incorporated into convenient dosage forms, such as capsules, impregnated wafers, tablets or preferably, injectable preparations. Solid or liquid pharmaceutically acceptable carriers may be employed. “Pharmaceutically acceptable,” such as pharmaceutically acceptable carrier, excipient, etc., means pharmacologically acceptable and substantially non-toxic to the subject to which the particular compound is administered.

Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Gennaro, AR, Remington: The Science and Practice of Pharmacy, Lippincott Williams & Wilkins Publishers; 21st Ed, 2005 (or latest edition).

The pharmaceutical preparations are made following conventional techniques of pharmaceutical chemistry involving such steps as mixing, granulating and compressing, when necessary for tablet forms, or mixing, filling and dissolving the ingredients, as appropriate, to give the desired products for oral, parenteral, topical, transdermal, intravaginal, intrapenile, intranasal, intrabronchial, intracranial, intraocular, intraaural and rectal administration. The pharmaceutical compositions may also contain minor amounts of nontoxic auxiliary substances such as wetting or emulsifying agents, pH buffering agents and so forth.

The present invention may be used in the treatment of any of a number of animal genera and species, and are equally applicable in the practice of human or veterinary medicine. Thus, the pharmaceutical compositions can be used to treat domestic and commercial animals, including birds and more preferably mammals, most preferably humans.

The term “systemic administration” refers to administration of a composition such as the peptides described herein, in a manner that results in the introduction of the composition into the subject's circulatory system or otherwise permits its spread throughout the body, such as intravenous (i.v.) injection or infusion. “Regional” administration refers to administration into a specific, and somewhat more limited, anatomical space, such as inhalation or instillation in the lung, the preferred route, intraperitoneal, intrathecal, subdural, or to a specific organ. Other examples include intranasal, which is one route that corresponds to instillation in the lungs, intrabronchial, intra-aural or intraocular, etc. The term “local administration” refers to administration of a composition or drug into a limited, or circumscribed, anatomic space, such as subcutaneous (s.c.) injections, intramuscular (i.m.) injections. One of skill in the art would understand that local administration or regional administration often also result in entry of a composition into the circulatory system, i.e., so that s.c. or i.m. are also routes for systemic administration. Instillable, injectable or infusible preparations can be prepared in conventional forms, either as solutions or suspensions, solid forms suitable for solution or suspension in liquid prior to injection or infusion, or as emulsions. Though the preferred regional routes of administration are into the lungs, the pharmaceutical composition may be administered systemically or topically or transdermally either separately from, or concurrently with, instillation into the lungs.

Other pharmaceutically acceptable carriers for compositions of the present invention are liposomes, pharmaceutical compositions in which the active polypeptide is contained either dispersed or variously present in corpuscles consisting of aqueous concentric layers adherent to lipidic layers. The active polypeptide is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

The therapeutic dosage administered is an amount which is therapeutically effective, as is known to or readily ascertainable by those skilled in the art. The dose is also dependent upon the age, health, and weight of the recipient, kind of concurrent treatment(s), if any, the frequency of treatment, and the nature of the effect desired.

### Therapeutic Methods

The methods of this invention may be used to treat lung conditions or inflammatory lung diseases such as COPD/emphysema, severe asthma, al anti-trypsin deficiency, cystic fibrosis, bronchiectasis, sarcoidosis, bronchiolitis obliterans, transplant rejection including that resulting from allograft fibrogenesis in a subject in need thereof.

The term “treating” is defined broadly to include, at least the following: inhibit, reduce, ameliorate, prevent, reduce the occurrence or recurrence, including the frequency and/or time to recurrence, or the severity of symptoms of the disease or condition being treated or prevented. This may occur as a result of inhibiting epithelial cell death, inhibiting fibroblast proliferation, any of the other biological or biochemical mechanisms such as telomere shortening that is disclosed herein as being associated with or responsible for the disease being treated.

By “block,” “reduce,” “attenuate” or “decrease” is also meant a reduction in a measurement of a physiological effect (e.g., telomere or cilia shortening, senescence, apoptosis, protein expression, cilia disassembly or ciliopathy, lung inflammation, mucus hypersecretion, etc.). Examples of proteins whose expression may be measured include Muc5Ac/M5Ac, IL-17A, beta-galactosidase, [activated] caspase-3, miR-34a, and tubulin. Expression may be quantitated, for example, by real-time PCR/qPCR, Western blot, immunohistochemistry (IHC), and/or fluorescence activated cell sorting (FACS). Other physiological effects can be examined by microscope, such as by counting cells or measuring cilia length.

Likewise, by “improve” or “increase” is meant an increase in measurement of one of these effects.

An increase or decrease may be considered significant if it is at least a 2%, 3%, 5%, 10%, 15%, 20%, 25%, 30%, 40%, 50%, 60%, 70%, 80%, 90% or 95% increase or decrease in the measured physiological effect.

Expression or other physiological effects may be considered significant if they result in at least a 1.5-fold, 2-fold, 3-fold, 4-fold, 5-fold or 10-fold increase or decrease in the measured physiological effect.

The CSP7 peptide or peptide derivative or pharmaceutically acceptable salt thereof is preferably administered in the form of a pharmaceutical composition as described above.

Doses of the compound preferably include pharmaceutical dosage units comprising an effective amount of the peptide. Dosage unit form refers to physically discrete units suited as unitary dosages for a mammalian subject; each unit contains a predetermined quantity of active material calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects

By an effective amount is meant an amount sufficient to achieve a regional concentration or a steady state concentration in vivo which results in a measurable reduction in any relevant parameter of disease.

The amount of peptide or derivative selected, the precise disease or condition, the route of administration, the health and weight of the recipient, the existence of other concurrent treatment, if any, the frequency of treatment, the nature of the effect desired, and the judgment of the skilled practitioner.

A preferred single dose, given once daily for treating a subject, preferably a mammal, more preferably human who his suffering from or susceptible to IPF, COPD or emphysema resulting therefrom is between about 0.2 mg and about 25 mg, preferably between about 0.2 mg and about 10 mg, for example, via instillation (by inhalation). Such a dose can be administered daily for anywhere from about 3 days to one or more weeks. Chronic administration is also possible. The foregoing ranges are, however, suggestive, as the number of variables in an individual treatment regime is large, and considerable excursions from these preferred values are expected. One preferred dose used in a Phase 1b study of CSP7 for IPF underway is 2.5 and 5 mg/person/day. See, Clinical Trial NCT05954988.

https://clinicaltrials.gov/study/NCT05954988?term=NCT05954988&rank=1

An effective concentration of the active compound for inhibiting or preventing inhibiting apoptosis in vitro is in the range of about 0.5 nM to about 100 nM, more preferably from about 2 nM to about 20 nM. Effective doses and optimal dose ranges may be determined in vitro using the methods described herein.

Treatment of COPD may also include the use of known agents and methods that are helpful in treating or alleviating the symptoms of COPD. These include:
(a) Bronchodilators, usually administered using an inhaler to relax the airway smooth muscles, help relieve coughing and shortness of breath and make breathing easier. Both short-acting and long-acting bronchodilators are useful. Short-acting bronchodilators include albuterol (ProAir HFA, Ventolin HFA, others), levalbuterol (Xopenex HFA), and ipratropium (Atrovent). The long-acting bronchodilators include tiotropium (Spiriva), salmeterol (Serevent), formoterol (Foradil, Perforomist), arformoterol (Brovana), indacaterol (Arcapta) and aclidinium (Tudorza).
(b) Inhaled corticosteroids examples of which are Fluticasone (Flovent HFA, Flonase and budesonide (Pulmicort Flexhaler, Uceris, others) reduce airway inflammation and help prevent exacerbations and are thus particularly useful for people with frequent exacerbations of COPD. Some medications combine bronchodilators and inhaled steroids. Salmeterol and fluticasone (Advair) and formoterol and budesonide (Symbicort) are examples.
(c) Oral corticosteroids in short courses are useful for people with moderate or severe acute exacerbation and prevent further worsening of COPD.
(d) Phosphodiesterase-4 inhibitors are a newer type of drug approved for severe COPD and symptoms of chronic bronchitis. One example is roflumilast (Daliresp) which decreases airway inflammation and relaxes the airways.
(e) Theophylline may help improve breathing and prevent exacerbations.
(f) Antibiotics are used to treat respiratory infections, such as acute bronchitis, pneumonia and influenza, which can aggravate COPD symptoms. Azithromycin was shown to prevents exacerbations.
(g) Oxygen therapy if the patient's blood oxygen is too low. Oxygen may be used during activities or while sleeping, or continuously.
(h) Pulmonary rehabilitation programs generally combine education, exercise training, nutrition advice and counseling

Treatment of cystic fibrosis (CF) may also include the use of known agents and methods that are helpful in treating or alleviating the symptoms of CF. The goals for these treatments include preventing and controlling infections that occur in the lungs, removing and loosening mucus from the lungs, treating and preventing intestinal blockage, providing adequate nutrition. Useful drugs/medications and methods include (a) antibiotics to treat and prevent lung infections (b) anti-inflammatory medications to lessen swelling in the airways; (c) mucus-thinning drugs to help cough up the mucus which can improve lung function; (c) inhaled bronchodilators that can help keep airways open by relaxing muscles around your bronchial tubes; (d) oral pancreatic enzymes to help digestive tract absorb nutrients. CF due to certain gene mutations may benefit from certain newer drugs like ivacaftor (Kalydeco) which improves lung function and weight, and reduces the amount of salt in sweat. For a certain gene mutation Orkambi combines ivacaftor with lumacaftor which may improve lung function and reduce the risk of exacerbations. Chest physical therapy is used to loosening thick mucus in the lungs. Mechanical devices including a vibrating vest or a tube or mask can help loosen lung mucus.

Having now generally described the invention, the same will be more readily understood through reference to the following examples which are provided by way of illustration, and are not intended to be limiting of the present invention, unless specified. The examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods for Examples 1-7

### Treatment

All studies involving mice were performed according to the approved protocols under the guidelines of Institutional Animal Care and Use Committee. C57BL/6 mice of wild type (WT) as well as two knockout strains, PAI-1^{-/-} and uPA^{-/-} on this genetic background (Jackson Laboratory Bar Harbor, ME) were used.

### Cigarette Smoke

To analyze the effect of cigarette smoke, mice were exposed to passive smoke from 40 research cigarettes over a 2-hour period once (2 h) or twice (4 h) daily for 5 days/week for 20 weeks (˜90 mg/m³ total solid particulates) using a mechanical smoking chamber (Teague Enterprises, Davis, CA). Control mice were exposed to ambient air. At the 18^{th} week, peptide treatment at a dosage of 30 μg/20 g body weight was initiated and continued on a daily basis for the next 14 days. At the end of the experiment, the mice were euthanized and used for the experiments.

### Bleomycin (BLM)

To analyze the effect of BLM, mice were exposed to BLM (40 μg/20 g body weight) once per two week for 16 weeks. Control mice were exposed to normal saline. At 14^{th} week the peptide treatment at a dosage of 30 μg/20 g body weight was initiated and continued on a daily basis for the next 14 days. At the end of the experiment, the mice were euthanized and used for the experiments.

Isolation of AECs from Lungs

AECs were isolated from C57BL/6 mice of wild type (WT) as well as PAI-1^{-/-} and uPA^{-/-} knockouts following the method of Corti et al. (Am J Respir Cell Mol Biol, 1996, 14:309-15) with minor modifications. AECs from human lungs were isolated by a method described by the present inventors' group (Marudamuthu et al., Am J Pathol 2015; 185:55-68). The AECs were plated on plastic culture dishes pre-coated with anti-CD32 and anti-CD45 antibodies for 2 h at 37° C. The non-adherent cells were collected. The purities of AEC cell preparations exceeded 90%, based on lithium carbonate staining for inclusion bodies. The cells were grown in poly-L-lysine coated plates in growth-supplemented AEC culture medium (AEpiCM) (Sciencell, Carlsbad, CA, USA) at 37° C. in an incubator supplied with 5% CO₂.

### Protein Analysis by Western Blotting

Changes in SP-C(cat. no. sc-13979; Santa Cruz Biotechnology, Santa Cruz, CA), p53 (cat. no. sc-6243; Santa Cruz), serine 15 phosphorylated p53 (p53^{S15P}, cat. no. 9284; CellSignaling Technology, Beverly, MA), lysine 379-acetylated p53 (p53^{Ac}, cat. no. 2570; CellSignaling Technology), caspase-3 (cat. no. ab32351; Abcam, Cambridge, MA), cleaved caspase-3 (cat. no. 9661; CellSignaling Technology), PAI-1 (cat. no. ab66705; Abcam) and β-actin (cat. no. 3700; CellSignaling Technology) expression levels were assessed by Western blotting of AEC-lysates using specific antibodies and enhanced chemiluminescence (ECL) (Thermo, Rockford, IL) detection as described previously by the present inventor's group (Shetty S et al., Am J Respir Cell Mol Biol 2012; 47:474-83).

### Detection of Telomerase Activity

Telomerase activity was detected using a PCR-based telomeric repeat amplification protocol (“TRAP”) method using the TRAPeze Telomerase Detection Kit® (Intergen, Purchase, NY, USA). Briefly, the cells were in lysed in CHAPS lysis buffer and quantified by BCA method, and equal quantity of the protein samples was combined with the reaction mix in RNase-free PCR tubes. The PCR amplification was then performed according to the protocol. The final PCR product was loaded onto a 12.5% non-denaturing PAGE gel. Following electrophoresis, the gel was stained with ethidium bromide, and documented using a gel-doc unit (Bio-Rad Laboratories). The relative quantities of telomerase activity for each sample were calculated according to the instructions provided in the kit-protocol.

### Measurement of Terminal Restriction Fragment (Telomeric) Length

For determination of telomeric length the TeloTTAGGG Telomere Length Assay Kit® (Roche Diagnostics GmbH) was used. Briefly, genomic DNA was isolated and digested with Hinf1/Rsa. The digested DNA fragments were then separated by electrophoresis on agarose gel followed by Southern blot transfer. The membrane was then hybridized with a telomere specific digoxigenin (DIG)-labelled probe, incubated with anti-DIG alkaline phosphatase, and documented with chemiluminescence detection in gel-doc unit (Bio-Rad Laboratories). Telomeric length was identified by comparing with the pre-labelled molecular weight marker. The relative telomere length was calculated according to the manufacturer's protocol.

Relative telomere length was also analyzed by qPCR analysis of the genomic DNA as described by Callicott R J et al., (Comp Med 2006; 56:17-22) and Cawthon RM (Nucleic Acids Res 2002; 30:e47-e47). The 36B34 gene was served as the control. The primer sequences are provided in the Table 1.

TABLE 1
List of primer sequences used for gene expression analysis by qPCR

| Target | SEQ ID NO: | Sequence (5′-3′) |
|---|---|---|
| miR-34a | FWD: 14 | FWD: TGGCAGTGTCTTAGCTGGTTGT |
| | RV: 15 | RV: GTGCAGCACTTCTAGGGCAG |
| U6 snRNA | FWD: 16 | FWD: CGCTTCGGCAGCACATATAC |
| | RV: 17 | RV: TTCACGAATTTGCGTGTCAT |
| Human | FWD: 18 | FWD: GGTTTTTGAGGGTGAGGGTGAGGGTGAGGGTGAGGGT |
| telomere | RV: 19 | RV: TCCCGACTATCCCTATCCCTATCCCTATCCCTATCCCTA |
| specific | | |
| Human | FWD: 20 | FWD: CAGCAAGTGGGAAGGTGTAATCC |
| 36B4u | RV: 21 | RV: CCCATTCTATCATCAACGGGTACAA |
| specific | | |
| Mouse | FWD: 22 | FWD: CGGTTTGTTTGGGTTTGGGTTTGGGTTTGGGTTTGGGTT |
| telomere | RV: 23 | RV: GGCTTGCCTTACCCTTACCCTTACCCTTACCCTTACCCT |
| specific | | |
| Mouse | FWD: 24 | FWD: ACTGGTCTAGGACCCGAGAAG |
| 36B4u | RV: 25 | RV: TCAATGGTGCCTCTGGAGATT |
| specific | | |

FWD is forward and RV is reverse

mRNA Quantitation by Real-Time qPCR

Total RNA was isolated from AECs using TRI reagent and reverse transcribed using impromII Reverse transcription Kit® (Promega, WI). The levels of the mRNAs were quantitated using an aliquot of reverse transcribed total RNA and gene-specific primers (Table 1) by real-time PCR as described earlier (Shetty et al., supra; Shetty et al., J Biol Chem 2008; 283:19570-80).

### Statistical Analysis

The statistical significance of differences between experimental values were analyzed by one-way ANOVA followed by Tukey's post-hoc test using GraphPad Prism 4.0 software (GraphPad Software Inc., San Diego, CA).

### Example 2

### Telomere Shortening in IPF and COPD Patients

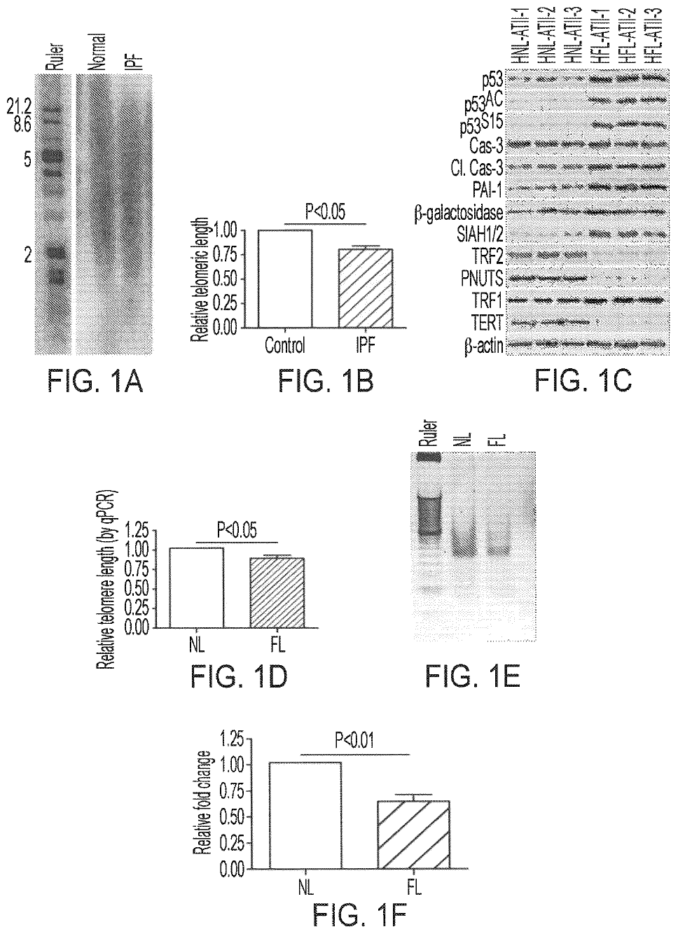

Shortening of the telomere was observed in ATII cells of both IPF and COPD patients. TeloTTAGGG assay showed a significant reduction in ATII-telomere length in IPF patients (FIGS. 1A-1F), which was substantiated by the qPCR (FIGS. 1A-1F). The protein expression analyzed by Western blot showed an increase in p53 expression, and p53 activation by acetylation as well as by serine 15 phosphorylation. Activated caspase-3 expression was also increased, implying an increase in apoptosis of ATII cells. Increase in β-galactosidase expression points to the possible increase in a senescence response in the ATII cells.

The expression of SIAH-1, a p53-inducible E3 ubiquitin ligase, known to down regulate the telomere repeat binding factor 2 (TRF2) expression, was also increased.

Downregulation of telomerase reverse transcriptase (TERT) was observed, and was correlated with the TRF2 expression.

Upregulation in the expression of TRF1 was observed; TRF1 is known to suppress the expression of TERT enzyme. The TERT enzyme activity was also significantly downregulated when analyzed by the TRAPeze enzyme assay method. Immunohistochemical analysis has shown that expression of TRF2 and TERT are downregulated, whereas p53 is upregulated in the IPF lung sections.

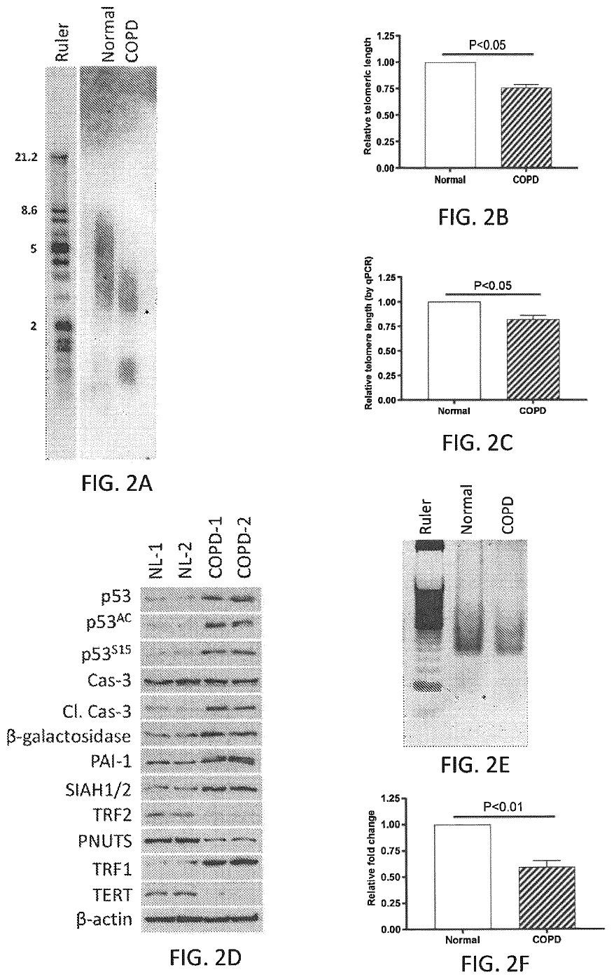

The ATII cells from the COPD patients has also shown a similar pattern of telomere shortening to that of the IPF patients. Telomere length analysis by TeloTAGGG assay and qPCR has shown significant reduction in telomere length (FIGS. 2A-2F). Protein expression of p53, and p53 activation was also observed with subsequent activation of caspase-3 pointing to the increase in apoptosis in ATII cells. Increase in β-galactosidase expression is pointing to the possible increase in the senescence that ATII cells are undergoing.

Up regulation of the SIAH-1 was observed that might have resulted in the inhibition of TRF2, and subsequent downregulation of the TERT expression observed. In addition, TERT enzyme activity shown significant reduction in ATII cells of COPD patients. Further, the lung sections, when analyzed by immunohistochemistry, has shown downregulation in TRF2 expression, while the p53 and1 PAI-1 shown increase in their expression.

### Example 3

### Cigarette Smoke and BLM Reduced Telomere in ATII Cells of WT Mice

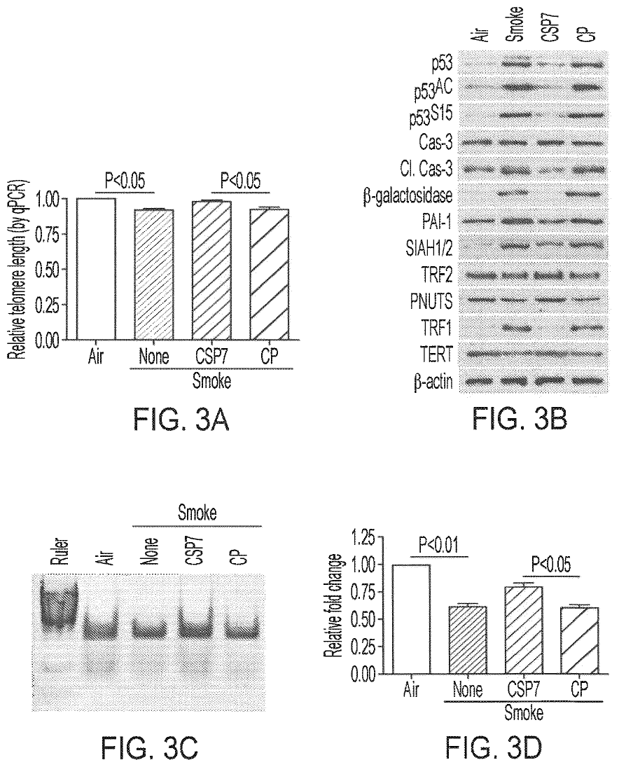

The ATII cells from WT mice treated with 20 weeks of smoke also showed a significant reduction in telomere length when analyzed by qPCR (FIGS. 3A-3D), though the extent of the telomere reduction was less severe than that observed in COPD and IPF patients.

Mice treated with the peptide CSP7 showed a significant resistance in telomere shortening when compared with that of the group received the control peptide CP.

Increased protein expression of p53, cleaved caspase-3 and β-galactosidase was observed, indicating progressive ATII cell death.

ATII cells from the CSP7-treated group showed a significant decrease in p53, cleaved caspase-3 and β-galactosidase expression versus the control CP-treated group. Downregulation of SIAH-1 was significantly more in CSP7 group compared to the control CP group.

The CSP7-treated group also showed restoration of TRF2 and TERT expression. The enzyme activity of TERT in CSP7-treated group was significantly higher than that of the control CP group.

Immunohistochemical analysis of lung sections also showed restoration of TRF2 expression with reduction in p53 and PAI-1 expression in the CSP7-treated group.

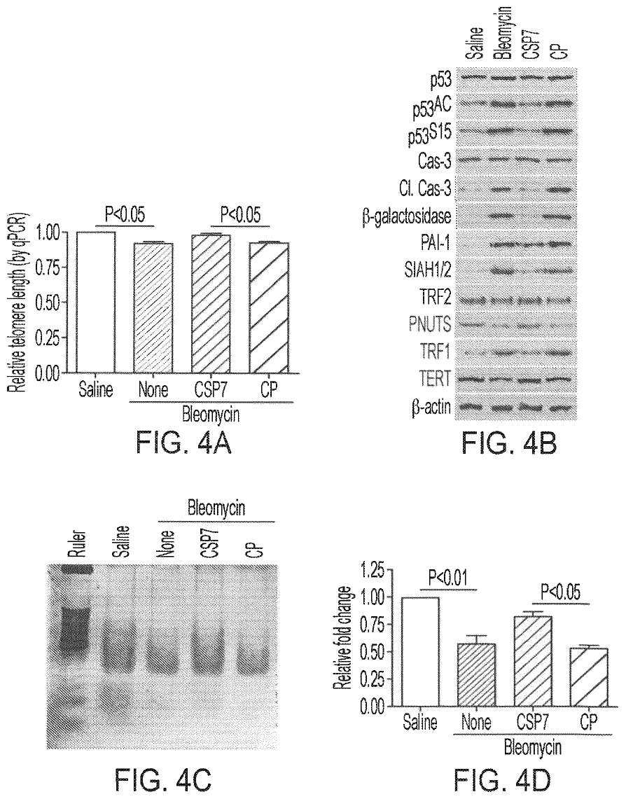

A similar pattern of expression was observed in WT mice whose lungs were damaged by repeated doses of BLM (See FIGS. 4A-4D).

### Example 4

### Resistance to Telomere Shortening in Mice Lacking miR-34a

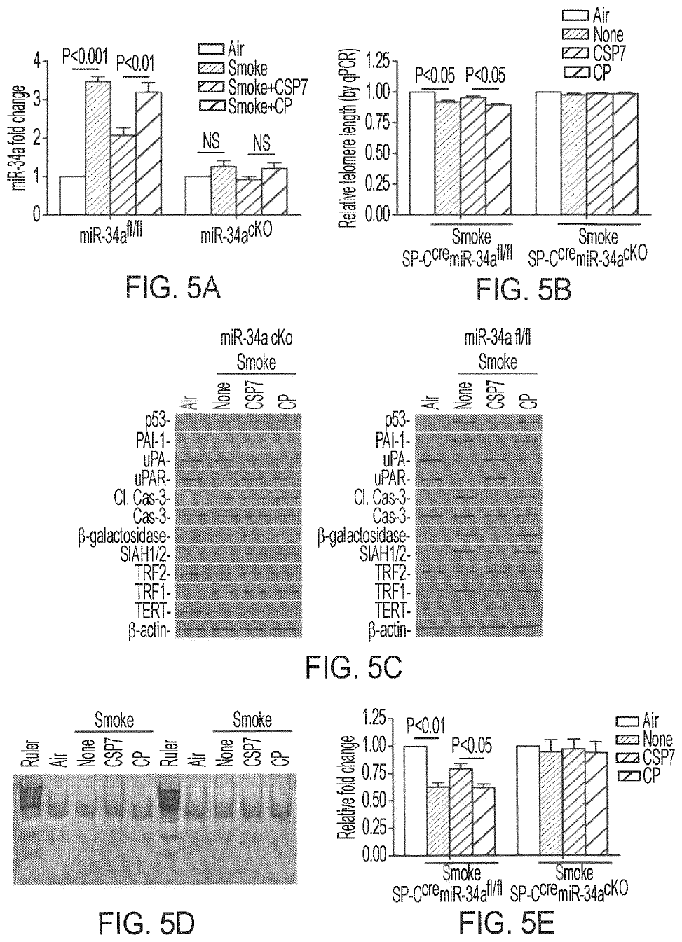

Mice deficient in miR-34a expression in ATII cells, by the CRE gene expression regulated by the SP-C promoter, (miR-34a^{cKO}) shown resistance towards telomere shortening when subjected to 20 week of smoke exposure (FIGS. 5A-5E). Control mice, which had retained the miR-34a gene (miR-34a^{fl/fl}) were susceptible to telomere shortening by smoke treatment.

CSP7 peptide protected from telomere shortening in miR-34a^{fl/fl} mice similar to that observed in WT mice.

Telomere length analyzed by qPCR, did not show any significant reduction in mice lacking miR-34a expression.

Expression of p53, PAI-1 and active caspase-3 were not upregulated and uPA and uPAR (uPA receptor) expression was not downregulated in miR-34a^{cKO} group in comparison with miR-34a^{fl/fl} group.

Expression of TERT as well as the telomerase enzyme activity was not affected by smoke exposure in miR-34a^{cKO}, whereas significant downregulation in telomerase activity was observed miR-34a^{fl/fl} mice.

CSP7 peptide treatment did not upregulate telomerase activity in miR-34a^{cKO} whereas significant upregulation was observed in miR-34a^{fl/fl} mice receiving CSP7.

### Example 5

### Telomere Shortening in uPA^{-/-} Mice Exposed to Smoke or BLM

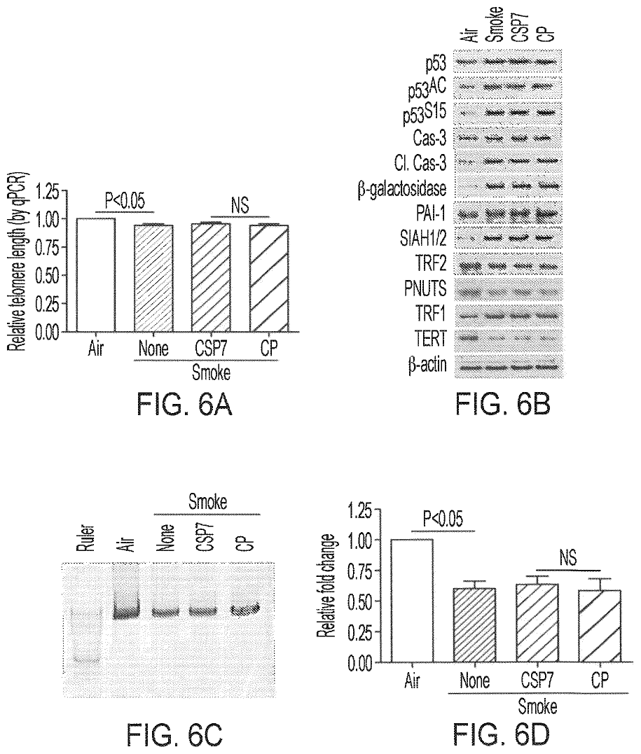

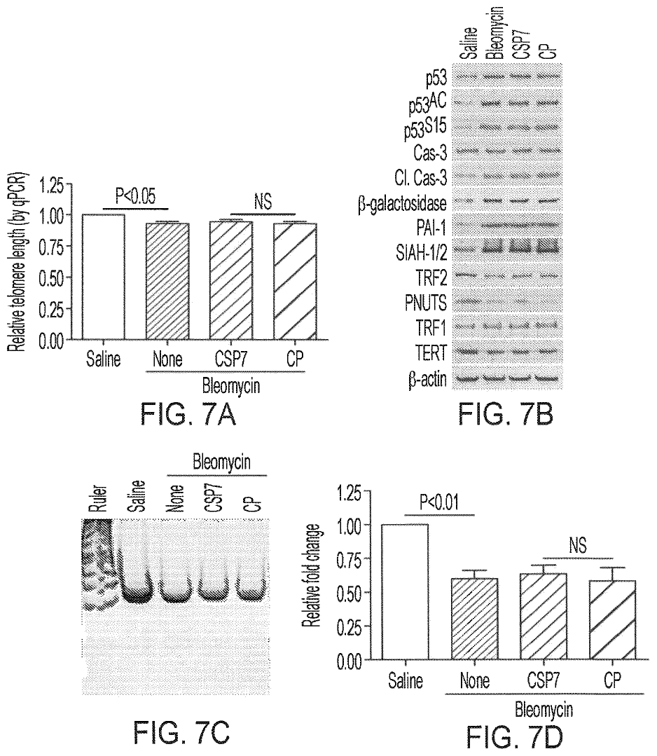

uPA^{-/-} mice exposed to 20 weeks of smoke (FIGS. 6A-6D) or repeated doses of BLM (FIGS. 7A-7D) and then treated with the CSP7 did not show significant difference in telomere shortening compared to the group treated with the control peptide CP.

Treatment with the CSP7 peptide failed to inhibit activation of p53, caspase-3 as well as β-galactosidase when analyzed by Western blotting for protein expression.

There was no significant change in the TRF1, TRF2 and TERT expression between the CSP7- and CP-treated groups.

CSP7 did not restore the telomerase enzyme activity when analyzed by the TRAPeze method.

### Example 6

### Telomere Shortening in PAI-1^{-/-} Mice Exposed to Smoke or BLM

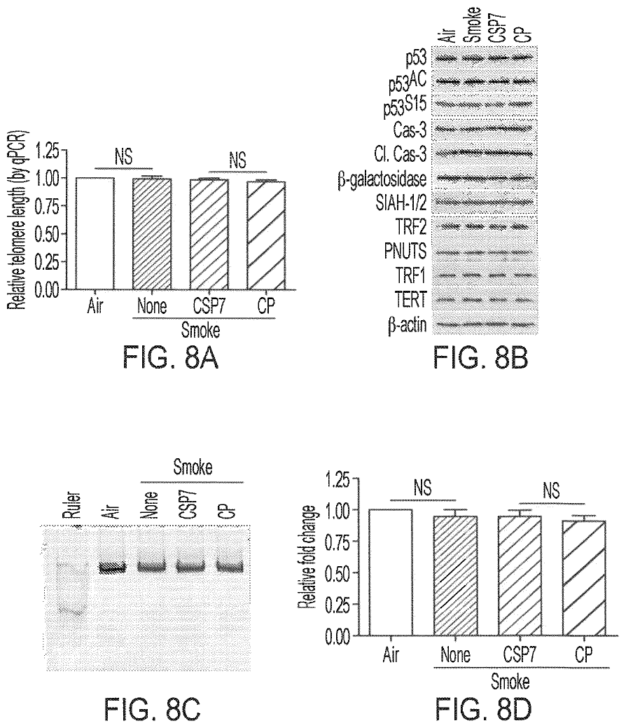

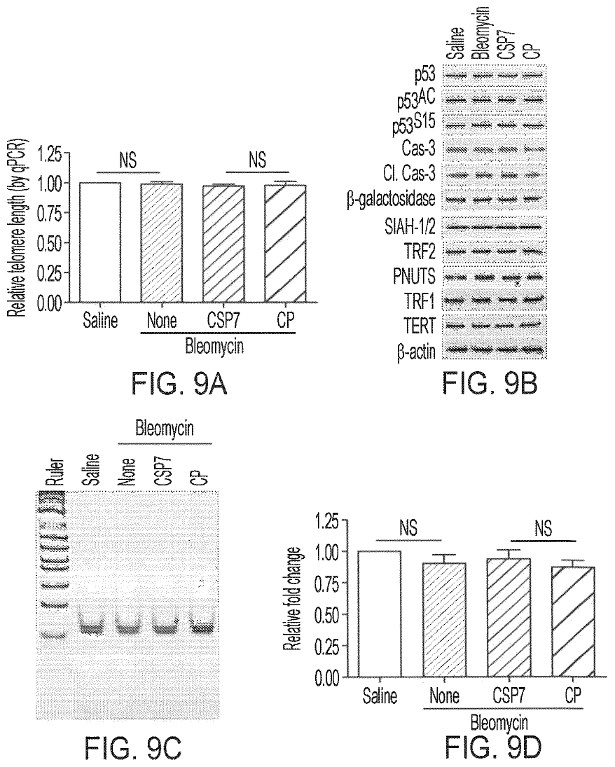

PAI-1^{-/-} mice exposed to 20 weeks of smoke (FIGS. 8A-8D) or repeated doses of BLM (FIGS. 9A-9D) were resistant to telomere shortening. There was no significant change in telomere length in the control groups as well as in those received the CSP7 and CP peptides.

Shortening of telomeres analyzed by qPCR did not show significant changes compared to control groups.

These mice also resisted the activation of p53, caspase-3 and β-galactosidase when analyzed by Western blot for protein expression.

Proteins directly associated with the telomere from the treated group, TRF1, TRF2 and TERT, did not shown any significant changes compared to the control saline/air groups.

Examining telomerase enzyme activity (by TRAPeze assay) also showed that the PAI-1^{-/-} mice were resistant to telomere shortening as there were no significant changes.

### Example 7

### IL-17A-mediated n53-miR-34a Feed Forward Induction of PAI-1 Contributes to PTS and Bleomycin Lung Injury

The following summarize the results of these studies.
1. Chronic passive tobacco smoke (PTS) exposure induced accumulation of CD4- and CD8-positive T cells, IL-17A, macrophages and neutrophils in the lungs of WT mice, which was resisted by both p53- and PAI-1-deficient (KO) mice.
2. Treatment of PTS-exposed mice with CSP or CSP7 inhibited pulmonary influx of CD4- and CD8-positive T cells, macrophages and neutrophils.
3. Treatment of WT mice with CSP or CSP7 inhibited PTS-induced accumulation of IL-17A in the lungs.
4. Mice exposed to 20 weeks of PTS show increased lung volume indicating emphysema-like condition, which was significantly reduced following treatment of PTS exposed WT mice with either CSP or CSP7.
5. IL-17A treatment induced expression of p53 and PAI-1, and apoptosis in A₂Cs both in vitro and in vivo. Further, the process involved acetylation and serine phosphorylation of p53 proteins in A₂Cs.
6. CSP7 inhibited PTS or IL-17A exposure induced p53 and p53-mediated downstream induction of PAI-1 expression, and apoptosis in A₂Cs both in vivo and in vitro. The process involved inhibition of p53 acetylation through suppression of miR-34a expression and restoration of Sirt1 expression in A₂Cs.
7. IL-17A-deficient mice exposed to PTS resisted induction of p53 or downstream PAI-1 expression or apoptosis in A₂Cs.
8. PTS exposure of IL-17A-deficient mice failed to induce expression of miR-34a or acetylated and total p53 in A2ECs. IL-17A-deficient mice also resisted PTS exposure induced suppression of baseline Sirt1 expression.
9. Loss of miR-34a expression in A₂Cs prevented PTS induced acetylation of p53 and PAI-1 expression, apoptosis or senescence.
10. Overexpression of miR-34a in A₂Cs alone induced p53 expression and apoptosis.
11. A₂C-specific inhibition of miR-34a expression prevented PTS-induced suppression of Sirt1 expression.

### Discussion of Examples 2-6

(1) The miR-34a and p53 feedback loop is essential for lung inflammation and A₂Cs apoptosis during PTS and IL-17A induced lung injuries.
(2) Elevated miR-34a increased acetylated and total p53, and decreased Sirt1 in WT mice.
(3) Like PTS-induced lung injury, exposure to IL-17A alone upregulated p53, PAI-1 and Cav1 expression, and apoptosis and reduced Sirt1 in WT mice.
(4) IL-17A prevented binding of mdm2 and p53 proteins due to increased acetylation and serine phosphorylation of p53, which results in increased steady state p53 protein level.
(5) IL-17A increased PAI-1 through miR-34a-p53 feed forward induction
(6) CSP7 treatment reduced miR-34a leading to increased
   Sirt1,
   Sirt1-mediated deacetylation of p53 and
   mdm2-mediated degradation of p53.
(7) PTS increased IL-17A and IL-17A receptor, and influx of PMNs and macrophages, and CD4- and CD8-positive T-lymphocytes; these effects were reversed after treatment with CSP7.
(8) PTS and IL-17A failed to induce pulmonary PMN and macrophage accumulation in p53-and PAI-1-deficient mice, suggesting their importance in lung inflammation.
(9) Treatment of mice with IL-17A or Pre-miR-34 caused two-fold increase in total BAL cells. The percentage of PMN in total BAL cells of these treated mouse were 11.27% (IL-17A) and 53.48 (Pre-miR-34a)
(10) Treatment of bronchial epithelial cells with CSP7 inhibited TS-induced MUC5A gene expression indicating that CSP7 is effective against mucus hypersecretion associated with chronic TS exposure.

In conclusion, a mouse model of IL-17A-induced lung injury, as well as comparison of WT and IL-17A-deficient mice exposed to 20 weeks of PTS showed an essential role of IL-17A in PTS-induced chronic lung injury, a process that involves miR-34a-p53 feed forward induction and downstream PAI-1 expression.

### Example 7

### CSP7 Inhibits Aging and Age-Associated Diseases by Blocking Telomere Shortening and Mucin Hypersecretion, Inflammation and Acute Lung and Injury and Remodeling

CSP7 inhibits intermediaries affecting telomere shortening/dysfunction in A₂Cs. These effects suggest that CSP7 could be beneficial for treatment of emphysema and aging.

Inhibition of mucin hypersecretion and airway remodeling by CSP7 is useful for treatment of CF, COPD and other diseases associated with excess mucus.

CSP7 would also be used treat wood smoke or other smoke inhalation induced lung injury. CSP7 also be used for bronchopulmonary dysplasia (BPD), hyperoxia induced lung injury, ventilator induced lung injury, silica and other particulate matter induced lung injury and other conditions in which baseline expression of p53 and PAI-1 and lung cell senescence and apoptosis are increased in the lungs.

### Example 8

### Positive Effects of CSP/CSP7 on Lung Dysfunction in a Sepsis Model (Cecal Ligation Puncture, CLP)

This model was described in detail in publication of the present inventor and colleagues, Gao, R et al., Am. J. Physiol. Mol. Physiol, 2015, 308:L847-L853.

Sepsis is initiated and perpetuated by excessive production of inflammatory cytokines and chemokines, resulting in multiple organ failure and death. Lung dysfunction is associated with multiple organ failure during sepsis. Alveolar inflammation, fibrin deposition and alveolar type II cell (A₂C) apoptosis typify acute lung injury (ALI) due to sepsis.

There is no effective treatment to reverse ALI. The present inventors found, in mice with polymicrobial sepsis-induced ALI, that IL-17A induced p53 and apoptosis in A₂Cs, where p53 augmented PAI-1 and inhibited surfactant protein (SP-C) expression. According to the present invention. IL-17A-mediated increases in p53 and PAI-1 in A₂Cs, promote alveolar inflammation and A₂C apoptosis, which are central to sepsis-induced ALI.

While the 20mer peptide CSP was used in the studies noted below, based on results obtained thus far, it is fully expected that CSP7 will have the same effect and is a preferred agent for use in this setting.

1. Induction of p53 and p53-Mediated Downstream Induction of PAI-1 Expression by IL-17A Regulates Sepsis-Induced ALI and ATII Cell Apoptosis.

IL-17A expression increases in the lungs during sepsis-induced ALI, and augments p53 expression in A₂Cs. p53 induces A₂C PAI-1 mRNA and protein expression, with concurrent induction of miR-34a and reciprocal suppression of SP-C expression.

CSP blocks A₂C apoptosis, p53 expression and p53-mediated induction of PAI-1 and ALI via cell surface signaling that involves caveolin-1, Q 1-integrin and uPAR. Applicant's new data show that sepsis-induced ALI and A₂C apoptosis can be reversed by interrupting this pathway with CSP.

### 2. CSP Inhibits Pulmonary IL-17A Levels, A₂C p53 and PAI-1 Expression, and Apoptosis During Sepsis-Induced ALI.

WT mice were injected IP with vehicle, 1.5 mg/kg of CSP or control peptide (CP) 24 h after CLP injury. Sham-operated mice served as controls. Total RNA isolated from the lungs of these mice 72 h after CLP was quantitated for IL-17A mRNA by real-time PCR.

A₂Cs were isolated from the lungs of these mice 72 h after CLP, and immunoblotted to assess changes in PAI-1, p53 and caspase-3 activation. All were increased by 72 h during CLP injury.

Treatment with CSP suppressed sepsis induced IL-17A in the lung tissues with concurrent inhibition of A₂C apoptosis and p53 and PAI-1 expression.

### 3. Increased Interaction Between Caveolin-1 and Protein Phosphatase 2A Catalytic Subunit (PP2A-C) in A₂Cs In Vivo.

Mice were injected IP with CSP or control peptide (CP) 24 h after CLP injury. A₂Cs isolated from these mice 72 h after CLP injury were immunoblotted for caveolin-1 and β-actin. Lysates of A₂Cs from WT mice exposed to the above conditions were immunoprecipitated (IP) for PP2A-C and immunoblotted (IB) for caveolin-1 (Cav1) to assess their interaction.

Results showed that CLP injury induced caveolin-1 expression in WT mice, and CSP inhibited the A₂C caveolin-1 interaction with PP2A-C.

These results indicate that CSP-mediated changes are associated with inhibition of the caveolin-1 interaction with PP2A-C, an ataxia telangiectasia mutated (ATM) kinase inhibitor that facilitates degradation of p53 by mdm2. This demonstrated a new, intricate link between p53-mediated induction of PAI-1 and apoptosis in A₂C after sepsis-induced ALI.

### 4. CSP Mitigates Lung Inflammation Through Inhibition of PMN Accumulation or Neutrophilia in WT Mice.

Mice treated with or without CSP or CP 24 h after CLP injury were euthanized 72 h later and. lung tissues subjected to H&E staining to assess changes in lung inflammation. Lung homogenates and BAL fluids were analyzed for myeloperoxidase (MPO) to access PMN accumulation.

Results showed that CSP significantly inhibited accumulation of PMN, confirming this aspect of CSP-mediated protection against sepsis-induced ALI.

5. CSP Inhibits Sepsis-Induced miR-34a Expression in Mouse Lung A₂Cs In Vivo.

miRNAs, a large group of conserved single stranded non-coding, abundant and short (˜21-25 nt) RNAs which suppress gene expression by targeting mRNAs for degradation or translation repression.

The present results suggest that both miR-34a levels and p53 acetylation are increased in A₂Cs after septic ALI.

CSP treatment of mice after CLP injury inhibited miR-34a by 7-fold in A₂Cs compared to those exposed to CLP or CLP+CP 3 days after injury.

These observations strongly suggest that CSP can reverse IL-17A-mediated induction of A₂C p53, PAI-1 expression, and apoptosis through inhibition of miR-34a.

6. CSP Inhibits CLP-Induced miR-34a Expression in Mouse Lung A2Cs In Vivo.

Mice were IP injected with CSP, CP or vehicle 24 h after CLP injury. Total RNA isolated from A₂Cs of WT mice with or without CLP, CLP+CSP or CLP+CP 72 h after CLP induced ALI was reverse transcribed and subjected to real time PCR.

miR-34a expression (after normalization for snRNA U6) was significantly reduced in the CSP-treatment group compared to sham-operated controls and peptide controls.

### 7. CSP Induced SP-C Expression by A₂Cs in Mice.

A₂Cs isolated from mice with CLP, CLP+CSP or CLP+CP were immunoblotted for SP-C, thyroid transcription factor-1 (TTF-1) and β-actin. TTF-1 controls transcription of SP-C.

CSP induced SP-C expression by A₂Cs in normal lungs. A₂Cs isolated from mice 72 h after IP injection with CSP or CP alone without CLP injury and the lysates were immunoblotted for changes in SP-C expression. Lysates of A₂Cs from uninjured (sham-operated) or CLP mice were also used for comparison.

The results indicated that that CSP-mediated induction of SP-C expression protects A₂Cs from apoptosis during sepsis-induced ALI.

Conclusions: Targeting of p53-mediated induction of PAI-1 and A₂C apoptosis to mitigate sepsis-induced ALI represents a promising novel interventional approach that is supported by the present inventor's recent publications and results described herein. The results further implicate the newly recognized contribution of increased IL-17A with induction of miR-34a, and reciprocal inhibition of SP-C to the outcome of ALI. CSP and CSP7 should reverse septic ALI in patients with sepsis. Thus CSP7 is also used to treat sepsis and streptomycin induced acute lung injury and fibrosis. Since about 40% of patients with ALI develop accelerated lung fibrosis, CSP7 is effective in treating fibrosis caused by Strep infection.

### Example 9

### Materials and Methods for Examples 10-18

### Cell Culture

Primary Airway Epithelial Cells (AECs); Normal, Human (ATCC® PCS-301-010™) and Primary Airway Epithelial Cells; COPD (ATCC® PCS-301-013™) were obtained from the ATCC and cultured in Airway Cell Basal Medium with glutamine, Extract P, HLL Supplement, and AEC Supplement. containing and 1% penicillin-streptomycin. The cells were maintained at 37° C. in a humidified atmosphere at 5% CO₂. All media, supplements, and antibiotics were purchased from ATCC.

### Preparation of TSE for In Vitro Experiments

Research cigarettes 2R4F were purchased from the Tobacco Health Research University of Kentucky (Lexington, KY). TSE extracts were prepared by burning research cigarettes in a side arm flask and the smoke generated was bubbled into phosphate-buffered saline at room temperature through an attached peristaltic pump as described earlier (Bhandary et al PloS One 10: e0123187, 2015) Tiwari et al. Am J Physiol Lung Cell Mol Physiol. 310:L496-506, 2016. An absorbance of 1.0 at 230 nm is considered 100%. TSE extract was filter sterilized by passing it through a 0.2-μm filter.

### Passive TSE Exposure of Mice

Wild-type (WT) and p53- and PAI-1-deficient mice of C57BL/6 background were bred in the inventor's facilities or were purchased from Jackson Laboratories. These mice were exposed to passive TSE from 40 research cigarettes over a 2 hour period 5 days/week for 20 weeks (^{˜}90 mg/m3 total solid particulates) by using a mechanical smoking chamber (Teague Enterprises, Davis, CA). Control mice were exposed to ambient air. Four weeks after initiation of passive TSE exposure, the mice were administered an intraperitoneal injection of CSP7 or scrambled control peptide (CP) (18.75 mg/kg body wt) once a week for 4 weeks (Marudamuthu et al. Am J Pathol 185: 55-68, 2015); Tiwari et al., supra). Mice were killed, and their lungs were used for further analyses (Bhandary et al., supra).

### Peptide Preparation

CSP7 and CP were dissolved in DMSO and diluted in HBSS for working concentration of 300 □g/2 ml. These peptides were used for the treatment of COPD in vitro and IP injection of mice. For nasal insufflation, the peptides were formulated as follows: 0.579 mg/ml of CSP7/CP was added to 15.456 mg/ml of lactose monohydrate in phosphate buffered saline and the pH was adjusted to 8.4 to give a clear solution. The solution was filtered through a 0.22-micron syringe filter (Bhandary et al. supra; Tiwari et al., supra).

### HE Staining

Three lung tissue sections were randomly Selected from each group. All sections were dewaxed with xylene and hydrated with ethanol. Sections were stained by Hematoxylin and differentiated by hydrochloric acid-ethanol solution. Next, they were counterstained by Eosin, and finally dehydrated by ethanol.

### Immunofluorescence Staining

Airway epithelial cells (AECs) were plated on sterilized coverslips. After treatment, the cells were washed with phosphate buffered saline 3 times, fixed with 4% paraformaldehyde for 20 min, permeabilized with 0.1% Triton X-100 (Biosharp) for 20 min, blocked with 3% bovine serum albumin for 1 h, and then incubated overnight with primary antibody. Subsequently, the cells were stained with FITC-conjugated secondary antibody (Alexis fluor). DAPI was used for nucleus staining (blue). Confocal images of HBE cells were captured with an inverted microscope (Carl Zeiss, Göttingen, Germany) using the Zeiss LSM program.

### Protein Isolation and Western Blot Assay

The cells were lysed with RIPA buffer (Pierce, USA) containing protease inhibitor cocktail (Roche, Germany) and phosphatase inhibitor cocktail (Sigma-Aldrich, USA) on ice for 30 min. After centrifugation at 12,000×g and 4° C. for 20 min, the supernatants were collected. The protein concentrations were determined using the BCA protein assay kit (Pierce, USA). Cell lysates were mixed with 5×SDS-PAGE sample buffer and boiled for 5 min. Thirty micrograms of protein was subjected to 10% SDS-PAGE electrophoresis and transferred to nitrocellulose membranes. The membranes were blocked with 5% milk and then incubated at 4° C. for 16 h with the following diluted primary antibodies (Tiwari et al., supra). The blots were then washed and probed with horseradish peroxidase-conjugated secondary IgG antibodies. The bound antibodies were visualized using SuperSignal™ Maximum Sensitivity Substrate (Thermo Fisher Scientific, USA). For normalization, the membranes were stripped with Restore Western blot stripping buffer and incubated with the following primary antibodies: anti-ERK (1:1000), anti-MUC5AC (1:1000), anti-HDAC6(1:1000), anti-SPDEF(1:1000), anti-FOXA2(1:1000), anti-FOXA3 (1:1000) anti-LC3(1:1000), anti-Beclin1(1:1000), anti-p62(1:1000), anti-p53(1:1000), anti-PAI-1(1:1000) and anti-GAPDH (1:1000).

### Measurement of Pulmonary Function Tests

Pulmonary function tests were performed immediately before CT imaging and before mice were killed, as previously described (DeCologne N et al., Eur Respir J. 35:176-85, 2010). Briefly, mice were anesthetized with a ketamine/xylazine mixture. Anesthetized mice were intubated by inserting a sterile, 20-gauge intravenous cannula through the vocal cords into the trachea. Elastance, compliance, and total lung resistance were then measured (SCIREQ, Tempe, AZ). The “snapshot perturbation method” was used to study lung function in the CBB injury model. This method measures total lung resistance, compliance, and elastance of the entire respiratory system. Increased total lung resistance in the CBB model may reflect lung contraction associated with pleural rind formation with concurrent distortion of the airways. The flexiVent was set to a tidal volume of 30 ml/kg at a frequency of 150 breaths/min against 2-3 cm H₂O positive end-expiratory pressure, according to manufacturer's specifications. The mice were maintained under anesthesia using isofluorane throughout the pulmonary function testing.

### CT Scans and Measurements of Lung Volume

After ketamine/xylazine injection, mice were anesthetized further using an isoflurane/02 mixture to ensure that mice remained deeply anesthetized and to minimize spontaneous breaths. The Explore Locus Micro-CT Scanner (General Electric, GE Healthcare, Wauwatosa, WI) was used for CT imaging. CT scans were performed during full inspiration and at a resolution of 93 mm. Lung volumes were calculated from lung renditions collected at full inspiration. Microview software was used to analyze lung volumes and render three-dimensional images (Tucker T A et al., Am J Respir Cell Mol Biol. 50:316-27, 2014).

PP2A Activity Determination and PP2A Inhibitors PP2A activity was determined using the Millipore PP2A activity assay (17-313; Millipore) (Nath S, et al., Am J Respir Cell Mol Biol. 59:695-705, 2018 December).

### Isolation of Mouse Tracheobronchial Epithelial Cells (MTEC)

Mice were euthanized, after that spray the animal carcasses with 70% ethanol solution to sterilize the yield. With clean surgical scissors and scalpel, skin around the tracheal area was removed, exposing the trachea. The abdomen was opened by cutting along the sternum, and the rib cage was removed exposing tissue up to the end of the trachea. Tracheas were excised and placed into a 50 mL conical tube containing 30 mL Ham's F12 media=antibiotics, on ice. In a sterile lamellar flow hood, tracheal tissue was transferred to a sterile 100 mm Petri dish containing 10 mL Ham's F12 media+antibiotics. Connective tissue was gently dissected with sterile forceps and surgical scissors. Tracheal tissue was placed in a new 100 mm Petri dish containing 10 mL Ham's F12 media+antibiotics to rinse. Tracheas were cut along the vertical axis to expose the lumen. Tracheas were transferred to a 50 mL tube containing 10 mL 0.15% Pronase solution and incubated overnight at 4° C.

DNAse I solution. To 18 mL of Ham's F12 Media+antibiotics, 2 mL of a 10 mg/mL Bovine Serum Albumin (BSA) stock solution was added, along with and 10 mg of crude pancreatic DNAse I. 1 mL aliquots were stored at −20° C. (thawed on ice before use).
Ham's F12 medium with antibiotics with 20% fetal bovine serum (FBS). To 200 mL Ham's F12 basal media (Invitrogen) 50 mL heat inactivated FBS, 2.5 mL of a 100× Penicillin/Streptomycin solution, and 250 μL of a 1000× Fungizone solution were added. MTEC Basic Medium containing antibiotics. To 475.5 mL DMEM/F12 basic media (Cellgro) 7.5 mL 1 M HEPES, 10 mL of 200 mM glutamine, 2 mL of a 7.5% NaHCO₃, 5 mL of a 100× penicillin/streptomycin, 500 μL of 1000× Fungizone were added. For MTEC medium/10% FBS. 5 mL heat inactivated FBS were added to 45 mL of MTEC basic medium+antibiotics, 10 mL Ham's F12 media containing 20% FBS and antibiotics were added to the tube and rocked 12 times.

### Trachea Preparation

Tracheas were removed from the Pronase solution, setting aside this solution on ice and transferred a conical tube containing Ham's F12; the tube was inverted 12 times and this process repeated twice. Pronase solution was combined with the three supernatants, and remaining tissue was discarded. Tubes were centrifuged at 1400 rpm for 10 min at 4° C., and supernatant discarded. The pellet was gently resuspended in 1 mL DNAse solution (100-200 μL/trachea) and incubated for 5 min on ice and then centrifuged at 1400 rpm for 5 min at 4° C., and the supernatant discarded. The cell pellet was resuspended in 8 mL MTEC medium with 10% FBS. Cell suspensions were plated and incubated at 37° C. in an atmosphere of 95% air, 5% CO₂ for 5 hrs. Cell suspension were collected from plates and the plates rinsed twice with 4 mL MTEC+10% FBS. Cell suspension and washes were pooled in a 50 mL conical centrifuge tube. 1 mL was set aside for cytospin and cell counting. Tubes were centrifuged in a tabletop centrifuge for 5 min at 5,000 rpm. 500 μL were removed and the pellet resuspended in remaining supernatant. 100 μL was taken for viable cell counting. 4 aliquots of 100 μL were set aside for cytospin analysis. The remaining 15 mL cell suspension were centrifuged at 1400 rpm, at 4° C. for 10 min (Lam H C, et al., J Vis Exp 48:2513, 2011).

Treatment of Human Lung Tissues with CSP7

Lung tissues from control subjects and patients with COPD were treated with or without CSP7 for 72 h ex vivo or in vitro. Lung homogenates were analyzed for immunoblot and Real time PCR.

### Statistical Analysis

All results are representative of at least five independent experiments which were quantified and plotted as the mean±standard deviation. Student's t-test was used for evaluating statistical significance of differences between experimental groups. Further, non-parametric tests for analysis amongst groups were also done using one-way ANOVA Kruskal-Wallis test, with Dunn's multiple group comparison tests as appropriate. Statistical analyses were done using the SPSS Statistics 20 (IBM SPSS software, version: 20.0, Chicago, IL, USA) and GraphPad Prism 5 (GraphPad Software, Inc., San Diego, CA, USA). The P value was defined as follows: not significant (ns): P>0.05. P values of *P<0.05; **P<0.01; ***P<0.001 and ****P<0.0001 were considered statistically significant.

### Example 10

### Airway Epithelial Cells (AECs) from Subjects with COPD Shows Differential Expression of MUC5AC, FOXA2, FOXA3, HDAC6, and SPDEF

The main problem in emphysema is that the walls of the air sacs are destroyed. The inner walls of the sacs weaken and burst, creating one large space for holding air instead of many small ones. Representative H&E staining of tissue sections of Normal (NL) and COPD and alongside bar graph showing increased mean linear intercept (MLI) observed in lung tissue sections. The mean linear intercept (chord) length (Lm) is a useful parameter of peripheral lung structure as it describes the mean free distance in the air spaces (FIG. 11A). Patients with GOLD 4 COPD had an increase in mean linear intercept compared with Normal (NL). MUC5AC as a deleterious and dispensable glycoprotein component of airway mucus. Consistent with prior studies of airway mucin gene expression in humans. In an attempt, the inventor examined whether on MUC5AC, a secreted-polymeric mucin, as it is highly expressed by airway surface mucus producing cells in COPD patients. There was an upsurge in MUC5AC and HDAC6 expression in COPD lung as compared to NL. Mucous cell metaplasia (MCM) is associated with decreased expression of the transcription factor FOXA2 and increased expression of the related transcription factor FOXA3 in COPD Patients.

Nuclear FOXA2 protein expression in airway epithelial cells was reduced during mucous metaplasia. These results indicate that the FOXA2 transgene was expressed in airway epithelial cells and that transgene expression persisted after allergen challenge.

FOXA3 affects mucus production, which might be involved in other aspects of allergic airway disease. Intense expression of FOXA3 was detected in airway goblet cells in tissue from patients with COPD in immunoblot. Histological analysis of COPD patient lung sections showed increased MUC5AC staining as compared to NL.

Immunoblot and Real time PCR were performed to investigate the expression of mucin hypersecretion related genes. The results revealed that decrease of FOXA2 and acetylated α Tubulin (Ac-Tub) levels and increased expression of MUC5AC, HDAC6, SPDEF, and FOXA3 in AECs isolated from human COPD patients compared to their basal expressions in NL AECs (FIGS. 11B-11C). Also observed were elevation in Cav1, PAI-1, p53 expression in COPD AECs as compared to NL (FIG. 11B).

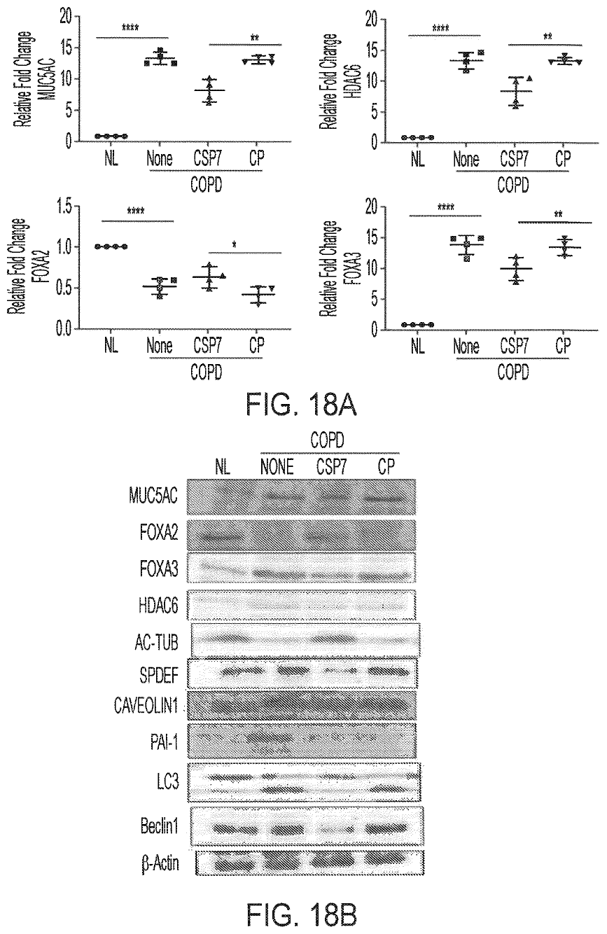

Human NL (n=4) tissues from control donors and COPD lung (n=4) tissues were treated with PBS or 10 μM CSP or CSP7 ex vivo in dishes for 72 h. Bar graphs showing increased expression of MUC5AC, HDAC6, Caveolin1 and FOXA3 mRNA, and decreased expression of FOXA2 mRNA was analysed by QPCR (FIG. 18A). Simultaneously, western Blot images shows increased MUC5Ac, HDAC6, SPDEF, and decrease in Acetylated Tubulin and FOXA2 level in the COPD lung homogenates being reverse by treatment with CSP or CSP7 (FIG. 18B).

### Example 11

### Histone Deacetylase 6 (HDAC6) Interceded Selective Autophagy Regulates COPD-Associated Ciliary Dysfunction

Autophagy refers to a dynamic process by which cytoplasmic organelles and proteins are sequestered into autophagosomes that subsequently fuse with lysosomes, leading to the degradation of cargo by lysosomal hydrolases (Mizushima N et al., Cell. 147:728-41, 2011; Yang Z et al., Cell 132:27-42, 2008)

The roles of HDAC6 in motile cilia of the airways, in cellular responses to TSE exposure, and in COPD pathogenesis have not been clarified. Therefore, the expression of HDAC6 in lung tissue obtained from COPD patients was assessed. HDAC6 expression was upregulated in lung tissue of COPD patients; increased HDAC6-positive staining was detected in airway epithelia of COPD patients relative to control (NL) subjects. HDAC6 has been shown to regulate primary cilia resorption in response to extracellular stress (Prodromou et al., J Cell Sci. 125(pt 18):4297-4305, 2012) as well as the autophagic pathway through autophagosome-lysosome fusion (Lee et al., EMBO J. 29:969-80, 2010). Moreover, ciliophagy, an HDAC6-dependent autophagic pathway, represents a novel pathway that is critical to cilia homeostasis in response to TSE exposure. Immunoblots were performed to check the expression of Cilia (acetylated α-tubulin) and diminution expression of acetylated α-tubulin in COPD tissue as compared to NL was found.

In initial experiment, a lysosomotropic agent, acridine orange, was used to detect acidic vesicles. Results indicated that isolated AECs from COPD showed increased late autophagic vacuoles, as evidenced by an increase in fluorescence intensity.

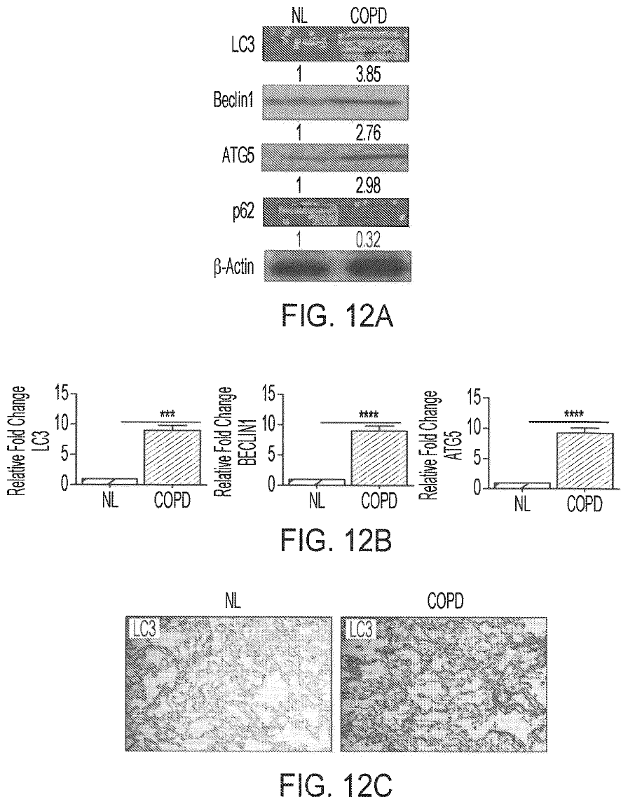

Immunoblot and Real time PCR were performed to examine at both mRNA and protein levels. Increased expression of LC3, Beclin1 and Atg5 were found in COPD lungs compare to normal (NL) (FIGS. 12B-12C).

Elevated levels of autophagy protein in COPD lung as compare to NL was observed. Histological analysis for MAP-LC3 showed increased expression in COPD lung tissue. The ratios of LC3B-II/I level, as well as the expression of Atg5 and beclin1, were increased in lung tissue from humans with COPD.

As a marker of autophagic flux, p62 is involved in the degradation of unfolded or misfolded proteins in cells, and the content of insoluble p62 is an indicator of autophagy activation Hua F et al., Nat Commun. 6:7951, 2015).

The amount of insoluble p62, but not soluble, p62 was significantly decreased in lung tissue in COPD as compared to NL, suggesting that it activates autophagy in lung tissue of COPD patients. Interestingly, inconsistent with the increment of LC3B-II level and reduction of insoluble p62 amount, beclin1 levels in COPD airway epithelial cells (AECs) indicate that COPD-induced autophagy also occurs in AECs (FIG. 12A).

### Example 12

### CSP7 Mitigates the Induction of Mucus Hypersecretion and Cilia Shortening in COPD AECs

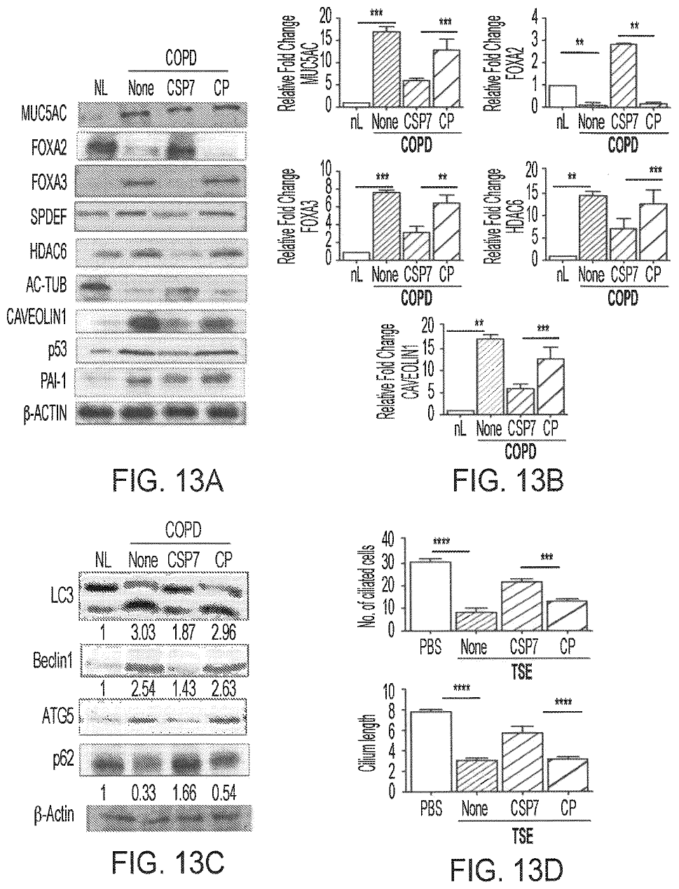

AECs were isolated from NL and COPD lungs. AECs from COPD lungs were treated with or without CSP7 or CP in vitro for 48 h. Western Blot images show increased expression of MUC5AC, HDAC6, PAI-1, p53, Cav1, SPDEF and decreased FOXA2, Ac-Tub (for cilia length) in AEC lysates from COPD lungs, and that these are reversed with CSP7 treatment (FIG. 13A). Quantitative PCR showing increased expression of MUC5Ac, HDAC6, and FOXA3 mRNA, and decreased expression of FOXA2 mRNA in COPD AECs, all of which were reversed by CSP7 treatment (FIG. 13B). The lysosomotropic agent, acridine orange was used to detect acidic vesicles; AECs isolated from COPD lungs shows increased late autophagic vacuoles, as evidenced by an increase in fluorescence intensity. This was reversed by CSP7. Further, changes in the expression of endogenous LC3-II in AECs were examined. Rapid accumulation of the LC3-II form (corresponding to characteristic lipidation of this protein during autophagosome formation) was observed in COPD, and was reverse with CSP7. Besides, immunoblots were used to analyze the expression of other autophagic proteins, including Beclin-1 and ATG5. Their elevated expression in COPD was mitigate by CSP7(FIG. 13C). Moreover, the expression of p62 was significantly elevated with CSP7 treatment in COPD AECs. Interestingly, immunofluorescence staining revealed increased co-localization of MUC5AC/HDAC6 and Ac-Tub/LC3 in COPD AECs; this co-localization was reversed by CSP7 (FIG. 13D).

### Example 13

### TSE Induces Mucus Hypersecretion and Cilia Dysfunction which is Reduced by CSP7

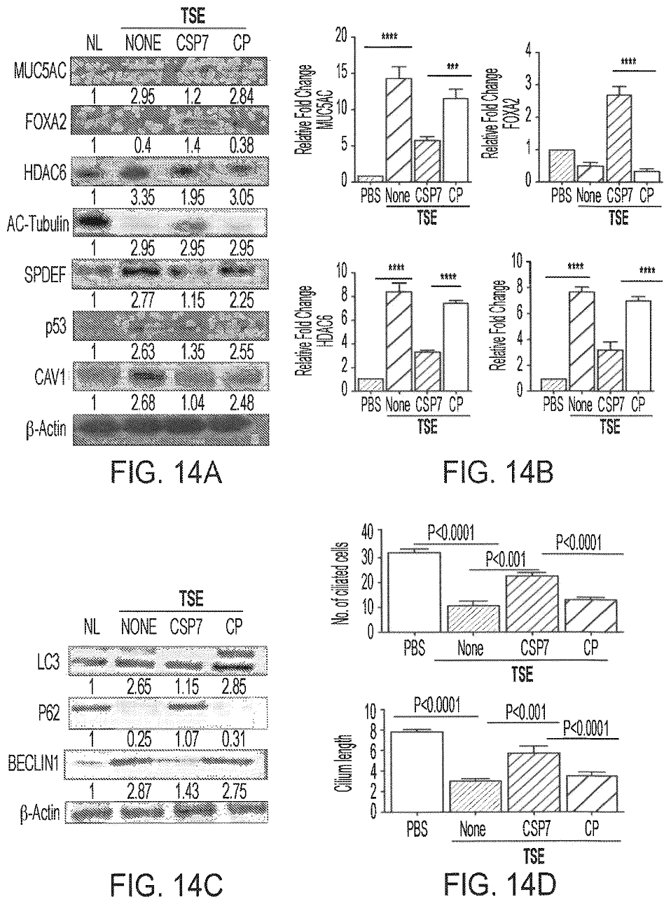

The combination of hypersecretion and ciliary impairment leads to disruption of mucociliary interaction, and, hence, the accumulation of secretions in the lower airways. Cigarette smoke appears to play a critical role in the pathogenesis of COPD associated mucociliary dysfunction. While the excessive lower airway secretions may have only minor effects on the natural course of airflow obstruction, they could transiently compromise airway function during acute exacerbations. Furthermore, western blot images showed increased expression of MUC5AC, HDAC6, SPDEF, FOXA3 and decreased expression of FOXA2 and Ac-Tub in AECs lysates from human NL AECs treated with TS extract (TSE) in vitro for 48 h, which was reversed with CSP7 treatment (FIG. 14A). Bar graphs (QPCR data) showed increased MUC5AC, HDAC6, FOXA3 and SPDEF and reduced FOXA2 mRNA expression in AECs isolated from NL treated with TSE; this increased expression was reversed by CSP7 treatment (FIG. 14B). Western Blots for Autophagy protein markers by TSE and was reversed by CSP7 (FIG. 14C).

Moreover, Immunofluorescence staining revealed increased co-localization of MUC5AC/HDAC6 and AC-Tub/LC3 in AECs exposed to TS vs diffused staining in PBS treated controls. TSE-treated AECs exposed to CSP7 showed reversal of the co-localization of the MUC5AC/HDAC6 and AC-Tubulin/LC3. Bar graphs depicting significant decrease in cilia length and number of ciliated cells in TSE AECs show that this was significantly improved after treatment with CSP7 (FIG. 14D).

### Example 14

### CSP7 Delivered by Nebulization (NEB) or Intraperitoneal (IP) Injection Mitigates TSE Lung Injury in Mice

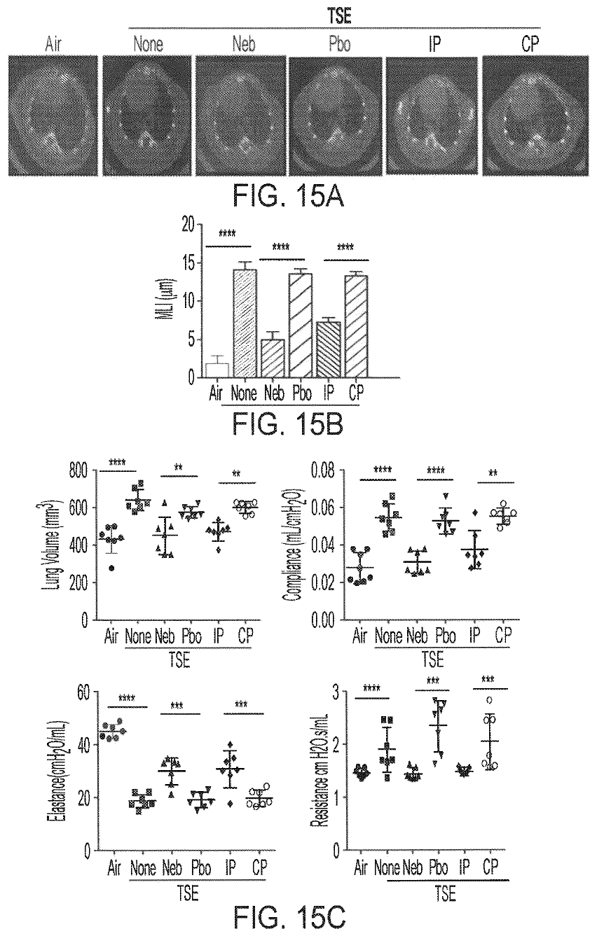

Wild type (WT) mice of the C57BL/6J strain (n=10/group) were kept in ambient air or were TSE for 4 hour/day, 5 days a week as described. After 16 weeks, TSE WT mice were left untreated (“None”) or given formulated CSP7 (5.8 mg) in 30 ml of PBS containing lactose monohydrate (154 mg) or placebo (Pbo) alone 2 h daily 5 days a week for 4 weeks using a Neb tower, or IP injection of 1.5 mg/kg of CSP7 or CP daily 5 days a week for 4 weeks. All mice were subjected to CT and lung volume measurements 20 weeks after TSE exposure (FIG. 15A). Results showed that systemic (IP) or local (Neb) administration of CSP7 reduced lung volume, compliance, elastance and resistance. Besides, representative H&E staining of tissue sections of 20 weeks TSE WT mice, which was reversed in CSP7 (Neb and IP) treated WT mice and bar graphs showing increased mean linear intercept (MLI) observed in lung tissue sections (FIGS. 15B-15C). Simultaneously, lung parameter of 20 weeks TSE WT mice, like Lung volume, Elastance, compliance and resistance show a trend of reversal with CSP7 (Neb and IP) treatment.

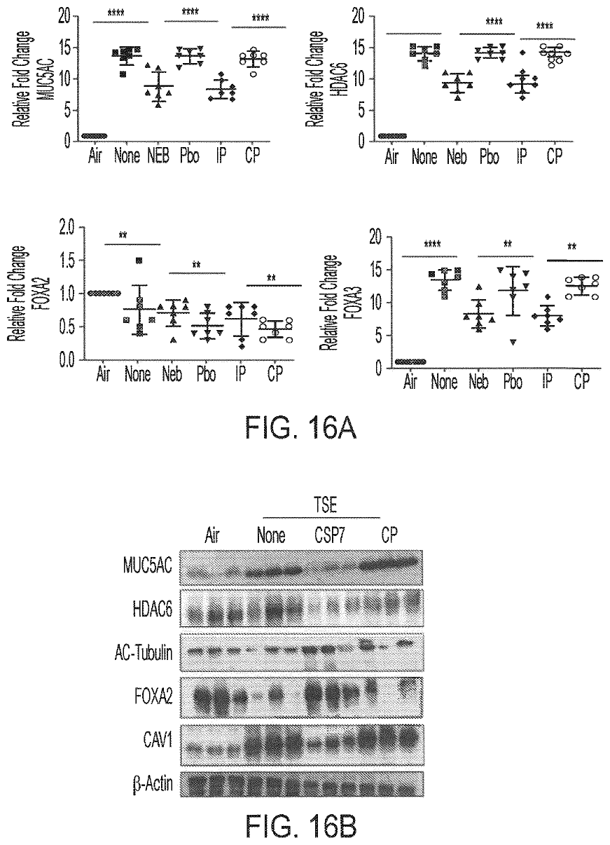

Moreover, CSP7 delivered by IP injection or Neb alleviated TSE MUC5AC and HDAC6 expression. Total lung homogenates were analysed for RNA and protein level for Mucus hypersecretion and autophagy marker (FIGS. 16A-16B). Histological analysis of lung sections also showed increased expression of MUC5AC and HDAC6 in lung sections of 20 week TSE WT mice, which was reversed by CSP7 (Neb and IP) treated WT mice.

The beneficial effects of locally and systemically delivered CSP7 against TSE induced lung injury provide strong rationale. Additionally, immunocytochemical (ICC) staining revealed increased co-localization of MUC5AC/HDAC6 in lung sections of 20 week TSE WT mice, which was reversed in CSP7 (Neb and IP) treated WT mice.

### Example 15

### CSP7 Delivered by Nebulization (NEB) or Intraperitoneal Injection (IP) Decreased Acetylated α-Tubulin and Increased LC3 Expression

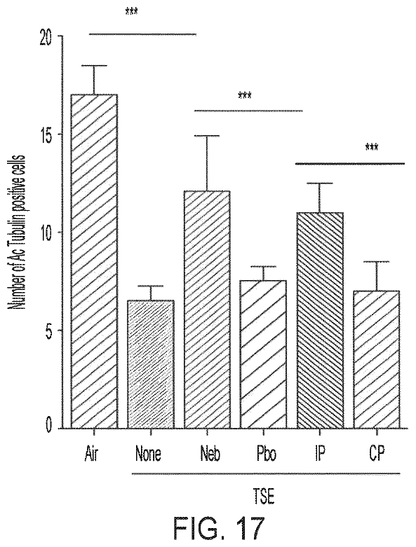

WT mice (n=10/group) were kept in ambient AIR or TSE for 4 hour/day 5 days a week as described. After 16 weeks, TSE WT mice were left untreated (None) or exposed to formulated CSP7 (as above, by NEB or IP injection) or placebo (PBO) alone. Immunohistochemistry (IHC) images showed decreased expression of Ac-Tubulin and increased LC3 expression in lung sections of 20 weeks TSE WT mice, which was reversed in CSP7 (Neb and IP) treated WT mice (FIG. 17). CSP7 delivered by IP injection or Neb mitigated TSE lung injury in mice. Staining for Ac-Tub in lung trachea sections of 20 weeks TSE WT mice was reversed in CSP7 treated WT mice.

Studies were done to better understand the mechanisms by which TSE exposure disrupts the function of ciliated epithelial cells of the respiratory tract and their impact on airway function. Mouse tracheal bronchial epithelial cells (MTEC) were isolated from 20 weeks TSE WT mice as well as those treated with CSP7 or CP. Interesting, immunofluorescence imaging using acetylated α-tubulin (cilia) after isolation of MTEC, demonstrated decreases in the number of ciliated cell (Ac-Tub isolated from 20 weeks TSE WT mice) and its reversal in CSP7 treated WT mice (Lam H C et al. J Clin Invest 123: 5212-30, 2013).

### Example 16

### Role of Cav1 in Mucin Hypersecretion and Ciliary Disassembly

Cav1, a component protein in the cell membrane, reportedly regulates airway inflammation and lung injury (Yu, Q. et al., Int J Mol Med. 35:1435-42, 2015)

A bar graph shows increased Cav1 mRNA expression in COPD as compare to NL. A goal of this study was to determine whether Cav1 modulates mucin hyperproduction induced by TSE. AECs were isolated from NL and COPD lungs. The cells from COPD lungs were treated with or without CSP7 or CP in vitro for 48 h. Bar graphs show increased expression of Cav1 mRNA, COPD AECs analysed by QPCR, which was reversed by CSP7 treatment.

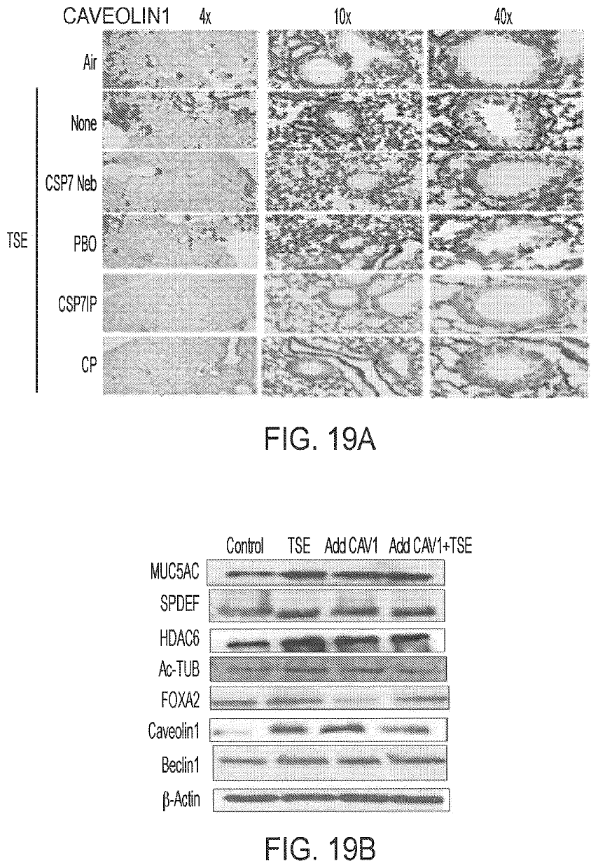

Increased Caveolin1 expression in AECs isolated from NL treated with TSE were observed, and this was reversed by CSP7. Histological analysis of lung sections showed increased expression of Cav1 in sections of 20 weeks TSE WT mice, which was overcome in CSP7 (Neb and IP) treated WT mice (FIG. 19A). Western blot analysis revealed that the overexpression of Cav1 induced in AECs by transduction of adenoviral vector expressing Cav1 (Ad-Cav1) caused a marked increase MUC5AC, HDAC6, SPDEF, FOXA3, and Cav1 and a decrease in FOXA2 and Ac-Tub (cilia). Immunoblot experiments were done to investigate CSP7 suppression of the over-expression of Cav1 (FIG. 19B). Interestingly, CSP7 can mitigate the mucus hypersecretion and cilia disassembly by inhibiting the role of overexpressed Cav1.

### Example 17

### Role of p53 and PAI-1 in TSE Induced Mucin Hypersecretion and Cilia Dysfunction in Mouse Model

To determine whether lung epithelial injury due to TSE induced p53 and if p53 played a pivotal role in the induction of PAI-1 expression in vivo, WT mice were exposed to ambient air or passive TS for 20 weeks. PAI-1 was analyzed in bronchial alveolar lavage (BAL) fluids and lung homogenates, whereas p53 was analyzed in lung homogenates. To determine whether CSP7 inhibited p53 and PAI-1 expression induced by TSE, WT mice were injected with (or without CSP7 or CP, 4 weeks after initiation of passive TSE. At the end of 20 weeks, BAL fluids and lung homogenates were analyzed for changes in p53 and PAI-1. Consistent with the outcomes of AECs in vitro, CSP7 treatment of mice in vivo significantly suppressed the expression of p53 and PAI-1.

To investigate the role of p53 and PAI-1 in TSE induced mucin hypersecretion and cilia dysfunction, p53^{-/-} and PAI-1^{-/-} mice (n=10/group) were kept in ambient air or TSE for 4 hour/day 5 days a week as described. Histological analysis of lung sections also showed increased expression of MUC5AC in the lung section of TSE (20 week) WT mice, which was suppressed in WT mice kept in ambient air, and in TSE p53^{-/-} and PAI-1^{-/-} mice.

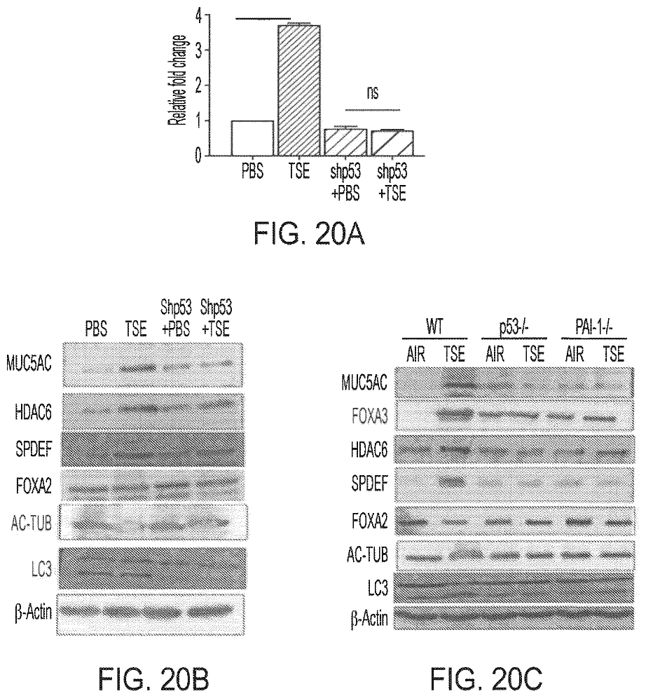

Western blot images showed increased Mucin related gene expression in the lung homogenates of TSE (20 weeks) WT mice, but resistance to this effect in p53^{-/-} and PAI-1 ^{-/-} (FIGS. 20B-20C) mice. Increased MUC5AC mRNA and protein expression in TSE-treated AECs, which was absent in TSE treated AECs transduced with Lvp53 shRNA. Thus, mucus associated protein and autophagy markers were elevated in expression in TSE treated animals and diminished in AECs that had been transduced with Lvp53 shRNA (FIG. 20A).

### Example 18

### Mechanism of CSP7 Attenuation of Mucus Hypersecretion and Ciliary Disassembly: AECs from Subjects with COPD have Reduced PP2A Signalling

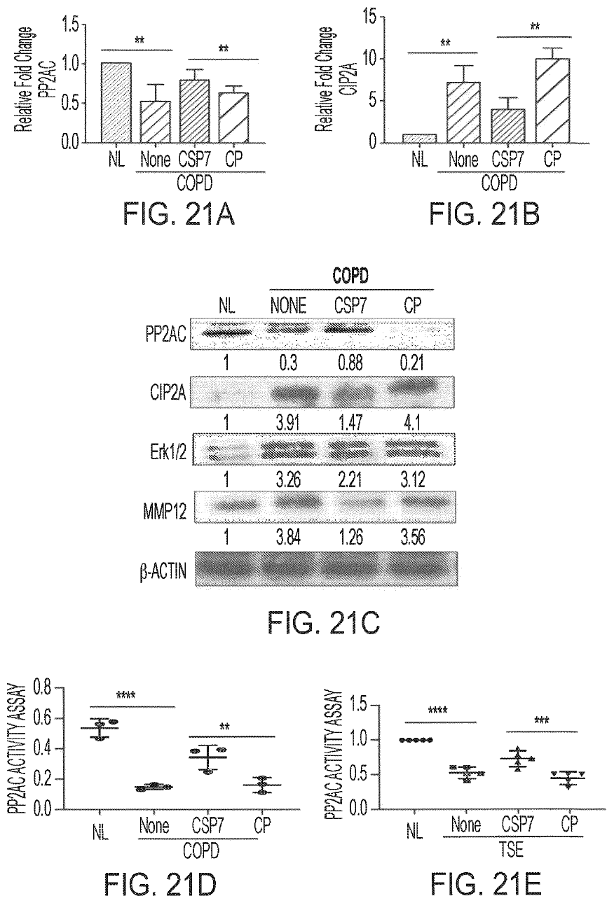

Protein phosphatase 2A (PP2A) activation is altered in emphysema lung samples. Therefore, PP2A activity levels were examined in AECs isolated from NL and from subjects with COPD. PP2A activity was significantly decreased in AECs from subjects with COPD. PP2A activity influences ERK phosphorylation, so the loss of PP2A activity was further examined by investigating ERK phosphorylation. Increased ERK expression was found in AECs from subjects with COPD, as confirmed by Western blot. Expression of PP2AC was also observed. Phosphorylation of PP2Ac in AECs from COPD patients compared with those from NL, indicating an alternate cause for decrease of PP2AC in the airway epithelium of subjects with COPD (FIGS. 12A-12C). CIP2A is an endogenous inhibitor of PP2AC. The expression CIP2A mRNA and protein levels were therefore investigated and were increased in COPD patient AECs compared to levels from NL (FIGS. 21B-21C). Increased CIP2A gene expression and protein levels in subjects with COPD was concluded to be a likely major cause of reduced PP2AC activity in COPD. CIP2A expression was increased in AECs isolated from subjects with COPD, which decreased PP2A activity and thus increased MMP12 expression and secretion. When CIP2A was inhibited by CSP7, increased activity of PP2AC was observed in COPD AECs. The increased PP2AC activity was further confirmed by a downstream decrease in ERK phosphorylation.

The proteases that are linked to the development of COPD and are regulated by PP2AC and MMP12 were investigated. CIP2A expression was increased in COPD AECs, which had decreased PP2AC activity and, thus, increased MMP12 secretion. The relative gene expression of MMP12 was decreased in NL AECs and from COPD AECs treated with CSP7. Therefore, CSP7 mitigate the effect on PP2AC, ERK, and MMP12 in COPD (FIG. 21C). COPD lung tissues exposed to CSP7 ex vivo had reduced PP2A signaling. Serine-threonine phosphatase activity for PP2A was determined for each individual and represented as picomoles of phosphate liberated per minute on the y-axis (FIG. 21D). WT mice (n=10/group) were kept in ambient air or exposed to TS for 4 h/days 5 day a week as. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 d a week for 4 weeks using a Neb tower, or IP injected with 1.5 mg/kg of CSP7 or CP daily 5 d a week for 4 weeks, TSE exposure reduced PP2A signaling and was reversed by CSP7. Serine-threonine phosphatase activity for PP2A was determined for each individual and represented as picomoles of phosphate liberated per minute on the y-axis (FIG. 21E). The above represents an important mechanism by which CSP7 attenuates the effect of mucus hypersecretion and ciliary disassembly.

### Discussion of Examples 11-18

The chronic airflow limitation of COPD is caused by a mixture of small airway disease and pulmonary emphysema, usually due to significant exposure to noxious particles or gases. TSE is the most common identifiable risk factor for COPD, with smokers known to have a higher COPD mortality rate than non-smokers (Kim, V. et al., PLoS One. 10(2): e0116108, 2015). Pulmonary emphysema is believed to result from epithelial cell death caused by smoking; therefore, COPD research has been substantially devoted to programmed cell death. In COPD patients, airway epithelium undergoes remodeling, leading to hyperplasia and metaplasia of airway cells, including goblet cells. Goblet cell hyperplasia and hypertrophy is consistently found in the large airways of smokers with airflow obstruction (Saetta M et al., Am J Respir Crit Care Med. 161:1016-21, 2000; Innes A L et al., Chest. 130:1102-8, 2006). Such changes to goblet cells results in mucus overproduction, hypersecretion, and, ultimately, mucus accumulation in the airway lumen with serious pathological outcomes (Ramos F L et al., Int J Chron Obstruct. Pulm. Dis. 9:139-50, 2014. Excessive production is a consequence of increased synthesis and secretion of mucins and is often associated with increase in number of goblet cells. Based on this and the knowledge that smoking induces metaplasia of MUC5AC-positive cells in the airway epithelium of smokers, and increased expression of MUC5AC in the airway epithelium of smokers, targeting mucus hypersecretion alleviates COPD exacerbation.

Studies described herein investigated the effects of TSE on changes lung MUC5AC mRNA and protein expression, and mucus hypersecretion in a model of mouse emphysema and type I human AECs. The present findings include augmented expression of MUC5AC in COPD AECs as compared to normal AECs. Interestingly, small airways of humans (<2 mm lumenal diameter) and all intrapulmonary airways of mice, have few or no visible ‘mucous’ or ‘goblet’ cells under baseline conditions. In allergic inflammation or TSE, there is a rapid and dramatic increase mucous cell metaplasia or goblet cells (Evans, C et al., Am. J. Respir. Cell Mol. Biol. 31:382-94, 2004).

A developmental transcriptional regulator of goblet cell hypertrophy and hyperplasia is a SAM-pointed domain-containing Ets-like factor (SPDEF). SPDEF expression is increased in airways of COPD patients (Chen G et al., 2009; supra) and in long-term smokers (Chen G et al., 2014, supra). SPDEF upregulates several goblet cell differentiation genes, including that encoding forkhead box A3 (FOXA3) (Chen et al. 2014, supra) and endoplasmic reticulum protein anterior gradient protein 2 homolog. FOXA3 was sufficient to induce goblet cell metaplasia in vivo and in vitro. The present inventors' in vitro and in vivo studies showed that FOXA3 was sufficient to cause goblet cell metaplasia in airway epithelium. FOXA3 bound to and induced SPDEF, a gene required for goblet cell differentiation in the airway epithelium. Hence, the observed effects of FOXA3 on mucus related gene expression are likely mediated, at least in part, by the ability to induce SPDEF. However, FOXA3 directly bound to, and induced, AGR2 and MUC5AC that are critical for airway mucus production/goblet cell metaplasia (Williams O W et al., Am J Respir Cell Mol Biol 34:527-36, 2006; Schroeder B W et al., Am J Respir Cell Mol Biol 47:178-85, 2012), functioning independently of SPDEF to regulate these genes in human airway epithelial cells. Disruption of FOXA2 in respiratory epithelial cells caused airspace enlargement, pulmonary neutrophil infiltrates, and mucous metaplasia. SPDEF and MUC5AC have previously been shown to be highly expressed in bronchial epithelium of COPD patients (Chen et al., 2014, supra), which agrees with the present findings of increased expression of SPDEF, MUC5AC, and FOXA3 and decreased FOXA2 expression in COPD when compared to controls. In the present studies, treatment with CSP7 was found to reduce the effect of mucus hypersecretion-related genes.

Emerging evidence suggests that autophagy plays an important role in pulmonary diseases (Patel A S et al., PLoS One. 7:e41394, 2012; Ryter S W et al., Annu. Rev. Physiol. 74:377-401, 2012; Wu Y F et al., Autophagy. 2019 Jun. 16, https://doi.org/10.1080/15548627.2019.16285360). Prior reports demonstrated that autophagy was critical in mediating tobacco smoke-induced apoptosis of lung epithelial cells and contributed to development of emphysema (Chen Z H et al., PLoS One 3:e3316, 2008; Chen Z H et al., Proc Natl Acad Sci USA 107:18880-85, 2010.) As recently reported, exposure to particulate matter inactivated mTOR (a mechanistic target of rapamycin kinase), enhanced macroautophagy/autophagy, and impaired lysosomal activity in human bronchial epithelial cells and in mouse airway epithelium (Wu et al., 2019, supra). Moreover, autophagy also mediates TSE-induced cilia shortening and mitochondrial dysfunction in airway epithelium (Cloonan S M et al., Autophagy 10:5324, 2014; Lam H C et al., J Clin Invest 123:5212-30, 2013).

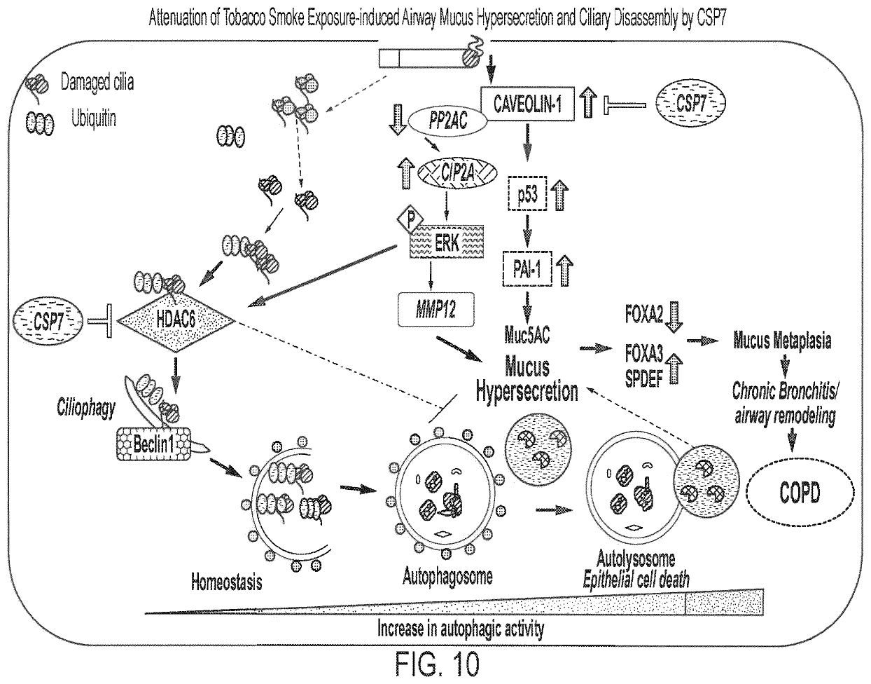

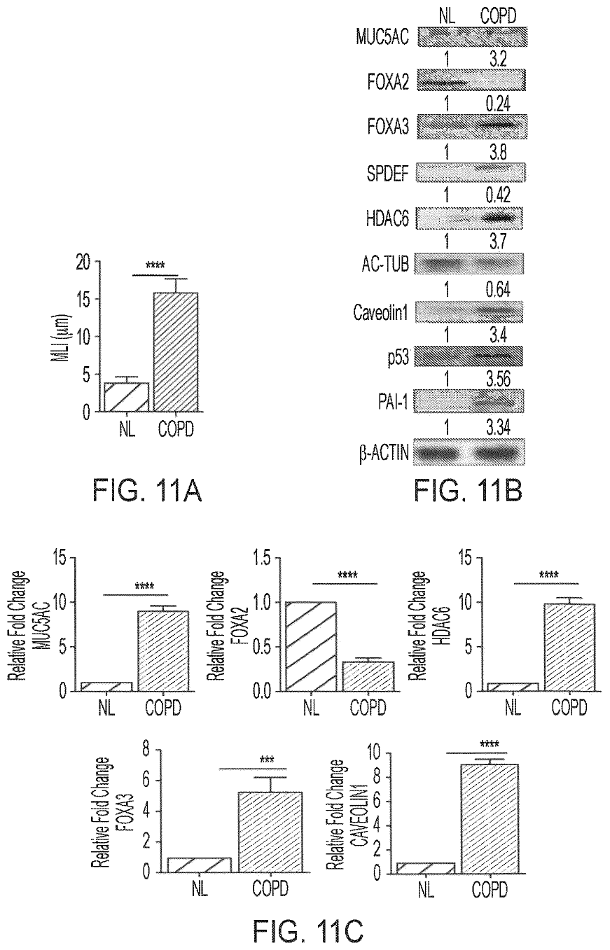

However, there is growing evidence that autophagy is a deleterious process that orchestrates various damage in airway epithelium during COPD pathogenesis. In the lungs, the “mucociliary escalator” acts as a primary innate defense mechanism, in which motile ciliated epithelial cells eliminate particles and pathogens trapped in mucus from the airways. Disruption of airway epithelial cell function as a result of TSE impairs mucociliary clearance (MCC). The mechanisms by which TSE-induced epithelial cell dysfunction leads to cilia shortening and altered airway function in vivo need further clarification. Among these mechanisms, cytosolic deacetylase HDAC6, which contains ubiquitin-binding and dynein-interacting domains, has emerged as a pleiotropic regulator of cellular function. HDAC6 controls diverse cellular processes through deacetylating and destabilizing microtubules (Pugacheva E N et al., Cell, 129:1351-63, 2007) facilitating retrograde transport of ubiquitinated proteins into aggresomes (Pandey U B, et al. Nature 447:859-63, 2007) and enhancing autophagosome-lysosome fusion Lee J Y, et al., EMBO J. 29:969-80, 2010). A role for HDAC6 has been found in motile cilia of the airways, in cellular responses to TSE exposure, and in COPD pathogenesis. This is illustrated schematically in FIG. 10. However, growing evidence indicates that HDAC6 recognizes ubiquitinated protein aggregates and delivers them to the autophagosome, a process dependent on the autophagy proteins LC3B and beclin 1. Ciliary proteins are delivered to the lysosome for degradation or recycling. In cases of chronic oxidative stress, ciliary proteins are degraded, resulting in a shortening of airway cilia that contributes to impaired mucociliary clearance. Interestingly the present inventors showed, in COPD and with TSE, increased HDAC6 and upregulation of autophagy markers leading to cilia shortening. They showed that HDAC6 increases, upregulation of autophagy molecules, and cilia shortening in COPD and co-TSE is reduced or attenuated by CSP7 treatment. Cilia components were shown to co-localize with autophagosomes based on Ac-Tub and LC3 co-localization. For the first time, interactions were found to occur between HDAC6 and MUC5AC in AECs in COPD, in TS-exposed AECs and in a murine emphysema model.

Caveolae are vesicular invaginations of the plasma membrane and the structural protein component of caveolae is Cav1. Cav1 participates in signal transduction processes—acting as a scaffolding protein that concentrates, organizes and functionally regulates signaling molecules within caveolar membranes. TS, a source of oxidants, is an environmental hazard that causes pulmonary emphysema. Over-expression of Cav1 was enough to induce mucus hypersecretion and ciliary disassembly. Subsequently in the present studies mucus hypersecretion related genes and cilia were shown to be upregulated when Cav1 protein was overexpressed.

Insights into the molecular mechanism underlying free radical activation of the ataxia telangiectasia-mutated (ATM)-p53 pathway and a suggestion that Cav1 may be a novel therapeutic target for the treatment and/or prevention of pulmonary emphysema was described by Volonte D et al., J Biol Chem. 284:5462-6, 2009.

The present inventors and their colleagues previously demonstrated that tumor suppressor protein p53 augmented PAI-1 expression in AECs during TSE-induced lung injury. Chronic lung inflammation with elevated p53 and PAI-1 expression in AECs and increased susceptibility to and exacerbation of respiratory infections are all associated with COPD. (See Tiwari et al., 2016, supra). The present inventors and colleagues demonstrated that preventing p53 from binding to the endogenous PAI-1 mRNA in AECs by either suppressing p53 expression or blockading p53 interactions with the PAI-1 mRNA mitigated mucus hypersecretion and ciliary disassembly. A previous report elucidated the premature senescence of lung fibroblasts induced by oxidative stress which occurred by activation of ATM)/p53-dependent pathway following sequestration into caveolar membranes of the catalytic subunit of protein phosphatase 2A (PP2A-C), an inhibitor of ATM, by Cav1. A previous study demonstrated that loss of PP2A expression enhanced TS induced MMP1 and MMP9 expression (Wallace A M et al., Toxicol Sci 126:589-99, 2012). Although caveolae were known to be highly immobile and non-endocytic under normal conditions, in stress conditions or as a result of TSE, endocytosis occurs via a Cav1-mediated process. PP2A activity was downregulated by chronic TSE and decreased in COPD, which subsequently modulated proteolytic responses. In addition, CIP2A is an inhibitor of PP2A. The present inventors showed that AECs from COPD subjects and active smokers had reduced PP2A activity as well as increased, CIP2A expression.

In the present studies, Cav1 bound to PP2AC and was downregulated PP2AC activity, leading to increased CIP2A expression. Increased CIP2A led to phosphorylation of ERK, and secretion of MMP12. The Cav1 elevated p53 and PAI-1 expression in AECs and increased susceptibility to and exacerbation of respiratory infections are associated with COPD. Moreover, Cav1 expression was required for activation of the p53-PAI-1 pathway following stimulation with TSE extracts in vitro. Thus, according to this invention, Cav-1 is a key player in a novel signaling pathway that links TSE to mucus hypersecretion and ciliary disassembly. A 7-mer peptide fragment of CSP, CSP7 (FTTFTVT, SEQ ID NO:1) mitigated cilia shortening and impaired mucociliary clearance (MCC) by inhibiting Cav1. CSP7 also significantly downregulated phosphorylation of ERK, expression levels of MMP-12, and CIP2A. These findings provide not only new insights on how CSP7 regulates complex interrelationships between p53, PAI-1, autophagy and primary cilia. CSP7 is useful for treatment of the ciliopathy-associated mucus hypersecretion. This is the first discovery of
(a) CSP7 markedly reducing mucus hypersecretion and attenuating ciliary disassembly,
(b) understanding the underlying cellular and molecular mechanisms of caveolin's important role in TSE-associated cilia shortening and mucus hypersecretion by an endocytic process and
(c) the mitigation of these effects by CSP7.
   According to the present invention a Cav1 scaffolding domain peptide (CSP), and preferably, its biologically active peptide CSP7, is a new therapeutic agent for improving airway function during chronic lung diseases such as COPD by reversing, preventing or attenuating cilia shortening and impaired mucociliary clearance.

### Example 19

The loss of alveolar epithelial regeneration in patients with idiopathic pulmonary fibrosis (IPF) and chronic obstructive pulmonary disease (COPD) has been attributed to telomere dysfunction in progenitor type II alveolar epithelial cells (A₂Cs). Aside from genetic factors, particulate matters, including tobacco smoke (TS) and aging contribute to telomere dysfunction in A₂Cs. The inventor sought to know whether improvement of telomere function is involved in the CSP7-mediated A₂C protection against ongoing senescence and death during PF and TS-induced alveolar injury. A significant telomere shortening in A₂Cs isolated from IPF and COPD lungs was found. Further, these cells showed increased p53 in addition to its post-translational modification with induction of activated caspase-3 and β-galactosidase, suggesting p53-mediated loss of A₂C renewal. SIAH-1, a p53-inducible E3 ubiquitin ligase that is known to down-regulate the telomere repeat binding factor 2 (TRF2) was also increased. Consistent with loss of TRF2 and upregulation of TRF1, telomerase reverse transcriptase (TERT) was down-regulated in A₂Cs. A2Cs from mice with multi-hit bleomycin-induced PF or chronic TS-induced lung injury showed reduction in telomere length along with induction of p53, PAI-1, SIAH1 and TRF1, and loss of TRF2 and TERT. This phenomenon was reduced in wild-type mice having bleomycin- or TS-induced lung injury treated with CSP7. Interestingly, PAI-1^{-/-} or IL-17A^{-/-} mice, or those lacking miR-34a expression in A2Cs resisted telomere dysfunction while uPA^{-/-} mice failed to respond to CSP7 treatment, suggesting p53-miR-34a feed-forward induction and p53-uPA system cross-talk contributes to telomere dysfunction.

### Introduction

Idiopathic pulmonary fibrosis (IPF) is one of the most common and lethal forms of interstitial lung disease (ILD) often associated with advanced aging and progressive destruction of the lung parenchyma. (Hutchinson et al., Ann Am Thorac Soc 2014, 11:1176-1185; Ley et al, Am J Respir Crit Care Med 2011, 183:431-440.) IPF is a fatal age-related ILD with an incidence of 93.7 cases/100,000 individuals (65 years or older) in the US annually and a median five-year survival of only 20%. FDA approved drugs such as Pirfenidone and Nintedanib slow the decline of lung function in patients with IPF but are not curative. (Saito et al, J Thorac Dis 2019, 11:S1740-S1754; Hughes et al, Journal of Clinical Medicine, Multidisciplinary Digital Publishing Institute, 2016, 5:78.) The pathogenesis of IPF is characterized by permanent cell cycle arrest (known as replicative senescence) and apoptosis of progenitor type II alveolar epithelial cells (A₂Cs), which result in proliferation and accumulation of activated myofibroblasts and fibrotic lung fibroblasts, and extracellular matrix deposition. The etiology of IPF is unknown, however recent studies have implicated aging, repetitive alveolar epithelial injury and telomere dysfunction, particularly in A₂Cs in the pathogenesis of progressive PF (Katzen and Beers, J Clin Invest, American Society for Clinical Investigation, 2020, 130:5088-5099), including human IPF.

Chronic obstructive pulmonary disease (COPD) is a debilitating progressive lung disease that affects about 24 million people and represents the third leading cause of death in the United States. (Hurd, Chest 2000, 117:1S-4S; Ford et al, Chest 2013, 144:284-305.) In COPD, chronic inflammation mediates alveolar wall destruction with air space enlargement (emphysema). Aging and chronic exposure to tobacco smoke (TS) or secondary TS exposure are two important determinants of susceptibility to emphysema. Chronic TS exposure alone evokes premature cellular aging. In addition, 30% of patients with emphysema develop PF (Ford et al, Chest 2013, 144:284-305), suggesting patients with TS exposure lung injury and COPD are predisposed to develop PF. A₂Cs are common targets for damage from TS and mediators/cytokines released from inflammatory cells. Importantly, the pathogenesis of COPD has been directly linked to a loss of alveolar structure due to A₂C senescence and apoptosis. (Shetty et al, Am J Respir Cell Mol Biol 2012, 47:474-483; Park et al, COPD 2007, 4:347-353; Tsuhi et al, Am J Respir Cell Mol Biol 2004, 31:643-649.) These reports (Shetty et al, 2012; Bhandry et al, Am J Physiol Lung Cell Mol Physiol 2016, 310:L496-506; Tiwari et al, Am J Physiol Lung Cell Mol Physiol 2016, 310:L496-506; Marudamuthu et al, Am J Pathol 2015, 185:55-68; Shetty et al, J Biol Chem 2008a, 283:19570-19580; Bhandary et al, Toxicol Appl Pharmacol 2015, 283:92-98; Shetty et al, Am J Pathol 2017, 187:1016-1034) indicate that TS-induced lung injury primarily increases A₂Cs senescence and apoptosis.

Increased expression of caveolin-1 (Cav1), the tumor suppressor protein, p53 and p53 downstream target; plasminogen activator inhibitor-1 (PAI-1) play a crucial role in induction of A₂C senescence and apoptosis, and lung injury in patients with COPD and ILDs, including IPF (Shetty et al, 2012; Tiwari et al, 2016; Shetty et al, 2017; Shetty et al, J Biol Chem 2008b, 283:19570-19580; Bhandary et al, Am J Pathol 2013, 183:131-143.) The expression of Cav1, p53 and PAI-1 increases through feedback induction and is often associated with increased A₂C senescence and apoptosis (Shetty et al, 2008a; Shetty et al, 2008b, Bhandary et al, 2013). Lung lavage fluids exhibit high levels of urokinase-type plasminogen activator (uPA) activity that contribute to alveolar proteolysis. However, most forms of lung injury, including COPD, and ILDs, including IPF are characterized by defective alveolar fibrinolysis. This is mainly attributable to local over-expression of p53, and PAI-1 (Chapman et al, Am Rev Respir Dis 1986, 133:437-443; Chapman, J Clin Invest 2004, 113:148-157; Hasday et al, Exp Lung Res 1988, 14:261-278; Bertozzi et al, N Engl J Med 1990, 322:890-897; Bachofen and Weibel, Clin Chest Med 1982, 3:35-56; Idell et al, J Clin Invest 1989, 84:695-705; Barazzone et al, J Clin Invest 1996, 98:2666-2673; Olman et al, J Clin Invest 1995, 96:1621-1630; Eitzman et al, J Clin Invest 1996, 97:232-237; Lardot et al, Am J Respir Crit Care Med 1998, 157:617-628; Xu et al, Exp Lung Res 2009, 35:795-805; Hu et al, Chin Med J 2009, 122:2380-23851 Zidovetzki et al, Stroke 1999, 30:651-655) by p53 binding through its C-terminal amino acid residues 296-393 with a 70-nucleotide destabilization determinant of PAI-1 3′untranslated region (3′UTR) (Bhandary et al, 2015; Shetty et al, 2008b; Shetty et al, Am J Respir Cell Mol Biol 2012, 47:474-483.) In addition, p53 binds the PAI-1 promoter and increases PAI-1 mRNA transcription. (Shetty et al, 2008a; Shetty et al, 2008c; Shetty et al, Mol Cell Biol 2007, 27:5607-5618.) Exposure of A2Cs to TS or bleomycin induces p53 and that p53-mediated upregulation of PAI-1, which in turn promotes A₂Cs senescence and apoptosis, lung injury and PF. Mice lacking either p53 or PAI-1 resist both TS and bleomycin exposure lung injury and PF (Tiwari et al, 2016; Bhandary et al, 2013). Interleukin-17A (IL-17A) levels were also significantly increased in the lungs of patients with severe COPD (GOLD stage III and IV). Elevated levels of IL-17A in lungs of wild-type (WT) mice exposed to TS for 20 weeks or bleomycin-induced PF were found, while IL-17A^{-/-} mice are protected against TS- or bleomycin-induced lung injury or PF. IL-17A also augments Cav1, p53 and PAI-1 expression that promotes senescence and apoptosis in A₂Cs. (Tiwari, 2016). The effects are clinically relevant and occur in patients with COPD or PF.

Evidence suggests that telomere dysfunction of A₂Cs increases susceptibility for development of PF and emphysema in mice and humans. Further, telomere dysfunction due to TS evokes replicative senescence and death of A₂Cs mediating alveolar wall damage and alveolar injury. As in aging, chronic TS exposure contributes to telomere shortening, which limits the proliferative recovery of lung epithelium especially A₂Cs, and which promotes cellular senescence and emphysema. (Stanley et al, J Clin Invest 2015, 125:563-570; Stanley et al, Ann Am Thorac Soc 2016, 13 Suppl 5:S447-S451.) Approximately 20% of IPF with causative mutations have been identified in the genes that regulate telomere function, protein folding and secretion. (Dai et al, Respirology 2015, 20:122-128; Lawson and Lloyd, PATS, 2006, 3:345-349.) PF associated with mutations in the gene that encodes telomerase is progressive and lethal with a mean survival of three years after diagnosis. (Leon et al, PLOS ONE, Public Library of Science, 2010, 5:e10680; Stuart et al, The Lancet Respiratory Medicine 2014, 2:557-565) and such mutations are found in approximately 37% of kindred with familial PF and 25% of patients with sporadic IPF without any family history of disease. (Cronkhite et al, Am J Respir Crit Care Med, American Thoracic Society —AJRCCM, 2008, 178:729-737.) Telomere shortening due to non-genetic causes, including exposure to oxidative damage and chronic TS exposure induce lung diseases.

Telomeres are the protective ends of linear chromosomes consisting of tandem repeats of short DNA sequences and shorten throughout the lifespan. The telomere proteome consists of more than 200 proteins for execution of a variety of telomeric functions such as protection, elongation, and DNA synthesis. Telomerase is a specialized enzyme responsible for maintenance of telomere length by synthesis of tandem TTAGGG telomere repeats. Telomerase has two essential subunits: a catalytic telomerase reverse transcriptase (TERT) and a telomerase RNA component (TERC). Maintenance of telomere length is paramount for proper cellular function. Telomere length in 25% of sporadic cases of PF is less than the 10^{th} percentile. (Cronkhite et al, 2008.) Shortening of telomeres in a subset of IPF patients also correlates with poor survival. (Stuart et al, 2014). Interestingly, examination of surgical biopsy of patients with IPF revealed predominantly shortened telomere in A₂Cs associated with the fibrotic lesion. (Naikawadi et al, JCI Insight 1:e86704). Telomere shortening promotes cellular senescence via activation of p53 and limits the proliferative recovery in lung epithelium especially in A₂Cs, which leads to age-dependent lung remodeling and PF. (Dai et al, 2015; Arimura-Omori et al, Asian Pac J Cancer Prev 2020, 21:667-673; Naikawadi et al, JCI Insight 1:e86704.) Further, p53 represses telomerase activity through down-regulation of TERT transcription. (Jin et al, Exp Mol Med 2010, 42:574-582.) Further, telomere dysfunction due to TS evokes replicative senescence and death of A₂Cs mediating alveolar wall damage and susceptibility to develop emphysema in mice and humans. In addition, increased expression of p53, PAI-1 and Cav1 by A₂Cs promotes TS exposure induced lung injury. As in aging, chronic TS exposure contributes to premature telomere shortening, which limits the proliferative recovery of lung epithelium especially in A₂Cs, and which promotes cellular senescence and emphysema. (Stanley et al, 2015; Stanley et al, 2016.) A₂Cs from lungs of patients with IPF, and WT mice with chronic lung injury or PF also exhibit telomere dysfunction by altering shelterin component telomere-repeat binding proteins (Trf1, Trf2), E3-ubiquitin ligase (SIAH1/2); a p53 target gene that degrades Trf2, protein phosphatase 1 nuclear targeting subunit (PNUTS) and TERT. Exposure of mice with conditional deletion of TERT in A₂Cs to bleomycin led to increased A₂Cs senescence and increased PF. (Liu et al, Journal of Biological Chemistry, Elsevier, 2019, 294:8861-8871.) Thus, telomere-targeting therapies provide new hope for treatment of COPD and PF.

The objective of this study was to investigate whether telomere dysfunction in A₂Cs is mediated by the changes in uPA-fibrinolytic system cross-talk through p53-microRNA-34a feed forward induction using mouse models of chronic lung injury induced by repetitive bleomycin or TS exposure. In this study, the effects of p53-mediated induction of PAI-1 and inhibition of uPA on telomere dysfunction, senescence and apoptosis in A₂Cs and the contribution to the development of bleomycin- or TS-induced PF or emphysema were determined. Cav1 scaffolding domain peptide (CSP7) improves telomere function and increases viability of A₂Cs via restraining p53-mediated induction of PAI-1 and inhibition of uPA expression to mitigate alveolar epithelial injury and development of PF. Mice lacking microRNA-34a or PAI-1 or IL-17A resist telomere dysfunction while those lacking uPA expression are highly susceptible to telomere dysfunction.

### Materials and Methods

Mouse models and CSP7 treatment. All experiments involving mice were performed according to the approved protocols under the guidelines of Institutional Animal Care and Use Committee. Wild type (WT), and PAI-1^{-/-} and uPA^{-/-} mice of C57BL/6 background purchased from Jackson Laboratory (Bar Harbor, ME) were used for the experiments. IL-17A^{-/-} mice were obtained from University of Tokyo, Japan.

To test the effect of chronic TS exposure induced lung injury, these mice were exposed to passive TS from 40 research cigarettes over a 4 h period 5 days/week for 20 weeks (˜90 mg/m3 total solid particulates) using a mechanical smoking chamber (Teague Enterprises, Davis, CA) as described earlier. (Bhandary et al, 2015). Control mice were kept in ambient air. After sixteen weeks of daily passive TS exposure, mice with TS exposure injury were treated with CSP7 at a dosage of 1.5 mg/kg body weight daily 5 days/week for the next four weeks. These mice were continued with daily exposure of 4 h of TS during CSP7 treatment. At the end of the experiment (after 20 weeks of TS exposure), the mice were euthanized, and lungs were utilized for further analyses. To expand the local delivery options of CSP7 to directly treat the damaged lung by air-jet milling for dry powder inhalation (DPI). Mice exposed to TSE were treated with CSP7 using a rotating brush generator for 15 min daily for 7 days. Fourteen days later, BLM mice were subjected to daily inhalation of micronized CSP7 (0.05 mg/kg/d) DP through air-jet-milling for seven consecutive days using a rotating brush exposure system. The rotating brush exposure system gave a consistent minimal chamber concentration of 0.1 mg/L. In order to match dosing of DP inhalation, animal exposure times were adjusted to approximately 15 minutes and an estimated dose of 0.05 mg/kg/d or 1.00 microgram/lung was administered.

To evaluate the effect of CSP7 against chronic BLM-induced PF, the mice were exposed to BLM (40 μg/20 g body weight) once every two weeks for 16 weeks as described elsewhere. (Tiwari et al, 2016). Control mice were exposed to normal saline. Fourteen weeks later the CSP7 at a dosage of 1.5 mg/kg body weight was administered by IP injection and continued daily basis for the next two weeks as described elsewhere. (Tiwari et al, 2016; Shetty et al, 2008b). At the end of the experiment, the mice were euthanized and lung tissues and isolated A₂Cs from the extracted lung tissues were used for further analysis.

Isolation of A₂Cs from mouse lungs. A₂Cs were isolated from WT, PAI-1^{-/-} and uPA^{-/-} following the method of Corti et al (Am J Respir Cell Mol Biol 1996, 14:309-315) with minor modifications as described elsewhere. (Marudamuthu et al, Am J Pathol 2015, 185:55-68; Callicott Comp Med 2006, 56:17-22.) The A₂Cs were plated on plastic culture dishes pre-coated with anti-CD-32 and anti-CD-45 antibodies for 2 h at 370 C. The non-adherent cells were collected. The purities of A₂C preparations were more than 90%, based on lithium carbonate staining for inclusion bodies as described earlier. (Shetty et al, 2012; Shetty et al, 2008c). The cells were maintained in poly-L-lysine coated plates in growth-supplemented A₂C culture medium (AEpiCM) (Sciencell, Carlsbad, CA, USA) at 37° C. in an incubator supplied with 5% CO₂.

Protein analysis by Western blotting. Changes in SP-C(cat. no. sc-13979; Santa Cruz Biotechnology, Santa Cruz, CA), p53 (cat. no. sc-6243; Santa Cruz), serine 15 phosphorylated p53 (p53^{S15P}, cat. no. 9284; Cell Signaling Technology, Beverly, MA), lysine 379 acetylated p53 (p53^{Ac}, cat. no. 2570; Cell Signaling Technology), caspase-3 (cat. no. ab32351; Abcam, Cambridge, MA), cleaved caspase-3 (cat. no. 9661; Cell Signaling Technology), PAI-1 (cat. no. ab66705; Abcam) and β-actin (cat. no. 3700; Cell Signaling Technology) expression levels were assessed by Western blotting of A₂C lysates using specific antibodies and enhanced chemiluminescence (ECL) (Thermo, Rockford, IL) detection as described previously. (Shetty et al, 2008c).

Detection of telomerase activity. Telomerase activity was detected using a PCR-based telomeric repeat amplification protocol (TRAP) method using the TRAPeze Telomerase Detection Kit (Intergen, Purchase, NY, USA). Briefly, the cells were lysed in CHAPS lysis buffer and quantified by BCA method, and equal quantity of the protein samples was combined with the reaction mix in RNase-free PCR tubes. The PCR amplification was then performed following instruction of the manufacturer. The final PCR product was loaded onto a 12.5% non-denaturing polyacrylamide gel and subjected to electrophoresis. The polyacrylamide gel was stained with ethidium bromide and documented using a gel-doc unit (Bio-Rad Laboratories). The relative quantities of telomerase activity for each sample were calculated according to the instructions provided in the kit.

Measurement of terminal restriction fragment (telomeric) length. For the determination of telomeric length, the TeloTTAGGG Telomere Length Assay Kit (Roche Diagnostics GmbH) was used. Briefly, genomic DNA was isolated and digested with Hinf1/Rsa. The digested DNA fragments were then separated by electrophoresis on agarose gel followed by Southern blot transfer. The membrane was then hybridized with a telomere specific digoxigenin (DIG)-labelled probe, incubated with anti-DIG alkaline phosphatase, and documented with chemiluminescence detection in gel-doc unit (Bio-Rad Laboratories). Telomeric length was identified by comparing with the pre-labelled molecular weight marker. The relative telomere length was calculated according to the protocol provided. In addition, relative telomere length was analyzed by qPCR analysis of the genomic DNA. (Cawthon, Nucleic Acids Res 2002, 30:e47-e47; Alder et al, Proc Natl Acad Sci USA 2015, 112:5099-5104.) The 36B4 gene was served as the control. The primer sequences are provided in Table 1.

mRNA quantitation by real-time qPCR. Total RNA was isolated from AECs using TRI reagent and reverse transcribed using impromII Reverse transcription kit (Promega, WI). The levels of the mRNAs were quantitated using an aliquot of reverse transcribed total RNA and gene-specific primers (Table 1) by real-time PCR as described earlier. (Bhandary et al, 2013; Shetty et al, 2008c).

Immunohistochemical (IHC) analysis of lung sections. Lung sections (5.0 μm) were subjected to immunostaining as previously described. (Liu et al, Journal of Biological Chemistry, Elsevier, 2019, 294:8861-8871; Puthusseri et al, American Physiological Society, 2017, 312:L783-L796.) Briefly, deparaffinized lung sections were rehydrated with xylene and decreasing alcohol gradient. Rehydrated lung sections were subjected to antigen heat-induced epitope retrieval by placing the slides in a slide glass container with a citrate buffer (0.1 M citric acid and 0.1 M sodium citrate, pH 6.0) in a water bath at 95° C. for 20 min. After cooling to room temperature, sections were blocked by solution of mouse kit (M.O.M. kit, Vector Laboratories) for 60 min. Then, tissue sections were incubated with primary antibodies diluted in blocking buffer at 4° C. for overnight in humidified chamber. After washing in PBS, tissue sections were incubated for 90 min at room temperature with fluorescent dye-conjugated secondary antibodies and Hoechest 33342 for nuclear staining.

Statistical analysis. The differences between experimental values were analyzed by one-way ANOVA followed by Tukey's post-hoc test using GraphPad Prism 4.0 software (GraphPad Software Inc., San Diego, CA).

### Results

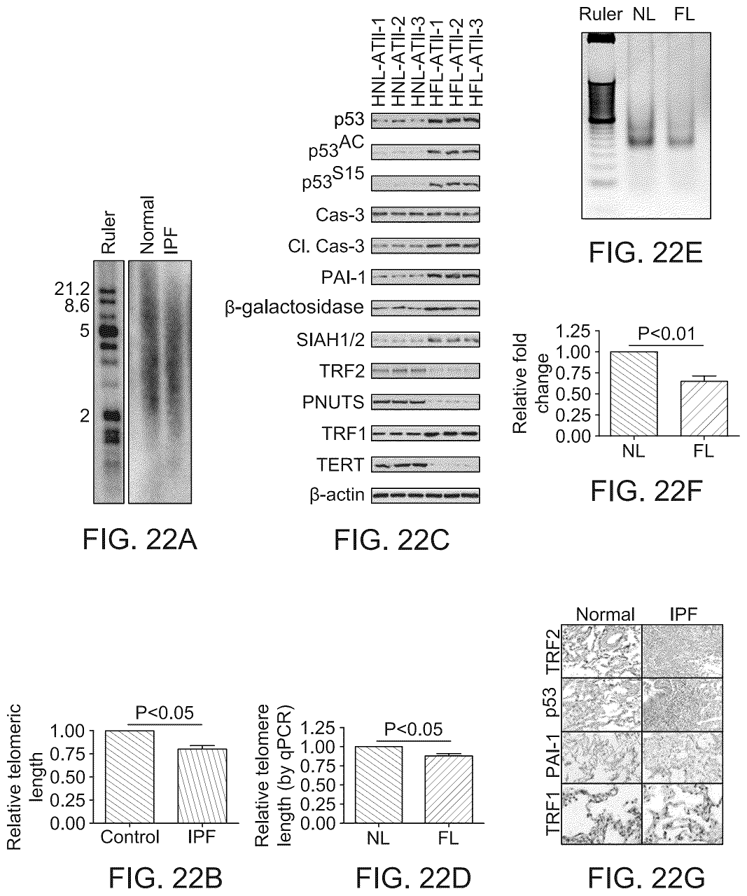

Shortening of the telomere was observed in A₂Cs of patients with both IPF and COPD. TeloTTAGGG assay revealed a significant reduction in A₂C-telomere length in IPF patients (FIGS. 22A-22B), which was further substantiated by the qPCR assay (FIG. 22D). This was further reflected by a significant down-regulation in TERT enzyme activity analyzed by the TRAPeze enzyme assay (FIGS. 22E-22F). Relative telomere length measurements by qPCR strongly correlated with results obtained by southern blot technique or Trapezes enzyme-based assay. As shown in FIG. 22C, Western blot analysis showed an increase in the expression of p53, and its activation through both acetylation of lysine 379 and phosphorylation of serine 15 residues in A2Cs isolated from IPF lungs when compared to their baseline expression in control donor lungs. Elevated expression of activated (cleaved) caspase-3 and β-galactosidase observed in A₂Cs from IPF lungs, imply reduced viability due to increase in both apoptosis and replicative senescence in these cells. Further, increased expression of SIAH-1, a p53-inducible E3 ubiquitin ligase that is known to down regulate the telomere repeat binding factor 2 (TRF2) was found. Consistent with reduction in telomere length and increased SIAH-1, marked downregulation of telomerase reverse transcriptase (TERT) and TRF2 expression, respectively were found. These changes were associated with upregulation of TRF1, which is known to suppress the expression of TERT enzyme. Immunohistochemical (IHC) analysis further showed reduced expression of TRF2, whereas p53, and its downstream target PAI-1 and TRF1 were upregulated in the IPF lung sections (FIG. 22G).

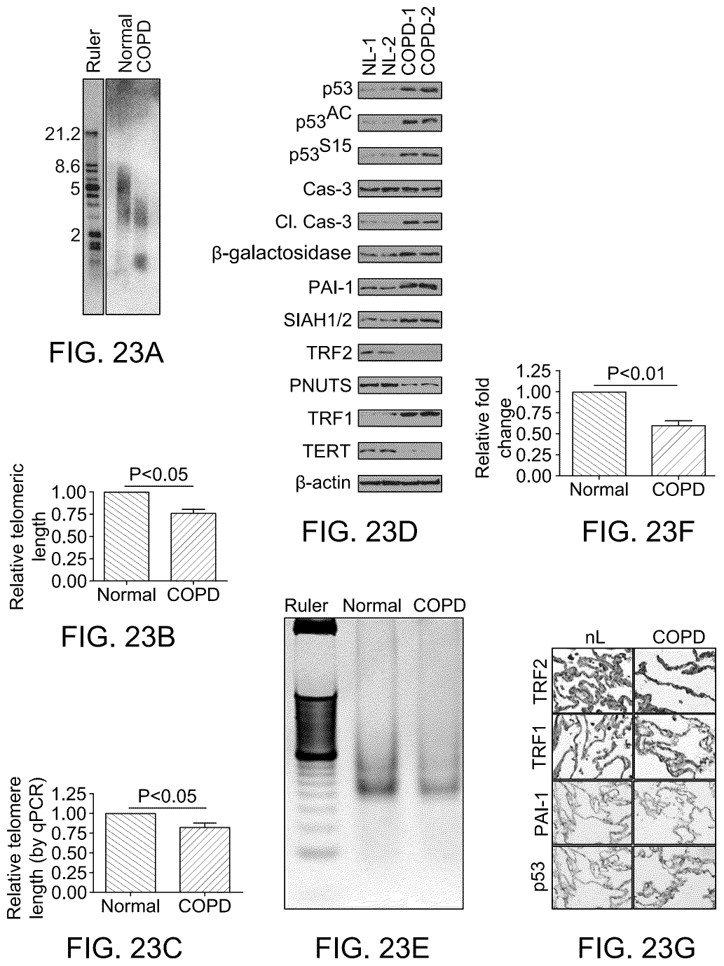

Next, the genomic DNA isolated from A₂Cs from the patients with COPD was analyzed, and a similar pattern of telomere shortening occurred when compared with the corresponding telomere length in A₂Cs from control subjects by TeloTAGGG assay (FIGS. 23A-23B). Telomere length analysis by qPCR had shown significant reduction in telomere length (FIG. 23C). In addition, TERT enzyme activity testing also showed a significant reduction in A₂Cs of COPD patients (FIGS. 23E-23F). A marked increase in total, acetylated and serine-15 phosphorylated p53 protein as well as activation of caspase-3 and β-galactosidase was also observed (FIG. 23D). These data indicate that the increased p53 due to post-translational modifications such as acetylation and serine phosphorylation of p53 protein may lead to the increase in apoptosis and senescence in A₂Cs from COPD lungs. The upregulation of the SIAH-1 observed in A₂Cs of patients with COPD might have resulted in the inhibition of TRF2, and subsequent downregulation of the TERT expression in these cells. As shown in FIG. 23G, IHC analysis of the lung sections showed downregulation in TRF2, while the expression of p53, PAI-1 and TRF1 were increased in COPD lungs.

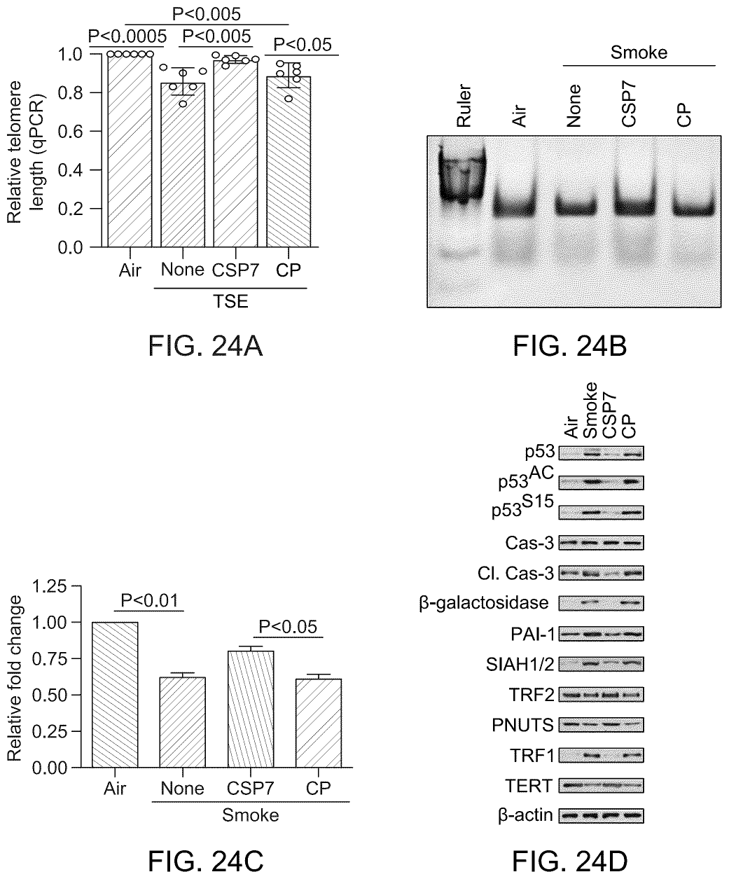

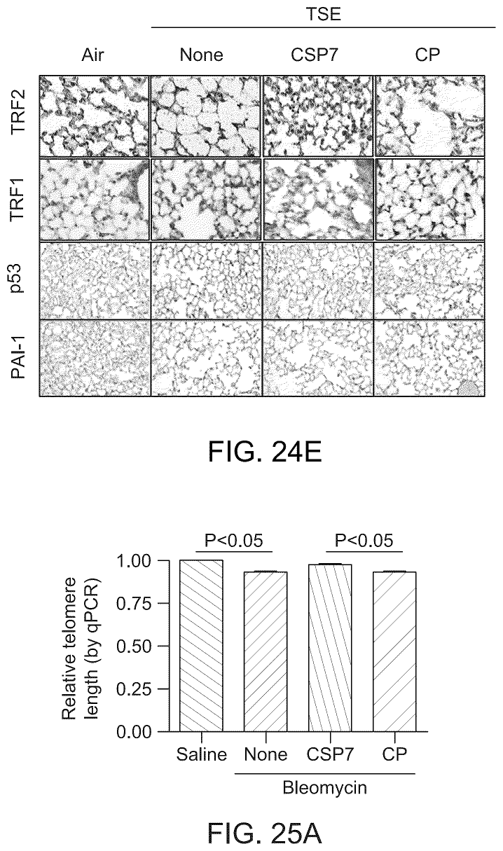

The A₂Cs from the WT mice exposed to 20 weeks of tobacco smoke also showed a significant reduction in telomere length when analyzed by qPCR (FIG. 24A), albeit to a lesser extent of telomere reduction than those observed in A₂Cs from human COPD and IPF lungs. Interestingly, treatment of mice exposed to TS with CSP7 showed a significant resistance when compared to post-TS telomere shortening in mice that received control peptide (CP). As shown in FIGS. 24B-24C, the enzyme activity of TERT in CSP7 treated mice with post-TS lung injury was also significantly higher than the corresponding activity in the CP group. Consistent with the changes observed in A₂Cs from human COPD or IPF lung tissues, immunoblotting of lysates of A₂Cs isolated from TS exposed WT mice showed marked increase in total, acetylated and serine phosphorylated p53 along with induction of cleaved caspase-3 and β-galactosidase (FIG. 24D), suggesting increased p53-mediated alveolar epithelial injury. This was markedly suppressed in A₂Cs of mice exposed to CSP7 but not in those subjected to post-TS treatment with CP. Consistent with loss of TS-induced p53, the expression of SIAH-1 was markedly downregulated in A2Cs of CSP7 treated mice while those exposed to CP still showed elevated p53 and SIAH-1. Mice exposed to CSP7 also showed restoration of TRF2 and TERT expression which were otherwise reduced due to TS exposure lung injury. IHC analysis of the lung sections has also showed restoration of TRF2 expression with reduction in p53, PAI-1 and TRF1 in the CSP7 group which was otherwise suppressed in mice with TS exposure lung injury (FIG. 24E).

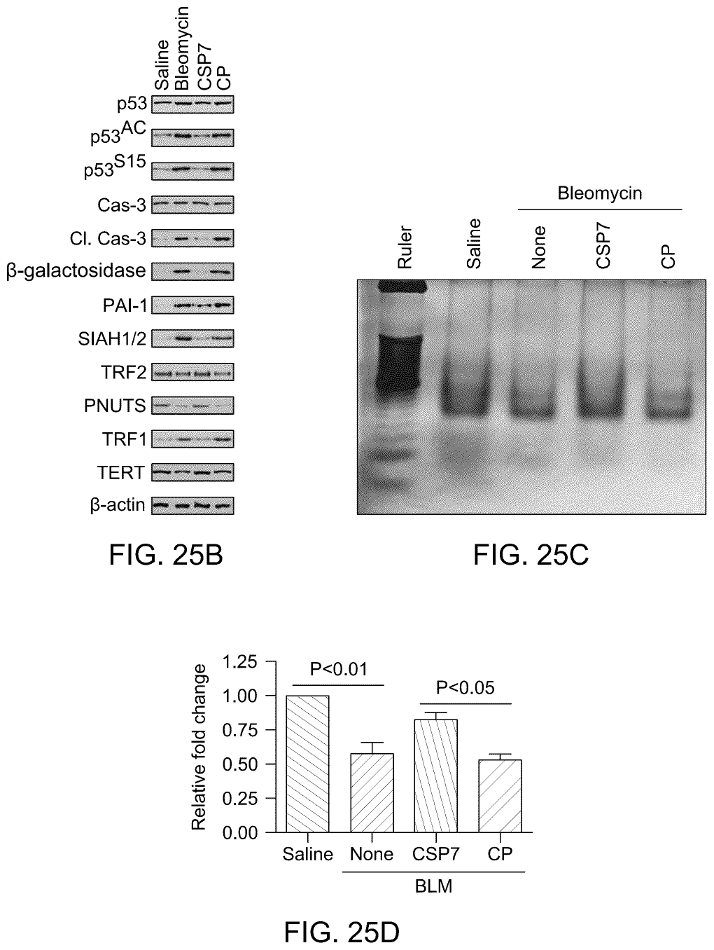

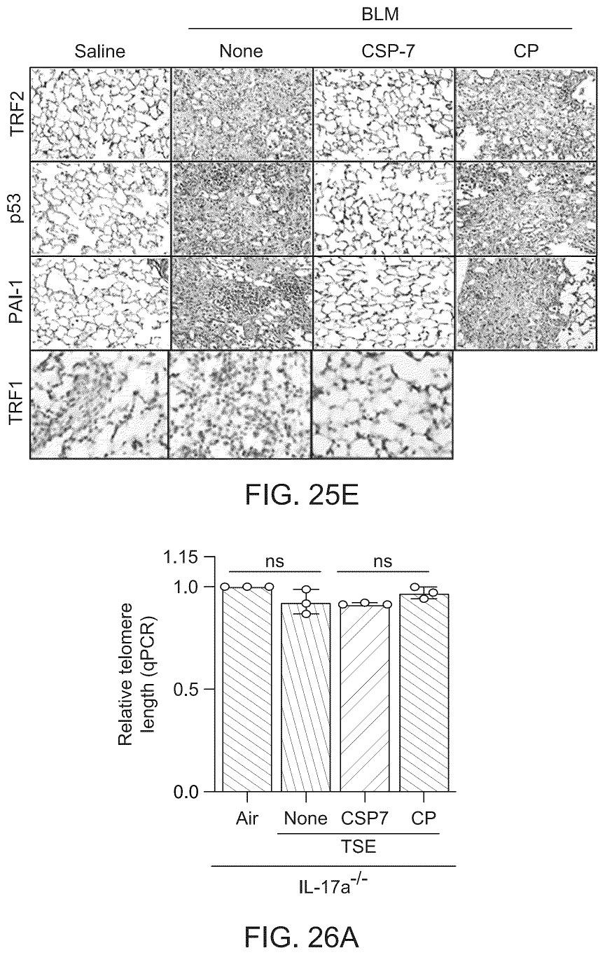

Since A₂Cs isolated from the human IPF lungs show telomere dysfunction and mouse model of PF induced by repeated (8×) exposure to BLM for four months is irreversible and more closely recapitulates the pathogenesis of human IPF (Tiwari et al, 2016), a mouse model of 8X-BLM-induced PF was developed and treated these mice harboring PF with CSP7 or CP as described elsewhere (Tiwari et al, 2016; Shetty et al, 2008b). Consistent with the changes seen in A₂Cs from the IPF lungs, qPCR analysis of A₂Cs isolated from mice with 8X-BLM induced PF showed a reduction in telomere length which was improved in mice treated with CSP7 for two weeks but not CP (FIG. 25A). Further, analysis of TERT enzyme activity showed a significant suppression in A₂Cs of mice with 8X-BLM-induced PF (FIGS. 25C-25D). This was significantly reversed in 8X-BLM-PF mice treated with CSP7, while those exposed to CP still displayed shortened telomere. This was consistent with suppression of p53, p⁵³ downstream targets PAI-1, SIAH1/2, activation of caspase-3, β-galactosidase (FIG. 25B), as indicators of inhibition of apoptosis and senescence. Consistent with the changes seen in A₂Cs of WT mice having TS exposure injury, lysates of A2Cs isolated from mice with 8X-BLM-PF showed marked increase in shelterin component telomere binding protein, TRF1, while suppressing the expression of TRF2, PNUTS and TERT. These changes were reversed following treatment of 8X-BLM-PF with CSP7 for two weeks whereas those exposed to CP still harbor A₂Cs with shortened telomere. IHC analysis of the lung sections has also showed restoration of TRF2 expression with reduction in p53, PAI-1 and TRF1 in the CSP7 group which was otherwise suppressed in mice with TS exposure lung injury (FIG. 25E).

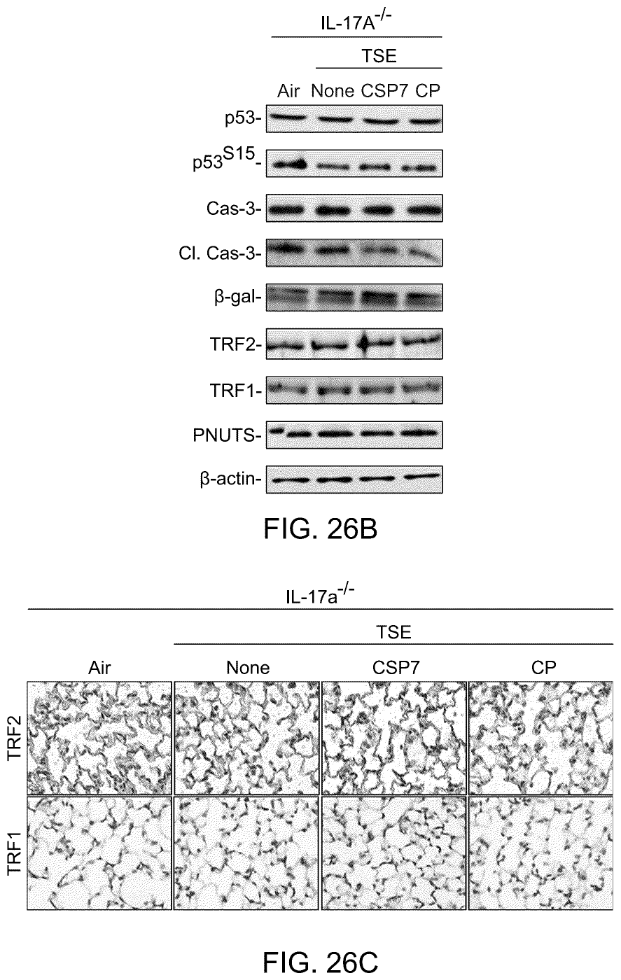

From recent reports (Stanley et al, 2016; Dai et al, 2015, Lawsone et al, 2006), IL-17A is markedly elevated in the lungs and sputum of patients with COPD. These observations are built upon prior work showing that the accumulation of lung lymphoid follicles and IL-17A-positive mast cells are associated with severe COPD (Roos et al, Am J Respir Crit Care Med 2015, 191:1232-1241). Therefore, IL-17A^{-/-} mice were exposed to 20 weeks of TS and A2Cs isolated from these mice and analyzed them for telomere shortening. As shown in FIG. 26A, 20 weeks of TS exposure failed to induce significant telomere shortening in A₂Cs of mice deficient in IL-17A. Consistent with resistance to telomere dysfunction and TS exposure injury, CSP7 or CP had minimal effect in these mice. This was further independently confirmed by minimal induction of p53 and serine phosphorylated p53, active caspase-3 or β-galactosidase in A₂Cs of IL-17A^{-/-} mice exposed to TS vs those kept in ambient AIR, suggesting resistance to apoptosis or senescence or TS exposure lung injury (FIG. 26B) confirming the resistance of IL-17A^{-/-} to telomere shortening. Further, TS exposure failed to affect the baseline expression of Trf1, Trf2 or PNUTS in A₂Cs of IL-17A-deficient mice. As shown in FIG. 26C, IHC analysis of lung sections of IL-17A-deficent mice exposed to 20 weeks of TS further revealed little change in Trf1 or Trf2 antigen staining vs ambient air-kept control mice or those exposed to TS and treated with CSP7 or CP thus corroborating the results of the immunoblotting of isolated A₂Cs.

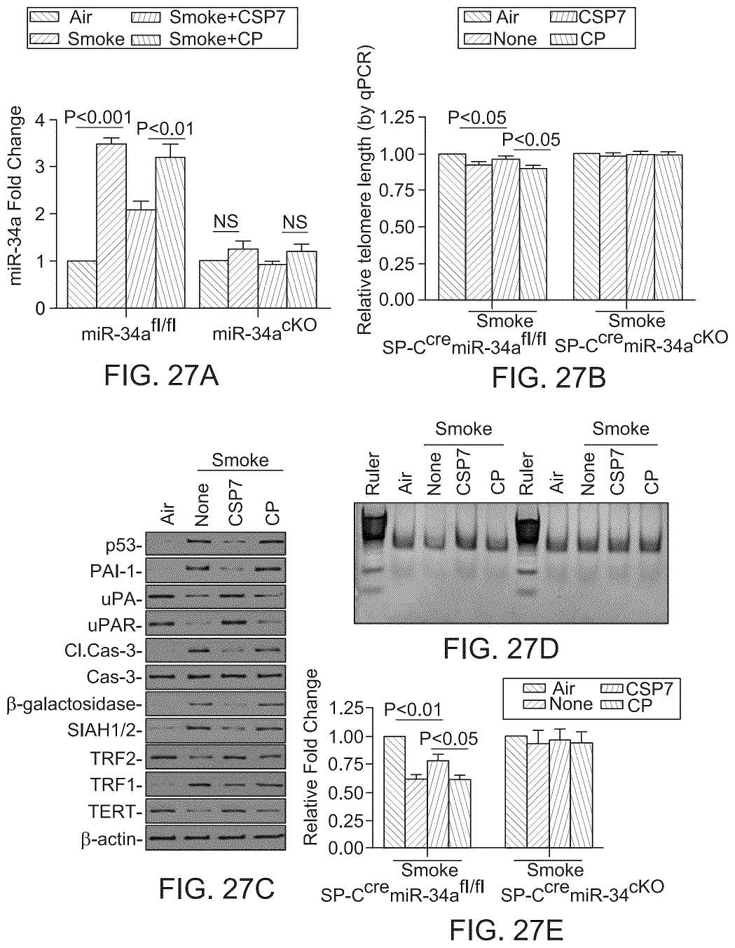

Since p53 induces miR-34a transcription while miR-34 augments p53 acetylation and total p53 expression through feedback induction in A₂Cs, miR-34a^{cKO} lacking its expression in A₂Cs were daily exposed to 20 weeks of TS. As shown in FIG. 27A, induction of p53 by TS exposure caused a significant increase in miR-34a expression A₂Cs of WT or miR-34a floxed (miR-34a^{fl/fl}) mice which is resisted by miR-34a^{cKO} mice. Further, qPCR analysis for telomere length did not show a significant reduction in miR-34a^{cKO} mice exposed to 20 weeks TS, unlike control WT or miR-34a^{fl/fl} mice which had retained the miR-34a gene (FIG. 27B). Further, CSP7 treatment showed significant protection against TS-induced telomere shortening in WT or miR-34a^{fl/fl} mice, while it had minimal effect in miR-34a^{cKO} mice. This was independently confirmed by assaying telomerase enzyme activity, which was significantly downregulated in A₂Cs of miR-34a^{fl/fl} mice exposed to TS. This was reversed following treatment with CSP7 while CP had little effect on TS induced inhibition of telomerase activity. Interestingly, miR-34a^{cKO} mice exposed to TS resisted telomere dysfunction and CSP7 treatment had limited effects on baseline telomerase activity in these mice (FIGS. 27D-27E). Western blotting of A₂C lysates indicated that expression of p53, PAI-1, active caspase-3 and β-galactosidase was upregulated while uPA and uPAR was downregulated in miR-34a^{fl/fl} mice exposed 20 weeks of TS (FIG. 27C). These changes were associated with parallel increase in the expression of SIAH1/2 and TRF1 while TRF2 and TERT expression was reduced in miR-34a^{fl/fl} mice exposed to TS. This was resisted by miR-34a^{cKO} mice, suggesting telomere dysfunction in A₂Cs due to chronic TS exposure lung injury.

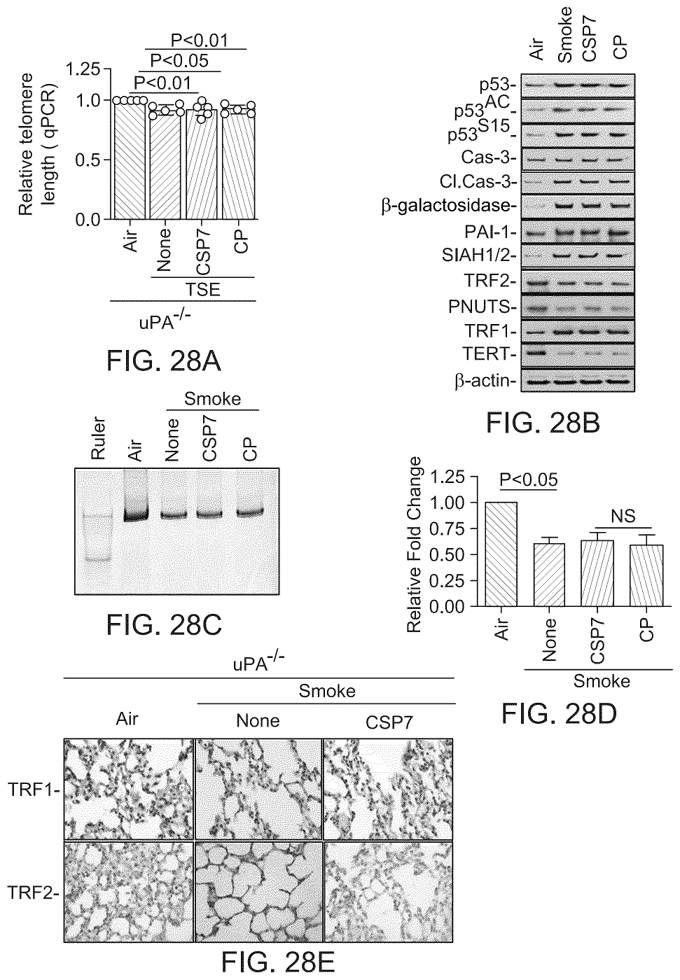

Since TS exposure injury induced PAI-1 and suppressed uPA expression in consistent with induction of p53 in A₂Cs, uPA^{-/-} mice were next exposed to 20 week of TS and subjected to CSP7 treatment. As shown in FIG. 28A, qPCR analysis revealed that TS exposure caused a significant shortening of telomere in A₂Cs of uPA-deficient mice as seen in WT mice exposed to 20 weeks TS. However, unlike WT mice, A₂Cs from mice lacking uPA expression exposed TS failed to respond to CSP7 treatment and exhibited telomere shortening associated lung injury due to TS like those exposed to TS or TS and CP. This was independently confirmed by testing the telomerase enzyme activity in isolated A₂Cs (FIGS. 28C-28D). Consistent with inability of CSP7 to reverse telomere shortening or improve telomerase activity, CSP7 failed to inhibit post-translational modification or induction of p53, active caspase-3 or β-galactosidase observed in A₂Cs of mice with TS exposure lung injury when analyzed by Western blotting for protein expression (FIG. 28B). There was no significant change in the expression of TRF1, TRF2 and TERT in A₂Cs isolated from uPA-deficient mice exposed to TS left untreated or treated with CSP7 or CP. IHC analysis lung sections of uPA-deficient mice exposed to TS for TRF1 showed minimal inhibition by CSP7 treatment (FIG. 28E).

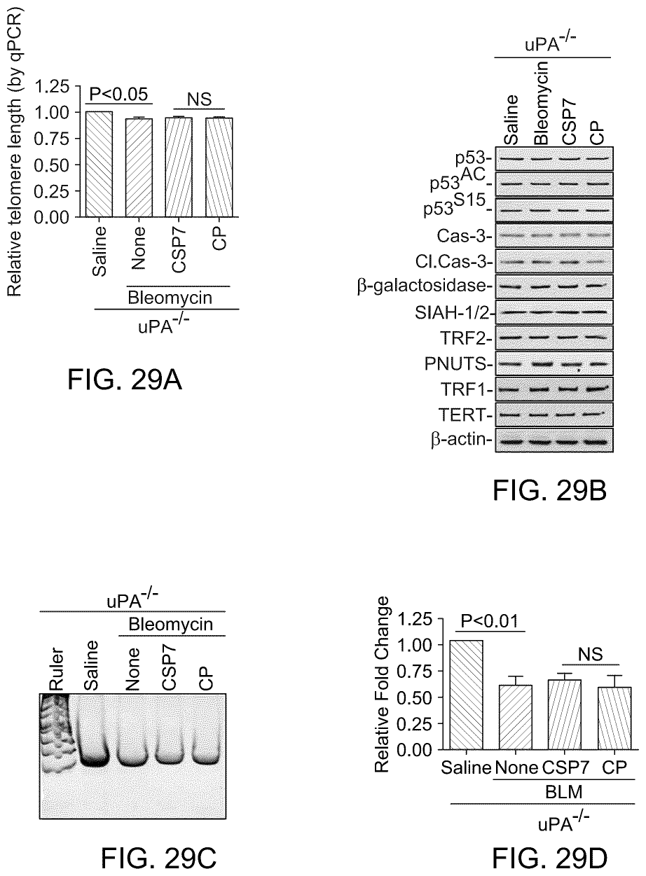

Similarly, CSP7 treatment of uPA-deficient mice with 8×BLM-induced PF failed to restore telomere length based on qPCR analysis of genomic DNA (FIG. 29A) or telomerase enzyme activity by the trapeze method (FIGS. 29C-29D) of isolated A2Cs. Western blotting of A₂Cs lysates from these mice confirmed that CSP7 treatment had minimal effect on otherwise changes the expression of p53, ^{AC}p53, ^{S15}p53, apoptosis or senescence or p53 target genes PAI-1, SIAH1/2, shelterin component binding proteins such as TRF1 and TRF2, or PNUTS or TERT induced by TS exposure injury in uPA^{-/-} mice (FIG. 29B). Upon testing WT and uPA deficient mice, it was found that p53 induces PAI-1 expression in A₂Cs of mice with TS- and 8×BLM-induced lung injury and CSP7 failed to suppress p53-induced PAI-1 expression or telomere dysfunction in uPA-deficient mice.

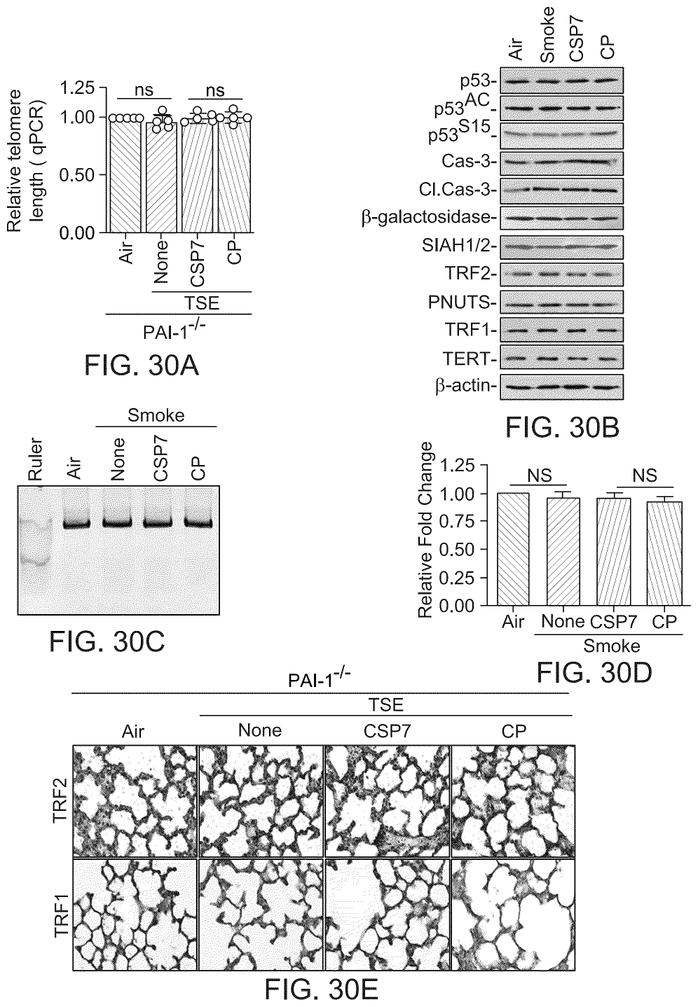

As shown in FIG. 30A, qPCR analysis of genomic DNA from isolated A₂Cs revealed that TS exposure for 20 weeks failed to cause a significant change in telomere length in PAI-1-deficient mice. Similarly, CSP7 or CP treatment of PAI-1-deficient mice exposed TS had minimal effect on telomere length. Telomerase enzyme activity analyzed by trapeze assay also showed no significant change activity between PAI-1^{-/-} mice kept in ambient AIR or exposed to TS or TS and CSP7 or CP (FIGS. 30C-30D). Western blotting of isolated A₂Cs lysates likewise confirmed minimal increase in total or post-translationally modified p53, cleaved caspase-3 or β-galactosidase, suggesting changes in apoptosis, senescence or the expression of SIAH1/2 and PNUTS or proteins that are directly associated with the telomere such as TRF1, TRF2 and TERT (FIG. 30B). IHC analysis of lung sections from these mice further confirmed minimal change in TRF1 or TRF2 antigen expression in PAI-1^{-/-} mice kept in ambient AIR vs those exposed to TS with or without CSP7 or CP treatment (FIG. 30E).

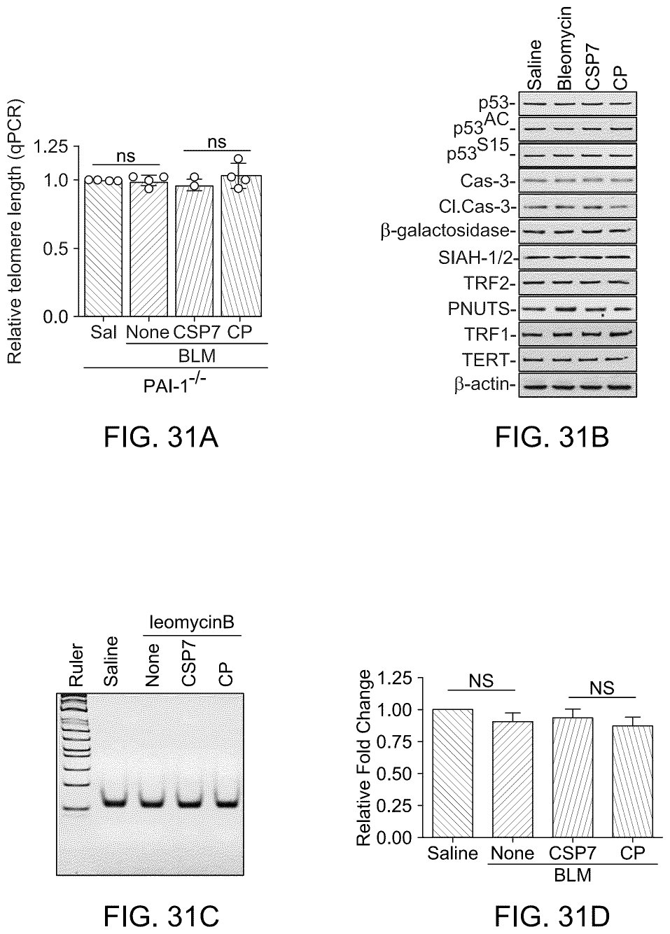

To directly test importance of increased PAI-1 expression in telomere dysfunction, PAI-1 ^{-/-} mice were exposed to 8×BLM. qPCR analysis of genomic DNA (FIG. 31A) or trapeze assay for telomerase enzymatic activity (FIGS. 31C-31D) of isolated A₂Cs revealed minimal effect in PAI-1-deficient mice exposed to 8X-BLM. Western blotting of A₂Cs lysates showed little or no difference in the expression of p53, ^{Ac}p53, ^{S15}p53, cleaved caspase-3, β-galactosidase, SIAH1/2, PNUTS, TRF1, TRF2 and TERT between PAI-1-deficient mice exposed to saline, 8X-BLM with or without CSP7 or CP (FIG. 31B), suggesting resistance of PAI-1-deficient mice to 8X-BLM-induced lung injury. Collectively, these novel findings indicate an intricate link between p53-mediated changes in major components of uPA-fibrinolytic system and telomere dysfunction A2Cs.

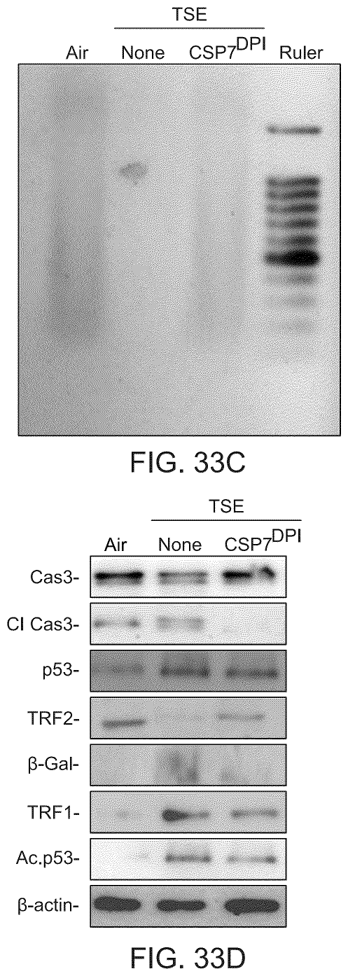

Consistent with systemic administration of CSP7 by IP injection, qPCR for telomere length revealed that airway delivery by inhalation of micronized CSP7 dry powder (CSP7^{DPI}) to mice with TSE-LI caused a significant increase in telomere length in A₂Cs (FIG. 33A). The protective effect of airway CSP7 was further confirmed by TRAPEZE assay for telomerase enzymatic activity (FIG. 33B) as well as TeloTAGGG assay for the telomere length of the isolated genomic DNA (FIG. 33C). Immunoblotting of isolated A₂Cs lysates likewise confirmed inhibition of p53, Ac-p53, cleaved caspase-3 and β-galactosidase, suggesting CSP7^{DPI} inhibits TSE induced A₂C apoptosis and senescence in WT mice. Consistent with protection against TSE-induced telomere shortening, treatment of mice with TSE-LI by CSP7^{DPI} restored A₂Cs TRF2 and inhibited TRF1 which are otherwise altered in mice with TSE-LI and left untreated compared to ambient air kept control mice (FIG. 33D). IHC analysis of the lung sections has also showed inhibition of TRF1 expression in the CSP7^{DPI} group which was otherwise increased in mice with TSE-LI (not shown).

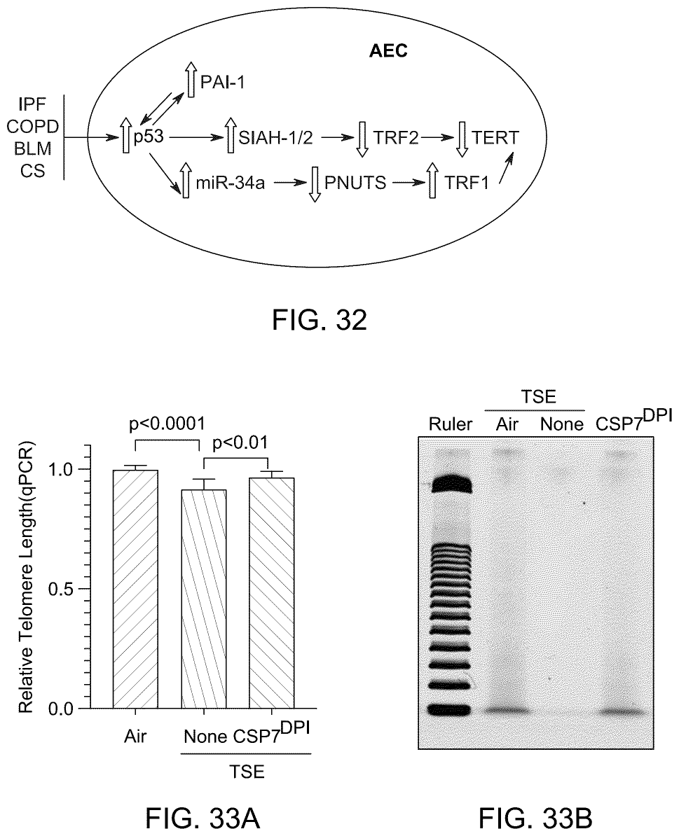

Collectively, these novel findings indicate an intricate link between p53-mediated changes in major components of uPA-fibrinolytic system and telomere dysfunction in A₂Cs due to induction of Cav1 often associated with chronic lung diseases such as IPF or COPD (FIG. 32). This can be reversed by systemic or airway delivered CSP7, which competes with increased Cav1 for signaling intermediaries that promotes Cav1-mediated induction of p53 and cascades of downstream events in injured A₂Cs.

### Discussion

The chronic lung diseases such as PF, including IPF and emphysema are debilitating diseases characterized by progressive decline in lung function, share common risk factors such as aging, premature aging due to particulates exposure or genetic factors. A₂Cs are the common targets of damage from progressive and persistent alveolar epithelial injury from chronic exposure to TS, other particulates and DNA damaging agents, and inflammatory cells in humans and in pre-clinical models, which induces senescence and apoptosis in A₂Cs leading to reduced A₂C renewal and development of PF or emphysema. (Shetty et al, 2012; Park et al, 2007; Bhandary et al, 2015). A₂Cs not only produce surfactant proteins but also divide and differentiate into type I alveolar epithelial cells, which are the most abundant epithelial cells in the lung parenchyma. Importantly, the pathogenesis of emphysema and ILDs, including human IPF has been directly linked to a loss of A₂C renewal capacity due to reduced viability, which limits alveolar epithelial regeneration due to stem cell exhaustion. (Shetty et al, 2012; Park et al, 2007; Tsuji et al, 2004). Although PF and emphysema exemplify distinct clinical-radiological and pathophysiological patterns, these diseases are associated with similar fates of vulnerable cell population i.e., A₂C senescence and apoptosis, telomere dysfunction. Further, literature suggest that about 30% of patients with emphysema also develop PF, suggesting common underlying trigger mechanism but divergent injury-repair responses most likely leading to distinct disease pathologies. This regenerative capacity of A₂Cs requires active telomere maintenance.

Telomeres consist of tandem TTAGGG repeats and associated shelterin components, the protein complex proteins that form a capping structure at the ends of chromosomes, which prevent them from degradation and activation of DNA repair responses. (Cawthon, 2002). Telomere attrition occurs with each cell division until a point cell with critically shortened telomere undergo senescence due to uncapping of telomeres, inducing a DNA damage response and activation of cell-cycle arrest programming. (Cawthon, 2002). Telomere shortening is proposed to be a primary molecular cause of aging and premature aging. Short telomeres block the proliferative capacity of stem cells, affecting their potential to regenerate tissues and trigger age associated diseases. Literature further suggests that telomere dysfunction leading to PF is specific to A₂Cs and not simply a consequence of any resident lung cells in the absence of lung injury. (Naikawadi et al). The patients with both PF and emphysema have also been noted to have abnormally short telomeres in A2Cs. (Liu et al, Am J Respir Cell Mol Biol 2013, 49:260-268). A subset of patients with age-associated pathology such as IPF manifests mutations in essential telomerase genes such as TERT, or TERC. (Armanios et al, N Engl J Med 2007, 356:1317-1326; Tsakiri et al, Proc Natl Acad Sci USA 2007, 104:7552-7557.)

Similarly, mutation in TERT and TR are associated with the pathogenesis of emphysema. (Stanley et al, 2015). The mutation in TERT can be familial as well as non-familial (Tsakiri, 2007), hinting that factors contributing to mutations can in turn affect telomere failure and associated pathologies. The mechanisms by which telomere defects provoke diverse types of lung diseases such as emphysema and IPF are not understood, but a number of observations have pointed to lung-intrinsic factors and epithelial cells dysfunction as candidate events. (Alder et al, 2015). For example, in telomerase-null mice, DNA damage preferentially accumulates in the air-exposed epithelium after environmentally induced injury, such as with chronic TS exposure. The additive effect of environmental injury and telomere dysfunction has been suggested to contribute to the susceptibility to emphysema seen in these mice. (Alder et al, 2015). In subjects carrying a TERT mutation, A₂C telomere length was significantly shorter than in control subjects and inclined with reduced survival. However, no such differences observed in surrounding cell telomere length (Leon et al, PLOS ONE, Public Library of Science, 2010, 5:e10680), suggesting that telomere shortening critically affects A₂Cs in fibrotic areas. Further, in sporadic IPF, A₂Cs telomere length was significantly longer in non-fibrotic areas than in fibrotic regions (Naikawadi et al, JCI Insight 1:e86704) thus implicating telomere dysfunction as a potential cause of fibrogenesis. Literature suggest that telomere related pathology plays a role in both familial PF caused by mutations in surfactant related genes or genes that influence telomere maintenance and sporadic IPF. (Garcia, Proc Am Thorac Soc, American Thoracic Society—PATS, 2011, 8:158-162). Further, familial patients with a TERT mutation show shorter telomeres than in sporadic IPF, which is associated with worse survival. (Leon et al, 2010). Lung lavage fluids exhibit high levels of uPA activity and alveolar fibrinolysis. (Idell et al, J Clin Invest 1989, 84:695-705; Barazzone et al, J Clin Invest 1996, 98:2666-2673; Olman et al, J Clin Invest 1995, 96:1621-1630. However, impaired fibrinolysis is mainly attributable to local over-expression of PAI-1 (major inhibitor of uPA) injury. (Chapman et al, 1986; Chapman 2004; Hasday et al, 1988; Bertozzi et al, 1990; Bachofen and Weibel, 1982; Idell et al, 1989; Eitzman et al, 1996; Lardot et al, 1998; Xu et al, 2009; Hu et al, 2009, Zidovetzki et al, 1999; Barazzone et al, J Clin Invest 1996, 98:2666-2673; Olman et al, J Clin Invest 1995, 96:1621-1630.)

p53, by binding through its C-terminal amino acid residues 296-393 with a 70-nucleotide (nt) destabilization determinant of PAI-1 3′UTR mRNA induces PAI-1 expression. (Shetty et al, 2008a; Shetty et al, 2008c; Shetty et al, 2007). p53 also binds PAI-1 promoter sequences and increases PAI-1 mRNA transcription. (Bhandary et al, 2013; Kunz et al, Nucleic Acids Res 1995, 23:3710-3717.) Furthermore, treatment of A2Cs with BLM or TS was found to increase both p53 and PAI-1 expression and reduces cellular viability. These effects are reversed by inhibition of p53 binding to endogenous PAI-1 mRNA or inhibition of p53 accumulation by treatment with either CSP or CSP7. (Shetty et al, 2012; Bhandary et al, 2015; Tiwari et al, 2016). Lung tissues from patients with IPF or COPD also show elevated levels of p53 and PAI-1 in A₂Cs. (Bhandary et al, 2015; Tiwari et al, 2016; Marudamuthu et al, 2015). The inventor recently reported increased expression of p53, Cav1 and PAI-1 in A₂Cs from the lungs of patients with IPF or COPD and in WT mice with BLM- or TS-induced lung injury. (Shetty et al, 2012; Tiwari et al, 2016). It was found that p53 induces PAI-1 expression in A₂Cs. (Shetty et al, 2012; Marudamuthu et al, 2015; Shetty et al, 2008a; Bhandary et al, 2015). Mice lacking either p53 or PAI-1 resist TS- or BLM-induced lung injury (Shetty et al, 2017; Bhandary et al, 2013) as well as development of PF resulting from BLM lung injury. Consistent with increased expression of p53 and PAI-1, the inventor and others (Liu et al, 2019; Liu et al, 2013; Armanios et al, 2007) show telomere dysfunction in A₂Cs from the lungs of patients with IPF or COPD, and WT mice with chronic TS or BLM exposure induced lung injury. These findings further support the targeting of Cav1 induced p53- and PAI-1-mediated pathways in A2Cs using Cav1 scaffolding domain peptide (CSP) or its 7-mer deletion fragment, CSP7, improves viability and inhibits lung injury and PF by blocking senescence and apoptosis caused by chronic TS or BLM exposure. CSP/CSP7 appears well-tolerated and inhibits lung injury in mice. (Shetty et al, 2012, Bhandary et al, 2015; Tiwari et al, 2016; Marudamuthu et al, 2015; Shetty et al, 2017).

The inventor further found that WT mice exposed to 8×BLM or 20 weeks of passive TS showed significant reduction relative telomere length suggesting telomere dysfunction. Further literature suggests that telomere length correlates with pulmonary function in patients with IPF or COPD. (Liu et al, 2013). These changes were associated with reduction in shelterin complex proteins such TRF2 while the expression of TRF1 was increased. Two telomere-specific DNA binding proteins, TRF1 and TRF2 are required for human telomere function. (Srinivas et al, 2020). TRF1 overexpression leads to progressive telomere shortening while its inhibition enhances telomere length suggesting TRF1 role in regulation of telomere length. TRF2 protects the chromosome ends. (Srinivas et al, 2020), There changes were associated with induction SIAH1 and loss of TERT and PNUTS expression suggesting that increased p53 is contributing telomere dysfunction. Further treatment of these mice with CSP7 prevented telomere attrition in A2Cs with reversal of shelterin complex proteins. This suggests that CSP7 protects against telomere dysfunction by restoring expression shelterin complex proteins to protect telomere ends. These changes are intricately linked to induction of p53 and PAI-1 in A2Cs.

Consistent with resistance of PAI-1-deficient mice to development of PF or TS-induced lung injury, TS or BLM lung injury failed to reduce relative telomere length in these mice. Similarly, no significant change in the basal expression of shelterin complex protein in PAI-1-deficient mice exposed to either TS or BLM was found. CSP7 also has minimal effect in expression of p53 in A₂Cs of these mice. Consistent with earlier findings and others (Marudamuthu et al, 2015; Bhandary et al, 2013; Gharaee-Kermani et al, Expert Opinion on Investigational Drugs, Taylor & Francis, 2008, 17:905-916), uPA-deficient mice are highly susceptible to BLM-induced lung injury and PF. A₂Cs isolated from uPA-deficient mice with TS-induced lung injury or BLM-induced PF also demonstrated telomere dysfunction. Further, CSP7 treatment failed to suppress both TS- or BLM-induced p53 or PAI-1 expression or telomere dysfunction, suggesting an intricate link between TS-or BLM-induced p53 and down PAI-1 expression and telomere dysfunction in A₂Cs. This possibility is further supported by the lack of BLM or TS exposure induced lung injury and resistance of tamoxifen inducible conditional knockout mice lacking miR-34a in A₂Cs to telomere dysfunction. These mice also showed minimal alteration in the baseline levels of shelterin complex proteins, p53, PAI-1, senescence and apoptosis, suggesting increased p53 through miR-34a-p53 feedback induction of PAI-1 contributes to telomere dysfunction and senescence in A₂Cs during lung injury. It was also found that IL-17A-deficent mice exposed TS resisted lung injury and p53 and PAI-1 expression, as well as telomere dysfunction otherwise seen in WT mice. These findings collectively suggest that TS or BLM induces IL-17A to increase p53 and PAI-1 that in turn promote telomere dysfunction in A₂Cs. Oxidative stress and chronic inflammation are the most likely factors thought to be contributing to the loss of telomere DNA. (Masi et al, Free Radical Biology and Medicine 2011, 50:730-735.) A deficiency of either p53 or PAI-1 makes mice highly resistant to experimentally induced alveolar epithelial injury and air space enlargement (Shetty et al, 2012; Bhandary et al, 2015), implying that changes in A₂C p53 and PAI-1 expression, subsequent telomere dysfunction, viability are important and clinically relevant, and occur in patients with parenchymal lung diseases such as emphysema and PF, including IPF.

### Example 20

Chronic obstructive pulmonary disease (COPD) is a debilitating lung disease with no effective treatment that can reduce mortality or slow the disease progression. COPD is the third leading cause of global death and is characterized by airflow limitations due to chronic bronchitis and alveolar damage/emphysema. Chronic tobacco smoke exposure (CS) damages airway and alveolar 32 epithelium and remains a major risk factor for the pathogenesis of COPD. Expression of caveolin-1, tumor suppressor protein; p53, and plasminogen activator inhibitor-(PAI-1), one of the downstream targets of p53, were markedly increased in airway epithelial cells (AECs) as well as in type II alveolar epithelial (AT2) cells from lungs of patients with COPD or wild-type mice with CS-induced lung injury. Moreover, p53- and PAI-1-deficient mice resisted CS-induced lung injury. Furthermore, treatment of AECs, AT2 cells or lung tissue slices from patients with COPD, or mice with CS-induced lung injury with seven amino acid caveolin-1 scaffolding domain peptide (CSP7) reduced mucus hypersecretion in AECs and improved AT2 cell viability. Notably, induction of PAI-1 expression via increased caveolin-1 and p53 contributed to mucus hypersecretion in AECs and reduced AT2 viability, due to increased senescence and apoptosis, which was abrogated by CSP7. In addition, treatment of wild-type mice having CS-induced lung injury with CSP7 by intraperitoneal injection or nebulization via airways attenuated mucus hypersecretion, alveolar injury, and significantly improved lung function. This study further validates the therapeutic role of CSP7 for treating CS-induced lung injury and COPD.

Chronic tobacco smoke exposure (CS) remains a major risk factor for the pathogenesis of COPD, a debilitating disease with no effective treatment. COPD is the third leading cause of global death and characterized by airflow limitations due to chronic bronchitis and alveolar damage/emphysema. Increased caveolin-1 contributes to CS-induced airway mucus-hypersecretion and alveolar damage. This is reversed by CSP7 in preclinical models, demonstrating the therapeutic use of CSP7 for treating CS-induced lung injury and COPD.

### Introduction

Chronic obstructive pulmonary disease (COPD) affects up to 24 million people and is the third leading cause of death in the United States. Acute exacerbations of COPD are the second leading cause of hospital stays and incur costs of more than 18 billion dollars annually in the United States. Chronic exposure to tobacco smoke (CS) due to active cigarette smoking or passive CS exposure is the most common risk factor for COPD, with cigarette smokers known to have greater COPD-related mortality than non-smokers (Kohansal et al. Am J Respir Crit Care Med 180: 3-10, 2009. doi: 10.1164/rccm.200901-00470C; Hogg et al. N Engl J Med 350: 2645-2653, 2004. doi: 10.1056/NEJMoa032158; Curran and Cohn, Am J Respir Cell Mol Biol 42:268-275, 2010. doi: 10.1165/rcmb.2009-0151TR; Decramer and Janssens, Eur Respir Rev 19:134-140, 2010. doi: 10.1183/09059180.00003610.) Despite considerable progress in the past decade, molecular pathogenesis of COPD remains poorly understood. There are currently no interventions to reverse the progression of COPD-related lung injury. In COPD, chronic lung inflammation leads to narrowing of small airways, airway obstruction and alveolar wall destruction. Airway epithelial cells (AECs) and alveolar epithelial cells are common targets for damage from chronic CS exposure and mediators and cytokines released from inflammatory cells (Mortaz et al. Biochim Biophys Acta. 12:1104-10, 2011. doi: 10.1016/j.bbadis.2011.06.002; Bruggemann et al. Front Immunol. 8:718, 2017. doi: 10.3389/fimmu.2017.00718; Xie et al. PLoS One. 13:e0193334, 2018. doi: 10.1371/journal.pone.0193334). The pathogenesis of COPD is directly associated with AEC metaplasia and airway inflammation leading to mucus-hypersecretion (MH) as well as loss of alveolar structure due to replicative senescence and apoptosis of alveolar epithelial cells, including progenitor type II alveolar epithelial (AT2) cells (Sun et al. Clin Sci (Lond). 133(13):1523-1536, 2019. doi: 10.1042/CS20190331;Yu et al., Clin Sci (Lond). 133(4):551-564, 2019. doi: 10.1042/CS20180912. PMID: 30733313; Ning et al., PLoS One. 8:e83429, 2013. doi: 10.1371/journal.pone.0083429; Chen et al., Oxid Med Cell Longev. 2020:8217642, 2020. doi: 10.1155/2020/8217642.) These cognate events consequently result in airway obstruction and emphysema respectively and contribute significantly to morbidity and mortality (Kohansal et al, 2009; Hogg et al, 2004).

Chronic bronchitis (CB) from longtime smoking is known to involve aberrant cellular and inflammatory responses of the airways to long time exposure to CS. This results in the disruption of the AEC function and has often been attributed to a reduction in AEC cilia length and death. These cellular changes are followed by re-epithelialization by goblet cells and excessive mucus production, leading to impaired mucociliary clearance (MCC). Clinically, mucolytic drugs have been shown to reduce COPD exacerbation and improve the quality of life in patients (Hogg, et al, 2004), thus, underscoring the importance of targeting MH in COPD therapy. Among the 21 genes that are ascribed to encode mucins in the human genome, mucin 5AC (MUC5AC) is highly expressed in the airways (Thornton et al, Annu Rev Physiol 70:459-486, 2008. doi: 10.1146/annurev.physiol.70.113006.100702.) Mucus may alter the normal structure and status of goblet cells after failing to incorporate with MUC5AC. Without the normal reaction between MUC5AC and mucus, airway visco-elasticity becomes susceptible to plugging (Woodruff et al., Am J Respir Crit Care Med 180: 796, 2009 doi: 10.1146/annurev.physiol.70.113006.100702.) Goblet cell differentiation is dictated by a large network of genes, in which transcription factors sterile alpha motif-pointed domain containing E26 transformation-specific like factor (SPDEF), and forkhead box protein A2 (FOXA2), are two key regulators. SPDEF is required for goblet cell differentiation and excessive mucus production, including secreted airway mucin, MUC5AC (Park et al., J Clin Invest 117: 978-988, 2007. doi: 10.1172/JCI29176; Rajavelu et al., J Clin Invest 125: 2021-2031, 2015. doi: 10.1172/JCI79422.) Whereas FOXA2 is a potent inhibitor of goblet cell differentiation in the lung (Wan et al, Development 131 953-964, 2004. doi: 10.1242/dev.00966; Chen et al., J Immunol 184: 6133-6141, 2010. doi: 10.4049/jimmunol.1000223), FOXA3 is highly expressed in airway goblet cells and induces SPDEF, MUC5AC, and AGR2 genes that are required for goblet cell metaplasia in the airway epithelium. The observed effects of FOXA3 on mucus-related gene expression are likely mediated, at least in part, by its ability to induce SPDEF (Chen et al., Am J Respir Crit Care Med 189: 301-313, 2014 doi: 10.1164/rccm.201306-11810C.)

The breakdown of ciliated cells also contributes to dysfunction of MCC. AECs exposed to CS show more than a 70% decrease in the number of ciliated cells as well as shortening of the cilia. One of the mechanisms under investigation involves autophagy that is dependent on histone deacetylase 6 (HDAC6). HDAC6 is upregulated in AECs of patients with COPD where it targets damaged and misfolded proteins for proteasomal degradation. In the case of ciliary shortening, HDAC6 was found to colocalize with acetylated alpha-tubulin. It then associates with LC3 which is a well-known essential molecule for autophagy. LC3 exists in two molecular forms; LC3-I is a cytosolic form, whereas LC3-II binds to autophagosomes (Levine and Klionsky, Dev Cell 6:463-477, 2004. doi: 10.1016/s1534-5807(04)00099-1; Roscioli et al, Respir Res 19:234, 742 2018.) The level of LC3-II directly relates to the number of autophagosomes, which is evidence of autophagy. This is consistent with a recent report implicating an increased expression of autophagy markers in the development of COPD (Kim et al., Autophagy 4:887-895, 2008. doi: 10.4161/auto.6767; Ryter et al., Autophagy 5:235-237, 2009. doi: 10.4161/auto.5.2.7495.) The pathogenesis of COPD involves progressive loss of alveolar epithelial regeneration due to AT2 cell dysfunction and apoptosis leading to alveolar wall damage and injury. AT2 cells plays a vital role in alveolar epithelial regeneration by dividing and differentiating into type I alveolar epithelial cells besides secretion of surfactant proteins. As in aging, chronic CS exposure limits the proliferative recovery of alveolar epithelium, especially AT2 cells, which leads to premature cellular aging and promotes senescence and apoptosis in AT2 cells.

Caveolin-1 (CAV1) is the structural protein component of caveolae which are vesicular invaginations of the plasma membrane. CAV1 participates in signal transduction by acting as a scaffolding protein that concentrates, and functionally regulates signaling molecules within caveolar membranes. CAV1 expression is prominently increased in AECs and AT2 cells during lung injury, which in turn is intricately connected with induction of p53 and PAI-1 expression (Shetty and Idell, Cells. 2023 Feb. 9; 12 (4):554. doi: 10.3390/cells12040554. PMID: 36831221; PMCID: PMC9953971; Galbiati et al., Mol Biol Cell. 2001 August;12 (8):2229-44. doi: 10.1091/mbc.12.8.2229. PMID: 11514613; PMCID: PMC58591). The effects are clinically relevant and occur in patients with COPD. The inventor's data and recent publications using AECs and AT2 cells, or lung sections of patients with COPD and a mouse model of chronic CS-induced lung injury (CS-LI) link these findings (Sun et al, 2019; Chen et al, 2020, Park et al, 2007, Wan et al, 2004). These studies show that p53-mediated induction of PAI-1 augments lung inflammation, MH by AECs, and senescence and apoptosis in AT2 cells (Shetty et al., Am J Respir Cell Mol Biol 47: 474-483, 2012.doi: 10.1165/rcmb.2011-03900C.). These changes predispose to respiratory infection and airway obstruction, alveolar wall damage and loss of elastic recoil, which often occur in COPD. Further, a deficiency in p53 or PAI-1 expression leaves mice resistant to CS-LI (Shetty, 2012). These studies, combined with the close association between lung inflammation, MUC5AC secretion, loss of alveolar structure and reduced AT2 cells viability, led us to hypothesize that induction of p53 and PAI-1 due to increased CAV1 may be involved in excess production of MUC5AC in AECs and increased AT2 cell senescence and apoptosis. The inventor further investigated whether blockade of p53 and PAI-1 by targeting CAV1 signaling using a seven amino acid peptide derived from CAV1 scaffolding domain, CSP7 may mitigate airway MH and distal lung damage. This study suggests concurrently targeting increased CAV1 signaling in AECs and AT2 cells using CSP7 as an effective therapeutic strategy to alleviate CB and emphysema associated in the pathogenesis of COPD. Augmented caveolin-1 influence to CS-induced airway mucus-hypersecretion and alveolar damage. CSP7 dramatically suppressed the mucin hypersecretion, cilia dysfunction and inflammatory cell. This study justifies the therapeutic role of CSP7 for remedying CS-induced lung injury and COPD.

### Materials and Methods

Cell culture. Primary human AECs from histologically normal and COPD lungs were purchased from ATCC. These cells were cultured in AEC Basal Medium with glutamine, extract P, HLL Supplement, and AEC supplement and 1% penicillin-streptomycin at 37° C. and 5% CO2. AT2 cells were isolated from C57BL/6 mice following the method of Corti and colleagues (Roscioli et al, 2018) with minor modifications. The cells were plated on plastic culture dishes precoated with anti-CD-32 and anti-CD-45 antibodies for 2 h at 370 C. Non-adherent cells were collected, and the purity of AT2 cell preparations was assessed by lithium carbonate staining for inclusion bodies (Shetty et al, 2012).

Preparation of CSE for the treatment of AECs in vitro. Research cigarettes 2R4F were purchased from the Tobacco Health Research, University of Kentucky (Lexington, KY). CSE were prepared by burning research cigarettes in a sidearm flask and the smoke generated was bubbled into phosphate-buffered saline (PBS) at room temperature through an attached peristaltic pump as described earlier (Shetty et al, 2012). An absorbance of 1.0 at 230 nm is considered 100%. CSE was filter sterilized by passing it through a 0.2-μm filter.

Peptide preparation. CSP7 and CP were dissolved in DMSO and diluted in HBSS as described elsewhere (Marudamuthu et al., SciTransl Med 11: eaat2848, 2019. doi: 10.1126/scitranslmed.aat2848). These peptides were used to treat AECs in vitro or IP injection of CS-exposed mice. For airway delivery by liquid nebulization, CSP7 (0.579 mg/ml) was added to PBS containing lactose monohydrate (15.456 mg/ml). The pH was adjusted to 8.4 to give a clear solution, filtered through a 0.22-micron syringe filter as described earlier (Marudamuthu et al., 2019.)

Therapeutic effect of CSP7 in mice with CS-LI. All experiments involving animals were performed in compliance with ARRIVE guidelines and in accordance with other relevant guidelines and regulations approved by the Institutional Animal Care and Use Committee (IACUC) of The University of Texas Health Science Center at Tyler (UTHSCT) through protocols 684,630 and 701. Wild-type (WT) C57BL/6 J, p53−/− and PAI-1−/− mice of six to 8 weeks old (weighing 20-25 g) were purchased from The Jackson Laboratory (Bar Harbor, ME). These mice (n=10/group) were exposed to passive CS from 40 research cigarettes over a 2 h period twice a day, 5 days/week for 20 weeks (˜90 mg/m3 total solid particulates) using a mechanical smoking chamber (Teague Enterprises, Davis, CA). Control mice were kept in ambient air. Sixteen weeks later, mice with CS-LI were IP injected with CSP7 or CP (1.5 mg/kg) or CSP7 was delivered via airways by nebulization once a day five days a week for 4 weeks along with exposure to CS (Shetty et al, 2012; Bhandary et al., PlosOne 10: e0123187, 2015. doi: 10.1371/journal.pone.0123187.) All mice were euthanized, and lungs were used for further analyses of 20 weeks post-CS-LI.

Micro-computed Tomography (pCT) scanning and measurements of lung volume and pulmonary function. Twenty weeks after daily CS exposure, ambient air kept control mice as well as mice with CS-LI and treated with or without CSP7 or control peptide of scrambled sequence (CP) were subjected to pCT scanning as described (Marudamuthu et al, 2019). Lung volumes were calculated from lung renditions collected at full inspiration using Microview software. Pulmonary function tests were performed immediately before pCT imaging and before mice were killed. Briefly, anesthetized mice were intubated by inserting a sterile, 20-gauge intravenous cannula through the vocal cords into the trachea. Elastance, compliance, and total lung resistance were measured using the SCIREQ flexiVent system (Tempe, AZ) with a tidal volume set to 30 ml/kg at a frequency of 150 breaths/min against 2-3 cm H2O positive end-expiratory pressure, according to manufacturer's specifications.

Hematoxylin and Eosin (H&E), immunohistochemical (IHC) and immunofluorescence staining of lung sections and AECs. Deparaffinized lung sections were stained with H&E. For immunofluorescence staining, AECs were plated on sterile coverslips and, subjected to designated treatments, subsequently, the cells were washed with PBS three times, fixed with 4% paraformaldehyde for 20 min, permeabilized with 0.1% Triton X-100 (Biosharp) for 20 min, blocked with 3% bovine serum albumin for 1 h, and then incubated overnight with primary antibody stained for MUC5AC (1:100, abcam, USA), HDAC6 (1:100, NOVUS Bio, USA), LC3B (1:100, abcam, USA) and acetylated Tubulin (1:100, Santa Cruz, USA) proteins as described in Table 2. The cells were later stained with FJTC-conjugated secondary antibody (Alexa fluor) and DAPI for staining nucleus. Fluorescent images of cells were captured using Confocal microscope (Carl Zeiss, Göttingen, Germany) and the Zeiss LSM program.

TABLE 2
Antibody source

| | | | | Western | |
|---|---|---|---|---|---|
| | Antibody | Source | Cat no | Blot | IF/IHC |
| 1 | Mucin | Abcam | ab24071 | 1:1000 | 1:200 |
| | 5AC(WB) | | | | |
| 2 | Mucin | Abcam | ab3649 | 1:1000 | 1:200 |
| | 5AC(IHC) | | | | |
| 3 | p53 | Cell signaling | #9282 | 1:1000 | 1:200 |
| 4 | Caveolin-1 | Cell signaling | #3238 | 1:1000 | 1:200 |
| 5 | FOXA2 | Abcam | ab60721 | 1:1000 | — |
| 6 | FOXA3 | Invitrogen | PA1-813 | 1:500 | — |
| 7 | HDAC6 | Novus | NBP1-69127 | 1:1000 | 1:200 |
| | | Biologicals | | | |
| 8 | PAI-1 | Cell signaling | D9C4 | 1:1000 | — |
| 9 | SPDEF | Novus | NBP1-74237 | 1:1000 | — |
| | | Biologicals | | | |
| 10 | PP2A C | Cell signaling | #2038 | 1:1000 | — |
| 11 | Acetylated α | Santa Cruz | sc-23950 | 1:1000 | 1:200 |
| | Tubulin | Biotechnology | | | |
| 12 | LC3B | Abcam | ab48394 | 1:1000 | 1:200 |
| 13 | Erk1/2 | Cell signaling | 9102 | 1:1000 | — |
| 14 | MMP12 | Santa Cruz | SC-39086 | 1:1000 | — |
| | | Biotechnology | | | |
| 15 | Cleaved | Cell signaling | #9661 | 1:1000 | — |
| | caspase3 | | | | |
| 16 | Caspase3 | Cell signaling | #9662 | 1:1000 | — |
| 17 | SPC | Santa Cruz | SC-7705 | 1:1000 | — |
| | | Biotechnology | | | |
| 18 | p53^{AC} | Cell signaling | #2527 | 1:1000 | — |
| 19 | p53⁵¹⁵ | Cell signaling | #9284 | 1:1000 | — |
| 20 | β-gal | Cell signaling | #27198 | 1:1000 | — |
| 21 | β-Actin | Cell signaling | #8457 | 1:1000 | — |

Periodic acid Schiff (PAS) staining: The formalin-fixed slides were deparaffinized, treated with 0.5% periodic acid solution for 5 minutes, rinse in distilled water, and incubated PAS reagent from mucin staining kit (Abcam, USA) for 15 minutes following the instruction of the manufacturer.

Determination of protein phosphatase 2A (PP2A) activity. PP2A activity was determined in isolated AECs and lung homogenates using the PP2A activity assay kit (Millipore) (Nath et al, Am J Respir Cell Mol Biol 59:695-705, 2018. doi: 10.1165/rcmb.2018-01730C.)

Isolation of mouse tracheobronchial epithelial cells (TECs). Mouse TECs were isolated by overnight digestion of minced tracheal tissue of mice in 0.15% Pronase solution at 4° C. Later, the tracheal tissues were removed from the Pronase solution, and the solution was set aside on ice. Tracheal tissues were transferred into a tube containing Ham's F12 media, mixed 12 times, and the process was repeated twice. The Pronase solution was combined with the F12 media supernatants and centrifuged at 1400 rpm for 10 min at 4° C. The pellet was resuspended in 1 mL DNAse solution and kept on ice for 5 min. The solution was then centrifuged at 1400 rpm for 5 min at 4° C. The cell pellet was resuspended in an 8 mL TEC medium containing 10% FBS and plated in 204 culture plates and incubated at 37° C. in an atmosphere of 95% air, 5% C02 for 5 h. The suspended cells from plates were collected and an aliquot was subjected to cytospin, Trypan Blue vital staining, and cell counting as described (Lam et al, J Vis Exp 21:2513, 2011. doi: 10.3791/2513.)

Treatment of human lung tissues with CSP7 ex vivo. For ex vivo studies, de-identified human COPD lung tissues and histologically “normal” lung (nL) tissues were obtained from transplant recipients and from the Gift of Life Donor Program (www.donors1.org) from Temple University, Philadelphia, PA, USA. The demographic data for COPD patients is provided in Table 3.

TABLE 3
Human demographic data

| Age | Sex | Race | Tissue | Grade |
|---|---|---|---|---|
| 70 | F | C | COPD | Gold 4 |
| 63 | M | C | COPD | Gold 4 |
| 68 | F | C | COPD | Gold 4 |
| 71 | F | C | COPD | Gold 4 |
| 67 | M | AA | COPD | Gold 4 |
| 68 | F | C | COPD | Gold 4 |
| 49 | M | C | NL | |
| 48 | F | C | NL | |
| 62 | F | C | NL | |
| 70 | M | C | NL | |
| 67 | M | C | NL | |

The Gift of Life Donor Program does not provide demographics for tissues from “normal” lung as these donors typically suffered fatal trauma as a cause of death. Ex vivo studies using human lung tissues without patient identity was approved by the Institutional (UTHSCT) Review Board (IRB) through exempt protocol #12-003. nL and COPD tissues (n=4-5) were treated with or without CSP7 for 72 h ex vivo as described previously (Marudamuthu et al, 2019). Total protein and RNA extracted from the lung tissues were analyzed by Western blotting and qPCR for MUC5AC protein and mRNA, respectively. In separate experiments, AT2 cells isolated from nL and COPD lungs were treated with CSP7 or CP and immunoblotted for changes in senescence or apoptosis, p53, PAI-1 and SP-C expression. Similarly, nL and COPD tissues were treated with or without CSP7 or CP for 72 h ex vivo and AT2 cells isolated from these tissues were analyzed for senescence or apoptosis, p53, PAI-1 and SP-C.

Expression of CAV1, p53, or PAI-1 in AECs. AECs isolated from nL were separately transduced with Ad-CAV1 or Ad-p53 or Ad-PAI-1. AECs transduced with Ad-Ev were used as controls. In a separate experiment, AECs from nL in culture dishes were treated with Lv-p53 or Lv-PAI-1 shRNA to suppress its baseline expression before treating these cells with CSE. Naive AECs and AECs exposed to non-specific control shRNA was used as controls.

siRNA transfection. AECs cultured in 60 mm Petri plate were exposed to CAV1 or non-specific control siRNA using Oligofectamine reagent (Invitrogen) following the manufacturer's instructions. After 48 h of transfection, these cells were treated with CSE. MH and cilia disassembly were studied.

Protein extraction and Western blot assay. AEC and AT2 cell lysates were prepared using RIPA buffer (Pierce, USA) containing a protease inhibitor cocktail (Roche, Germany) and phosphatase inhibitor cocktail (Sigma-Aldrich, USA). AEC and AT2 cell lysates were subjected to Western blotting using ERK, MUC5AC, HDAC6, SPDEF, FOXA2, FOXA3, LC3, p53, PAI-1, Acp53, S15p53, cleaved caspase-3, β-galactosidase and R-actin antibodies at 1:500-1:1000 dilution (Table 1). The membranes were stripped and subjected to Western blotting using an antibody to assess loading equality. Whole lung homogenates and isolated cell lysates were separated on SDS-PAGE and electroblotted onto a nitrocellulose membrane. After blocking with 1% bovine serum albumin (BSA) for 1 h, membranes were incubated with primary antibodies at respective dilutions at 4° C. overnight, followed by washing and incubation with donkey anti-rabbit/Mouse/Goat horseradish peroxidase conjugated secondary antibody at 1:2000 dilution for 1 h at room temperature in 5% milk buffer. After washing, the protein was visualized using enhanced chemiluminescence detection method. List of antibodies and dilution used are provided in the online supplement S1. Quantifications has been done using image J.

RNA isolation and quantitative real time PCR (qPCR). Total RNA extracted from lung homogenates and cell lysates was reverse transcribed and subjected to qPCR for MUC5AC, HDAC6, FOXA2, FOXA3 and CAV1 transcripts. qPCR primers were purchased from Bio-Rad (Hercules, CA) (Bhandary et al, 2015).

Statistical analysis. All data are presented as the mean±SD of at least five independent experiments. The student's t-test was used to evaluate statistical differences between two groups. Non-parametric tests for statistical analysis amongst three or more groups were performed by one-way ANOVA Kruskal-Wallis test, with Dunn's multiple group comparison tests as appropriate. A *P-value, <0.05 or less was considered statistically significant.

Study approval. Animals were housed in pathogen-free conditions according to protocols approved by the IACUC of the UT Health Science Center Tyler, Texas.

### Results

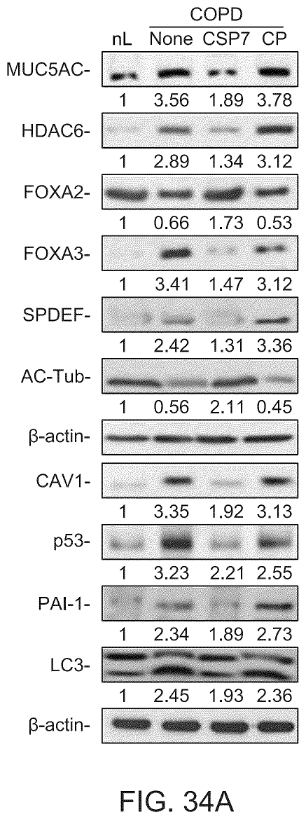

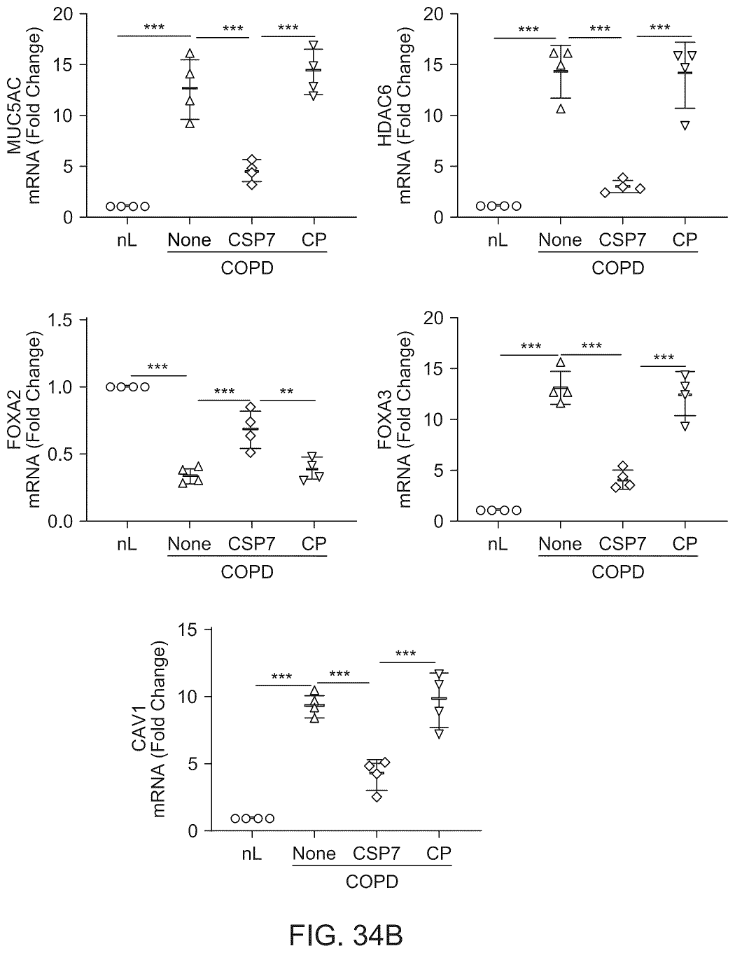

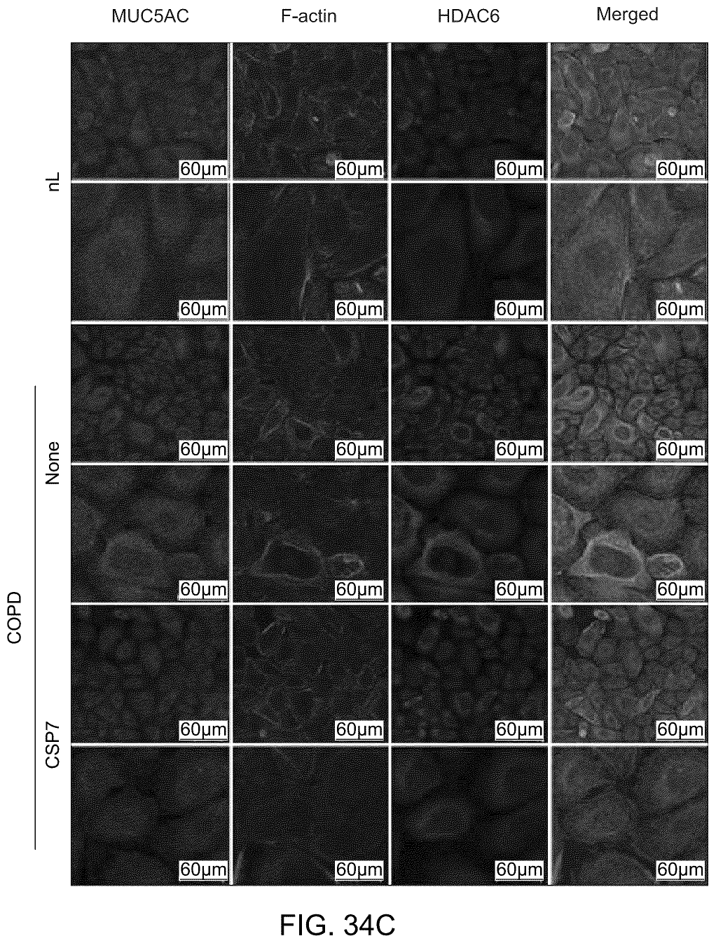

CSP7 mitigates mucus hypersecretion and cilia shortening in AECs from COPD lungs. To understand the mechanism of airway secreted-polymeric mucin expression, the expression of MUC5AC and FOXA3 in AECs isolated from the lungs of COPD patients and control subjects was analyzed. A surge in MUC5AC and FOXA3 expression in AECs from lungs of patients with COPD compared to AECs from nL of control subjects was found (FIG. 34A). Consistent with increased MUC5AC and FOXA3, elevated levels of SPDEF and HDAC6 in AECs from COPD lungs were found. As shown in FIG. 34A, treatment of AECs isolated from COPD lungs with CSP7 reduced the expression of MUC5AC, HDAC6, FOXA3, SPDEF and, LC3 which are otherwise increased in COPD AECs. These changes were associated with the restoration of FOXA2 and acetylated alpha-tubulin, and inhibition of CAV1, p53 and PAI-1 in AECs of COPD lungs exposed to CSP7. Analysis of AECs obtained from COPD lungs, demonstrated augmented MUC5AC, HDAC6 and FOXA3 mRNA expression, with a parallel increase in CAV1 mRNA levels. These changes were significantly suppressed with a concurrent elevation of baseline FOXA2 mRNA expression in AECs from COPD lungs treated with CSP7 (FIG. 34B). This suggests that increased p53 and PAI-1, due to the induction of CAV1, contributes to enhanced MUC5AC expression. The AECs from COPD lungs exposed to CP or left untreated, continued to exhibit elevated levels of MUC5AC, HDAC6, FOXA3, and CAV1 mRNA with low FOXA2 mRNA, compared to AECs from nL. These findings were independently confirmed by immunofluorescence staining analysis and colocalization for MUC5AC, F-actin, and HDAC6 (FIG. 34C).

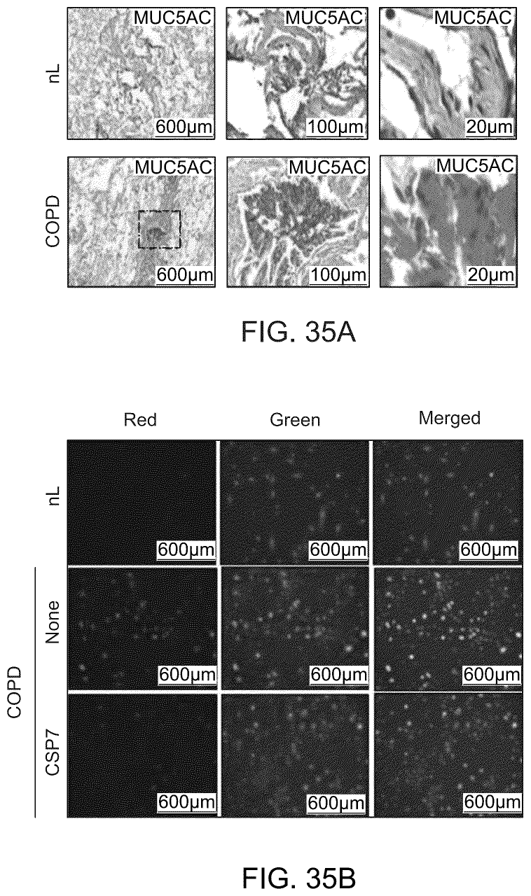

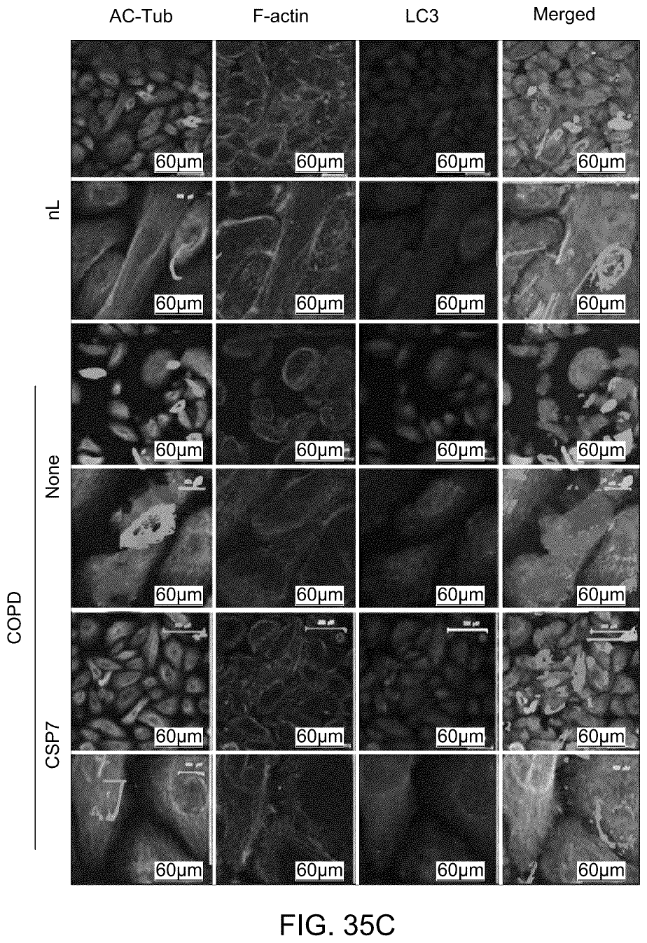

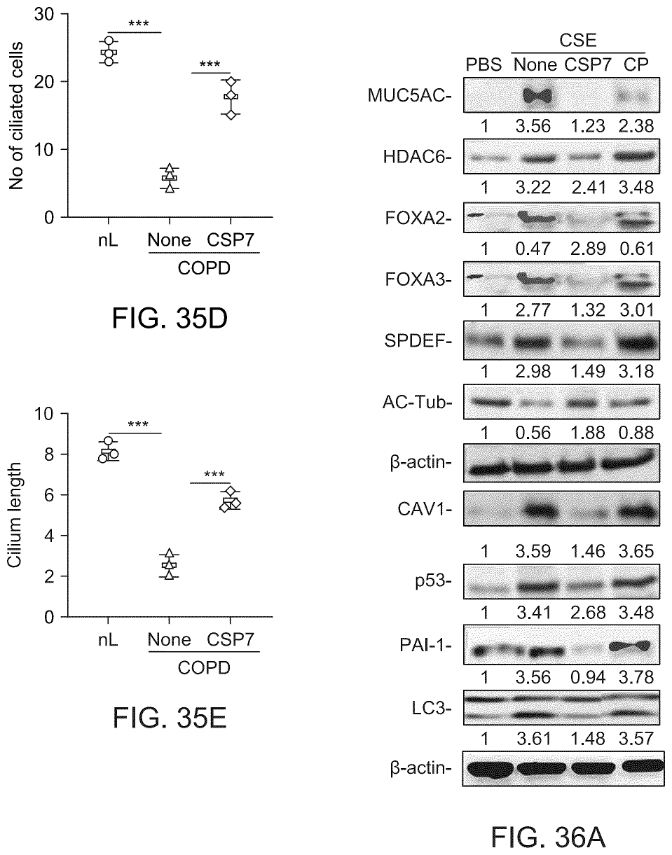

Further, IHC analysis of COPD lung sections showed increased MUC5AC staining compared to their intensity in nL sections (FIG. 35A). Reduced acetylated alpha-tubulin, and elevated HDAC6 levels in AECs of patients with COPD were found. HDAC6 has been shown to regulate primary cilia resorption in response to extracellular stress (Prodromou et al., J Cell Sci 125:4297-4305, 2012. doi: 10.1242/jcs.100545) as well as the autophagic pathway through autophagosome-lysosome fusion (Lee et al., EMBO J 29:969-980, 2010. doi: 10.1038/emboj.2009.405.) Ciliophagy, an HDAC6-dependent autophagic pathway is critical to cilia homeostasis and airway cilia motility in response to CS, and COPD pathogenesis. Evaluation after acridine orange staining, a lysosomotropic agent used for tracking acidic vesicles, showed an increase in red fluorescence intensity (FIG. 35B) in AECs from COPD lung, indicative of increased late autophagic vacuoles. Acridine orange staining revealed a marked reduction in red fluorescence in AECs from COPD lungs treated with CSP7. The inventor also examined the changes in the expression of endogenous LC3-II in AECs and found that rapid accumulation of LC3-II (corresponding to characteristic lipidation of this protein during autophagosome formation) in AECs from COPD lungs, was reversed with CSP7. Interestingly, immunofluorescence staining revealed increased co-localization of MUC5AC and HDAC6 (FIG. 35C), and acetylated alpha-tubulin and LC3 (FIG. 35C) in AECs of COPD lungs. COPD caused a significant reduction in the number of ciliated AECs (FIG. 35D) as well as cilia length in individual AECs (FIG. 35E). These ciliary defects were restored following treatment of AECs from COPD lungs with CSP7. The cilia lengths were measured in AECS by assessing the positive staining of acetylated alpha-tubulin, with 20 μm used as the standard for measurement. The mean value of the cilia length was measured using image J software and calculated from 20 measurements per area.

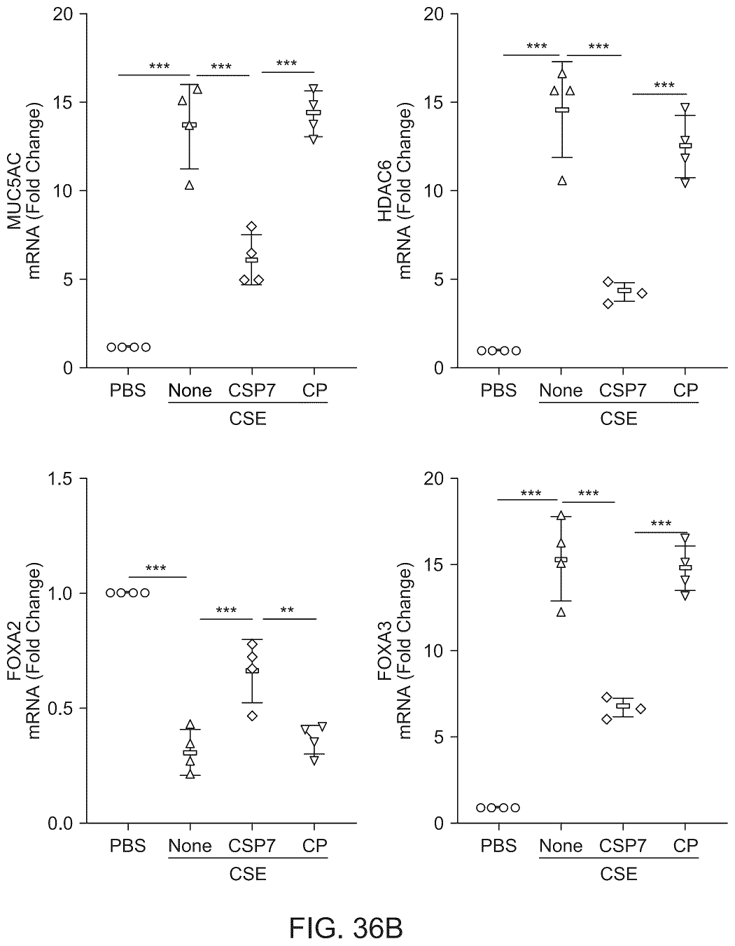

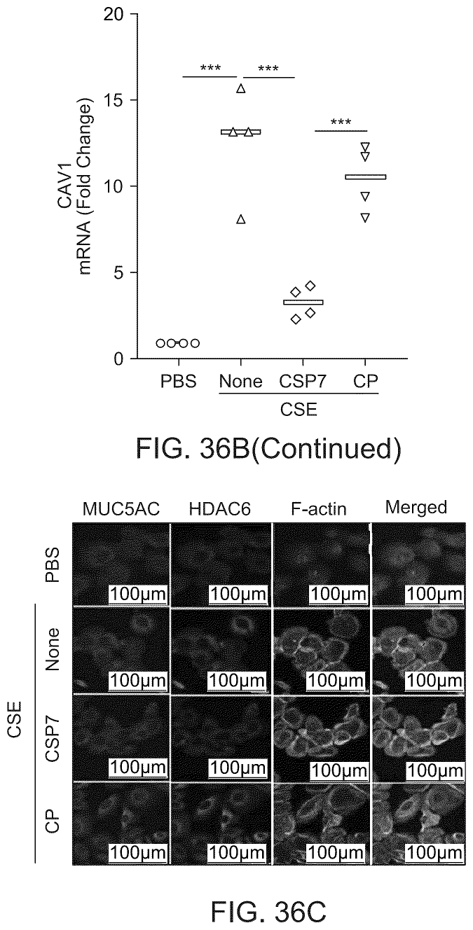

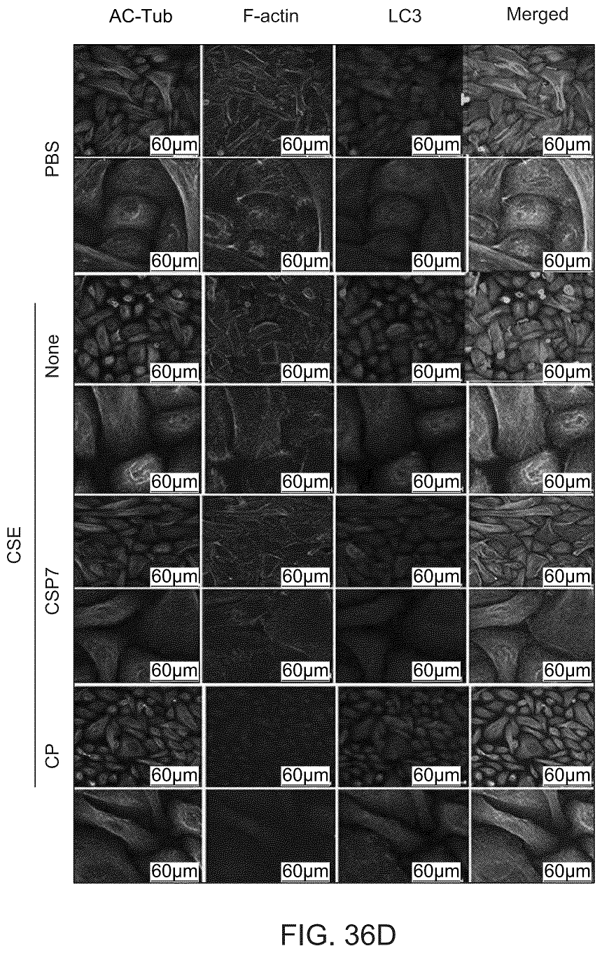

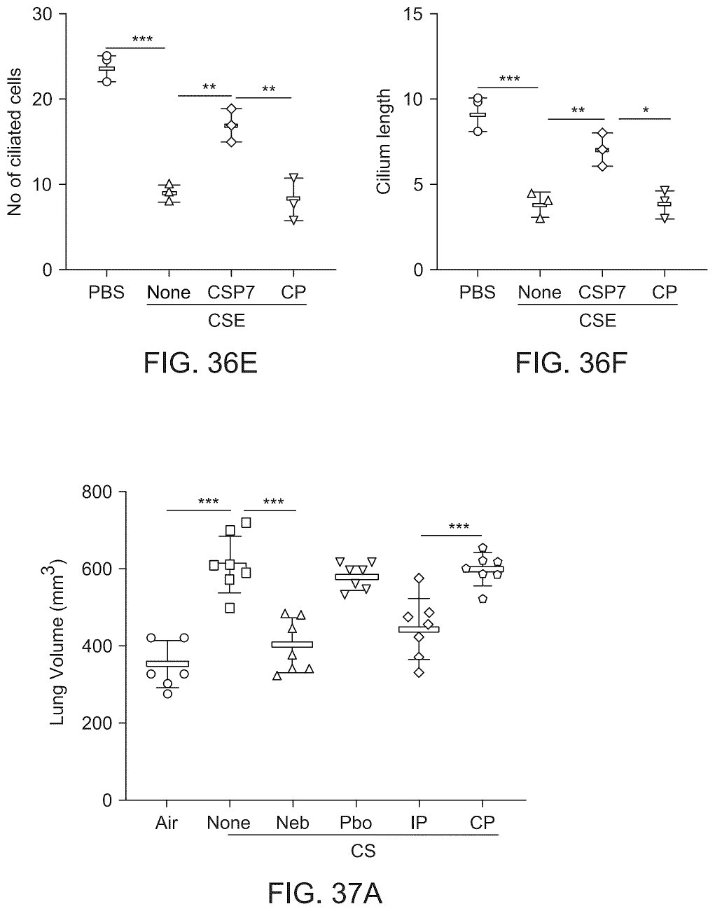

CSP7 inhibits MH and cilia dysfunction in AECs exposed to CS extract (CSE). To further confirm the above findings, AECs isolated from human nL were exposed to CSE in vitro for 48 h and analyzed for MUC5AC, HDAC6, FOXA2, FOXA3, SPDEF, acetylated alpha-tubulin, p53, PAI-1 and LC3. As shown in FIG. 36A, treatment of AECs from nL with CSE increased MUC5AC, HDAC6, FOXA3, SPDEF, CAV1, p53, PAI-1 and LC3, while reducing FOXA2 and acetylated alpha-tubulin expression, which were reversed following the treatment with CSP7. The changes at protein level allied with corresponding changes at the mRNA levels (FIG. 36B). Further, immunofluorescence staining of AECs exposed to CSE revealed increased co-localization of MUC5AC and HDAC6 (FIG. 36C), and acetylated alpha-tubulin and LC3 (FIG. 36D) vs diffused staining in control AECs from nL. CSE induced co-localization of MUC5AC and HDAC6, and acetylated alpha-tubulin and LC3 were inhibited following treatment with CSP7. Immuno-fluorescence staining further revealed a decrease in acetylated alpha-tubulin levels in CSE treated AECs that was improved with CSP7 treatment. Treatment of AECs with CSE also caused a significant (P<0.01) reduction in the total number of ciliated cells (FIG. 36E), as well as cilia length (FIG. 36F) in individual AECs. These changes were significantly reversed in AECs exposed to CSE and treated with CSP7, while CP treated control cells failed to respond.

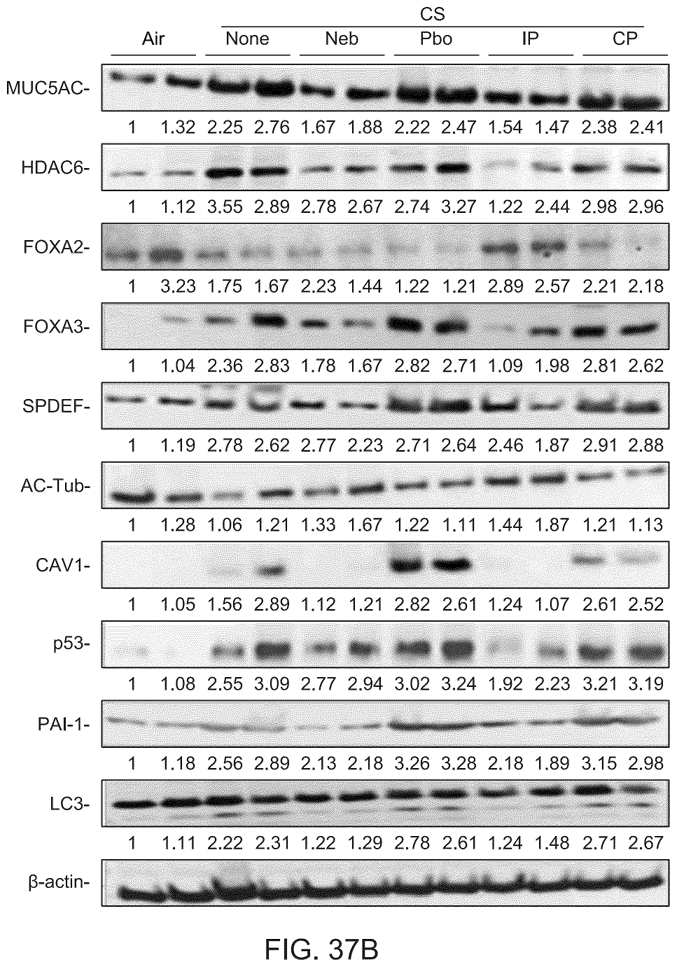

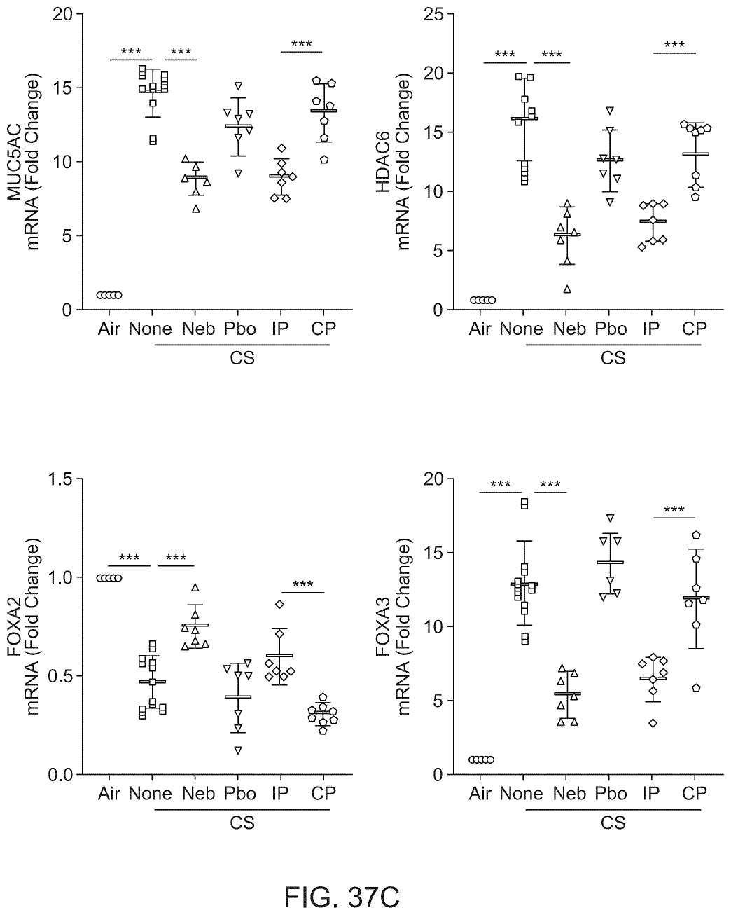

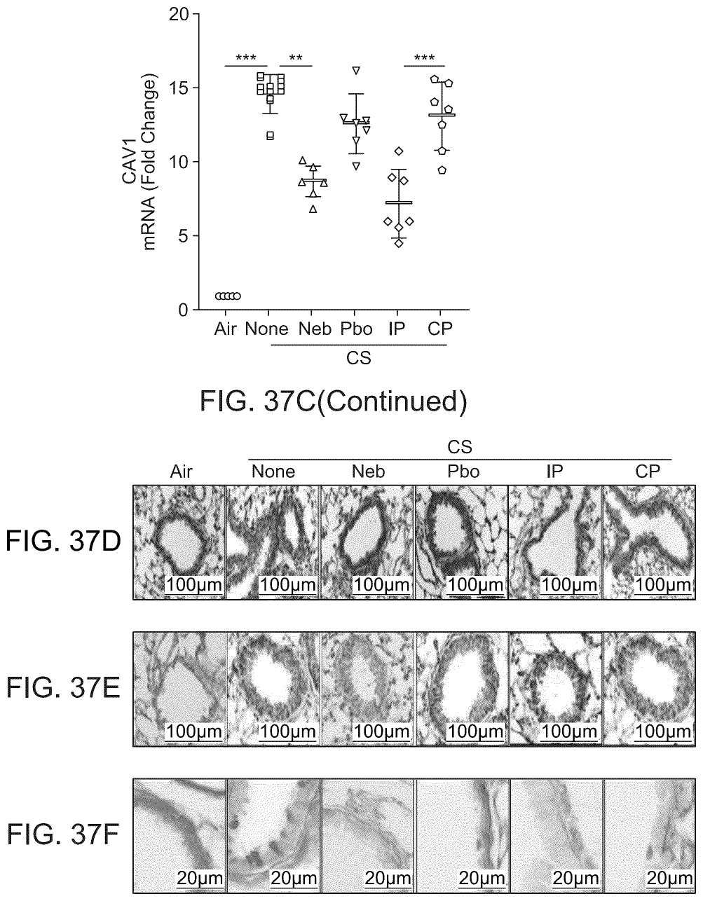

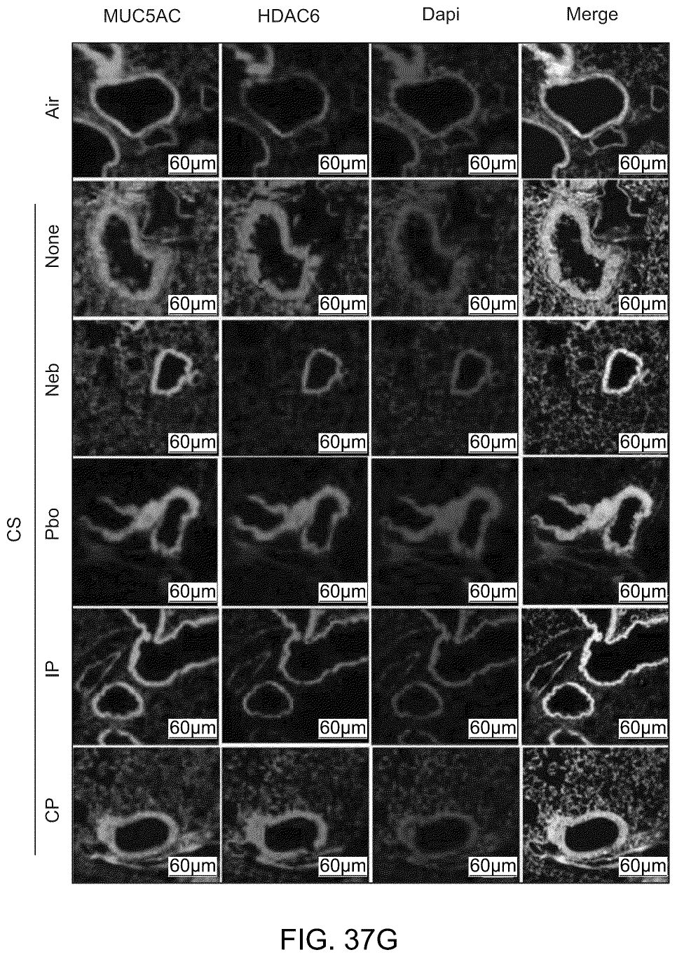

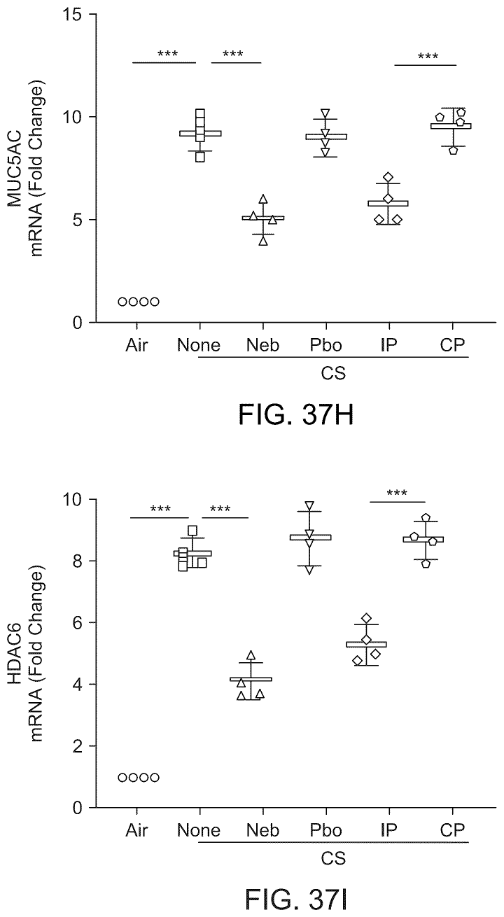

CSP7 inhibits chronic CS exposure induced lung injury in mice. Local delivery of a drug often minimizes target dose requirements and lessens the chance for off-target effects associated with systemic administration. Local delivery can also be more convenient for patients with chronic diseases such as COPD. Therefore, whether CSP7 delivered via airways in liquid formulation mitigates chronic CS-induced lung injury was investigated. To test this mode of delivery, wild-type (WT) mice (n=10/group) were exposed to CS for 4 h/day 5 days a week. After 16 weeks, WT mice with CS-LI were left untreated or exposed to formulated CSP7 (5.8 mg) in 30 ml of PBS containing lactose monohydrate (154 mg) or placebo alone 2 h daily 5 days a week for 4 weeks using a nebulization tower. WT mice with CS-LI were also treated with 1.5 mg/kg of CSP7 or CP by intraperitoneal (IP) injection daily 5 days a week for 4 weeks for comparison. Twenty weeks post-CS exposure, mice were subjected to quantitative chest μCT (not shown). Lung volumes were calculated from μCT renditions at full inspiration (FIG. 37A). Pulmonary function testing by SCIREQ suggested alveolar damage reflected by increased lung volume. These changes were significantly improved in mice with CS-LI and treated with CSP7. However, control CS-LI mice exposed to CP showed persistently high lung volumes, suggesting CS-induced lung damage.

The inventor sought to understand the mechanisms by which CS disrupts AECs in the respiratory tract and their impact on mucin secretion and airway function. Therefore, lung homogenates of WT CS-LI mice left untreated or treated with CSP7 or CP were analyzed for MUC5AC. Western blotting showed that CS-LI caused increased MUC5AC expression in WT mice (FIG. 37B). These changes were associated with reduced FOXA2 and acetylated alpha-tubulin, with augmented expression of HDAC6, SPDEF, LC3 which were reversed following CSP7 treatment, either locally by nebulization or systemically by IP injection. Lung homogenates were next analyzed for CAV1, p53, PAI-1 and LC3 expression. Consistent with changes in AECs from COPD lungs, CS-LI in mice also increased CAV1, p53, and PAI-1. However, CS-LI mice treated with CSP7 via airways or systemically, caused a marked inhibition of CAV1, p53, and PAI-1 (FIG. 37B). These changes were consistent with a significant reduction in MUC5AC, HDAC6, FOXA3, and CAV1 mRNA in CS-LI mice treated with CSP7, while increasing FOXA2 mRNA (FIG. 37C). Further, IHC analysis of lung sections showed intense MUC5AC staining and thickening of the major airway in CS-LI mice. This was remarkably improved in CS-LI mice treated with CSP7 for four weeks (FIG. 37D). This was further confirmed by visualization of goblet cells by PAS staining of the lung sections (FIG. 37E). Increased staining for HDAC6 was found, which was markedly reduced in WT mice with CS-LI exposed to CSP7 by either nebulization or IP injection (FIG. 37F). These changes were not evident in control CS-LI mice treated with CP. Immunofluorescence staining also revealed co-localization of MUC5AC and HDAC6 to thickened major airways in CS-LI mice, which was markedly reduced in those treated with CSP7, either via airways or by IP injection (FIG. 37G). Increased staining and co-localization of MUC5AC and HDAC6 in major airways was evident in mice exposed to placebo via airways, or CP by IP injection. Analysis of TECs isolated from CS-LI WT mice showed increased expression of MUC5AC and HDAC6 mRNA and was significantly reduced in CS-LI mice receiving CSP7 treatment (FIGS. 37H-37I).

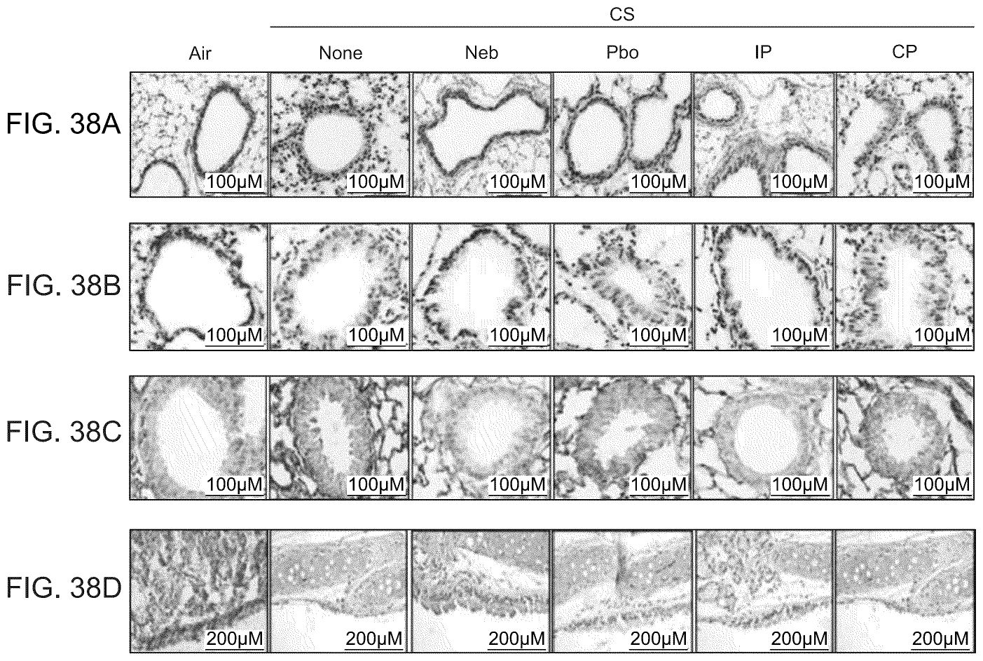

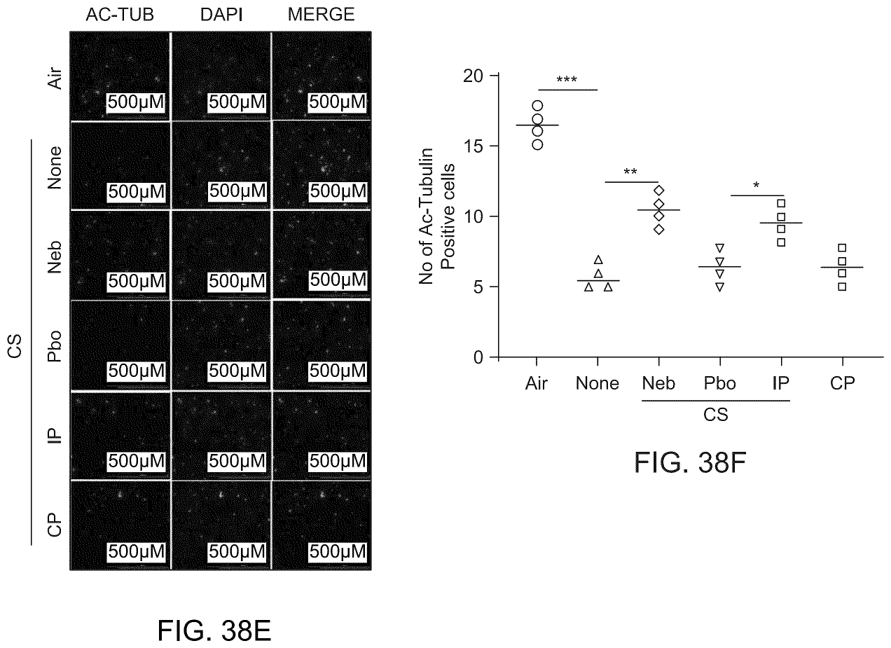

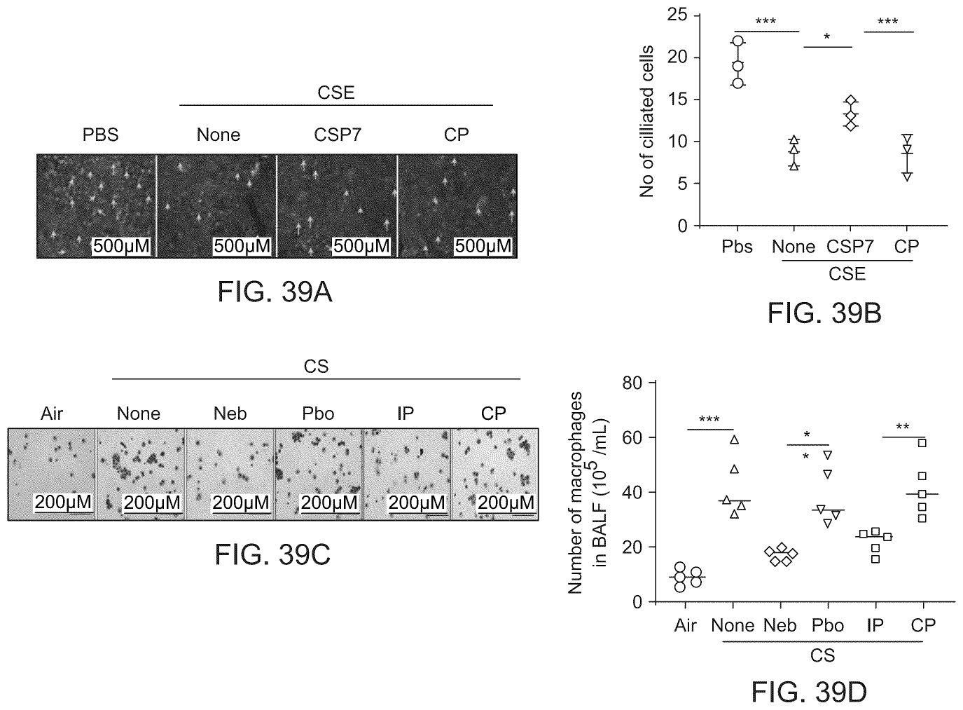

IHC analysis depicted reduced acetylated alpha-tubulin (FIG. 38A), and increased LC3 (FIG. 38B) and CAV1 (FIG. 38C) antigen staining, in lung sections of CS-LI WT mice. This was reversed after treatment of CS-LI mice with CSP7. IHC analyses of tracheal sections showed a decrease in acetylated alpha-tubulin staining in WT mice with CS-LI, which was improved following treatment of these mice with CSP7 (FIG. 38D). Similarly, a marked decrease in the number of acetylated alpha-tubulin positive AECs in WT CS-LI mice was found compared to their levels in ambient AIR kept control mice, suggesting loss of ciliated cells due to lung injury. This trend was significantly reversed after treatment of CS-LI WT mice with CSP7. CSP7 treatment also increased the number of ciliated TECs in WT mice with CS-LI (FIGS. 38E-38F). Immunofluorescence staining further revealed a decrease in acetylated alpha-tubulin in AECs treated with CSE that was reversed with CSP7 treatment (FIGS. 39A-39B). Staining of bronchoalveolar lavage cells from these mice revealed increased accumulation of macrophages in the lungs of mice with CS-LI (FIGS. 39C-39D), which was significantly reduced in those treated with CSP7.

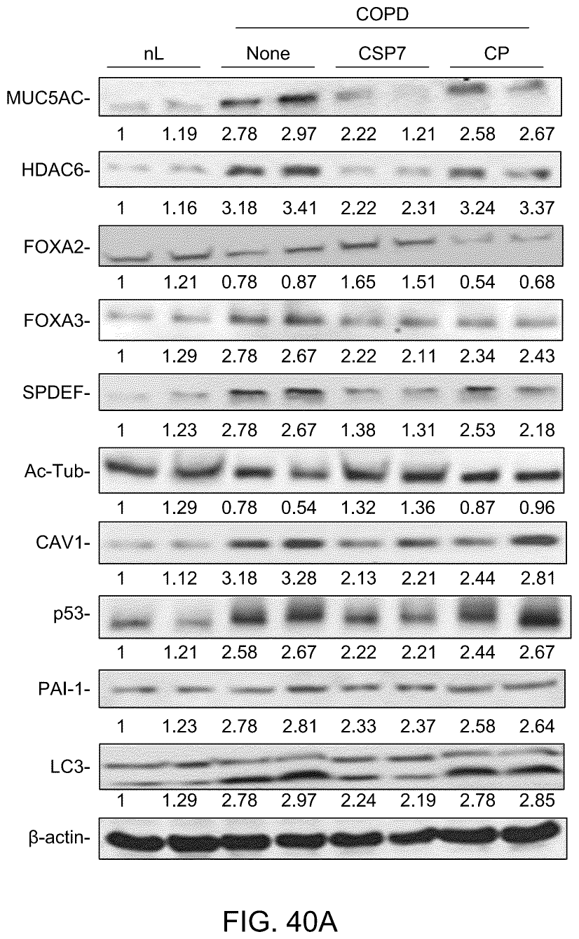

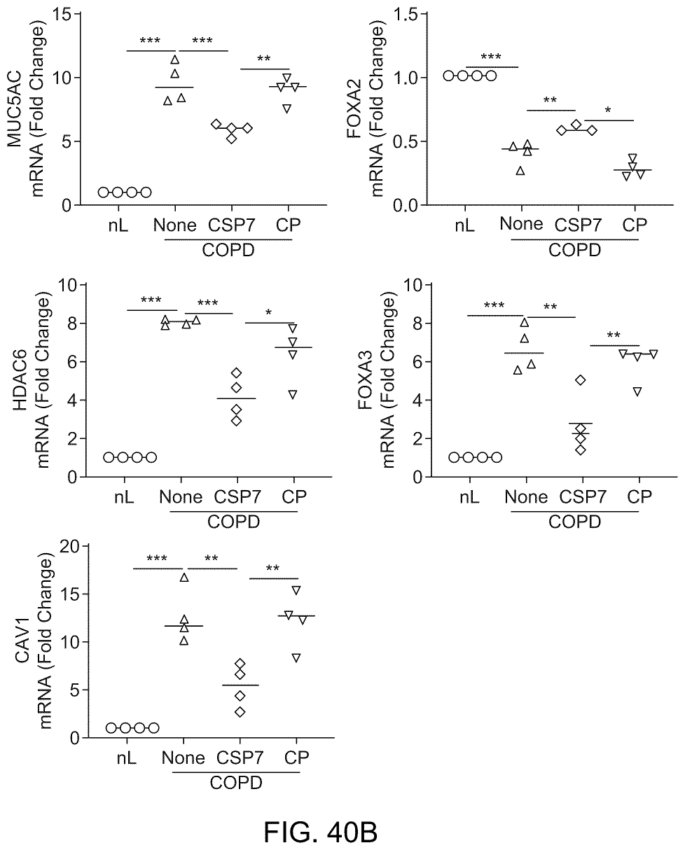

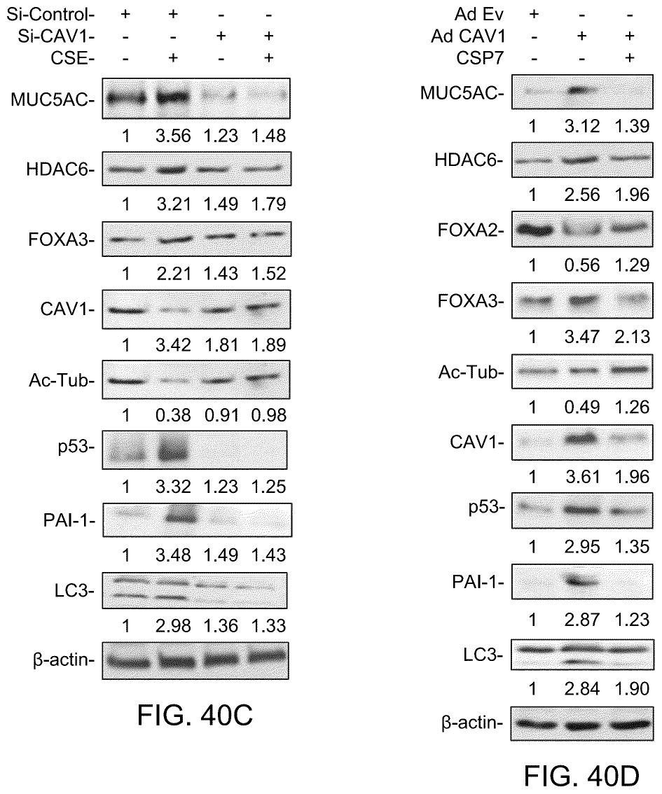

Effect of CSP7 on MH and cilia dysfunction in COPD lung tissues. Human COPD lung tissues were treated with PBS or 10 μM CSP7 or CP ex vivo in culture dishes for 72 h. As shown in FIG. 40A, basal expressions of MUC5AC, HDAC6, SPDEF, FOXA3 and LC3 were elevated, while expression of FOXA2 and acetylated alpha-tubulin was reduced, in COPD tissues compared to their levels in nL tissues. Treatment of COPD tissues with CSP7 reduced MUC5AC and other MCM markers. These changes were associated with suppression of baseline CAV1, p53, and PAI-1, suggesting that CSP7-mediated inhibition of p53 and downstream PAI-1 may provide a beneficial response. This was further confirmed by parallel inhibition of MUC5AC, HDAC6, FOXA3 and CAV1 mRNA with an increase in FOXA2 mRNA in CSP7 treated COPD tissues (FIG. 40B). However, COPD tissues failed to respond to CP treatment. AECs from COPD tissues showed increased CAV1, p53, and PAI-1 (FIG. 34A), suggesting CAV1-mediated induction of p53 and PAI-1 augments MUC5AC expression and MCM. To confirm this possibility, AECs from nL were transduced with CAV1 siRNA to block CAV1 expression. These cells were later treated with CSE. As shown in FIG. 40C, CSE failed to induce MUC5AC, HDAC6, and FOXA3 in AECs exposed to CAV1 siRNA. CSE also failed to inhibit acetylated alpha-tubulin in CAV1 siRNA treated AECs. Consistent with the lack of acetylated alpha-tubulin, CAV1 siRNA treated AECs, exposed to CSE failed to induce p53 and PAI-1 expression. To confirm whether increased CAV1 can induce MUC5AC and MCM, CAV1 was overexpressed in AECs by adenovirus expressing CAV1 (Ad-CAV1). As shown in FIG. 40D, transduction of AECs with Ad-CAV1 increased MUC5AC, HDAC6, FOXA3, and SPDEF while reducing FOXA2 and acetylated alpha-tubulin, suggesting MCM. These changes were associated with the induction of p53 and PAI-1. Since CAV1 induces both p53 and PAI-1, and CSP or CSP7 inhibits CS-induced p53, PAI-1, and MUC5AC, Ad-CAV1 treated AECs were treated with CSP7 and found that CSP7 reduced CAV1-induced MUC5AC, HDAC6, FOXA3, and LC3 while restoring FOXA2 and acetylated alpha-tubulin. In addition, CSP7 treatment of Ad-CAV1 transduced also reduced the expression of p53 and PAI-1.

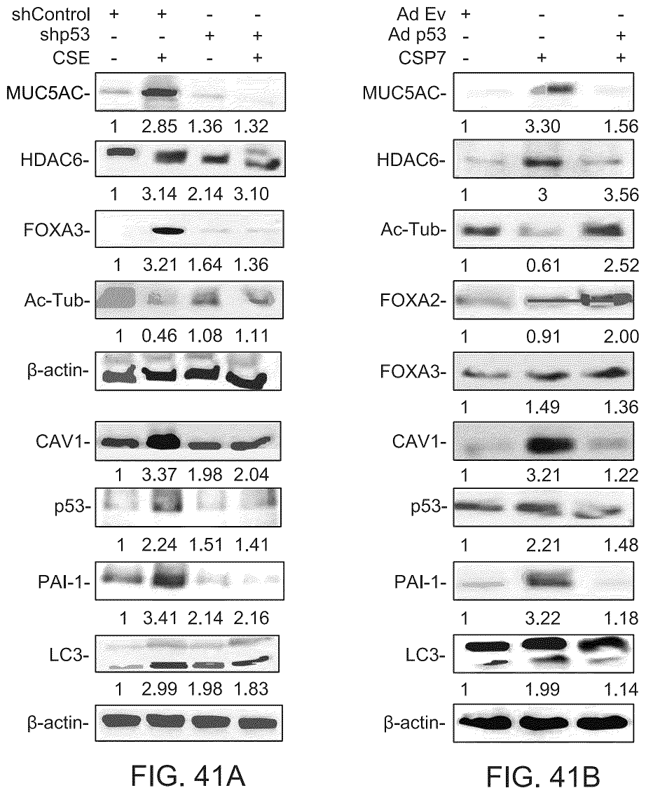

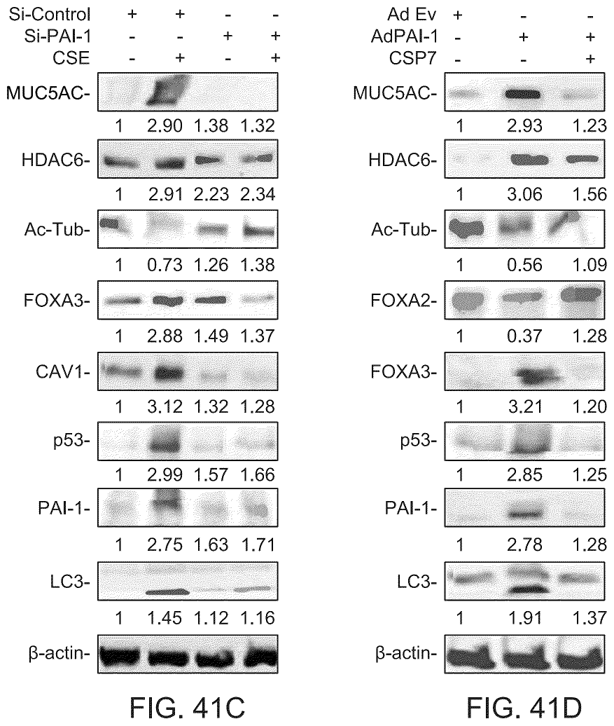

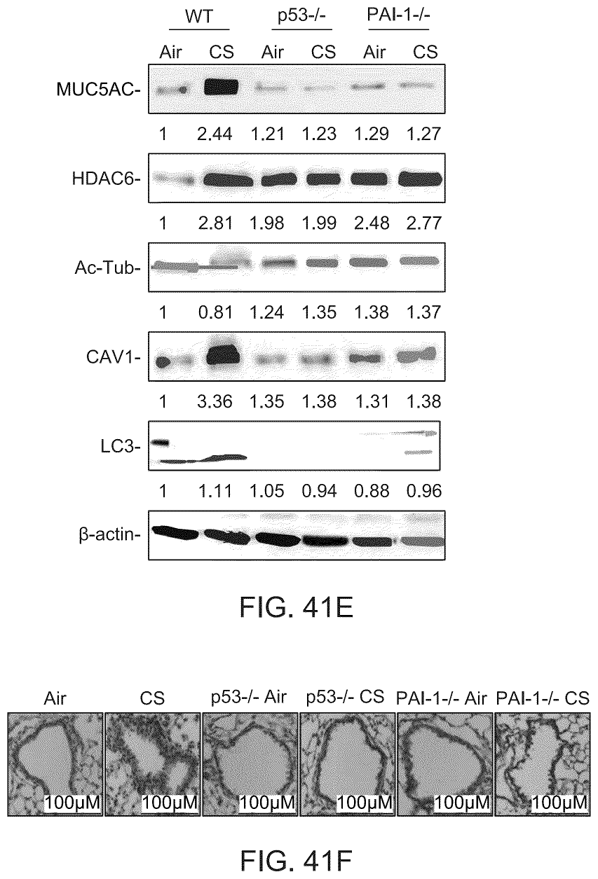

Role of p53 and PAI-1 in CS-LI induced MH and cilia dysfunction. Since CS and CAV1 induce p53 expression in AECs, the inventor investigated whether blockade of p53 mitigates MUC5AC and MCM. As shown in FIG. 41A, exposure of AECs from nL to CSE increased MUC5AC, HDAC6, 400 FOXA3, acetylated alpha-tubulin, CAV1, p53, PAI-1 and LC3. However, transduction of AECs with lentivirus (Lv), expressing p53 shRNA (Lv-p53 shRNA), reduced CSE induced MUC5AC, HDAC6, FOXA3, and LC3 along with suppression of CAV1, p53, and PAI-1. These changes were associated with the restoration of acetylated alpha-tubulin, suggesting a reversal of MCM. Because CS-LI augments p53, and inhibition of p53 mitigates MUC5AC in AECs exposed to CSE, the inventor sought whether overexpression of p53 mimics responses of CS exposure injury in AECs. As shown in FIG. 41B, overexpression of p53 in III AECs from nL induced MUC5AC and inhibited acetylated alpha-tubulin and FOXA2. These changes were associated with a parallel increase in baseline CAV1 and PAI-1 in Ad-p53 treated cells. However, treatment of Ad-p53 transduced AECs with CSP7, reduced MUC5AC and restored acetylated alpha-tubulin and FOXA2, while inhibiting CAV1, p53, and PAI-1. p⁵³ augments PAI-1 expression by upregulating both mRNA transcription and stabilization. We, therefore, treated AECs with Lv-PAI-1 shRNA to prevent CSE induced PAI-1. Inhibition of PAI-1 reduced MUC5AC, HDAC6, FOXA3, and LC3 in AECs exposed to CSE, while restoring acetylated alpha-tubulin (FIG. 41C). These changes were associated with inhibition of CAV1 and p53 in PAI-1-shRNA treated AECs. To directly confirm increased PAI-1 contributes to MCM and MH, AECs isolated from nL were transduced with Ad-PAI-1. Control cells were exposed to Ad-Ev. As shown in FIG. 41D, overexpression of PAI-1 alone induced MUC5AC, HDAC6, FOXA3, p53, and LC3, and reduced acetylated alpha-tubulin. These changes were reversed by treatment of Ad-PAI-1 transduced AECs with CSP7. To determine whether increased p53 or p53-mediated downstream induction of PAI-1 augments MUC5AC expression, the inventor tested AECs isolated from WT and p53- and PAI-1-deficient mice exposed to CS for MUC5AC and MCM markers (HDAC6, FOXA3, and SPDEF) were tested, and the responses compared with AECs extracted from ambient AIR kept control mice. CS induced MUC5AC and MCM markers in WT mice. This induction was not observed in mice lacking p53 or PAI-1 expression (FIG. 41E). These changes were associated with resistance of p53- and PAI-1-deficient mice to CS-induced inhibition of LC3. IHC analysis of lung sections further confirmed resistance of p53- and PAI-1-deficient mice to CS induced MH (FIG. 41F).

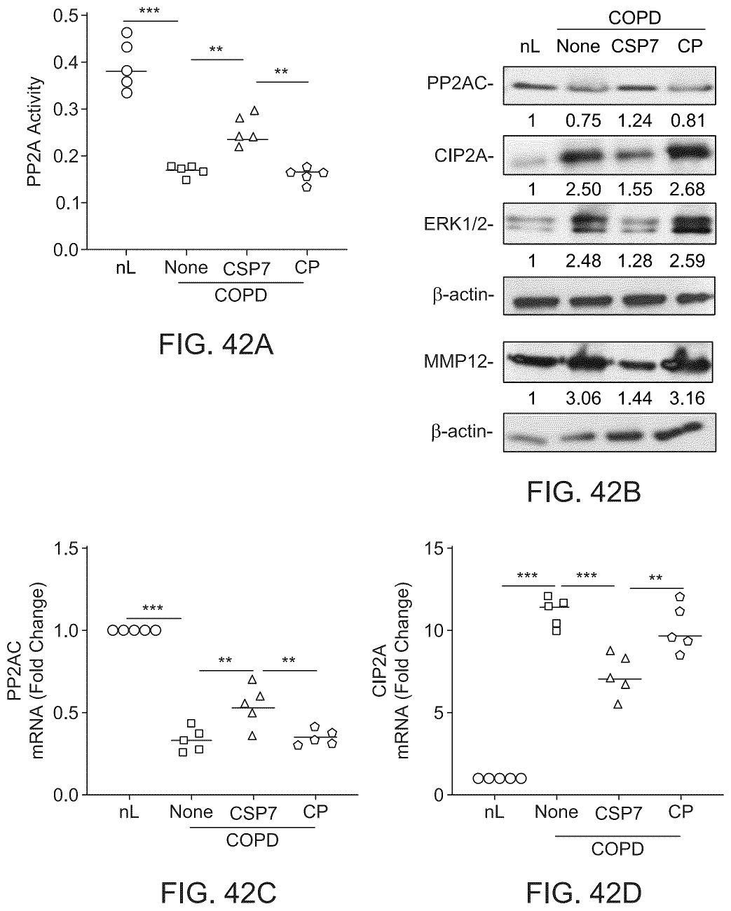

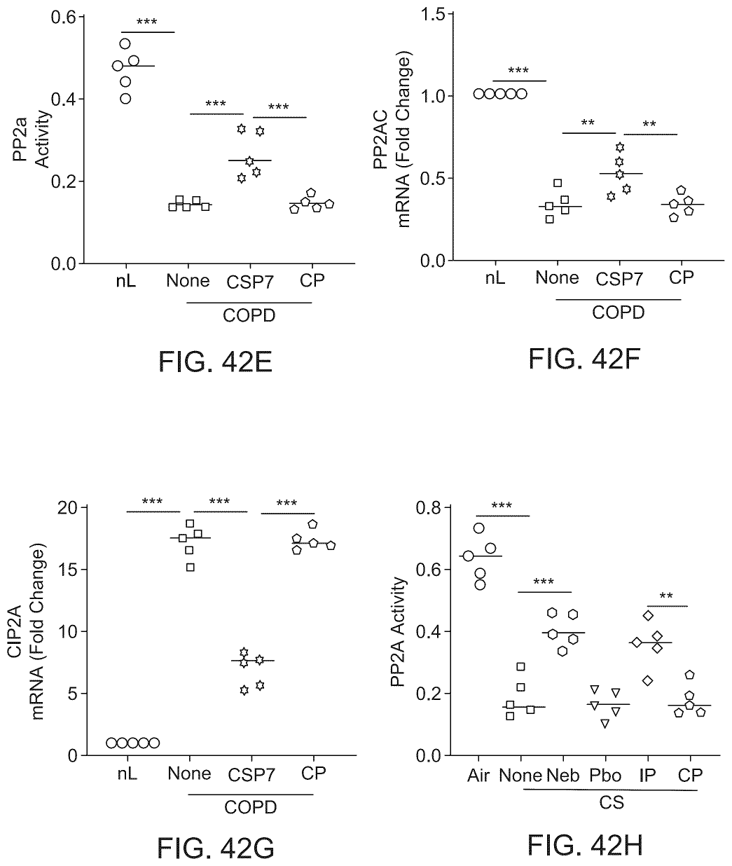

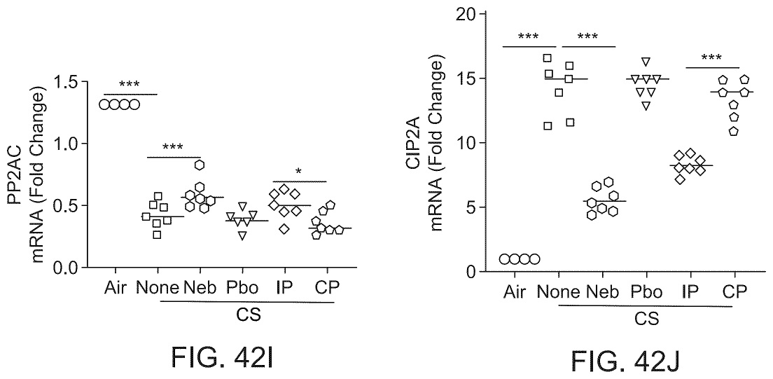

Role of PP2A in CSP7-mediated inhibition of CS-LI induced MH and ciliary disassembly. Since PP2A regulates ATM kinase activity and p53 expression during lung injury, PP2A activity was assessed and a significant decrease in PP2A activity in AECs from COPD lungs compared to AECs from nL was found (FIG. 42A). This was significantly improved following treatment with CSP7, while treatment with CP failed to increase the PP2A activity in these cells. Western blotting of AEC lysates revealed a reduction in PP2A protein levels in AECs of COPD lungs, compared to AECs from nL (FIG. 42B). In addition to the reduction in PP2A protein, there was a marked increase in the expression of CIP2A; an endogenous inhibitor of PP2A. This was reversed by the treatment of AECs with CSP7. Consistent with a loss of PP2A function, ERK phosphorylation was increased in AECs from COPD lungs. This was reversed in AECs exposed to CSP7, while those treated with CP still showed elevated phospho-ERK1/2. These observations suggest that both ERK-mediated phosphorylation of PP2A, and CIP2A-mediated inhibition of PP2A activity, contribute to loss of PP2A function during CS-LI. Since COPD pathogenesis is intricately linked to pulmonary MMP12 440 levels, whether MMP12 were affected by treatment of AECs from COPD lungs with CSP7 was also investigated. Increased expression of MMP12 in AECs from COPD lungs was found, and treatment with CSP7 markedly reduced the MMP12 level. This increase was markedly reduced after the treatment of AECs with CSP7. To further confirm the loss of PP2A expression in AECs from COPD lungs, the inventor analyzed AECs for PP2A mRNA. Consistent with the suppression of PP2A protein, significant inhibition of PP2A mRNA in AECs from COPD lungs was found. This was improved in AECs treated with CSP7 (FIG. 42C). A significant suppression of CIP2A mRNA following the treatment of AECs from COPD lungs with CSP7 was found, which was otherwise increased in AECs of COPD lungs (FIG. 42D). This suggests that increased CIP2A likely reduced PP2A activity in AECs of COPD lungs.

Human lung tissues were then analyzed and PP2A activity was also reduced in lung tissues of patients with COPD compared to nL tissues from control donors. However, treatment of COPD lung tissues with CSP7 ex vivo caused a significant improvement in PP2A activity while CP had minimal effect in these tissues (FIG. 42E). Since reduced PP2A and increased CIP2A expression lowers PP2A activity in AECs isolated from COPD lungs (FIGS. 42A-42B), total RNA from nL and COPD tissues treated with or without CSP7 or CP was analyzed for PP2A and CIP2A mRNA. Consistent with loss of PP2A activity, PP2A mRNA levels were significantly reduced, while CIP2A mRNA was increased in COPD tissues compared to their level in nL (FIGS. 42F-42G). Further, treatment of COPD tissues with CSP7 significantly reversed both PP2A and CIP2A mRNA levels.

Consistent with loss of PP2A activity in COPD tissues, WT mice with CS LI demonstrated a significant reduction in PP2A activity compared to Ambient AIR kept control mice (FIG. 42H). Further, WT mice with CS-LI, treated with CSP7 either locally using a nebulization tower, or by IP injection for 4 weeks, improved PP2A activity. Lungs of WT mice with CS-LI, exposed to placebo or CP, still showed loss of PP2A activity. Analysis of lung RNA for PP2A or CIP2A mRNA revealed that CS-LI inhibited PP2A mRNA while increasing CIP2A mRNA (FIGS. 42I-42J). This was reversed in CS-LI mice that received CSP7.

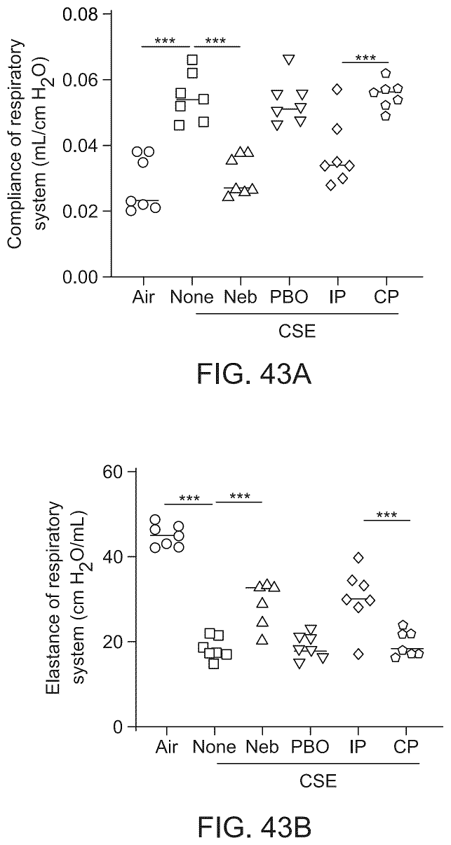

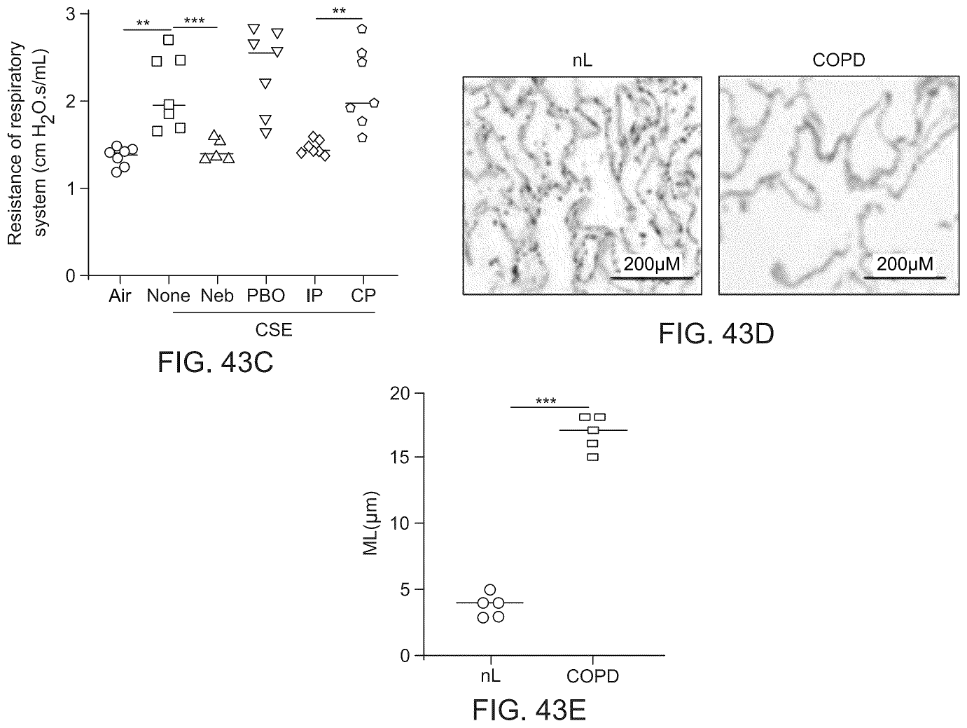

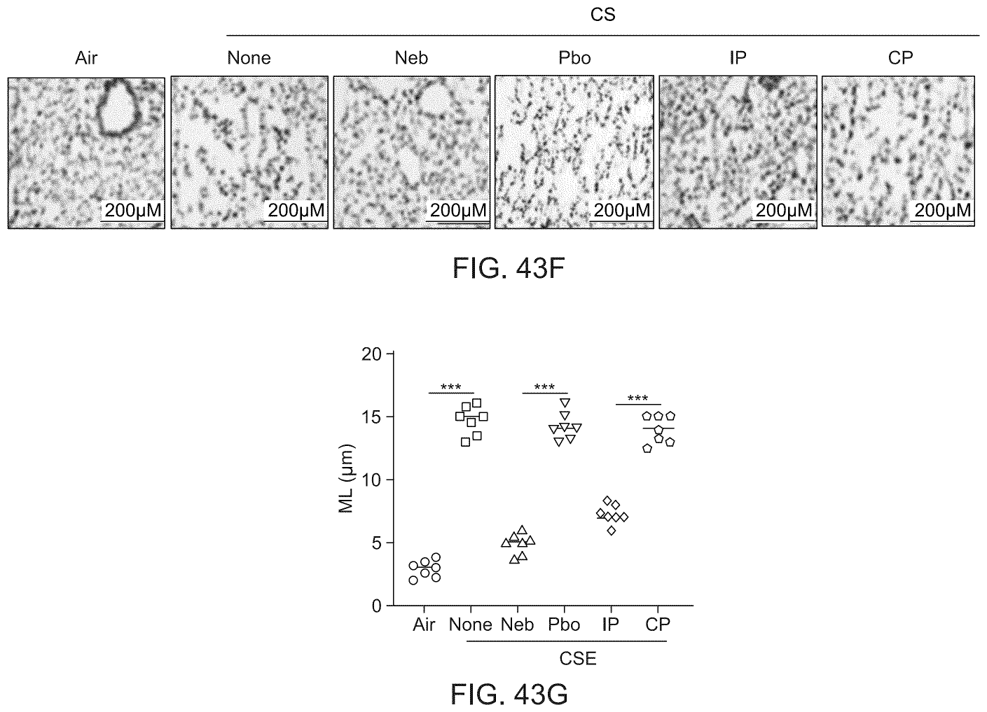

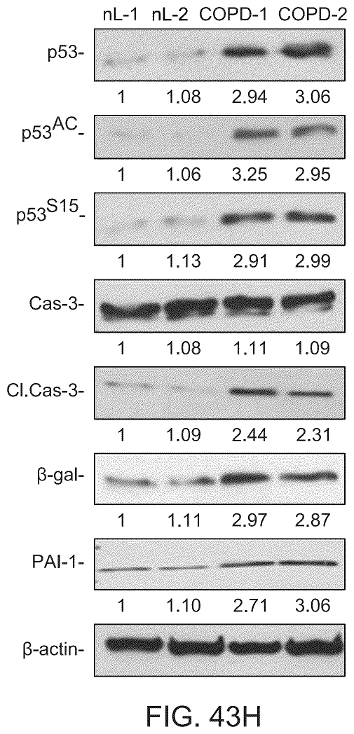

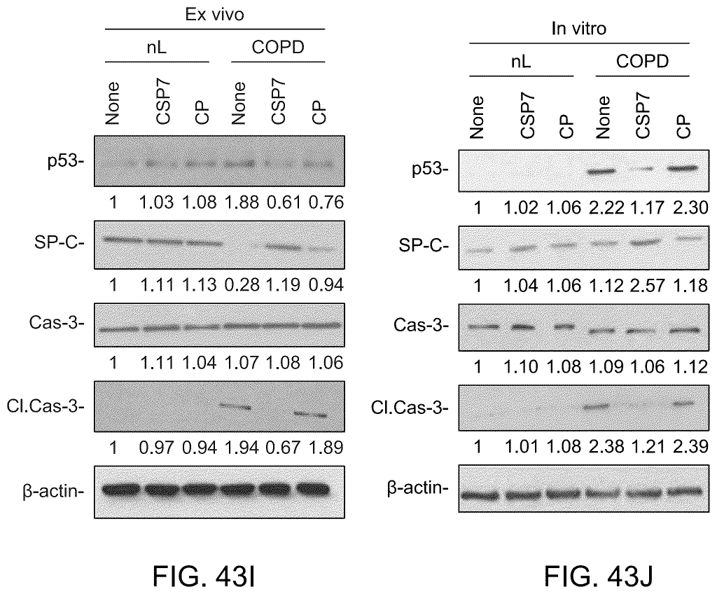

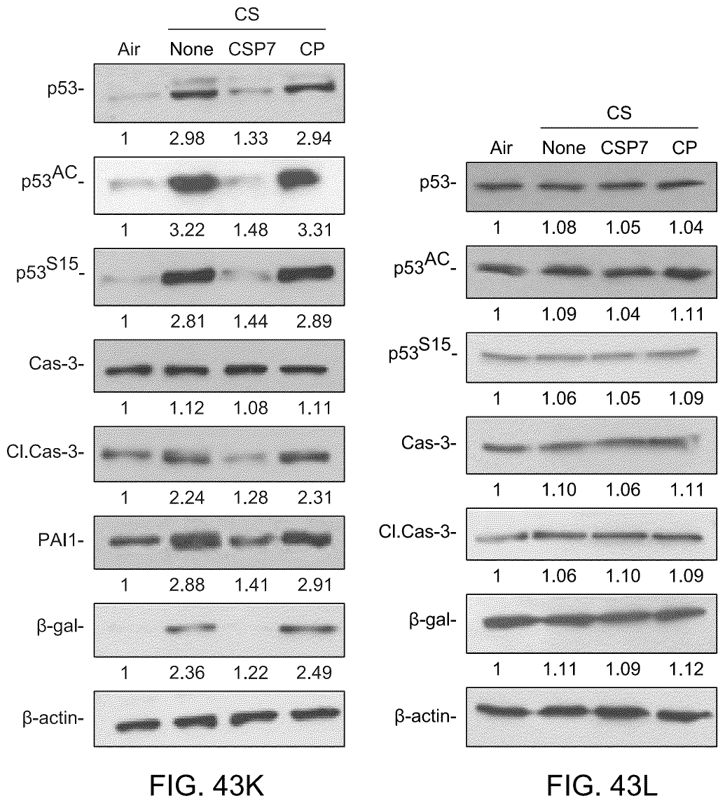

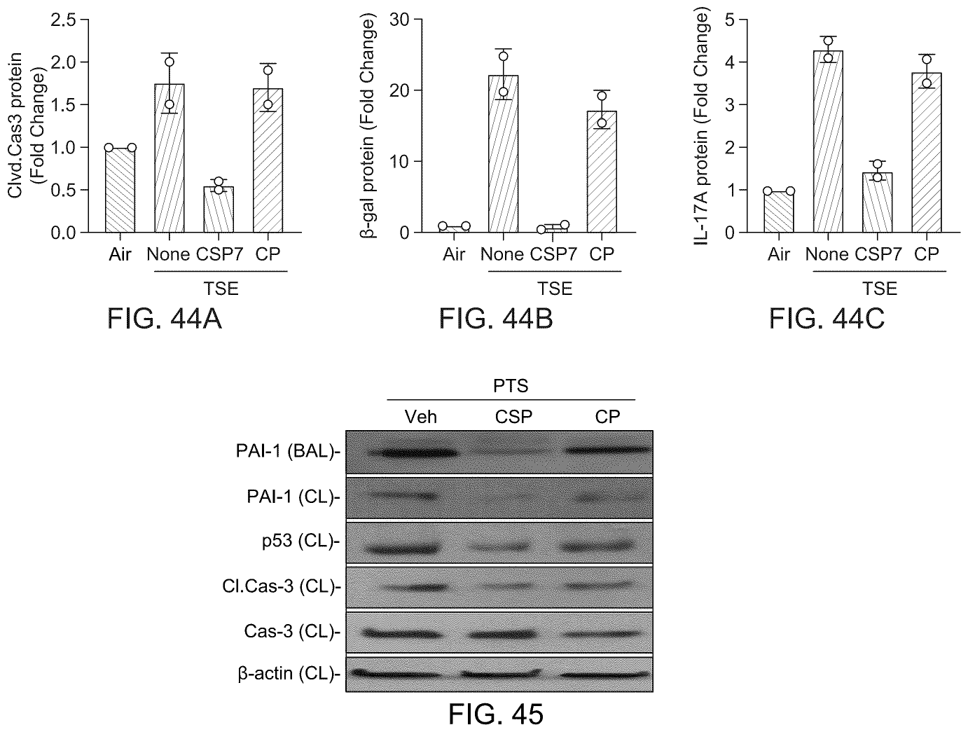

CSP7 inhibits p53 and increases A2C viability in COPD lung tissues. Since mice subjected to quantitative chest pCT twenty weeks post-exposure to CS showed increased lung volume, suggesting distal lung damage (FIG. 37A), compliance, elastance, and resistance were measured (FIGS. 43A-43C). Pulmonary function testing by SCIREQ suggested alveolar damage reflected by increased lung volume and compliance, and reduced elastance. These pulmonary functions were significantly improved in mice with CS-LI following treatment with CSP7. Increased alveolar damage was reflected by the significant increase in the mean linear intercept (MLI) of COPD lung sections compared to the corresponding MLI of nL (FIGS. 43E-43F). Consistent with alveolar damage in human COPD, analysis of H & E-stained lung sections not shown), and measurement of MLI (FIGS. 43F-43G) revealed a significant (P<0.0001) alveolar damage in WT mice with CS-LI. Alveolar damage reflected by increased MLI in CS-LI mice was significantly (P<0.0001) reduced after treatment with CSP7. However, CS-LI mice treated with CP continued to show 479 alveolar damage.

Next, AT2 cells isolated from the patients with COPD were analyzed, which showed a marked increase in the total, acetylated and serine 15 phosphorylated p53 protein as well as activation of caspase-3 and β-galactosidase, thus, implicating induction of p53 due to stabilization through post-translational modifications such as acetylation and serine phosphorylation to increased apoptosis and senescence in AT2 cells (FIG. 43H). Human COPD lung tissues were next treated with CSP7 ex vivo and compared the effects with similarly treated nL tissues from control subjects. AT2 cells isolated from COPD lungs treated with CSP7 ex vivo showed reduction in p53 while restoring baseline SP-C expression (FIG. 43I). Similarly, ex vivo treatment of COPD lung tissues with CSP7 reduced active caspase-3 in AT2 cells, which are otherwise increased in AT2 cells isolated from COPD lung tissues left untreated or treated with CP, suggesting protection of AT2 cells through inhibition of apoptosis. Interestingly, CSP7 had minimal effect in nL tissues treated with CSP7 ex vivo. To further confirm that CSP7 inhibits p53 and apoptosis in injured AT2 cells, AT2 was isolated from COPD lung tissues and exposed to CSP7 in vitro. Consistent with changes observed in FIG. 43J, p53 expression and activation of caspase 3 was markedly increased in AT2 cells isolated from COPD lung tissues compared to their levels in AT2 cells from nL tissues (FIG. 43J). Further, treatment of AT2 cells isolated from COPD lung tissues with CSP7 in vitro caused marked suppression of p53 and apoptosis, thereby restored baseline SP-C level. However, p53 level and apoptosis remained elevated in AT2 cells from COPD lung tissues that were left untreated or treated with CP. AT2 cells isolated from nL tissues from subjects without COPD failed to respond to CSP7 treatment, suggesting its target specificity in injured AT2 cells with altered expression of p53.

Consistent with the changes observed in AT2 cells from human COPD lung tissues, immunoblotting of lysates of AT2 cells isolated from WT mice exposed to 20 weeks of CS showed a marked increase in total, acetylated and serine phosphorylated p53 along with induction of cleaved caspase-3 and β-galactosidase (FIG. 43K), suggesting reduced viability due to increased p53 in AT2 cells. This was markedly suppressed in AT2 cells of CS-LI mice exposed to CSP7 but not in those subjected to CP treatment. Consistent with loss of CS-induced p53, expression of PAI-1 was reduced in AT2 cells of WT mice with CS-LI treated with CSP7. The expression of p53 and PAI-1 is increased in AT2 cells of mice following CS-LI; and p53 induces PAI-1 expression. Therefore PAI-1−/− mice were exposed to CS for 20 weeks to test the importance of increased PAI-1 expression in AT2 cells injury. As shown in FIG. 43L, Western blotting of isolated AT2 cells lysates showed little or no difference in the expression of p53, Acp53, S15p53, cleaved caspase-3, β-galactosidase in PAI-1-deficient mice kept in ambient AIR versus those exposed to CS with or without CSP7 or CP treatment, suggesting resistance of PAI-1-deficient mice to CS-LI. These findings suggest an intricate link between p53-mediated induction of major inhibitor urokinase-type plasminogen activator (uPA)-fibrinolytic system and alveolar epithelial damage.

### Discussion

Airway MH, airflow limitation due to small airway disease, and pulmonary emphysema typify the pathogenesis of COPD. CS is the most common risk factor for the pathogenesis of COPD (Kim V et al., PLoS One 10: 772 e0116108, 2015. doi: 10.1371/journal.pone.0116108.) In COPD, airway epithelium undergoes remodeling, leading to hyperplasia and metaplasia of AECs, including the goblet cells. These changes in goblet cells result in MH and mucin accumulation in the airway lumen leading to airway plugging (Ramos et al, Int J Chron Obstruct Pulmon Dis 9:139-150, 2014. doi: 10.2147/COPD.S38938.) Goblet cell hyperplasia and hypertrophy are often observed in the large airways of smokers with limited airflow (Saetta et al., Am J Respir Crit Care Med 161:1016-1021, 2000. doi: 10.1164/ajrccm.161.3.9907080; Innes et al., Chest 130:1102-1108, 2006. doi: 10.1378/chest.130.4.1102.) Excessive accumulation of mucins due to increased synthesis and secretion, is often associated with an increase in the number of goblet cells.

Interestingly, small (<2 mm luminal diameter) airways of humans, and all intrapulmonary airways of mice, have few or no visible ‘mucous’ or ‘goblet’ cells under baseline conditions. The consequential disruption in mucociliary function may render the host susceptible to infection and particulates-triggered lung injury, which in turn may promote CB and emphysema (Leopold et al., PloS One 4: e8157, 2009. doi: 10.1371/journal.pone.0008157.) Excessive airway secretions overwhelm the function of the mucociliary transport system, thereby raising the risk of acute exacerbations of the disease, which contributes to the development of the obstructive ventilatory disorder. Although it is well established that CS-LI causes MH and adversely impacts cilia length, number, and function in the airways (Sisson et al., Am J Respir Crit Care Med 149:205-213, 1994. doi: 10.1164/ajrccm.149.1.8111584; Abdi et al., Am Rev Respir Dis 142:1436-1439, 1990. doi: 10.1164/ajrccm/142.6_Pt_1.1436. PMID: 2252264), the underlying mechanisms remain incompletely delineated.

In the present study, the therapeutic potential of the novel peptide CSP7, a 7-mer deletion fragment of CAV1 was investigated. The inventor examined whether and how it alleviates MH and improves MCC using human AECs from patients with COPD and pre-clinical mouse models of CS-LI. Elevated expressions of MUC5AC, SPDEF and FOXA3 was observed, while FOXA2 is markedly downregulated in AECs from COPD lungs. MUC5AC, a glycoprotein that constitutes a major component of airway mucin and is highly expressed by airway surface mucus producing cells in patients with COPD. SPDEF, a transcriptional regulator of goblet cell hypertrophy and hyperplasia, upregulates several goblet cell differentiation genes, including FOXA3 (Galbiati et al, 2001), and endoplasmic reticulum protein anterior gradient protein2 homolog. SPDEF expression is increased in the airways of long-term smokers and patients with COPD (Wan et al, 2004; Chen et al, 2010). FOXA3 affects mucus production and is involved in allergic airway hyper-responsiveness and is often detected in airway goblet cells from patients with COPD. In addition, FOXA3 binds and induces SPDEF transcription. The observed effects of FOXA3 on mucus related gene expression are likely mediated, at least in part, by its ability to induce SPDEF despite FOXA3 being sufficient to induce goblet cell metaplasia. FOXA3 also directly binds to and induces AGR2 and MUC5AC that are critical for goblet cell metaplasia and mucin production in the airways (Williams et al., Am J Respir Cell Mol Biol 34:527-536, 2006. doi: 10.1165/rcmb.2005-0436SF; Schroeder et al., Am J Respir Cell Mol Biol 47:178-185, 2012. doi: 10.1165/rcmb.2011-04210C.) Disruption of FOXA2 in respiratory epithelial cells causes airspace enlargement, pulmonary neutrophilic infiltration, and MCM. SPDEF and MUC5AC have previously been shown to be highly expressed in the bronchial epithelium of patients with COPD. Treatment of AECs from COPD lungs, or those exposed to CSE with CSP7 targeted genes most significantly associated with MCM.

Emerging evidence suggests that autophagy plays an important role in lung diseases, including COPD (Ryter et al., Annu Rev Physiol 74: 377-401, 2012. doi: 10.1146/annurev-physiol-020911-153348; Wu et al., Autophagy 16: 435-450, 2020. doi:10.1080/15548627.2019.1628536.) Recently, it has been reported that particulate matter exposure inactivated the mechanistic target of rapamycin kinase (mTOR), leading to enhanced autophagy and impaired lysosomal activity in AECs (Sisson et al, 1994). Cilia shortening and mitochondrial dysfunction, due to CS-induced autophagy in airway epithelium, is linked to AEC damage and impaired MCM associated with the pathogenesis of COPD (Cloonan et al., Autophagy 10:532-534, 2014. doi: 10.4161/auto.27641; Lam et al., J Clin Invest 123: 5212-5230, 2013. doi: 10.1172/JCI69636.) HDAC6 controls diverse cellular processes via deacetylating and destabilizing microtubules, thus facilitating the retrograde transport of ubiquitinated proteins into aggresomes (aggregation of misfolded proteins) and enhancing autophagosome-lysosome fusion (Pugacheva et al., Cell 129:1351-1363, 2007. doi: 10.1016/j.cell.2007.04.035; Pandey et al., Nature 447:859-863, 2007. doi: 10.1038/nature05853.) Lam and colleagues (Lam et al, 2013) demonstrated that CS shortens cilia through an autophagy-dependent process termed “ciliophagy” mediated by HDAC6. In this study, CSP7 inhibited the expression of HDAC6 and the autophagy pathway. In chronic oxidative stress, ciliary proteins are delivered to the lysosome for degradation or recycling, resulting in a shortening of airway cilia that contributes to impaired MCC (Lam et al, 2013). An increase in HDAC6 and upregulation of autophagy markers were observed, with cilia shortening in AECs of COPD and CS exposed mouse lungs. Immunofluorescence imaging showed the co-localization of acetylated alpha-tubulin and LC3, suggesting an interaction of cilia components and autophagosomes. This is further evidence of interactions between HDAC6 and MUC5AC in AECs during CS-LI, which is reversed following the treatment with CSP7.

CS induces AT2 cell damage. The process involves increased expression of CAV1 through activation of the ATM-p53-p21 pathway (Volonte and Galbiati, Aging (Albany NY). 1:831-835, 2009. doi: 10.18632/aging.100079.) Additionally, CS induced activation of EGFR and AKT through phosphorylation can contribute to AEC proliferation and metaplasia. The down regulation of CAV1 inhibits MUC5AC production; while conversely, CAV1 enhances CS induced MH through activation of EGFR and AKT (Yu et al., Int J Mol Med 35: 1435-1442, 2015. doi: 10.3892/ijmm.2015.2133). CAV1, reportedly regulates airway inflammation and aggravates lung injury. In this study, the inventor sought to determine whether CAV1 modulates MH and ciliary dysfunction induced by CS. These results revealed that elevated expression of CAV1 alone augments MH and ciliary disassembly in AECs often associated with COPD, suggesting CAV1 as a novel therapeutic target. It was also found that forced expression of CAV1 enhanced MUC5AC and MCC related genes. Similarly, a decrease in acetylated alpha-tubulin and an increase in autophagy related gene were observed following overexpression of CAV1. Conversely, the inhibition of CAV1 using siRNA reduced MH and ciliary dysfunction in AECs exposed to CSE.

A study by Saganaki and colleagues (Siganaki et al., Respir Res 11:46, 2010. doi: 10.1186/1465-9921-11-46) reported an increase in p53 protein levels in COPD lungs. Also, serum levels of PAI-1 were higher in COPD patients than in controls. This data demonstrate increased p53 and PAI-1 levels in COPD lungs. Increased p53 and PAI-1 expression in in vitro and in vivo models of CS exposure induced injury were observed. A gain-of-function experiment using a p53 and PAI-1 expressing adenoviral transfection, demonstrated that overexpression of either p53 or PAI-1 in the AECs increased MUC5AC and MCM related genes. A loss-of-function experiment using transfection with p53, and PAI-1 shRNA decreased CS induced secretion of MUC5AC. Inhibition of p53 from binding to the endogenous PAI-1 mRNA in AECs, by either suppressing the expression of p53 or blockade of p53 interactions with the PAI-1 mRNA, mitigates CS-LI (Shetty et al, 2012, Bhandary et al, 2015). Further, earlier reports and others reveal that CAV1 expression is required for CS-induced activation of the p53 and PAI-1 pathways (Volonte et al., J Biol Chem 284:5462-5466, 2009. doi: 10.1074/jbc.C800225200; Shetty et al., J Biol Chem 283:19570-19580, 2008. doi: 10.1074/jbc.M710268200.) CAV1 is therefore presumed to be a key player of a novel signaling pathway that links CS-LI to MH and ciliary disassembly. CSP7 mitigates cilia shortening and improves MCC by interfering with CAV1-mediated induction of p53 and downstream PAI-1 expression by increased p53 in AECs.

AECs from subjects with COPD have reduced PP2A activity as well as increased CIP2A expression. PP2A activity was reduced in CSE treated AECs. This was associated with increased expression of CIP2A. An increase in CIP2A leads to phosphorylation of ERK, and secretion of MMP12, with loss of PP2A activity (Li et al., Mol Cell Biol 23:9389-9404, 2003. doi: 10.1128/MCB.23.24.9389-9404.2003; Nath et al, 2018). Moreover, increased CAV1 induced the expression p53 and PAI-1 in AECs. This is often associated with COPD pathogenesis. CSE elicits binding of CAV1 to the catalytic subunit of PP2A, which in turn could downregulate PP2A activity (Bhandary et al., Toxicol Appl Pharmacol 283(2)92-98, 2015. doi.org/10.1016/j.taap.2015.01.007 PMID: 25596429). In addition, enhanced CIP2A suppresses PP2A activity in AECs. Further, phosphorylation of ERK, and secretion of MMP12 ensue. Also, CS induced stress can lead to cilia damage, ubiquitination, and the formation of intracellular protein aggregates. Moreover, HDAC6 recognizes damaged ciliary proteins and delivers them to the autophagosome, a process reliant on autophagy proteins. The damaged ciliary proteins are eventually delivered into the lysosome for degradation or recycling. As a result of the stress induced by CS, ciliary proteins are degraded, resulting in a shortening of airway cilia that contributes to impaired MCC. CAV1 is therefore presumed to be a key player of a novel signaling pathway that links CS to MH and ciliary disassembly.

A limitation of previous studies is the lack of animal models that accurately represent all cardinal manifestations of human COPD. Therefore, the beneficial effects of CSP7 on human primary AECs treated in vitro, human COPD lung explants treated ex vivo, and mice model in vivo were evaluated. Based on reports of the inventor and other groups CSP7 may mitigate CS-LI via effects on multiple cell types and attenuation multiple pathways. For example, CSP7 suppressed the influx of inflammatory cells into the lungs by inhibiting leucocyte recruitment. In vitro or ex vivo response of isolated AECs or COPD tissues treated with CSP7 could differ from their responses in live individuals. Nonetheless, these findings provide a strong predicate for further exploration of the complex mechanisms that underlie the salutary effects of CSP7.

This study clearly reveals that CSP7 mitigates cilia shortening and impaired MCC by interfering with CAV1-mediated AEC signaling pathways. This includes the downregulation of the ERK phosphorylation, expression of MMP12, and the inhibitor of PP2A, CIP2A. These findings provide not only new insights on how CSP7 regulates complex interrelationships between p53, PAI-1, autophagy, and primary cilia, but also the possibilities for the treatment of the ciliopathies often associated with MH. These findings suggest that CSP7 could be a potential new therapeutic intervention to improve airway dysfunction associated with COPD pathogenesis.

Multiple studies have deciphered that AEC and AT2 cell apoptosis and defective alveolar fibrinolysis due to a disproportionate increase in PAI-1 expression are often associated with CS-LI, including COPD. Similarly, PAI-deficient mice also resisted AT2 cell apoptosis, and the responses of p53- and PAI-1-deficient mice to 20 weeks of CS exposure were very similar. The proportion of AT2 cells undergoing apoptosis and senescence are markedly increased in lungs of patients with COPD compared to healthy subjects. CS augments both PAI-1 and p53 expression in AT2 cells. Treatment of isolated AT2 cells from COPD tissues in vitro as well as ex vivo treatment of COPD tissues with CSP7 improved viability by inhibiting senescence and apoptosis. The process involves suppression of CS-induced p53 and downstream PAI-1 expression. These findings not only deeply clarify the potential mechanisms of CS induced alveolar and airway damage, but also extend the understanding about how CSP7 mitigates the airway and alveolar epithelial injury. Considering the potential characteristics of CSP7, this research provides several lines of evidence for the application of CSP7 in treatment of COPD and CS-LI. Based on the inventor's in vitro and ex vivo studies using AECs, AT2 cells and human COPD tissue explants, and mouse model of CS-LI in vivo suggest that CSP7 has potential to target MH and distal lung damage often associated with COPD.

In the present study, elevated expression of CAV1 caused MCM and MH through induction of p53 and PAI-1 expression in AECs from the lungs of patients with COPD and mice with CS-LI. Further, CS-LI down regulated PP2A activity and increased the expression of CIP2A leading to increased phosphorylation of ERK, and secretion of MMP12. In addition, increased expression of p53 and PAI-1, and reduced viability due to induction of senescence and apoptosis in AT2 cells from lungs of patients with COPD or mice with CS-LI. These changes are all reversed by a seven amino acid (FTTFTVT) CAV1 scaffolding domain peptide; CSP7. CSP7 mitigates cilia shortening and improves MCC. These findings provide not only new insights on how CSP7 regulates complex inter-relationships between p53 and PAI-1 expression, autophagy, and primary cilia in AECs and loss of AT2 cells viability, but also the possibilities for the treatment of MH and associated ciliopathies and alveolar damage. These findings suggest dual role of CSP7 on AECs and AT2 cells as a potential new treatment to improve lung function during chronic lung diseases such as COPD, through the maintenance of AEC proteostasis and modulation of the autophagic pathway and alveolar epithelial regeneration. Further, these findings demonstrate that CS-LI can be reduced by systemic or airway delivery of CSP7.

### Example 21

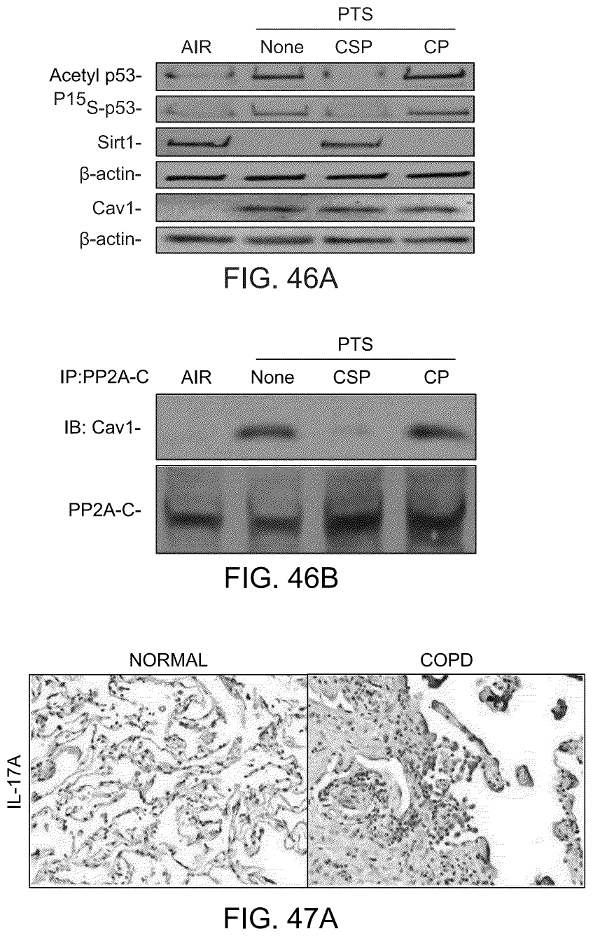

A 20 amino acid peptide (CSP) inhibits passive tobacco smoke (PTS) induced lung injury with inhibition of p53 and PAI-1 in A₂ECs. WT mice exposed to PTS show increased caveolin-1 (Cav1) expression in A₂ECs (FIG. 46A). Cav1 in turn interacts with the PP2Ac (FIG. 46B) and sequesters PP2Ac to caveoli. The sequestration of PP2Ac by Cav1 leads to stabilization of p53 due to serine-15 phosphorylation of p53 (^{p15S}p53), and A₂EC apoptosis and lung injury. To reverse PTS induced lung injury, CSP was developed as a competitor for Cav1. Treatment of WT mice with CSP inhibited the PTS-induced binding of Cav1 with PP2Ac (FIG. 46B) without affecting Cav1 expression in A₂EC, which is otherwise increased after PTS injury and blocked ^{p15S}p53 (FIG. 46A). Besides ^{p15S}p53, acetylation of p53 and down regulation of Sirt1 (FIG. 46A) is associated with increased miR-34a expression (FIG. 46A, 5A or 27A) suggesting these changes could contribute to p53-mediated induction of PAI-1 expression and A₂EC apoptosis. Further, CSP inhibits acetylation and the process involves restoration of Sirt1 (FIG. 46A) and suppression of miR-34a expression (FIGS. 5A, 27A).

### Example 22

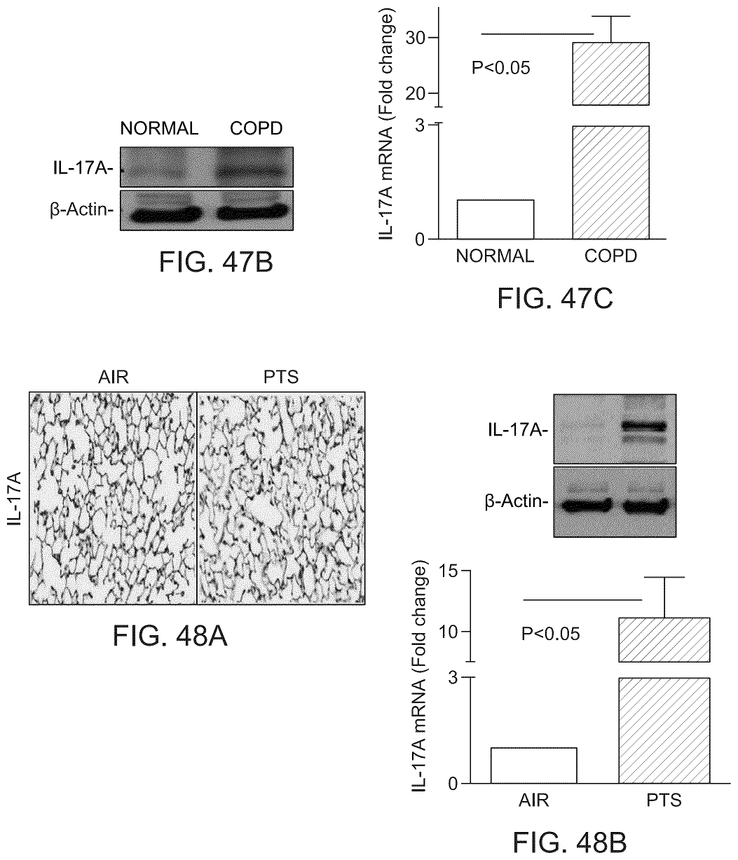

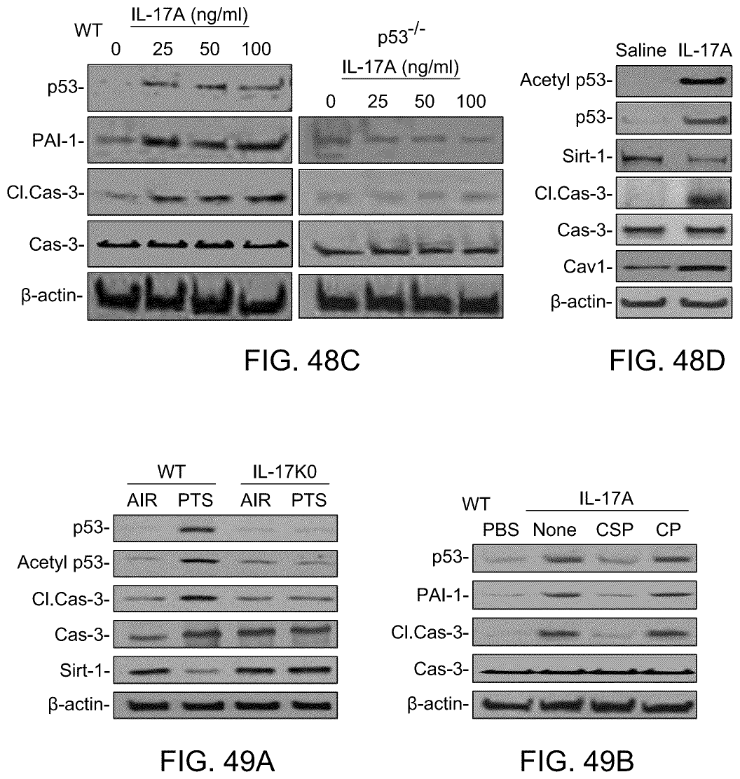

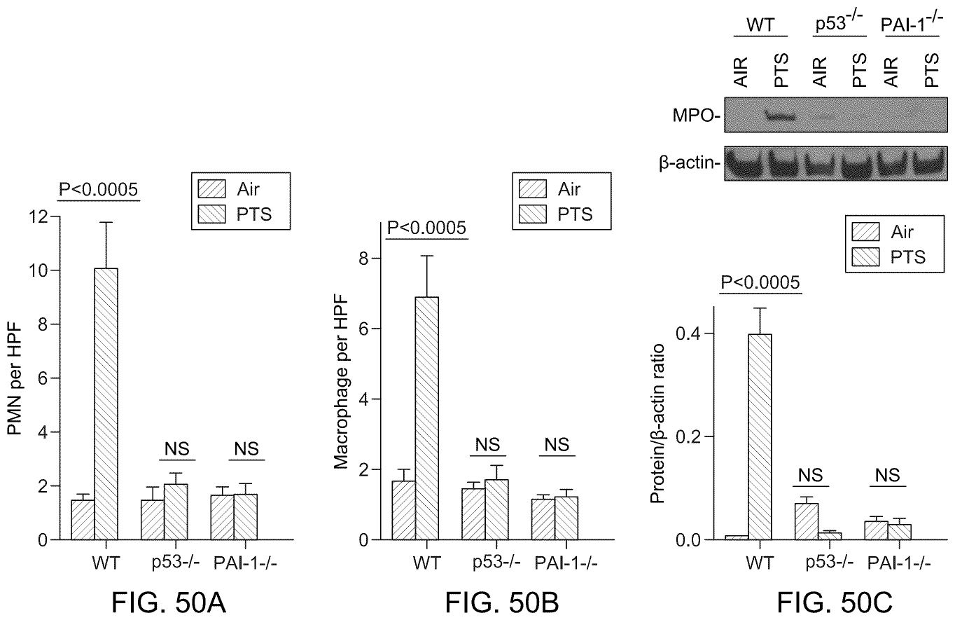

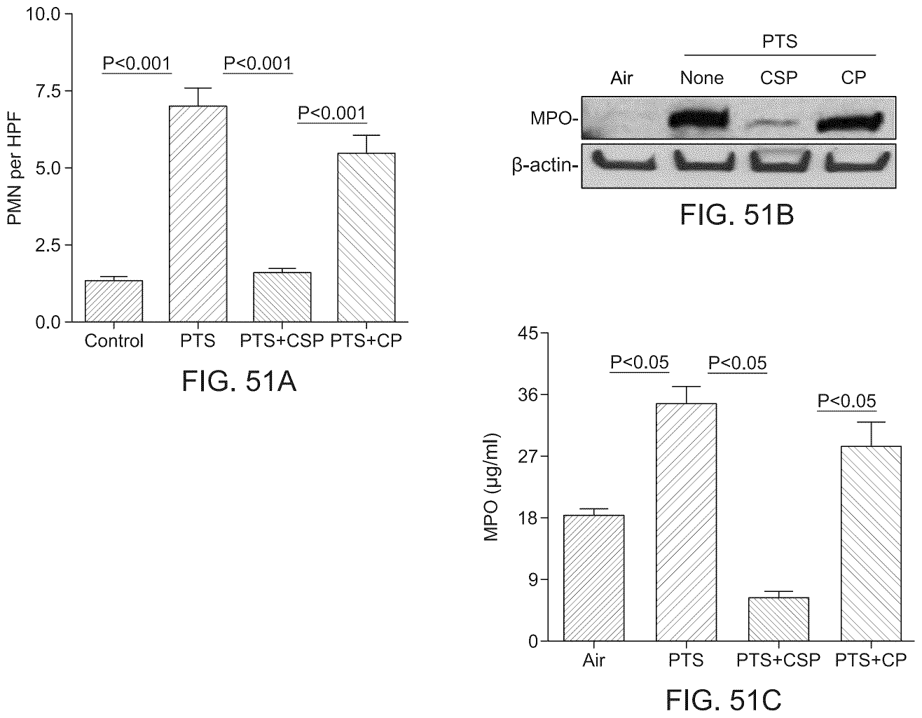

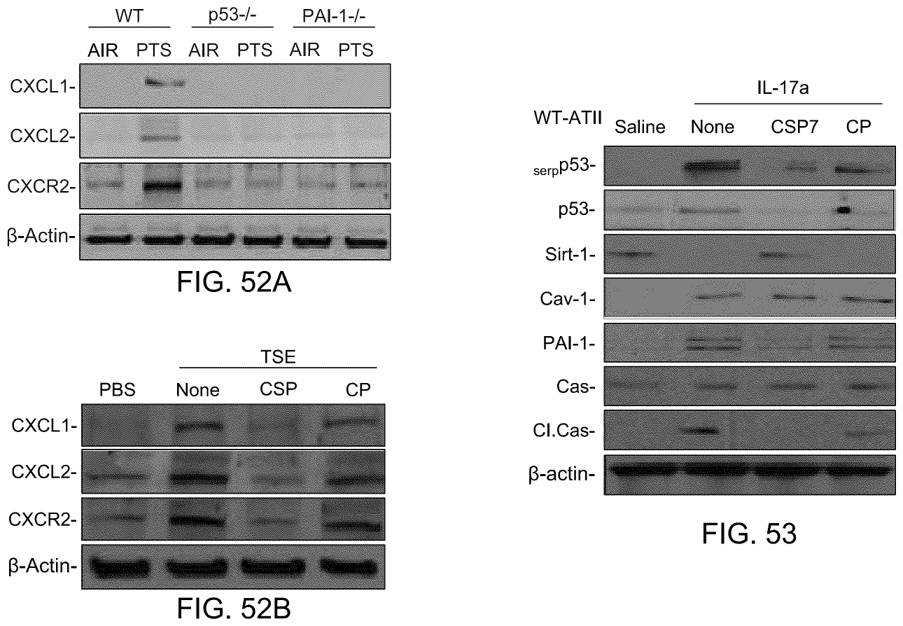

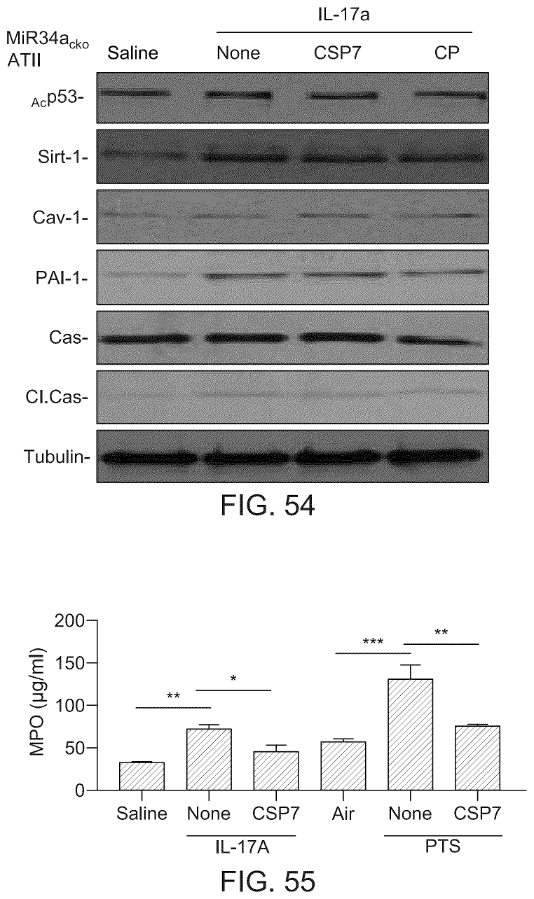

IL-17A contributes to PTS-induced lung injury by directly inducing p53 and PAI-1 and apoptosis in A₂ECs. Preliminary data reveal that IL-17A is elevated in the lungs of patients with COPD (FIG. 47). A recent report indicates that the majority (60%) of tryptase-positive mast cells produce IL-17A in severe COPD. IL-17A augments inflammation by promoting neutrophil/PMN influx and activation through induction of chemokines. PMN elastase contributes to tissue destruction and inflammation in severe COPD. IL-17A is increased in the lungs of mice exposed to 20 weeks of PTS (FIGS. 48A-48B). IL-17A induces p53 and Cav1, and activates caspase-3, indicating apoptosis of A2ECs in vitro (FIG. 48C) and in vivo (FIG. 48D), while mice lacking IL-17A resists PTS-induced p53 or PAI-1, and apoptosis in A₂EC (FIG. 49A). In primary wild type (WT) A₂ECs, IL-17A induces p53, PAI-1 expression, and apoptosis, while p53^{-/-} A₂ECs failed to respond to exogenous IL-17A (FIG. 48C), suggesting that IL-17A-mediated induction of p53 by A₂ECs, and downstream PAI-1 expression due to induction of Cav1 contribute to A₂EC apoptosis and lung injury.

Treatment of mice with PTS (FIG. 46A) or IL-17A (FIG. 48D) increased acetylated p53 and reduced Sirt1 in A₂ECs, while apoptosis was increased in PTS⁵ and IL-17A (C) mice, providing evidence that PTS acts by increasing IL-17A. CSP inhibits these effects of PTS (FIG. 45) and IL-17A (FIG. 49B), confirming that lung injury can be reduced.

These data suggest an intricate link between induction of IL-17A and TSE induced lung injury. CSP and CSP7 inhibit TSE-induced IL-17A, inhibit TSE- and IL-17A induced lung injury, and inhibit IL-17A induced A₂EC apoptosis (FIGS. 49B and 53).

### Example 23

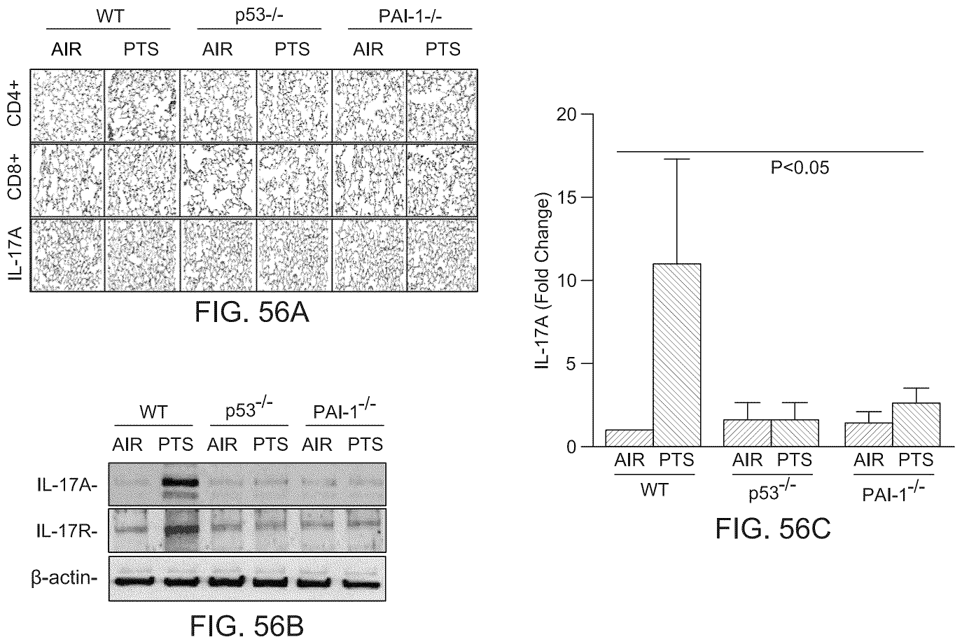

Immunohistochemical (IHC) analyses of lung sections, Western blotting of lung homogenates, and analyses of RNA by reverse transcription quantitative real-time PCR (qRT-PCR) revealed that IL-17A protein and IL-17A mRNA levels are markedly elevated in the lung tissues of patients with COPD. The wild type (WT) mice were therefore exposed to passive tobacco smoke (PTS or TSE) for 20 weeks and analyzed the lung sections, the lung homogenates and total RNA from the lungs for CD4, CD8 and IL-17A. WT mice exposed to 20 weeks of PTS showed increased staining for CD4, CD8 and IL-17A antigens indicating influx of T-lymphocytes. WT mice exposed to PTS also showed elevated levels of IL-17A protein and mRNA compared to those isolated from control mice in ambient air. IL-17 receptor (IL-17R) protein expression is also markedly increased in the lungs of WT mice with PTS lung injury. However, PTS exposure failed to induce IL-17A or IL-17R expression in either p53- or PAI-1-deficient mice (FIG. 56).

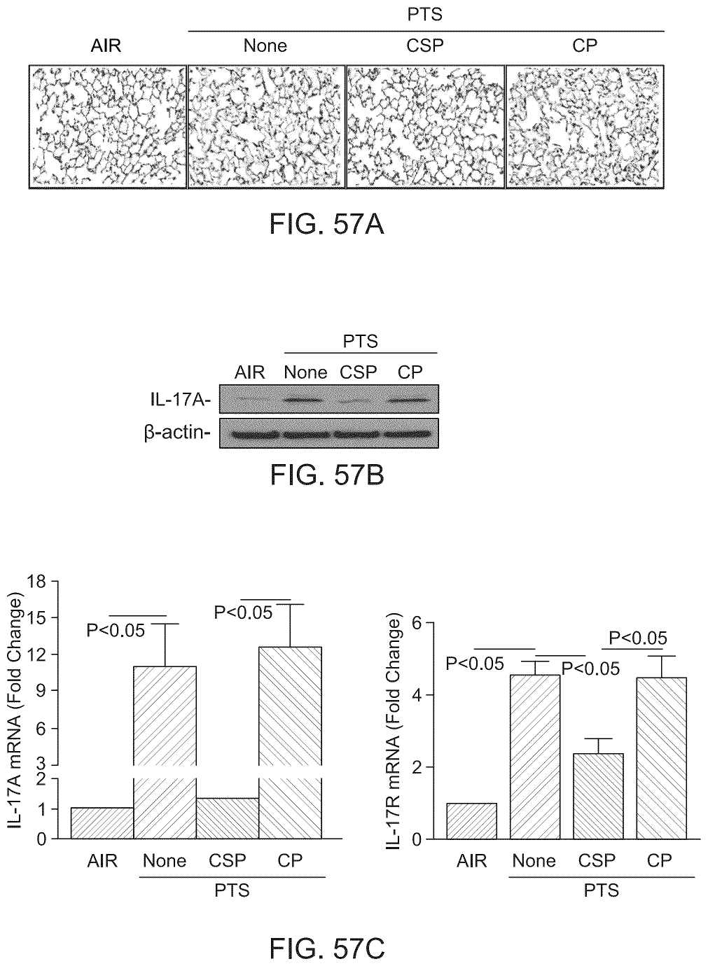

WT mice were next exposed to PTS for 20 weeks with or without caveolin-1 (Cav1) scaffolding domain peptide (CSP) harboring CSP7 (FTTFTVT) sequence. PTS exposed control mice were treated with scrambled control peptide (CP). CSP significantly inhibited PTS-induced IL-17A and IL-17R levels in WT mice (FIG. 57). 20 weeks of PTS exposure induced A₂EC apoptosis and pulmonary IL-17A in WT mice. Further, the process involves induction of p53 and p53-mediated downstream induction of PAI-1 in A₂ECs of WT mice. Primary A₂ECs were therefore treated with varying concentration of IL-17A. Consistent PTS-induced A₂EC injury, IL-17A treatment alone induced expression of both p53 and PAI-1, and also apoptosis in mouse and human A₂ECs. IL-17A concurrently induced acetylation of lysine 379 residues of p53 (p53^{AC}) and phosphorylation of serine 15 residues of p53 (p53^{S15}) indicating stabilization of p53 protein at the posttranslational level contributes to its increased steady expression. IL-17A induced p53 expression in A₂ECs was associated with concurrent increase in Cav1 and inhibition of Sirt1 expression. To confirm whether IL-17A-mediated induction of p53 contributes to increased PAI-1 expression and apoptosis in A₂ECs, A₂ECs lacking p53 expression were treated with varying concentration of IL-17A. Consistent with resistance to PTS induced lung injury by p53 deficiency, IL-17A failed to induce PAI-1 expression or apoptosis in A₂ECs lacking p53 expression. The WT mice were next exposed to recombinant IL-17A and control mice to vehicle, A₂ECs were isolated 24 h later. The A₂EC lysates were tested for induction of acetylated/total p53 and baseline Cav1 expression and apoptosis. Consistent with PTS induced lung injury in WT mice, recombinant IL-17A treatment caused marked increase in total and acetylated p53, apoptosis and induced Cav1 expression in A₂ECs, while IL-17A failed to induce PAI-1 expression or apoptosis in p53-deficient mice. These findings indicate that IL-17A mimics the effect of PTS exposure induced lung injury. Further these results reveal that PTS-induced IL-17A play a pivotal role in p53-mediated A₂EC apoptosis and lung injury.

WT mice were next exposed to PTS for 20 weeks with or without CSP. A₂ECs were isolated and A₂EC lysates were analyzed for changes in p53^{AC} and p53^{S15}. Both were increased in A₂ECs of mice exposed to 20 weeks of PTS. Immunoprecipitation also revealed increased interaction of acyltransferase p300 with p53^{S15}, which further increased p53^{AC}. These changes reduced binding and degradation of p53^{AC} and p53^{S15} by mdm2. This in turn increased p53 and p53-mediated downstream induction PAI-1 expression, and apoptosis in A₂ECs. PTS lung injury also induced Cav1 and reduced Sirt1 expression in A₂ECs suggesting inhibition of Sirt1-mediated deacetylation contributes to induction of p53^{AC}, PAI-1 and apoptosis. In addition, treatment of PTS exposed WT mice with CSP or CSP7 reduced p53^{AC}, p53^{S15} and total p53 levels, and restored baseline Sirt1 expression without inhibiting PTS-induced Cav1 in A₂ECs. However, mice exposed to PTS and treated with control peptide (CP) still showed elevated p53^{AC}, p53^{S15}, total p53 and PAI-1 in A₂ECs. PTS or IL-17A exposure augmented ATM kinase activity through increased phosphorylation of serine 1981 of ATM kinase. Further, CSP reduced p53^{AC}, p53^{S15} and total p53 without inhibiting PTS- or IL-17A-induced Cav1 expression demonstrating Cav1-mediated sequestration of catalytic unit of protein phosphatase 2A (PP2Ac) augments p53^{S15} by ATM kinase. A₂EC lysates were therefore immunoprecipitated using anti-PP2Ac antibody and immunoblotted for Cav1. Both PTS or IL-17A exposure augmented Cav1 expression, and Cav1 binding with PP2Ac, which in turn sequestered PP2Ac from the cytoplasm to the caveoli. This led to activation of ATM kinase and downstream ATM kinase-mediated serine phosphorylation and acetylation of p53 leading to induction of p53. This was completely reversed by treatment of PTS exposed mice with CSP, which inhibited Cav1 binding with PP2Ac.

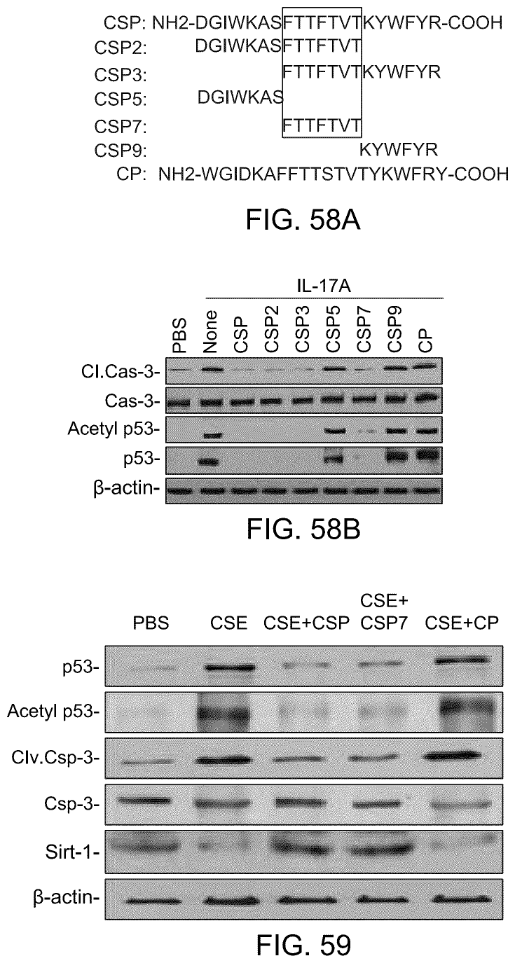

To identify the minimal active component of CSP, A₂ECs exposed to IL-17A were treated with full length CSP or its overlapping deletion fragments of CSP2, CSP3 and CSP7, which contained the amino acids, FTTFTVT, inhibited p53 and caspase-3 activation in A₂ECs treated with IL-17A, whereas CSP5 and CSP9, which did not include this fragment, had no effect. CSP7 inhibits lung injury due to chronic PTS or IL-17A exposure (FIG. 58).

To further confirm CSP7 mimics CSP effect, primary A₂ECs were treated with cigarette smoke extract (CSE) prepared in PBS in the presence of CSP or CSP7. The A2ECs treated with CSE and CP or PBS alone were used as controls. The A₂EC lysates were tested for acetylated and total p53, apoptosis and Sirt1 expression. CSE induced total and acetylated p53 while inhibiting Sirt1 expression in A₂ECs indicating inhibition of Sirt1-mediated deacetylation of p53 contributes to induction of acetylated and total p53 and A₂EC apoptosis. Treatment with either CSP or CSP7 inhibited CSE-induced apoptosis and the process involves Sirt1-mediated deacetylation and degradation of p53 A₂ECs (FIG. 59).

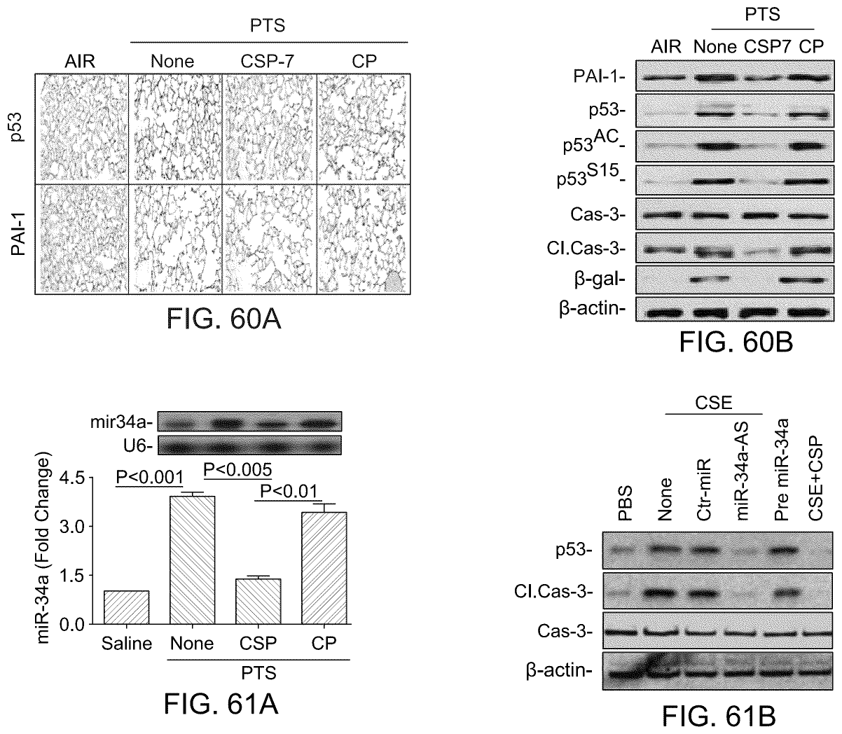

To confirm whether CSP7 mimics the effect of CSP in vivo, WT mice exposed to PTS were treated with or without CSP7. Lung sections and isolated A₂ECs were tested for PAI-1, p53^{AC}, p53^{S15}, apoptosis (cleaved Caspase-3) and senescence (beta-galactosidase). All were increased in mice exposed to 20 weeks of PTS. This was markedly reduced following treatment of WT mice exposed to PTS with CSP7 (FIG. 60). Further analysis of lung injury using quantitative micro-CT revealed that end-expiratory lung volumes of PTS exposed WT mice for 20 weeks were significantly (p<0.05) larger than those exposed to ambient AIR indicating development of emphysema-like condition. Treatment of PTS exposed mice with CSP or CSP7 significantly (p<0.05 or p<0.01) reduced lung volumes suggesting mitigation of lung injury.

Increased p53 and apoptosis in A₂ECs of mice exposed to PTS or IL-17A provided evidence that PTS acts by increasing IL-17A. To confirm that increased IL-17A contributes to the induction of p53 and PAI-1 expression, and apoptosis in A2ECs during PTS-induced lung injury, IL-17A-deficient mice were exposed to 20 weeks of PTS and control mice were kept in ambient air. A2ECs were tested for p53^{AC}, p53 and Sirt1 expression, and apoptosis. The responses were compared with changes in A₂ECs isolated from WT mice exposed to 20 weeks of PTS or AIR. There was marked induction of p53^{AC} and p53 levels, and apoptosis with parallel suppression of Sirt1 expression in WT mice exposed to 20 weeks of PTS. However, PTS failed to induce p53^{AC} or p53, PAI-1 and apoptosis or inhibit Sirt1 expression in A₂ECs indicating resistance by mice lacking IL-17A expression. To confirm mitigation of PTS induced lung injury by CSP or CSP7 involves suppression of IL-17A-induced p53, PAI-1 and apoptosis, A₂ECs isolated from WT mice were next treated with IL-17A (100 ng/ml) with or without CSP, CSP7 or CP. Control cells were treated with vehicle (PBS) alone. The A₂EC lysates were tested for p53 and p53-mediated downstream induction of PAI-1, and apoptosis. Both CSP and CSP7 inhibited IL-17A-induced p53 and PAI-1 expression, and apoptosis in A₂ECs. Similarly, treatment of WT mice exposed to IL-17A with either CSP or CSP7 reduced A₂EC p53, PAI-1 and apoptosis in vivo. miR-34a and p53 levels are increased in A₂ECs after PTS lung injury, which is significantly inhibited by CSP in WT mice (FIG. 61). Further, induction of miR-34a increased p53 and apoptosis in A2ECs without exposure to tobacco smoke, while inhibition of miR-34a using miR-34a antisense (miR-34a-AS) reduced cigarette smoke induced p53 expression and apoptosis. Results of both in vitro and in vivo experiments support an intricate link between miR-34a, p53^{AC}, p53 and p53-mediated induction of PAI-1 expression, and A₂EC apoptosis during PTS lung injury.

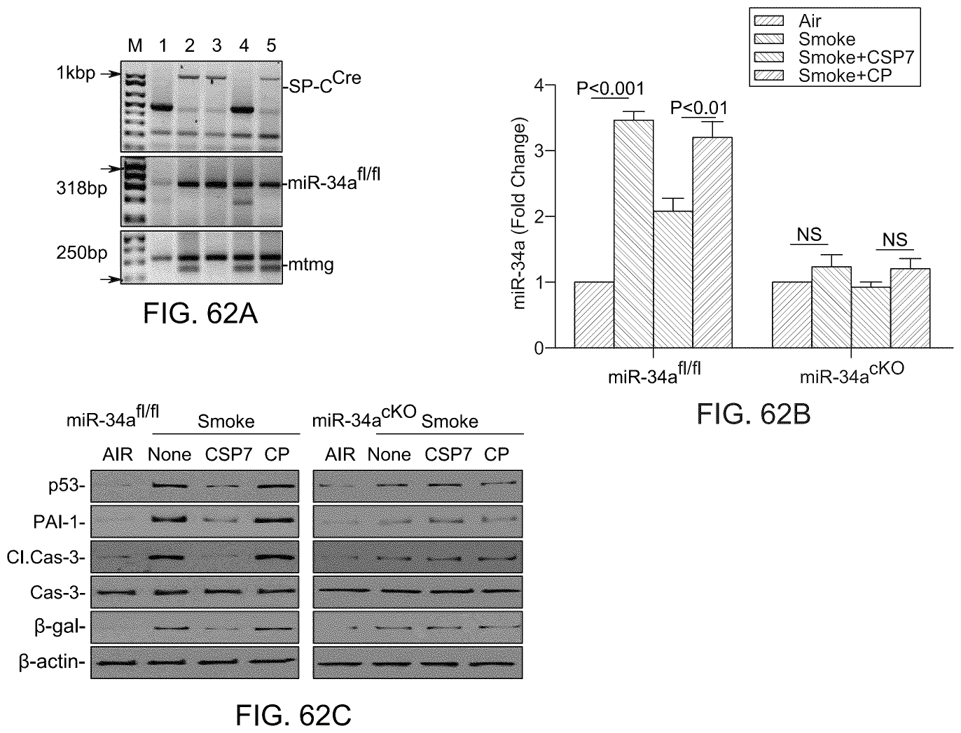

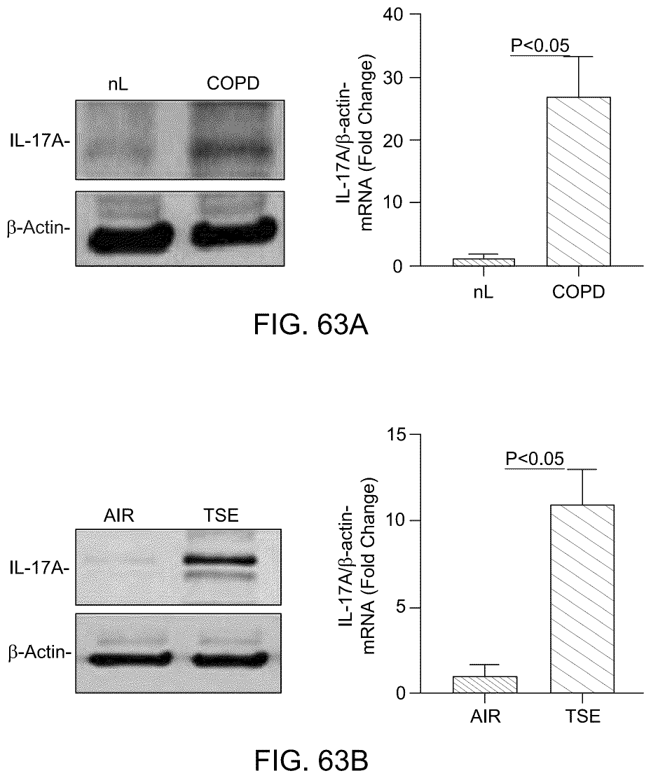

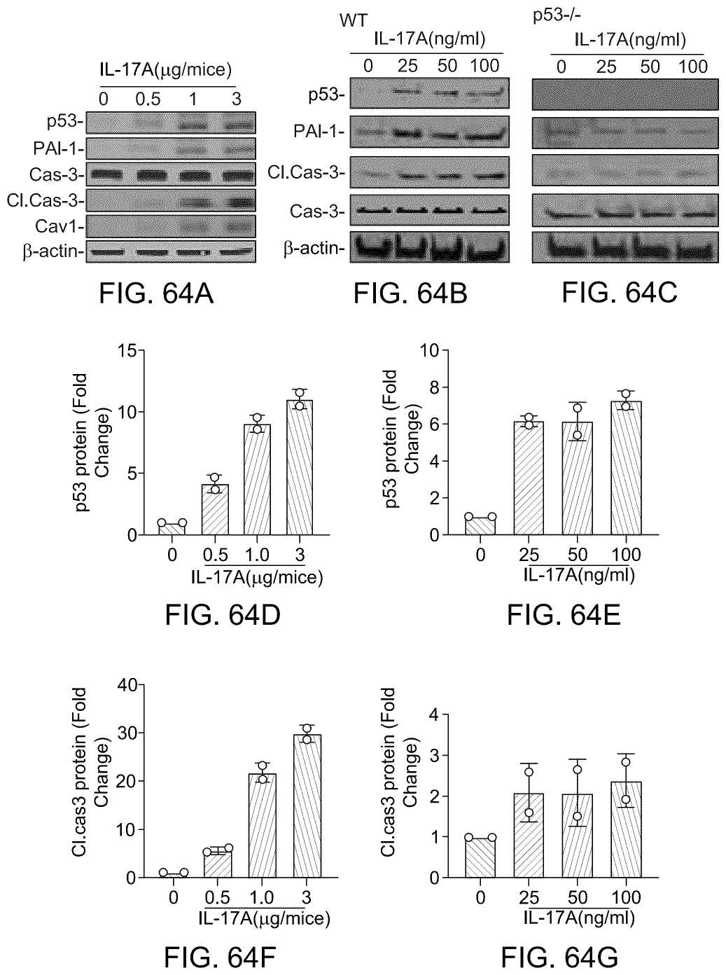

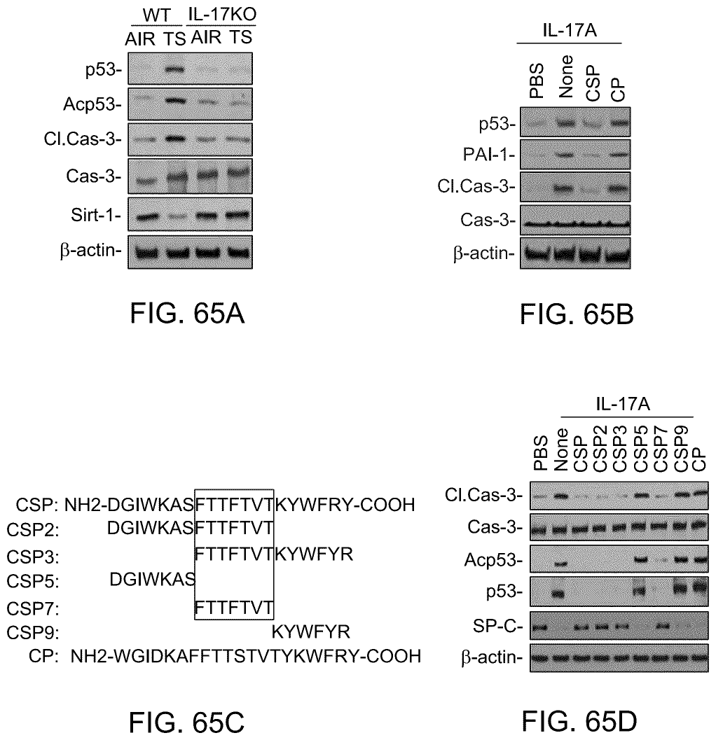

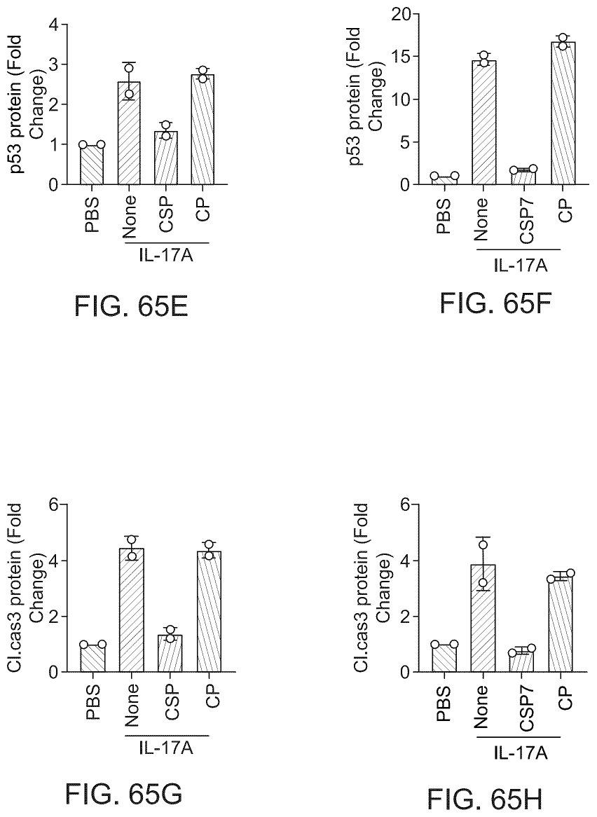

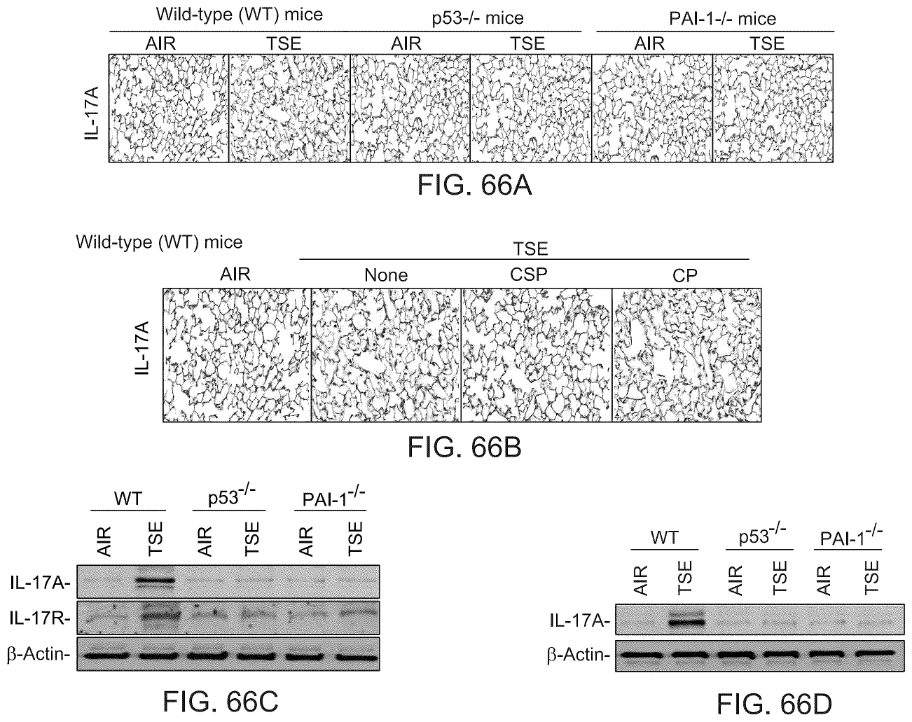

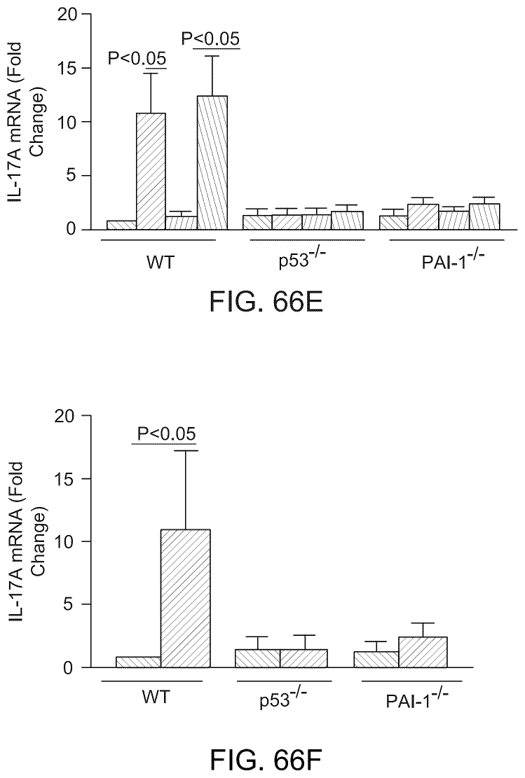

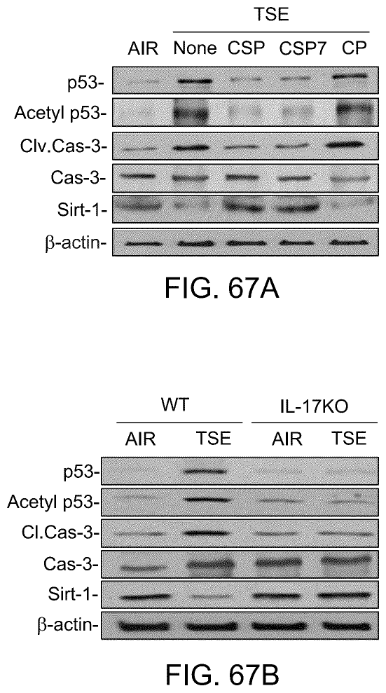

Inducible conditional knockout (miR-34a^{cKO}) mice lacking miR-34a expression in A₂ECs were generated by crossing miR-34a^{fl/fl} mice with SP-C^{Cre/mTmG} mice (FIG. 62A). RNA from A₂ECs of miR-34a^{fl/fl} or miR-34a^{cKO} mice treated with tamoxifen were analyzed for miR-34a. Results revealed 82% reduction (p<0.001) in baseline miR-34a in A₂ECs by miR-34a^{cKO} mice. miR-34a^{cKO} mice were next exposed to PTS for 20 weeks with or without IP injection of CSP7 or CP. After 20 weeks, A₂ECs were isolated from these mice and analyzed for changes in miR-34a. A₂ECs of WT or miR-34a^{fl/fl} exposed to PTS showed 3.5-fold induction of miR-34a expression compared to control mice in AIR. Further, treatment of PTS exposed WT or miR-34a^{fl/fl} mice with CSP7 reduced miR-34a expression. However, PTS failed to induce miR-34a expression in miR-34a^{cKo} mice and CSP7 has minimal effect in miR-34a^{cKo} mice exposed to 20 weeks of PTS (FIG. 62B). A₂ECs from miR-34a^{fl/fl} and miR-34a^{cKo} mice were next isolated and tested the lysates for changes in p53 and PAI-1 expression, and apoptosis. PTS induced p53 and PAI-1 expression in A₂ECs of miR-34a^{fl/fl} mice. These changes were associated increased of A₂EC apoptosis and senescence. Treatment of miR-34a^{fl/fl} mice with CSP7 inhibited p53, PAI-1 and A₂EC apoptosis and senescence. Consistent with lack of miR-34a induction, PTS failed to induce p53 or PAI-1 expression in A₂ECs of miR-34a^{cKO} mice. These mice also resisted A₂ECs apoptosis or senescence (FIG. 62C). These findings implicate miR-34a in PTS induced lung injury and further show that, through inhibition of miR-34a, CSP7 reverses PTS or IL-17A-induced p53 and downstream PAI-1 and apoptosis in A₂ECs.

Some of the data discussed above reveal the following conclusions. Chronic PTS exposure induces accumulation of CD4- and CD8-positive T cells, IL-17A, macrophages and neutrophils in the lungs of WT mice, which is resisted by both p53- and PAI-1-deficient mice. Treatment of PTS exposed mice with CSP or CSP7 inhibits pulmonary influx of CD4- and CD8-positive T cells, macrophages and neutrophils. Treatment of WT mice with CSP or CSP7 inhibits PTS-induced accumulation of IL-17A in the lungs. Mice exposed to 20 weeks of PTS show increased lung volume indicating emphysema-like condition, which was significantly reduced following treatment of PTS exposed WT mice with either CSP or CSP7. IL-17A treatment induces expression of p53 and PAI-1, and apoptosis in A2ECs both in vitro and in vivo. Further, the process involves acetylation and serine phosphorylation of p53 proteins in A₂ECs. CSP7 inhibits PTS or IL-17A exposure induced p53 and p53-mediated downstream induction of PAI-1 expression, and apoptosis in A₂ECs both in vivo and in vitro. The process involves inhibition of p53 acetylation through suppression of miR-34a expression and restoration of Sirt1 expression in A₂ECs. IL-17A-deficient mice exposed to PTS resist induction of p53 or downstream PAI-1 expression or apoptosis in A₂ECs. PTS exposure of IL-17A-deficient mice failed to induce expression of miR-34a or acetylated and total p53 in A₂ECs. IL-17A-deficient mice also resist PTS exposure induced suppression of baseline Sirt1 expression. Loss of miR-34a expression in A₂ECs prevents PTS induced acetylation of p53 and PAI-1 expression, apoptosis or senescence. Overexpression of miR-34a in A₂ECs alone induced p53 expression and apoptosis. A₂EC-specific inhibition of miR-34a expression prevents PTS-induced suppression of Sirt1 expression.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A method for blocking, reducing or attenuating:
(i) telomere dysfunction;
(ii) senescence and apoptosis in alveolar type II epithelial cells (A₂Cs);
(iii) mucus hypersecretion mediated by overexpression of M5Ac or by IL-17A in airway epithelial cells (AECs); or
(iv) disassembly, shortening or ciliopathy of airway cilia;
comprising administering by DP inhaler to A₂Cs or AECs in a subject a composition comprising:
(a) 0.2 mg to 10 mg/day of a peptide having the amino acid sequence FTTFTVT (SEQ ID NO:1); or
(b) 0.2 mg to 10 mg/day of an addition variant of (a) that includes 1-5 amino acids of additional sequence at the N- and/or C-terminus.

2. The method of claim 1, further comprising reducing lung inflammation in said subject.

3. The method of claim 1, wherein the peptide is FTTFTVT (SEQ ID NO:1).

4. The method of claim 1, wherein the subject is a human.

5. The method of claim 1, wherein telomere dysfunction is measured by measuring telomere shortening by TeloTAGGG assay or qPCR.

6. The method of claim 5, wherein there is at least a 3% increase in telomere length in A₂Cs after 6 months of administration.

7. The method of claim 1, wherein reducing senescence in A₂Cs is determined by measuring beta-galactosidase in A₂Cs after 6 months of administration.

8. The method of claim 7, wherein there is at least a 5% reduction in beta-galactosidase in A₂Cs.

9. The method of claim 1, wherein reducing senescence in A₂Cs is determined by measuring beta-galactosidase activity by colorimetric assay, X-gal staining, flow cytometry or beta-galactosidase staining using an antibody in A₂Cs.

10. The method of claim 9, wherein there is at least a 3% reduction in activated caspase-3 expression after 6 months of administration.

11. The method of claim 1, wherein apoptosis in A₂Cs is determined by measuring activated caspase-3 expression.

12. The method of claim 11, wherein there is a 3% reduction in activated caspase-3 expression after 6 months of administration.

13. The method of claim 1, wherein mucus hypersecretion mediated by overexpression of Muc5Ac in AECs is measured by periodic acid Schiff (PAS) staining for mucin, immunohistochemistry using an antibody for Muc5Ac or mucin, Western blotting, or real-time PCR.

14. The method of claim 13, wherein there is at least a 20% reduction in Muc5Ac expression after 6 months of administration.

15. The method of claim 1, wherein mucus hypersecretion is mediated by overexpression of IL-17A in AECs and is measured by periodic acid Schiff (PAS) staining for mucin, immunohistochemistry using an antibody for Muc5Ac or mucin, Western blotting, or real-time PCR.

16. The method of claim 15, wherein there is at least a 10% reduction in IL-17A expression after 6 months of administration.

17. The method of claim 1, wherein lung and airway inflammation is analyzed by immunohistochemistry using an antibody against myeloperoxidase (MPO) or measuring MPO activity by colorimetric assay, using an antibody against airway and alveolar polymorphonucleocytes (PMN), using an antibody against airway and alveolar inflammatory macrophages, or an antibody against airway and alveolar CD4+ and CD8+cells.

18. The method of claim 1, wherein apoptosis and senescence are mediated by overexpression of IL-17A in A2Cs.

19. The method of claim 18, wherein there is at least a 40% reduction in IL-17A expression after 6 months of administration.

20. The method of claim 19, wherein the IL-17A expression is measured by ELISA, Western blotting, or real-time PCR.

21. The method of claim 1, wherein miR-34a expression is inhibited.

22. The method of claim 21, wherein there is at least a 15% reduction in miR-34a expression after 6 months of administration.

23. The method of claim 22, wherein the miR-34a expression is measured by real-time PCR.

24. The method of claim 1, wherein disassembly of airway cilia is measured by measuring number of ciliated cells.

25. The method of claim 24, wherein there is at least a 20% increase in the number of ciliated cells after 6 months of administration.

26. The method of claim 1, wherein shortening of airway cilia is measured by microscope.

27. The method of claim 26, wherein there is at least a 20% increase in cilia length after 6 months of administration.

28. The method of claim 1, wherein ciliopathy of airway cilia is measured by measuring number of Ac-tubulin positive cells.

29. The method of claim 28, wherein there is at least a 20% increase in Ac-tubulin positive cells after 6 months of administration.

30. The method of claim 1, comprising administering 2.5 mg/day, 5 mg/day, or 10 mg/day of said peptide or addition variant thereof.


SEQUENCE LISTING (25 sequences)

SEQ ID NO: 1 - protein, 7 aa, Homo sapiens
FTTFTVT

SEQ ID NO: 2 - protein, 20 aa, Homo sapiens
DGIWKASFTTFTVTKYWFYR

SEQ ID NO: 3 - protein, 178 aa, Homo sapiens
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

SEQ ID NO: 4 - protein, 17 aa, Homo sapiens
NYHYLESSMTALYTLGH

SEQ ID NO: 5 - protein, 20 aa, synthetic construct
WGIDKAFFTTSTVTYKWFRY

SEQ ID NO: 6 - protein, 16 aa, Homo sapiens
RQIKIWFQNRRMKWKK

SEQ ID NO: 7 - protein, 16 aa, synthetic construct
RQIKIFFQNRRMKWKK

SEQ ID NO: 8 - protein, 16 aa, synthetic construct
RQIKIWFQNRRMKFKK

SEQ ID NO: 9 - protein, 16 aa, synthetic construct
RQIKIFFQNRRMKFKK

SEQ ID NO: 10 - protein, 27 aa, Homo sapiens
GWTLNSAGYLLGKINLKALAALAKKIL

SEQ ID NO: 11 - protein, 21 aa, Homo sapiens
KETWWETWWTEWSQPKKKRKV

SEQ ID NO: 12 - protein, 12 aa, Homo sapiens
KETWWETWWTEW

SEQ ID NO: 13 - protein, 6 aa, Homo sapiens
KKKRKV

SEQ ID NO: 14 - DNA, 22 bp, synthetic construct
  source 1..22: mol_type = other DNA
tggcagtgtcttagctggttgt

SEQ ID NO: 15 - DNA, 20 bp, synthetic construct
  source 1..20: mol_type = other DNA
gtgcagcacttctagggcag

SEQ ID NO: 16 - DNA, 20 bp, synthetic construct
  source 1..20: mol_type = other DNA
cgcttcggcagcacatatac

SEQ ID NO: 17 - DNA, 20 bp, synthetic construct
  source 1..20: mol_type = other DNA
ttcacgaatttgcgtgtcat

SEQ ID NO: 18 - DNA, 37 bp, synthetic construct
  source 1..37: mol_type = other DNA
ggtttttgagggtgagggtgagggtgagggtgagggt

SEQ ID NO: 19 - DNA, 39 bp, synthetic construct
  source 1..39: mol_type = other DNA
tcccgactatccctatccctatccctatccctatcccta

SEQ ID NO: 20 - DNA, 23 bp, synthetic construct
  source 1..23: mol_type = other DNA
cagcaagtgggaaggtgtaatcc

SEQ ID NO: 21 - DNA, 25 bp, synthetic construct
  source 1..25: mol_type = other DNA
cccattctatcatcaacgggtacaa

SEQ ID NO: 22 - DNA, 39 bp, synthetic construct
  source 1..39: mol_type = other DNA
cggtttgtttgggtttgggtttgggtttgggtttgggtt

SEQ ID NO: 23 - DNA, 39 bp, synthetic construct
  source 1..39: mol_type = other DNA
ggcttgccttacccttacccttacccttacccttaccct

SEQ ID NO: 24 - DNA, 21 bp, synthetic construct
  source 1..21: mol_type = other DNA
actggtctaggacccgagaag

SEQ ID NO: 25 - DNA, 21 bp, synthetic construct
  source 1..21: mol_type = other DNA
tcaatggtgcctctggagatt